SEQUENCE LISTING (4 sequences)

SEQ ID NO: 1 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 2 - protein, 641 aa, Mus musculus
MAKNTAIGIDLGTTYSCVGVFQHGKVEIIANDQGNRTTPSYVAFTDTERLIGDAAKNQVALNPQNTVFDAKRLIGRKFGDAVVQSDMKHWPFQVVNDGDKPKVQVNYKGESRSFFPEEISSMVLTKMKEIAEAYLGHPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVSHFVEEFKRKHKKDISQNKRAVRRLRTACERAKRTLSSSTQASLEIDSLFEGIDFYTSITRARFEELCSDLFRGTLEPVEKALRDAKMDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQTFTTYSDNQPGVLIQVYEGERAMTRDNNLLGRFELSGIPPAPRGVPQIEVTFDIDANGILNVTATDKSTGKANKITITNDKGRLSKEEIERMVQEAERYKAEDEVQRDRVAAKNALESYAFNMKSAVEDEGLKGKLSEADKKKVLDKCQEVISWLDSNTLADKEEFVHKREELERVCSPIISGLYQGAGAPGAGGFGAQAPKGASGSGPTIEEVD

SEQ ID NO: 3 - protein, 175 aa, Homo sapiens
MTPLGPASSLPQSFLLKCLEQVRKIQGDGAALQEKLCATYKLCHPEELVLLGHSLGIPWAPLSSCPSQALQLAGCLSQLHSGLFLYQGLLQALEGISPELGPTLDTLQLDVADFATTIWQQMEELGMAPALQPTQGAMPAFASAFQRRAGGVLVASHLQSFLEVSYRVLRHLAQP

SEQ ID NO: 4 - protein, 208 aa, Mus musculus
MAQLSAQRRMKLMALQLLLWQSALWSGREAVPLVTVSALPPSLPLPRSFLLKSLEQVRKIQASGSVLLEQLCATYKLCHPEELVLLGHSLGIPKASLSGCSSQALQQTQCLSQLHSGLCLYQGLLQALSGISPALAPTLDLLQLDVANFATTIWQQMENLGVAPTVQPTQSAMPAFTSAFQRRAGGVLAISYLQGFLETARLALHHLA

(19) United States
(12) Patent Application Publication
(10) Pub. No.: US 2024/0287457 A1
(43) Pub. Date: Aug. 29, 2024

(54) COMPOSITIONS AND CELL CULTURE MEDIA FOR INCREASING NEUTROPHIL LIFESPAN

(71) Applicants: THE CHILDREN'S MEDICAL CENTER CORPORATION, Boston, MA (US); INSTITUTE OF HEMATOLOGY AND BLOOD DISEASES HOSPITAL, CHINESE ACADEMY OF MEDICAL SCIENCES AND PEKING, Tianjin (CN)

(72) Inventors: Hongbo R. LUO, Boston, MA (US); Yuping FAN, Tianjin (CN); Fengxia MA, Tianjin (CN); Yan TENG, Boston, MA (US)

(73) Assignees: THE CHILDREN'S MEDICAL CENTER CORPORATION, Boston, MA (US); INSTITUTE OF HEMATOLOGY AND BLOOD DISEASES HOSPITAL, CHINESE ACADEMY OF MEDICAL SCIENCES AND PEKING, Tianjin (CN)

(21) Appl. No.: 18/569,895

(22) PCT Filed: Jun. 14, 2022
(86) PCT No.: PCT/US2022/033358

§ 371 Date: Dec. 13, 2023

Related U.S. Application Data
(60) Provisional application No. 63/210,375, filed on Jun. 14, 2021.

(51) Int. Cl. C12N 5/0787; A61K 35/15; A61P 37/02

(52) U.S. Cl. CPC C12N 5/0642; A61K 35/15; A61P 37/02; C12N 2500/33; C12N 2501/22; C12N 2501/998; C12N 2501/999

(57) ABSTRACT

The technology described herein is directed to compositions and cell culture media for increasing the lifespan of neutrophils. Also described are methods and kits for culturing neutrophils using the compositions or cell culture media. Such cultured neutrophils can be used to treat disorders such as neutropenia or microbial infections.

### CROSS-REFERENCE TO RELATED APPLICATIONS

This application claims benefit under 35 U.S.C. § 119(e) of U.S. Provisional Application No. 63/210,375 filed Jun. 14, 2021, the contents of which are incorporated herein by reference in their entirety.

### GOVERNMENT SUPPORT

This invention was made with government support under Grant No. HL095489 awarded by the National Institutes of Health. The government has certain rights in the invention.

### SEQUENCE LISTING

The instant application contains a Sequence Listing which has been submitted in ASCII format via EFS-Web and is hereby incorporated by reference in its entirety. Said ASCII copy, created on Jun. 14, 2022, is named 701039-190350WOPT_SL.txt and is 14,748 bytes in size.

### TECHNICAL FIELD

The technology described herein relates to compositions and cell culture media for increasing neutrophil lifespan.

### BACKGROUND

Neutropenia and associated bacterial and fungal infections are dose-limiting consequences for patients receiving chemotherapy, radiotherapy, or hematopoietic cell transplantation (HCT). Without prompt medical intervention, infections can become life-threatening, and not all patients respond to antibiotic therapy. An alternative preventative approach is granulocyte colony-stimulating factor (G-CSF) treatment. However, this therapy often does not work before the bone marrow has recovered and is associated with side-effects such as bone pain, headache, fatigue, and nausea. Long term use of G-CSF also potentially increases the risk of leukemia. Granulocyte transfusion (GTX), also known as neutrophil transfusion, is a therapeutic option for life-threatening bacterial and fungal infections in severely neutropenic patients. Large doses of neutrophils can be easily obtained from healthy donors who have been stimulated with G-CSF. Nevertheless, the clinical benefit of GTX is still compromised by short ex vivo shelf lives and rapid in vivo death of granulocytes. GTX is of value in appropriate clinical settings, such as when the absolute neutrophil count is <500 cells/μL, when there is evidence of bacterial or fungal infection, or in the case of unresponsiveness to antimicrobial treatment, generally for at least 48 hours. The general consensus is that further research and development into granulocyte biology directed at improving neutrophil survival and function in GTX are urgently required. See e.g., White et al., Hematol Oncol Clin North Am 31, 981-993 (2017); Wingard et al., Curr Opin Hematol 19, 21-26 (2012); Bennett et al., N Engl J Med 368, 1131-1139 (2013); Anderlini et al., Blood 88, 2819-2825 (1996); Dale et al., Curr Opin Hematol 16, 1-2 (2009); Netelenbos et al., Transfusion 59, 160-168 (2019); Price et al., Blood 126, 2153-2161 (2015); the contents of each of which are incorporated herein by reference in their entireties.

Neutrophil death is a heterogeneous process mediated by both apoptotic and lytic death pathways. Reactive oxygen species (ROS) accumulate in aging neutrophils and ROS-induced phosphatidylinositol 3,4,5 triphosphate (PtdIns(3,4,5)P3) signaling deactivation mediates neutrophil death. These ROS have also been shown to be responsible for membrane damage and initiation of cellular demise pathways. Caspase-3 cleavage and activation are independent of canonical caspase-8 or caspase-9-mediated pathways in aging neutrophils; caspase-3 cleavage and activation are instead mediated by cytosolic serine protease PR3, which is sequestered in granules in fresh neutrophils and released to the cytosol during aging by ROS-induced lysosomal membrane permeabilization (LMP), leading to pro-caspase-3 cleavage and apoptosis. Lytic neutrophil death can be mediated by gasdermin D (GSDMD), which is cleaved and activated to produce an N-terminal fragment (GSDMD-NT) to trigger membrane rupture and subsequent pyroptosis. Necroptosis is another programmed form of necrosis, but its role in neutrophil biology is less understood. Human neutrophils were recently reported to undergo necroptosis after exposure to granulocyte-macrophage colony-stimulating factor (GM-CSF) followed by ligation of adhesion receptors such as CD44, CD11b, CD18, or CD15. Thus, there is great need to improve the efficacy of neutrophil transfusion by leveraging these multiple neutrophil death mechanisms and prolonging neutrophil survival without compromising their function. See e.g., Teng et al., Am J Hematol 92, E156-E159 (2017); Zhu et al., Proc Natl Acad Sci USA 103, 14836-14841 (2006); Xu et al., Proc Natl Acad Sci USA 107, 2950-2955 (2010); Loison et al., J Clin Invest 124, 4445-4458 (2014); Kambara et al., Cell reports 22, 2924-2936 (2018); Wang et al., J Immunol. 197, 4090-4100 (2016); the contents of each of which are incorporated herein by reference in their entireties.

### SUMMARY

Specifically described herein are compositions and methods that improve the efficacy of neutrophil transfusion by leveraging multiple neutrophil death mechanisms and prolonging neutrophil survival without compromising their function. Pharmacological screening revealed a combined treatment that delayed neutrophil death by simultaneously targeting multiple death pathways. CLON-G (caspases-LMP-oxidant-necroptosis inhibition plus G-CSF) treatment altered neutrophil fate, increasing the ex vivo shelf life of collected neutrophils without affecting their in vivo function, improving GTX efficacy in clinically relevant murine models of granulocyte transfusion in neutropenic hosts. These results provide a clinical strategy for the storage and application of neutrophils in transfusion medicine, demonstrating CLON-G treatment as a therapeutic approach for improving GTX efficacy.

Accordingly, in one aspect described herein is a composition comprising: (a) a caspase inhibitor; (b) a lysosomal membrane permeabilization (LMP) inhibitor; (c) an antioxidant; and (d) a growth factor.

In some embodiments of any of the aspects, the caspase inhibitor is a pan-caspase inhibitor.

In some embodiments of any of the aspects, the pan-caspase inhibitor is selected from the group consisting of: Q-VD-Oph; Z-VAD-FMK; Emricasan; and Ac-DEVD-CHO.

In some embodiments of any of the aspects, the pan-caspase inhibitor is Q-VD-Oph.

In some embodiments of any of the aspects, Q-VD-Oph is at a concentration of at least 50 μM.

In some embodiments of any of the aspects, the pan-caspase inhibitor is Emricasan.

In some embodiments of any of the aspects, Emricasan is at a concentration of at least 10 μM.

In some embodiments of any of the aspects, Emricasan is at a concentration of at least 25 μM.

In some embodiments of any of the aspects, the LMP inhibitor is Heat Shock Protein 70 (Hsp70) and/or deferoxamine mesylate (DFO).

In some embodiments of any of the aspects, Hsp70 is at a concentration of at least 10 pM.

In some embodiments of any of the aspects, DFO is at a concentration of at least 1 μM.

In some embodiments of any of the aspects, the antioxidant is N-acetyl cysteine (NAC).

In some embodiments of any of the aspects, NAC is at a concentration of at least 10 μM.

In some embodiments of any of the aspects, the growth factor is a stimulator of the phosphoinositide 3-kinase (PI3K) and Akt pathway.

In some embodiments of any of the aspects, the stimulator of the PI3K/Akt pathway is granulocyte colony-stimulating factor (G-CSF).

In some embodiments of any of the aspects, G-CSF is at a concentration of at least 10 ng/mL.

In some embodiments of any of the aspects, the composition further comprises a necroptosis inhibitor.

In some embodiments of any of the aspects, the necroptosis inhibitor is Nec-1s.

In some embodiments of any of the aspects, Nec-1s is at a concentration of at least 10 μM.

In some embodiments of any of the aspects, the composition further comprises cell culture medium and/or serum.

In some embodiments of any of the aspects, the cell culture medium comprises RPMI.

In some embodiments of any of the aspects, the serum is fetal bovine serum (FBS).

In one aspect described herein is a cell culture medium comprising: (a) a caspase inhibitor; (b) a lysosomal membrane permeabilization (LMP) inhibitor; (c) an antioxidant; (d) a growth factor; and (e) a necroptosis inhibitor.

In some embodiments of any of the aspects, the cell culture medium comprises RPMI.

In some embodiments of any of the aspects, the serum is fetal bovine serum (FBS).

In some embodiments of any of the aspects, the FBS is at a concentration of at least 20%.

In one aspect described herein is a cell culture medium for increasing the lifespan of human neutrophils, the cell culture medium comprising: (a) at least about 50 μM of a pan-caspase inhibitor; (b) at least about 1 μM of a first LMP inhibitor; (c) at least about 10 pm of a second LMP inhibitor; (d) at least about 10 μM of an antioxidant; (e) at least about 10 ng/mL a growth factor; and (f) at least about 10 μM of a necroptosis inhibitor.

In one aspect described herein is a cell culture medium for increasing the lifespan of neutrophils, the cell culture medium comprising effective concentrations of: Q-VD-Oph; DFO; Hsp70; NAC; G-CSF; and Nec-1s.

In one aspect described herein is a cell culture medium for increasing the lifespan of neutrophils, the cell culture medium comprising effective concentrations of: Q-VD-Oph; DFO; Hsp70; NAC; and G-CSF.

In one aspect described herein is a cell culture medium for increasing the lifespan of neutrophils, the cell culture medium comprising effective concentrations of: Emricasan; DFO; Hsp70; NAC; G-CSF; and Nec-1s.

In one aspect described herein is a cell culture medium for increasing the lifespan of neutrophils, the cell culture medium comprising effective concentrations of: Emricasan; DFO; Hsp70; NAC; and G-CSF.

In one aspect described herein is a cell culture medium for increasing the lifespan of human neutrophils, the cell culture medium comprising: (a) at least about 50 μM of Q-VD-Oph; (b) at least about 1 μM of DFO; (c) at least about 10 pm of Hsp70; (d) at least about 10 μM of NAC; (e) at least about 10 ng/mL of G-CSF; and (f) at least about 10 μM of Nec-1s.

In one aspect described herein is a cell culture medium for increasing the lifespan of human neutrophils, the cell culture medium comprising: (a) about 50 μM of Q-VD-Oph; (b) about 1 μM of DFO; (c) about 10 pm of Hsp70; (d) about 10 μM of NAC; (e) about 10 ng/mL of G-CSF; and (f) about 10 μM of Nec-1s.

In some embodiments of any of the aspects, the composition as described herein or the cell culture medium as described herein is in combination with a neutrophil.

In some embodiments of any of the aspects, the neutrophil is a human neutrophil.

In one aspect described herein is a kit comprising a composition as described herein or a cell culture medium as described herein.

In one aspect described herein is a method of increasing the lifespan of a neutrophil, the method comprising: contacting the neutrophil, or population thereof, with a composition as described herein or a cell culture medium as described herein.

In some embodiments of any of the aspects, the neutrophil, or population thereof, is a peripheral blood neutrophil (PMN).

In some embodiments of any of the aspects, the neutrophil, or population thereof, is isolated from the peripheral blood of a subject.

In some embodiments of any of the aspects, the neutrophil, or population thereof, is isolated from blood of a subject using apheresis.

In some embodiments of any of the aspects, the neutrophil, or population thereof, is isolated from the bone marrow of a subject.

In some embodiments of any of the aspects, the neutrophil, or population thereof, is contacted with the composition or the cell culture medium for a sufficient amount of time to increase neutrophil lifespan.

In some embodiments of any of the aspects, the sufficient amount of time is at least 3 days.

In some embodiments of any of the aspects, the sufficient amount of time is at least 1 hour.

In some embodiments of any of the aspects, the method further comprises removing the composition or the cell culture medium from the neutrophil, or population thereof, after the sufficient amount of time to increase neutrophil lifespan.

In some embodiments of any of the aspects, the half-life of the neutrophil, or population thereof, is increased to at least 5 days.

In some embodiments of any of the aspects, the neutrophil, or population thereof, is contacted with the composition or culture medium ex vivo.

In some embodiments of any of the aspects, contacting comprises culturing.

In one aspect described herein is a neutrophil produced by any one of the methods described herein

In one aspect described herein is a pharmaceutical composition comprising a neutrophil of as described herein, or population thereof, and a pharmaceutically acceptable carrier.

In some embodiments of any of the aspects, the pharmaceutical composition as described herein is for use in treating neutropenia in a subject.

In some embodiments of any of the aspects, the pharmaceutical composition as described herein is for use in treating a microbial infection in a subject.

In one aspect described herein is a method of treating neutropenia or a neutropenia-associated disease or disorder, the method: comprising administering an effective amount of a neutrophil as described herein, or population thereof, or a pharmaceutical composition as described herein to a recipient subject in need thereof.

In some embodiments of any of the aspects, the neutrophil is obtained from a human.

In some embodiments of any of the aspects, the neutrophil is obtained from the subject.

In one aspect described herein is a method of treating a microbial infection, the method comprising: administering an effective amount of a neutrophil as described herein, or population thereof, or a pharmaceutical composition as described herein to a recipient subject in need thereof.

In some embodiments of any of the aspects, the neutrophil is obtained from a human.

In some embodiments of any of the aspects, the neutrophil is obtained from the subject.

In one aspect described herein is a method of treating neutropenia or a neutropenia-associated disease or disorder, the method comprising: (a) isolating a neutrophil or population thereof from a donor subject; (b) contacting the neutrophil, or population thereof, with a composition as described herein or a cell culture medium as described herein; and (c) administering an effective amount of the cultured neutrophil, or population thereof, to a recipient subject in need thereof.

In some embodiments of any of the aspects, the donor subject is the recipient subject.

### BRIEF DESCRIPTION OF THE DRAWINGS

FIG. 1A-1H are a series of images and graphs showing that CLON-G, a treatment that simultaneously targets multiple death mechanisms, delayed neutrophil death and increased neutrophil half-life from less than 1 day to greater than 5 days. FIG. 1A is a series of images showing freshly isolated human peripheral blood neutrophils were cultured in RPMI-1640 plus 20% FBS with or without CLON-G. The morphologies of untreated (UT) or CLON-G treated (Treated) cells were observed after 1 day by light microscopy. White arrowheads indicate dying cells. FIG. 1B-1C show neutrophils that were stained with FITC-Annexin-V (A-V, which fluoresced green) and PI (which fluoresced red) after culturing for 1 day. Cell death was assessed by confocal fluorescence microscopy (FIG. 1B) and flow cytometry (FIG. 1C). FIG. 1D is a line graph showing the total number of healthy neutrophils calculated in UT and CLON-G-treated cell populations at the indicated time points as the number of remaining cells (intact cells in FIG. 11A) multiplied by the proportion of double-negative cells by flow cytometry (shown in FIG. 1C). FIG. 1E is a series of images showing the morphologies of untreated (“UT”) or CLON-G treated (“Treated”) murine neutrophils observed after 1 day by light microscopy. White arrowheads indicate dying cells. FIG. 1F-1G show murine neutrophils stained with FITC A-V and PI after culturing for 1 day. Cell death was assessed by confocal fluorescence microscopy (FIG. 1F) and flow cytometry (FIG. 1G). FIG. 1H is a line graph showing the total number of healthy neutrophils calculated in UT and CLON-G-treated cell populations at the indicated time points. All data are presented as mean±SD of three experiments. **indicates P<0.001 compared to the corresponding UT group.

FIG. 2A-2G are a series of schematics and graphs showing that CLON-G-treated mouse neutrophils displayed a similar or even longer lifespan than fresh neutrophils after drug removal. FIG. 2A is a schematic showing mouse neutrophils treated with CLON-G for 3 days in vitro followed by removing CLON-G by changing the cell culture medium to normal medium (CLON-G-treated, 3 days). At the same time, freshly isolated neutrophils were cultured in the normal medium without CLON-G (“Fresh neutrophils”). FIG. 2B is a bar graph showing the total numbers of intact neutrophils counted at the indicated time points. All data are presented as mean±SD of three experiments. * indicates P<0.01. FIG. 2C is a series of flow plots and a line graph showing cells that were stained with FITC A-V and PI and then analyzed by flow cytometry. The percentage of healthy neutrophils was assessed accordingly. All data are presented as mean±SD of three experiments. FIG. 2D is a schematic showing the experimental scheme for assessing the relative in vivo death rate of transplanted fresh, G-CSF alone-treated, and CLON-G-treated neutrophils in a mouse peritonitis model. TG, thioglycolate; i.p., intraperitoneal. FIG. 2E is a plot showing cells in peritoneal lavage fluid stained with PE-CD45.1 and analyzed by flow cytometry. FIG. 2F shows flow cytometry analysis of donor neutrophils in peritoneal lavage in mice transplanted with the indicated neutrophil populations at each time point. Shown are representative flow cytometry plots from one of three independent experiments. FIG. 2G is a bar graph showing the ratio of indicated transplanted neutrophil populations in peritoneal lavage fluid. All data are presented as mean±SD of three experiments. *indicates P<0.01 compared to 0 hours.

FIG. 3A-3E are a series of images and graphs showing that CLON-G treatment did not impair the migration and bacterial killing capability of mouse neutrophils. Freshly isolated mouse bone marrow neutrophils were cultured as described in FIG. 1 and FIG. 13. The function of UT, G-CSF-treated, and CLON-G-treated neutrophils was assessed at indicated time points. FIG. 3A is a series of images, migration paths, and bar graphs showing the chemotactic migration of neutrophils to fMLP (1 μM) was assessed using an EZ-TAXISCAN device. Migration velocity and directionality were calculated. FIG. 3B is series of images and bar graphs showing the in vitro phagocytosis capacity of neutrophils (APC-Ly6G, which fluoresced red) measured using zymosan (S. cerevisiae) bioparticles (which fluoresced green). Phagocytosis efficiency was expressed as the percentage of neutrophils that engulfed at least one bioparticle. Phagocytosis index was expressed as the average number of internalized particles per cell. At least 200 cells were assessed for each sample. FIG. 3C is a line graph showing fMLP-induced NADPH oxidase activation in mouse neutrophils assessed over time by luminol chemiluminescence. FIG. 3D is series of images showing in vitro killing of E. coli by mouse neutrophils. The bacterial killing capabilities were reflected by the decrease in cfus. 1, 2, 3, and 4 indicate serial dilution in the bacterial colony assay (1, 10× dilution; n, 10ⁿ×dilution). Control: bacterial suspension without any neutrophil cells; 0 h: fresh neutrophils. FIG. 3E is a bar graph showing the relative bacterial killing measured as the proportion of bacteria killed in 60 minutes. All data are presented as mean±SD of three experiments. *indicates P<0.05 and **indicates P<0.001 compared to freshly isolated neutrophil group (0 h); ^{#}indicates P<0.05 compared to UT (or G-CSF alone-treated) neutrophils cultured for 1 day; ^{##}indicates P<0.001 compared to UT (or G-CSF alone-treated) neutrophils cultured for 1 day.

FIG. 4A-4D are a series of schematics and graphs showing that CLON-G treatment did not impair neutrophil recruitment in vivo in a mouse neutropenia-related pneumonia model. The fresh, untreated, G-CSF alone-treated, and CLON-G-treated neutrophils were prepared as described in FIG. 2D. FIG. 4A is a schematic showing neutropenic recipient mice that were challenged with E. coli (5×10³ cfu) and subsequently received transfer of the indicated neutrophil populations. FIG. 4B is a series of plots showing the relative recruitment of transfused CFSE⁺ or SNARF-1⁺ fresh, untreated, G-CSF alone-treated, and CLON-G-treated neutrophils, as assessed by flow cytometry. FIG. 4C is a bar graph showing the total neutrophil number presented for each group as total cell number multiplied by the percentage of Ly6G⁺ cells calculated by flow cytometry. FIG. 4D is a bar graph showing the relative recruitment of fresh, untreated, G-CSF alone-treated, and CLON-G-treated neutrophils calculated as the relative ratio of CFSE⁺ to SNARF-1⁺ cells. The ratio of CFSE⁺ to SNARF-1⁺ cells in each sample was determined by flow cytometry and normalized to the sample collected from the mice transfused with CFSE⁺ fresh neutrophils and SNARF-1⁺ fresh neutrophils. Data are presented as mean±SD of three experiments. ns: no significant difference.

FIG. 5A-5H are a series of schematics, images, and graphs showing that transfusion with stored CLON-G-treated neutrophils enhanced host defenses and alleviated infection-induced lung damage as effectively as transfusion with untreated fresh neutrophils. The fresh, untreated, G-CSF alone-treated, and CLON-G-treated neutrophils were prepared as described in FIG. 2D. FIG. 5A is a schematic showing neutropenic mice that were challenged with E. coli (5×10³ cfu) and subsequently received transfer of the indicated neutrophil populations. FIG. 5B is a series of images and bar graph. BALF was collected and serially diluted (1, 10× dilution; n, 10ⁿ×dilution) with sterile cold water. Aliquots were spread on LB agar plates and incubated overnight at 37° C. Live bacteria were quantified as cfu/lung to determine bacterial killing. Data are presented as mean±SD of three experiments. ns, no significant difference; **, P<0.001. FIG. 5C is a series of flow cytometry plots and bar graphs. The total number of cells in the lungs was counted with a hemocytometer. Differential cell counts were determined by flow cytometry analysis. Cells were stained with F4/80 FITC and Ly6G APC. Total number of polymorphonuclear neutrophils (PMNs) recruited was calculated as follows: number of PMNs=cell density×volume×% PMN. Data are presented as mean (±SD). n≥4 mice in each group. ns, no significant difference; *, P<0.05. FIG. 5D is a series of images showing H&E staining of lung tissues, which showed pulmonary edema formation in infected lungs. FIG. 5E is a bar graph showing pulmonary edema formation quantified as the percentage of edema area in the total parenchymal region using IMAGEJ software. Data are presented as mean±SD of three experiments. ns, no significant difference; *, P<0.05. FIG. 5F is a bar graph showing protein accumulation in BALF measured using a protein assay kit. The standard curve was constructed using bovine serum albumin (BSA). Data are presented as mean±SD of three experiments. ns, no significant difference; *, P<0.05. FIG. 5G is a series of bar graphs showing BALF chemokine and cytokine concentrations determined by ELISA. Data are presented as mean±SD of three experiments. ns, no significant difference; *, P<0.05. FIG. 5H is a series of plots showing the rate of mortality due to E. coli-induced pneumonia in mice transfused with indicated neutrophil population, as shown by a Kaplan-Meier plot. Log-rank tests were used to analyze survival rates. *indicates P<0.01 as compared to PBS+2% FBS control treatment.

FIG. 6A-6K are a series of schematics, images, and graphs showing that CLON-G treatment delayed spontaneous death of human neutrophils isolated from granulocyte apheresis concentrates without impairing their migration and bacterial killing capability. FIG. 6A is a schematic showing granulocyte apheresis concentrates collected and processed following a standard clinical GTX protocol. FIG. 6B-6C are a series of images and plots showing neutrophil death assessed by confocal fluorescence microscopy (FIG. 6B) and flow cytometry (FIG. 6C). Neutrophils isolated from granulocyte apheresis concentrates were cultured in the presence of indicated compounds for indicated time period, and then stained with FITC A-V and PI. Representative flow cytometry plots from one of three independent experiments are shown in FIG. 6C. FIG. 6D is a line graph showing the percentage of healthy neutrophils calculated in UT, G-CSF-treated, and CLON-G-treated cell populations at the indicated time points. Data are presented as mean±SD of three experiments. **, P<0.001 compared to the corresponding UT group. ##, P<0.001 compared to the corresponding G-CSF-treated group. FIG. 6E is a line graph showing the total number of healthy neutrophils calculated as the number of remaining cells (intact cells counted using a hemocytometer) multiplied by the proportion of double-negative cells (shown in FIG. 6D). Data are presented as mean±SD. *P<0.001 compared to the corresponding UT group. ##, P<0.001 compared to the corresponding G-CSF-treated group. FIG. 6F is a series of images showing the in vitro phagocytosis capacity of neutrophils (APC-CD16, which fluoresced red) measured using fluorescein-conjugated PHRODO E. coli bioparticles (which fluoresced yellow). FIG. 6G is a series of bar graphs showing phagocytosis efficiency, which was expressed as the percentage of neutrophils that engulfed at least one bioparticle, and phagocytosis index, which was expressed as the average number of internalized particles per cell. At least 200 cells were assessed for each sample. Data are presented as mean±SD of three experiments. *indicates P<0.05 and **indicates P<0.001 compared to freshly isolated neutrophil group (0 h); ^{#}indicates P<0.05 and ^{##}indicates P<0.001 compared to UT (or G-CSF alone-treated) neutrophils cultured for 1 day. FIG. 6H-6I are a series of images, migration paths, and bar graphs showing the chemotactic migration of neutrophils to fMLP (100 nM), assessed using an EZ-TAXISCAN device (FIG. 6H) and calculated migration velocity and directionality (FIG. 6I). Data are presented as mean±SD of three experiments. *indicates P<0.05 and **indicates P<0.001 compared to freshly isolated neutrophil group (0 h); ^{#}indicates P<0.05 and ^{##}indicates P<0.001 compared to UT (or G-CSF alone-treated) neutrophils cultured for 1 day. FIG. 6J is a series of images showing the bacterial killing capabilities of neutrophils in vitro, reflected by the decrease in cfus of E. coli. Control: bacterial suspension without any cells; 0 h: fresh neutrophils. FIG. 6K is a bar graph showing the relative bacterial killing measured as the proportion of bacteria killed in 60 minutes. Data are presented as mean±SD of three experiments. *indicates P<0.05 and **indicates P<0.001 compared to freshly isolated neutrophil group (0 h); ^{#}indicates P<0.05 and ^{##}indicates P<0.001 compared to UT (or G-CSF alone-treated) neutrophils cultured for 1 day.

FIG. 7A-7J are a series of schematics, images, and graphs showing that CLON-G treatment prolonged the shelf-life of granulocyte apheresis concentrates. FIG. 7A is a schematic showing that granulocyte apheresis concentrates were collected and processed following a standard clinical GTX protocol. The apheresis products were treated with CLON-G without other manipulation. For functional analysis, neutrophils were purified after the CLON-G treatment. FIG. 7B is a line graph showing total neutrophil counts in granulocyte apheresis concentrates at indicated time points. Neutrophils isolated from granulocyte apheresis concentrates were counted using a hemocytometer. All data are presented as mean±SD of three experiments. **, P<0.001 compared to the corresponding UT group. ##, P<0.001 compared to the corresponding G-CSF-treated group. FIG. 7C is a series of plots showing cell death assessed by flow cytometry. Neutrophils isolated from granulocyte apheresis concentrates were stained with FITC A-V and PI. Shown are representative flow cytometry plots from one of three independent experiments. FIG. 7D is a line graph showing the percentage of healthy neutrophils calculated in UT, G-CSF-treated, and CLON-G-treated granulocyte apheresis concentrates at the indicated time points. All data are presented as mean±SD of three experiments. **, P<0.001 compared to the corresponding UT group. ##, P<0.001 compared to the corresponding G-CSF-treated group. FIG. 7E is a line graph showing the total number of healthy neutrophils calculated as the total number of neutrophils (shown in FIG. 7B) multiplied by the proportion of double-negative cells (shown in FIG. 7D). Data are presented as mean±SD. *P<0.001 compared to the corresponding UT group. ##, P<0.001 compared to the corresponding G-CSF-treated group. FIG. 7F is a bar graph showing the in vitro phagocytosis capacity of neutrophils measured using fluorescein-conjugated PHRODO E. coli bioparticles. Phagocytosis index was expressed as the average number of internalized particles per cell. At least 200 cells were assessed for each sample. Data are presented as mean±SD. **, P<0.001 compared to the corresponding UT group. #indicate P<0.05 and ##indicate P<0.001 compared to the corresponding G-CSF-treated group. FIG. 7G-7H are a series of images, migration paths, and bar graphs showing the chemotactic migration of neutrophils to fMLP (100 nM) assessed using an EZ-TAXISCAN device (FIG. 7G) and calculated migration velocity and directionality (FIG. 7H). Data are presented as mean±SD. **, P<0.001 compared to the corresponding UT group. ##, P<0.001 compared to the corresponding G-CSF-treated group. FIG. 7I is a series of images showing the bacterial killing capabilities of neutrophils in vitro, reflected by the decrease in cfus of E. coli. FIG. 7J is bar graph showing the relative bacterial killing measured as the proportion of bacteria killed in 60 minutes. Data are presented as mean±SD of three experiments. *indicates P<0.05 and **indicates P<0.001 compared to freshly isolated neutrophil group (0 h); ^{#}indicates P<0.05 and ^{##}indicates P<0.001 compared to UT (or G-CSF alone-treated) neutrophils cultured for 1 day.

FIG. 8A-8H are a series of schematics and graphs showing that pre-treatment with CLON-G prolonged half-life of transfused human neutrophils in vivo in NSG mice. FIG. 8A is a schematic showing the experimental scheme for assessing the recruitment of transfused fresh, untreated, G-CSF alone-treated, and CLON-G-treated human neutrophils in NSG mice in a mouse peritonitis model. Human neutrophils were isolated from granulocyte apheresis concentrates as described in FIG. 6 and cultured in the presence of indicated compounds for indicated time period. Peritonitis in neutropenic NSG mice was induced with 3% thioglycolate (TG). FIG. 8B is a series of plots showing the recruitment of transfused human neutrophils to inflamed peritoneal cavities as assessed by flow cytometry. FIG. 8C is a bar graph showing the total recruited human neutrophil number presented as total cell number multiplied by the percentage of transfused human neutrophils in peritoneal cavities as calculated by flow cytometry. All data are presented as mean±SD of four experiments. ** indicates P<0.001 compared to the corresponding “Fresh neutrophils” group. ns, no significant difference. FIG. 8D is a schematic showing the experimental scheme for assessing the in vivo phagocytosis capability of transfused human neutrophils in NSG mice in the peritonitis model. FIG. 8E is a series of plots showing the engulfment of pHrodo-E. coli by transfused human neutrophils as analyzed by flow cytometry. FSC-H, forward scatter height; FSC-A, forward scatter area; SSC-A, side scatter area. FIG. 8F is a bar graph showing the phagocytosis index calculated for indicated samples as mean fluorescence intensity (MFI) fold increase (MFI of treated or untreated human neutrophils at 1 hour/MFI of the corresponding human neutrophils at time 0). All data are presented as mean±SD of four experiments. *indicates P<0.05 and **indicates P<0.001 compared to “Fresh neutrophils” group. ns, no significant difference (P>0.05). FIG. 8G is a schematic showing the experimental scheme for assessing the in vivo death rate of transplanted fresh, untreated, G-CSF alone-treated, and CLON-G-treated human neutrophils in NSG mice in a mouse peritonitis model. FIG. 8H is a series of bar graphs showing the percentage of human neutrophils in peritoneal cavities calculated by flow cytometry. The number of human neutrophils left at each indicated time point was calculated as total cell number multiplied by the percentage of human neutrophils in the peritoneal cavity. Relative death of transplanted human neutrophils was calculated as the proportion of cells left at each time point. All data are presented as mean±SD of four experiments. *indicates P<0.05 and **indicates P<0.001 compared to the corresponding “Fresh neutrophils” group. ns, no significant difference (P>0.05).

FIG. 9 is a schematic showing that neutrophil death is mediated by multiple pathways.

FIG. 10A-10B are a series of images and graphs showing that Rac1 inhibition effectively suppressed chemoattractant-elicited neutrophil polarization. FIG. 10A is a series of images showing chemoattractant-induced neutrophil ruffling. Neutrophils (2×10⁵) purified from wild-type (WT) mice were plated on LABTEK chamber slides and cultured in RPMI-1640 in the presence or absence of Rac1 inhibitor for 60 minutes. Cells were then uniformly stimulated with N-formyl-met-leu-phe (fMLP, 1 μM). Images were taken 5 minutes after the fMLP stimulation (400×). FIG. 10B is a bar graph showing the percentage of cells that ruffled or extended pseudopods, calculated from images captured 5 minutes after stimulus was added. At least 200 cells were assessed for each sample in each experiment. Data are represented as mean±SD of three experiments. **p<0.001 versus untreated neutrophils.

FIG. 11A-11F is a series of graphs and images showing that CLON-G treatment increased the half-life of both human and mouse neutrophils from less than 1 day to greater than 5 days. FIG. 11A is a line graph showing freshly isolated human peripheral blood neutrophils cultured in RPMI-1640 with or without CLON-G (caspases-LMP-oxidant-necroptosis inhibition plus granulocyte colony-stimulating factor (G-CSF)). The number of intact neutrophils in the culture was counted using a hemocytometer. FIG. 11B is a series of images showing human neutrophils stained with FITC-Annexin-V (A-V, which fluoresced green) and PI (which fluoresced red) after culturing for the indicated time in vitro. Cell death was assessed by confocal fluorescence microscopy. Scale bars, 30 μm. FIG. 11C is a series of plots and a line graph. showing the death of human neutrophils was assessed by flow cytometry. The percentage of healthy neutrophils was calculated in untreated (UT) and CLON-G-treated cell populations at the indicated time points. All data are presented as mean±SD of three experiments. **, P<0.001 compared to the corresponding UT group. FIG. 11D is a line graph showing the number of intact murine cells in the culture. FIG. 11E is a series of images showing murine neutrophil death assessed by confocal fluorescence microscopy. Scale bars, 30 μm. FIG. 11F is a series of flow plots and a line graph showing the death of cultured mouse neutrophils assessed by flow cytometry. All data are presented as mean±SD of three experiments. **P<0.001 compared to the corresponding UT group.

FIG. 12 is a series of schematics, flow plots, and bar graphs showing that CLON-G treatment did not influence the proliferative capacity of contaminated neutrophil progenitors in culture. Human or mouse neutrophils were cultured in CLON-G-containing RPMI-1640 medium in the presence or absence of EdU (10 μM) for 24 hours. The frequency of EdU-positive cells was analyzed by flow cytometry. All data are represented as mean±SD of three experiments. ns, no significant difference, FSC-H, forward scatter-height; SSA, side scatter.

FIG. 13A-13H are a series of images and graphs showing that CLON-G was more effective in prolonging neutrophil survival than G-CSF alone. FIG. 13A is a series of images showing freshly isolated human peripheral blood neutrophils cultured in RPMI-1640 plus 20% fetal bovine serum (FBS) with or without G-CSF. The morphologies cells were observed after 1 day by light microscopy as described in FIG. 1. White arrows indicate dying cells. FIG. 13B-13C are a series of images and flow plots showing human neutrophils stained with FITC-Annexin-V (AV, which fluoresced green) and PI (which fluoresced red) after culturing for 1 day. Cell death was assessed by confocal fluorescence microscopy (FIG. 13B) or flow cytometry (FIG. 13C). FIG. 13D is a line graph showing the total number of healthy neutrophils calculated in UT, G-CSF-treated, and CLON-G-treated cell populations at the indicated time points as described in FIG. 1. All data are represented as mean±SD. FIG. 13E is a series of images showing the morphologies of untreated (UT) or G-CSF-treated (Treated) mouse neutrophils observed after 1 day by light microscopy as in FIG. 13A. FIG. 13F-13G are a series of images and flow plots showing murine neutrophils stained with FITC-Annexin-V (AV, which fluoresced green) and PI (which fluoresced red) after culturing for 1 day. Cell death was assessed by confocal fluorescence microscopy (FIG. 13F) or flow cytometry (FIG. 13G) as in FIG. 13B and FIG. 13C. FIG. 13H is a line graph showing the total number of healthy neutrophils calculated in UT, G-CSF-treated, and CLON-G-treated cell populations at the indicated time points. All data are presented as mean±SD. **, P<0.001 compared to the corresponding UT group. ##, P<0.001 compared to the corresponding G-CSF-treated group.

FIG. 14A-14B are a series of images and graphs showing that CLON-G treatment inhibited both apoptotic and lytic cell death of mouse neutrophils. FIG. 14A is a series of images and a bar plot showing freshly isolated mouse neutrophils cultured in vitro for 24 hours or 72 hours as in FIG. 1. Untreated (UT) or CLON-G treated (Treated) cells were stained with FITC-Annexin V (A-V, which fluoresced green) and PI (which fluoresced red). Classification of cell types was based on A-V/PI staining and morphology by confocal fluorescence microscopy. The bar graph shows the percentage of each cell type at 0 hour (fresh), 24 hours, and 72 hours; the top-down order of the stacked bars for each condition is the same as the top-down order for the legend. FIG. 14B is a series of flow plots and a bar plot showing cells collected at 0 hour, 24 hours, and 72 hours followed by staining for the different types of cell death by flow cytometry. Data are represented as mean±SD of three experiments. In this experiment, to eliminate the noise cause by late-stage fragmented dead cells (PI⁺A-V⁻ or PI⁺A-V⁺), cells were gated and analyzed with normal forward scatter (FSC) and side scatter (SSC). In the bar graph, the top-down order of the stacked bars for each condition is the same as the top-down order for the legend.

FIG. 15 is a series of flow plots and graphs showing that CLON-G treatment inhibited both apoptotic and lytic cell death of human neutrophils. Freshly isolated human neutrophils were cultured in vitro for 24 or 72 hours as described in FIG. 1. Untreated (UT) and CLON-G treated (Treated) cells were stained with FITC-Annexin V (A-V) and PI. Cells were collected at 0, 24, and 72 hours post treatment. The different types of cell death based on A-V and PI staining were calculated by flow cytometry. Data are represented as mean±SD of three experiments. In this experiment, to eliminate the noise cause by late-stage fragmented dead cells (PI⁺A-V⁻ or PI⁺A-V⁺), cells were gated and analyzed with normal forward scatter (FSC) and side scatter (SSC). In the bar graph, the top-down order of the stacked bars for each condition is the same as the top-down order for the legend.

FIG. 16A-16F are a series of images and graphs showing that drug removal did not accelerate death of CLON-G-treated neutrophils. FIG. 16A is a schematic showing human neutrophils treated with CLON-G for 3 days in vitro followed by removing CLON-G by replacing the cell culture medium to normal medium (CLON-G-treated, 3 Days). At the same time, freshly isolated neutrophils were cultured in the same medium without CLON-G (Fresh neutrophils). FIG. 16B is a bar graph showing the total numbers of intact neutrophils counted at the indicated time points. Data are presented as mean±SD of three experiments. *, P<0.05 and **, P<0.001 compared to the corresponding UT (fresh neutrophil) group. FIG. 16C is a series of plots and line graph showing cells stained with FITC-Annexin V (A-V) and PI analyzed by flow cytometry. The percentage of healthy neutrophils was assessed. Data in the line graph are presented as mean±SD of three experiments. **, P<0.001 compared to the corresponding UT (fresh neutrophil) group. FIG. 16D is a schematic showing human and mouse neutrophils treated with CLON-G for 1 hour in vitro followed by removing drugs by replacing the drug-containing medium to normal medium. FIG. 16E-16F are a series of line graphs and bar graphs showing the number of intact cells, the percentage of healthy (A-V⁻PI⁻) cells, and the total number of healthy cells in UT and CLON-G-treated human (FIG. 16E) or mouse (FIG. 16F) neutrophil populations that were measured and calculated. All data are represented as mean±SD of three experiments. *, P<0.05 and **, P<0.001 compared to the corresponding UT group.

FIG. 17A-17B shows cyclophosphamide (CPM)-induced neutropenia in mice. FIG. 17A shows a schematic of the experimental scheme. FIG. 17B is a line graph showing the peripheral blood neutrophil counts in normal and CPM-treated mice. Neutrophil number in the peripheral blood (PB) was assessed using a HEMAVET 850 hematology system. Data are represented as mean±SD of three experiments.

FIG. 18A-18E is a series of images and graphs showing that CLON-G treatment did not impair migration and bacterial killing capability of human neutrophils. Freshly isolated human neutrophils were cultured as described in FIG. 1 and FIG. 13. The function of UT, G-CSF-treated, and CLON-G-treated neutrophils was assessed at the indicated time points. FIG. 18A is series of images, migration paths, and bar graphs showing the chemotactic migration of human neutrophils to fMLP (100 nM) assessed using an EZ-TAXISCAN device. Migration velocity and directionality were calculated. FIG. 18B is series of images and bar graphs showing the in vitro phagocytosis capacity of human neutrophils (APC-CD16+, which fluoresced red) was measured using fluorescein-conjugated PHRODO E. coli bioparticles (which fluoresced yellow). At least 200 cells were assessed for each sample. The engulfed bioparticles are indicated with white arrowheads. FIG. 18C is a line graph showing fMLP-induced ROS production in human neutrophils. FIG. 18D is series of images showing in vitro killing of E. coli by human neutrophils. The bacterial killing capabilities were reflected by the decrease in colony forming units (cfu). 1, 2, 3, and 4 indicate serial dilutions in the bacterial colony assay. Control: bacterial suspension without any cells; 0 h: fresh neutrophils. FIG. 18E is a bar graph showing the relative bacterial killing measured as the proportion of bacteria killed in 60 minutes. All data are represented as mean±SD of three experiments. *indicates P<0.05 and **indicates P<0.001 compared to freshly isolated neutrophil group (0 h); ^{#}indicates P<0.05 compared to UT (or G-CSF alone-treated) neutrophils cultured for 1 day; ^{##}indicates P<0.001 compared to UT (or G-CSF alone-treated) neutrophils cultured for 1 day.

FIG. 19A-19D are a series of schematics and graphs showing the effect of CLON-G-treatment on the recruitment of transfused neutrophils in a mouse peritonitis model. FIG. 19A is a schematic showing the experimental scheme for assessing the relative in vivo recruitment of transfused neutrophils in a mouse peritonitis model. The peritonitis was induced by 3% TG (i.p.). The trafficking of transfused cultured green fluorescent protein (GFP)+ and fresh CD45.1⁺ neutrophils was assessed in the same neutropenic CD45.2 recipient mouse. The fresh, untreated, G-CSF alone-treated, and CLON-G-treated neutrophils were prepared as described in FIG. 2D. FIG. 19B is a bar graph showing the total cell number in the peritoneal cavity (PC). The cells were stained with APC-Ly6g antibody and the percentage of neutrophils was analyzed by flow cytometry. Neutrophil number in peritoneal lavage was calculated as the product of percentage of neutrophils and total cell number. FIG. 19C is a series of plots showing flow cytometry analysis of recruited transfused neutrophils in peritoneal lavage. Cells in peritoneal lavage fluid were stained with PE-CD45.1. Representative flow cytometry plots from one of three independent experiments are shown. FIG. 19D is a series of bar graphs showing the ratio of the indicated transplanted neutrophil populations in peritoneal lavage fluid. All data are represented as mean±SD of three experiments. **indicates P<0.001, compared to input. ns. not statistically significant (P>0.05).

FIG. 20A-20H are a series of schematics, images, and graphs showing testing of a clinically relevant mouse E. coli pneumonia model. FIG. 20A is a schematic showing that pneumonia was induced by intrarectal instillation of 1×10⁶ cfu E. coli. Lung inflammation and pathology were assessed 24 hours after E. coli challenge. FIG. 20B is a series of images and bar graphs showing macrophages and neutrophils in bronchoalveolar lavage fluid (BALF) of untreated, saline control, and E. coli-challenged mice assessed by morphometric analysis. The morphology of BALF cells was analyzed by Wright-Giemsa staining. Macrophages and neutrophils were identified by morphometric analysis and their percentage was determined accordingly. At least 200 cells were examined for each sample. Scale bars, 20 μm. FIG. 20C is a series of plots and bar graphs showing the macrophages and neutrophils in BALF of untreated, saline control, and E. coli-challenged mice assessed by flow cytometry. The cells in BALF were stained with FITC-F4/80 and APC-CD11b antibodies. The percentages of alveolar macrophage (CD11b⁻F4/80⁺), inflammatory macrophages (CD11b⁺F4/80⁺), and polymorphonuclear cells (PMN, CD11b⁻F4/80⁻) were analyzed by flow cytometry. FIG. 20D is a series of images showing H&E staining of lung tissues from saline control or E. coli-challenged mice. Scale bar, 50 μm. FIG. 20E is a bar graph showing emigrated neutrophils in alveolar air spaces quantified as volume fraction of the alveolar air space using standard point-counting morphometric techniques. The relative volumes of the parenchymal regions occupied by emigrated neutrophils were calculated by investigators blinded to the identities of the mice and were expressed as the percentage of the total parenchymal region volume (including both tissue and air space). FIG. 20F is a bar graph showing pulmonary edema formation quantified as the percentage of edema area in the total parenchymal region. FIG. 20G is a bar graph showing protein accumulated in the BALF measured using a protein assay kit. FIG. 20H is a series of bar graphs showing BALF chemokine and cytokine concentrations determined using enzyme-linked immunosorbent assay (ELISA) kits. Data are represented as mean±SD of three experiments. *, P<0.05; **, P<0.001.

FIG. 21A-21B show that transfusion of fresh or CLON-G treated neutrophils did not induce tissue damage in unchallenged or lipopolysaccharides (LPS)-challenged neutropenic mice. FIG. 21A is a schematic showing neutropenic mice that were or were not challenged by intratracheal instillation of LPS (5 mg/kg body weight), and then transfused with fresh or CLON-G treated neutrophils. Pathological examination of heart, spleen, kidney, lung, and liver was performed 24 hours after neutrophil transfusion. FIG. 21B is a series of images showing histopathologic assessment of the heart, spleen, kidney, lung, and liver of mice transfused with indicted amounts of fresh or CLON-G treated neutrophils. Representative H&E-stained sections of indicated tissues are shown. Scale bars are indicated in each panel.

FIG. 22A-22F are a series of schematics, images, and graphs showing that transfusion with stored CLON-G treated neutrophils enhanced host defenses as effectively as transfusion with untreated fresh neutrophils in neutropenia-related fungal infection. FIG. 22A is a schematic showing neutropenic mice challenged with C. albicans (1×10³ cfu). FIG. 22B is a bar graph showing neutrophil cell counts in the kidney. On day 3, kidney tissues were harvested by grinding and filtration; the total cell number was counted with a hemocytometer. The harvested cells were stained with CD45-PE-Cy7 and Ly6G-APC; the percentage of PMNs (CD45⁺Ly6G⁺) was evaluated by flow cytometry. Neutrophil cell counts in the kidney were calculated accordingly. Data are represented as mean±SD of three experiments. *indicates P<0.01. FIG. 22C is a bar graph showing the fungal burden in kidney tissue. Data are represented as mean±SD of three experiments. *, P<0.05; **, P<0.001. FIG. 22D is a bar graph showing the body weights of C. albicans-challenged mice. Data are represented as mean SD of three experiments. *indicates P<0.01. FIG. 22E shows the survival curves for C. albicans-challenged mice transfused with fresh or CLON-G-treated neutrophils. Survival was analyzed using a Log-rank test. P-values are indicated. FIG. 22F is a series of images showing H&E staining of kidney tissues. Representative images of three experiments are shown. Scale bars are indicated in each panel.

FIG. 23 is a schematic and bar graph showing the culture of murine bone marrow (BM) neutrophils using various cell culture media, including medium comprising Emricasan (see e.g., “E10R,” “E10,” or “E25”). The RIPK1 inhibitor was Nec-1s. The following components were present in the cell culture media as indicated in FIG. 23, with concentrations as follows: 50 μM Q-VD-Oph; μM or 25 μM Emricasan; 10 μM Nec-1s; 1 mM NAC; 10 pm Hsp70; 1 μM DFO; and/or 10 ng/mL G-CSF.

### DETAILED DESCRIPTION

Described herein are compositions and methods that improve the efficacy of neutrophil transfusion by leveraging multiple neutrophil death mechanisms and prolonging neutrophil survival without compromising their function. Pharmacological screening revealed a combined treatment that delayed neutrophil death by simultaneously targeting multiple death pathways. CLON-G (caspases-LMP-oxidant-necroptosis inhibition plus G-CSF) treatment altered neutrophil fate, increasing the ex vivo shelf life of collected neutrophils without affecting their in vivo function, improving GTX efficacy in clinically relevant murine models of granulocyte transfusion in neutropenic hosts. These results provide a clinical strategy for the storage and application of neutrophils in transfusion medicine, demonstrating CLON-G treatment as a therapeutic approach for improving GTX efficacy.

Specifically, the technology described herein is directed to compositions and cell culture media for increasing the lifespan of neutrophils. Also disclosed are methods and kits for culturing neutrophils using the compositions or cell culture media as described herein. Such cultured neutrophils can be used to treat disorders such as neutropenia or microbial infections.

### Neutrophils

In multiple aspects, described herein are compositions and methods for prolonging the lifespan of neutrophils. In one aspect, described herein is a neutrophil, or population thereof, produced by any of the methods described herein. In one aspect, described herein is a neutrophil, or population thereof, produced using a cell culture medium, composition, or method described herein. In one aspect, described herein is a combination comprising a neutrophil and a composition or cell culture medium as described herein.

Neutrophils (also known as neutrocytes or heterophils) are the most abundant type of granulocytes and make up 40%-70% of all white blood cells in humans; 10-25% of circulating mouse leukocytes are neutrophils. Neutrophils are formed from myeloid precursors in the bone marrow and differentiated into subpopulations of neutrophil-killers and neutrophil-cagers. Neutrophils are short-lived and highly mobile, as they can enter parts of tissue where other cells or molecules cannot. Neutrophils can be subdivided into segmented neutrophils and banded neutrophils (or bands). They form part of the polymorphonuclear cells family (PMNs) together with basophils and eosinophils; neutrophils are granule-containing, polymorphonuclear leukocytes. Neutrophils are a type of phagocyte and are typically found in the bloodstream. During the beginning (acute) phase of inflammation, particularly as a result of bacterial infection, environmental exposure, or some cancers, neutrophils are one of the first responders of inflammatory cells to migrate toward the site of inflammation. Neutrophils migrate through the blood vessels and then through interstitial tissue, following chemical signals, including but not limited to interleukin-8 (IL-8), C5a, fMLP, Leukotriene B4, and H₂O₂ via chemotaxis. Neutrophils are recruited to the site of injury within minutes following trauma and are the hallmark of acute inflammation. Neutrophils play a central role in the innate immune response by destroying foreign particles either intracellularly in phagosomes or extracellularly by releasing neutrophil extracellular traps (NETs), and promoting acute inflammation.

Although neutrophils can be visually identified based on the shape of their nuclei and cytoplasmic granularity, they can also be identified by flow cytometry. Mouse neutrophils are commonly identified based on the cell surface expression of Ly-6G and CD11b/Integrin alpha M. Since mouse granulocytic myeloid-derived suppressor cells can also express these markers, neutrophils are frequently distinguished from these cells in mice based on their lack of expression of M-CSF R/CD115 and CD244/SLAMF4, along with an absence of immunosuppressive properties. In humans, neutrophils are distinguished from eosinophils and monocytes based on the expression of both CD15 and CD16/Fc gamma RIII on human neutrophils, along with the lack of expression of CD14. In addition, CD66b/CEACAM-8, CD11b/Integrin alpha M, CD33, and the cytoplasmic marker, myeloperoxidase, are other common markers that are used to identify human neutrophils. Accordingly, in some embodiments of any of the aspects, at least one of the following markers, or any combination thereof, is used to identify neutrophils produced by the methods described herein: Ly-6G, CD11b/CD18 (also referred to as Integrin alpha M, ITGAM, Mac-1, LFA-1, or ITGB2), M-CSF R/CD115, CD244/SLAMF4, CD15, CD16/Fc gamma RIII, CD14^{lo/-}, CD66b/CEACAM-8, CD11b/Integrin alpha M, CD33, and/or myeloperoxidase. Non-limiting examples of neutrophil activation markers include: cell free DNA (cfDNA), neutrophil elastase (NE), myeloperoxidase (MPO), and citrullinated Histone 3 (H3cit); cfDNA and H3cit are surrogate markers of NET formation.

In some embodiments of any of the aspects, the neutrophil, or population thereof, is a mammalian neutrophil. In some embodiments of any of the aspects, the neutrophil, or population thereof, is a human neutrophil. In some embodiments of any of the aspects, the neutrophil, or population thereof, is a mouse neutrophil. In some embodiments of any of the aspects, the neutrophil, or population thereof, is a peripheral blood neutrophil (PMN). Methods of isolating PMNs from blood samples are known in the art and non-limiting examples include: white blood cell filters (e.g., IMUGARD® III Leukocyte Filter System) and density gradient centrifugation (e.g., HISTOPAQUE-1119; FICOLL-HYPAQUE; sodium metrizoate and DEXTRAN 500). In some embodiments of any of the aspects, the neutrophil, or population thereof, is isolated from a subject using apheresis, which is the removal of blood plasma from the body of a subject by the withdrawal of blood, its separation into plasma and cells, the isolation of the cells, and the reintroduction of the non-isolated components (e.g., plasma, red blood cells) back into the bloodstream of the subject. In some embodiments of any of the aspects, the subject from which the neutrophils are isolated (e.g., by blood sample or apheresis) are stimulated prior to the neutrophil isolation in order to increase the number of circulating neutrophils. For example, the subject can be stimulated with G-CSF (e.g., 250 ug) and dexamethasone (e.g., 8 mg). In some embodiments, the neutrophils are isolated from the subject at about 12 hours after stimulation (e.g., about 4 hours, about 6 hours, about 8 hours, about 10 hours, about 11 hours, about 12 hours, about 13 hours, about 14 hours, about 16 hours, about 18 hours, about 20 hours, about 22 hours, about 24 hours, or more). In some embodiments of any of the aspects, the neutrophil, or population thereof, is bone marrow neutrophil.

As described further herein, the neutrophils produced as described herein can be administered to a subject in need thereof. The neutrophils can be autologous/autogenic (“self”) or non-autologous (“non-self,” e.g., allogeneic, syngeneic or xenogeneic) in relation to the recipient of the cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In some embodiments, the neutrophils are allogeneic. In various embodiments, the neutrophils to be implanted into a subject in need thereof is autologous or allogeneic to the subject. In various embodiments, the neutrophils described herein can be derived from one or more donors, or can be obtained from an autologous source. In some embodiments, the neutrophils are expanded in culture prior to administration to a subject in need thereof.

In some embodiments of any of the aspects, the neutrophil, or population thereof is a mature neutrophil, which can be characterized by a segmented nucleus and/or maturation markers such as CD16^{hi}, CXCR2^{hi}, CXCR4^{low}, and CD62L^{hi}. In some embodiments of any of the aspects, the neutrophil, or population thereof, is derived from a stem cell, such as an induced pluripotent stem cell (iPSC), cord blood, a hematopoietic stem cell, a common myeloid progenitor cell, or a granulocyte-monocyte progenitor cell (GMP). In some embodiments of any of the aspects, the neutrophil, or population thereof, is derived from an immature neutrophil, such as a myeloblast, promyelocyte, myelocyte, metamyelocyte, or band neutrophil. The compositions, cell culture media, and methods described herein can be applied during or after differentiation of the stem or immune neutrophil into a mature neutrophil.

In some embodiments of any of the aspects, the half-life of the neutrophil, or population thereof, produced using the compositions, cell culture media, and/or methods described herein is at least 5 days. In some embodiments of any of the aspects, the half-life of the neutrophil, or population thereof, produced using the compositions, cell culture media, and/or methods described herein is at least 1 day, at least 2 days, at least 3 days, at least 4 days, at least 5 days, at least 6 days, at least 7 days, or more. In some embodiments of any of the aspects, the half-life of the neutrophil, or population thereof, produced using the compositions, cell culture media, and/or methods described herein is at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, at least 49 hours, at least 50 hours, at least 51 hours, at least 52 hours, at least 53 hours, at least 54 hours, at least 55 hours, at least 56 hours, at least 57 hours, at least 58 hours, at least 59 hours, at least 60 hours, at least 61 hours, at least 62 hours, at least 63 hours, at least 64 hours, at least 65 hours, at least 66 hours, at least 67 hours, at least 68 hours, at least 69 hours, at least 70 hours, at least 71 hours, at least 72 hours, at least 73 hours, at least 74 hours, at least 75 hours, at least 76 hours, at least 77 hours, at least 78 hours, at least 79 hours, at least 80 hours, at least 81 hours, at least 82 hours, at least 83 hours, at least 84 hours, at least 85 hours, at least 86 hours, at least 87 hours, at least 88 hours, at least 89 hours, at least 90 hours, at least 91 hours, at least 92 hours, at least 93 hours, at least 94 hours, at least 95 hours, at least 96 hours, at least 97 hours, at least 98 hours, at least 99 hours, at least 100 hours, at least 101 hours, at least 102 hours, at least 103 hours, at least 104 hours, at least 105 hours, at least 106 hours, at least 107 hours, at least 108 hours, at least 109 hours, at least 110 hours, at least 111 hours, at least 112 hours, at least 113 hours, at least 114 hours, at least 115 hours, at least 116 hours, at least 117 hours, at least 118 hours, at least 119 hours, at least 120 hours, at least 121 hours, at least 122 hours, at least 123 hours, at least 124 hours, at least 125 hours, at least 126 hours, at least 127 hours, at least 128 hours, at least 129 hours, at least 130 hours, at least 131 hours, at least 132 hours, at least 133 hours, at least 134 hours, at least 135 hours, at least 136 hours, at least 137 hours, at least 138 hours, at least 139 hours, at least 140 hours, at least 141 hours, at least 142 hours, at least 143 hours, at least 144 hours, at least 145 hours, at least 146 hours, at least 147 hours, at least 148 hours, at least 149 hours, at least 150 hours, at least 151 hours, at least 152 hours, at least 153 hours, at least 154 hours, at least 155 hours, at least 156 hours, at least 157 hours, at least 158 hours, at least 159 hours, at least 160 hours, at least 161 hours, at least 162 hours, at least 163 hours, at least 164 hours, at least 165 hours, at least 166 hours, at least 167 hours, at least 168 hours, at least 169 hours, at least 170, or more. In some embodiments of any of the aspects, the half-life of the neutrophil, or population thereof, produced using the compositions, cell culture media, and/or methods described herein is 1 day to 2 days, 2 days to 3 days, 3 days to 4 days, 4 days to 5 days, 5 days to 6 days, 6 days to 7 days, 3 days to 7 days, or 5 days to 7 days.

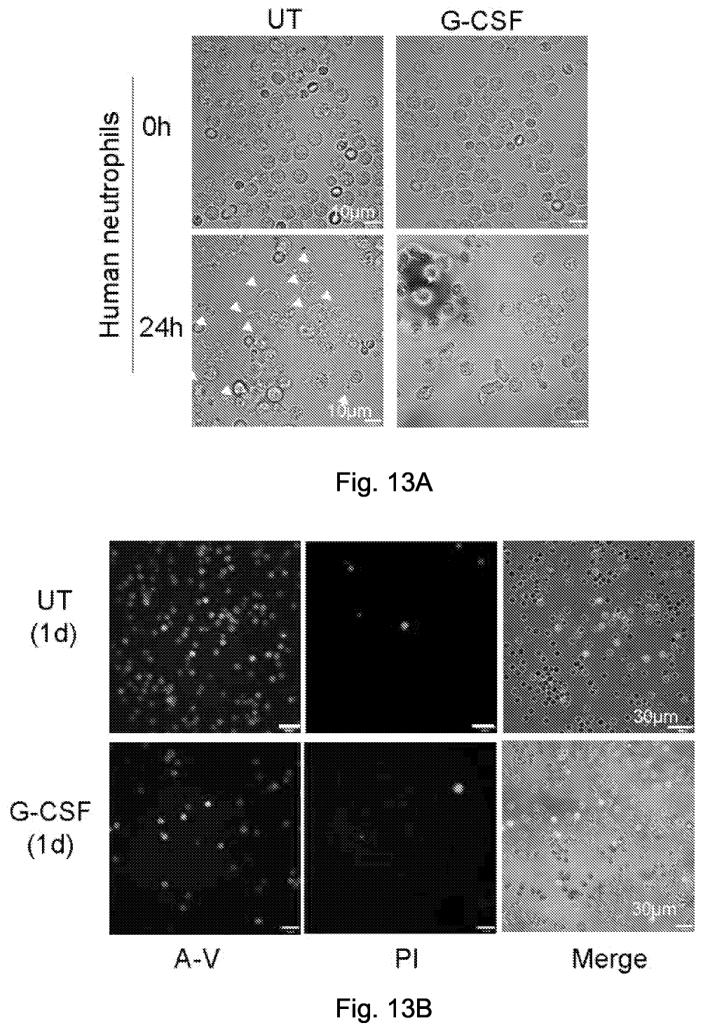

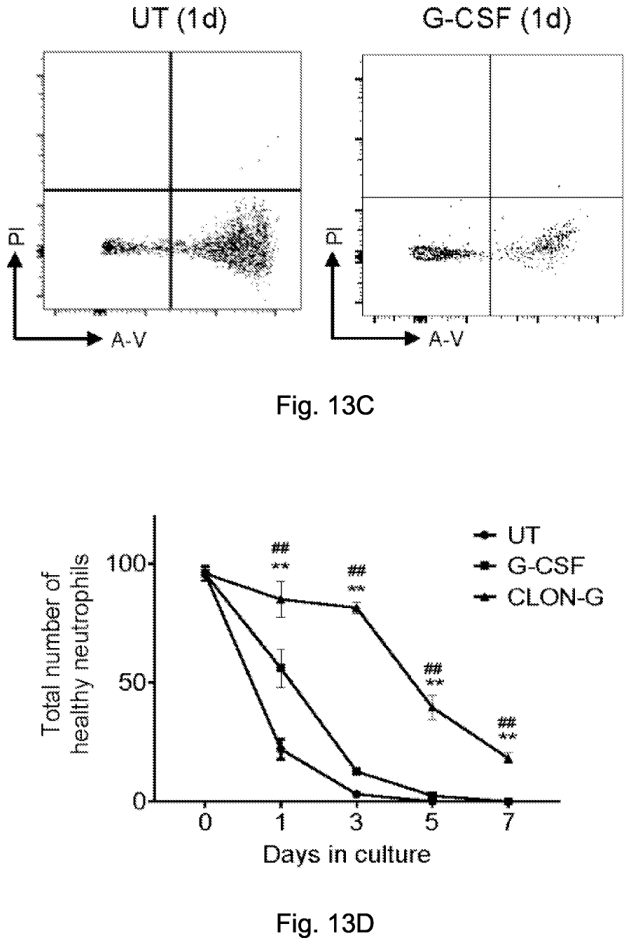

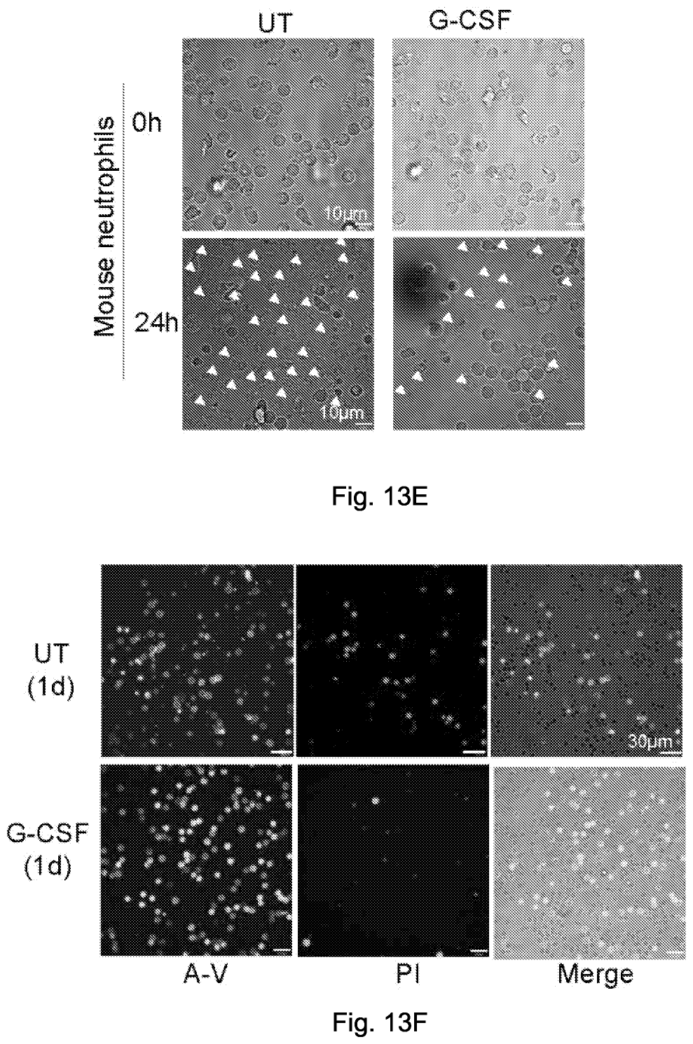

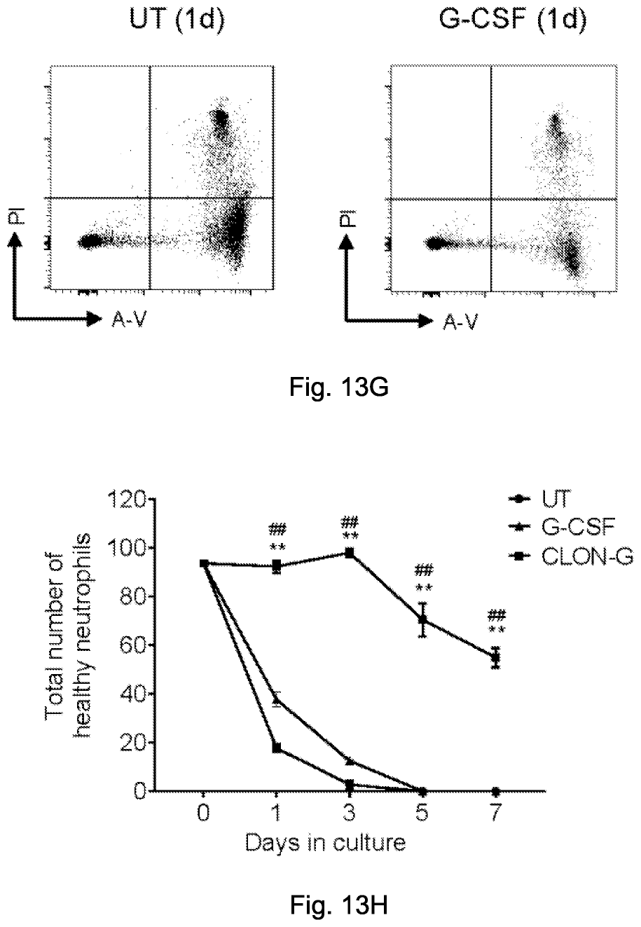

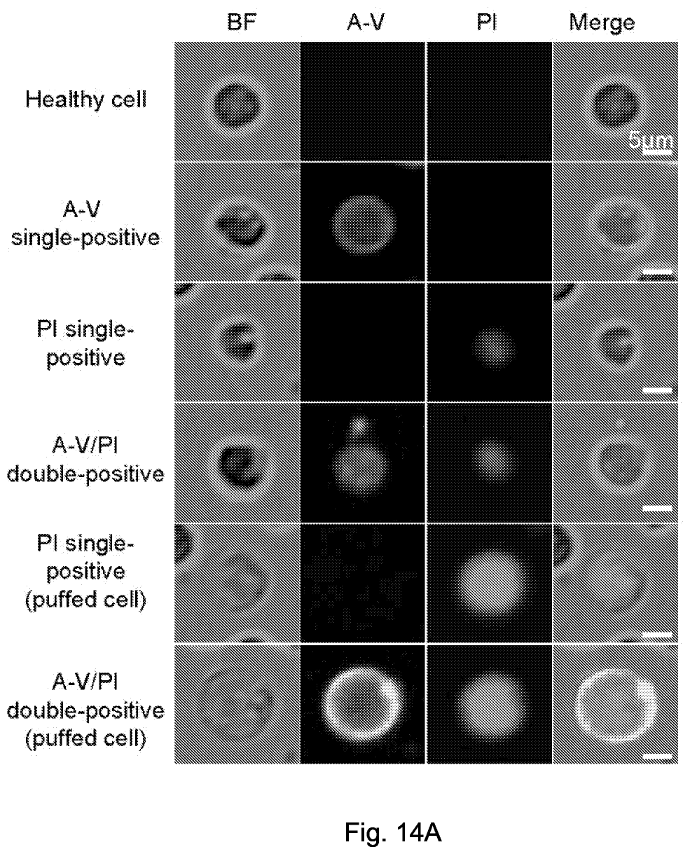

In some embodiments of any of the aspects, the half-life of the neutrophil, or population thereof, produced using the compositions, cell culture media, and/or methods described herein is increased at least 4-fold compared to untreated neutrophils. In some embodiments of any of the aspects, the half-life of the neutrophil, or population thereof, produced using the compositions, cell culture media, and/or methods described herein is increased at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, at least 500%, 100%-200%, 200%-300%, 300%-400%, 400%-500%, or more, compared to untreated neutrophils or compared to neutrophils treated with G-CSF alone (see e.g., FIG. 13D).

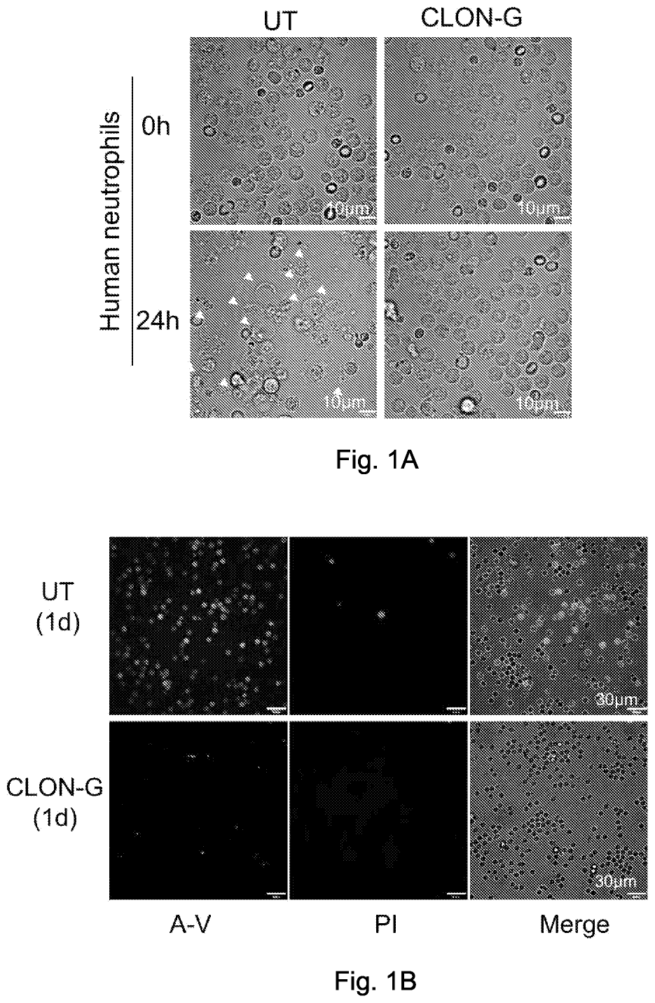

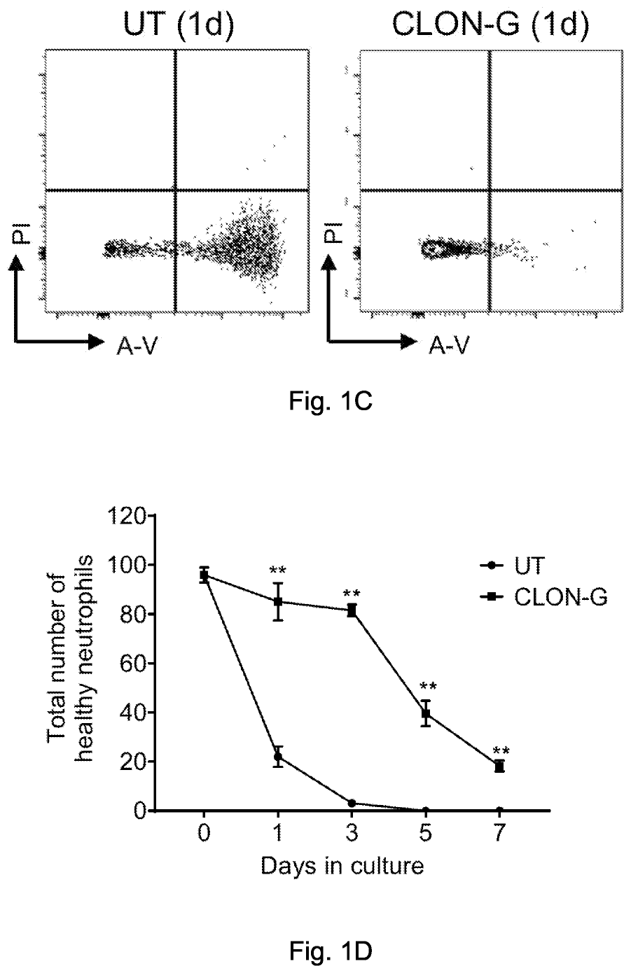

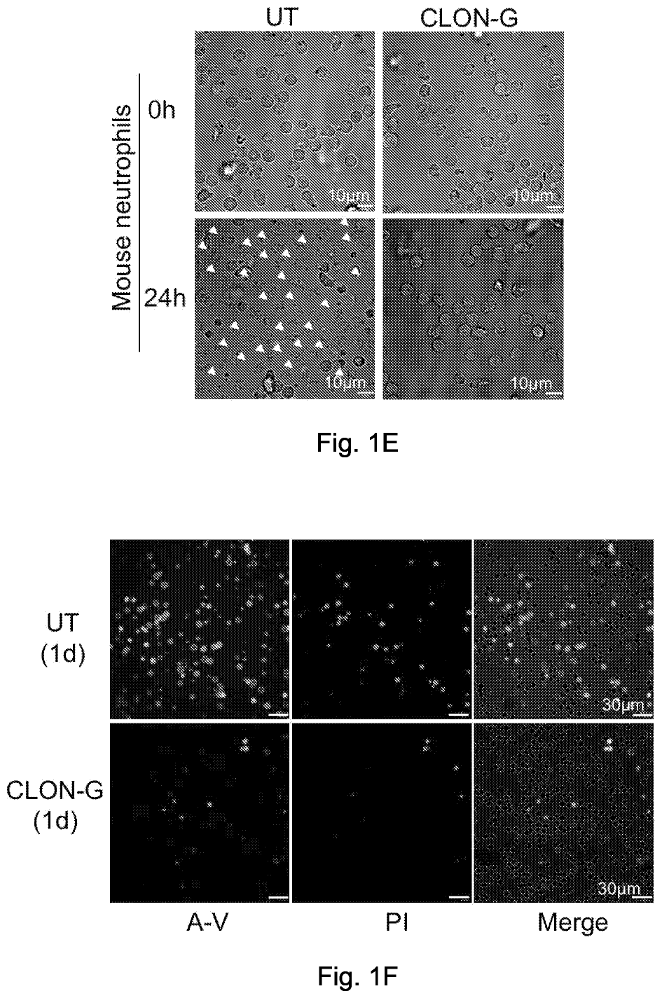

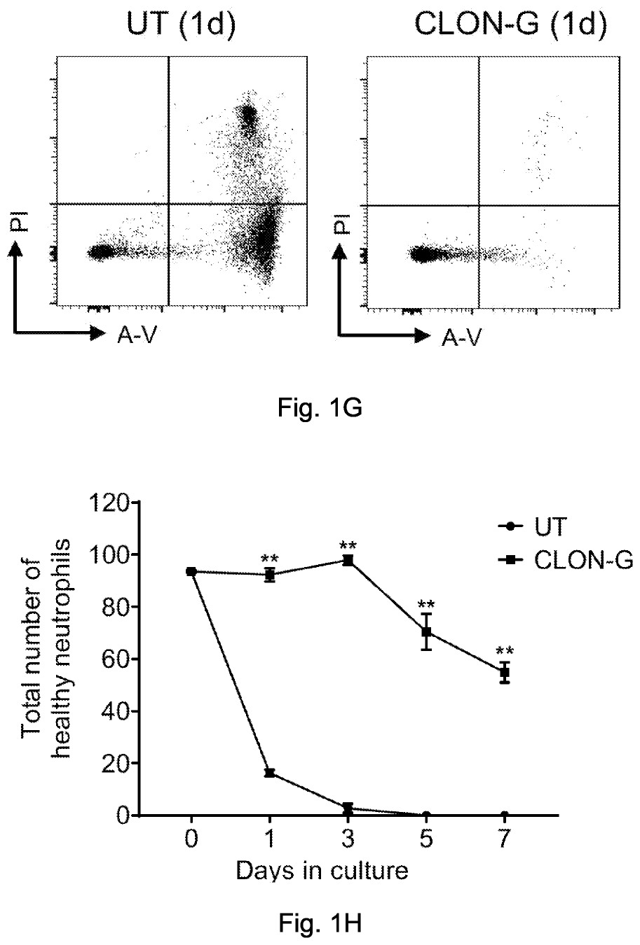

In some embodiments of any of the aspects, the survival (i.e., % viable) of the neutrophil population produced using the compositions, cell culture media, and/or methods described herein is at least 90% (see e.g., Tables 1-2, FIG. 1D). In some embodiments of any of the aspects, the survival of the neutrophil population produced using the compositions, cell culture media, and/or methods described herein is at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or more. In some embodiments of any of the aspects, the survival of the neutrophil population is measured after 1 hour to 3 days of using the compositions, cell culture media, and/or methods described herein.

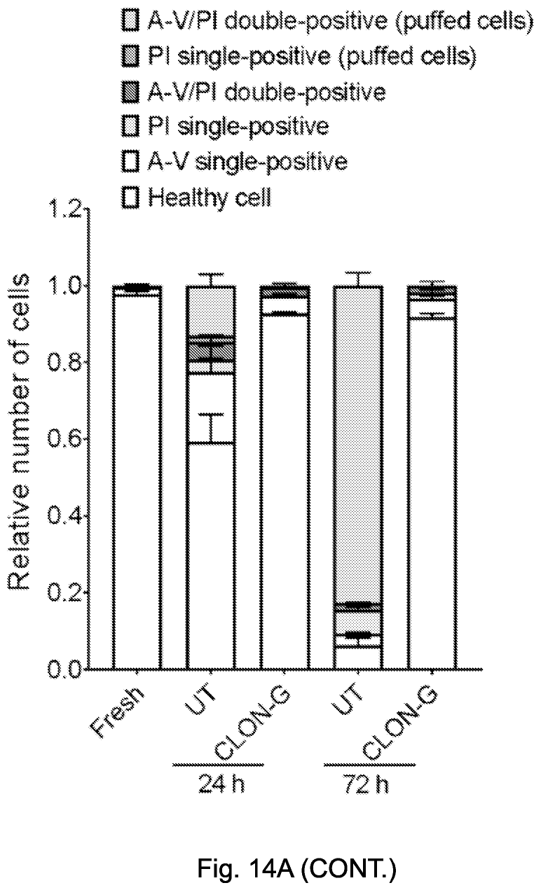

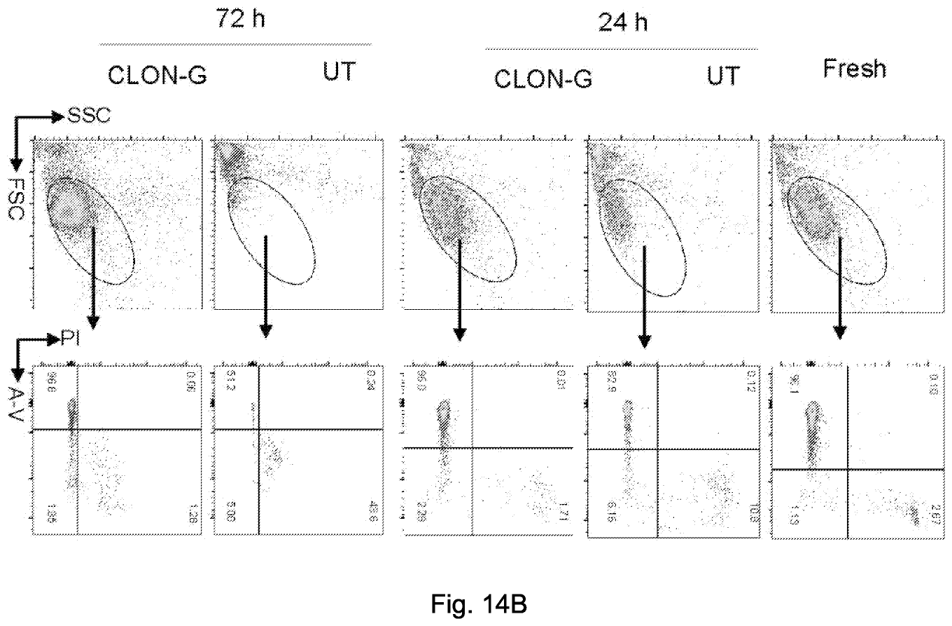

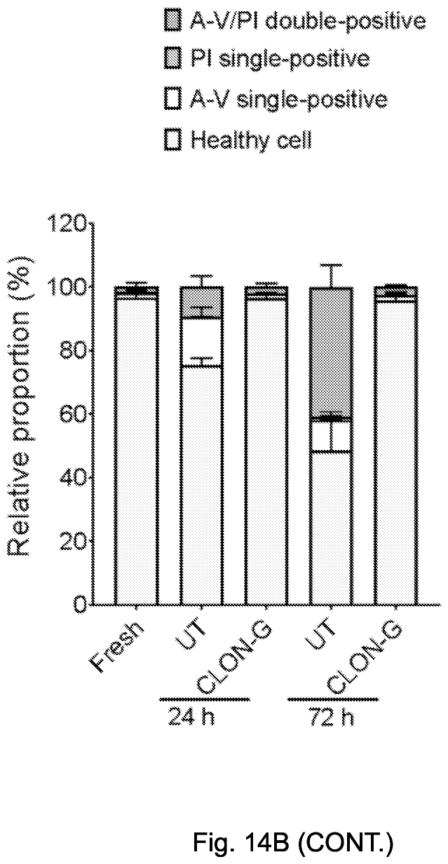

In some embodiments of any of the aspects, the proportion of the neutrophil population produced using the compositions, cell culture media, and/or methods described herein that is apoptotic and/or lytic is at most 10% (see e.g., FIG. 14A). In some embodiments of any of the aspects, the proportion of the neutrophil population produced using the compositions, cell culture media, and/or methods described herein that is apoptotic and/or lytic is at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20%. In some embodiments of any of the aspects, the apoptotic and/or lytic proportion of the neutrophil population is measured after 1 hour to 3 days of using the compositions, cell culture media, and/or methods described herein.

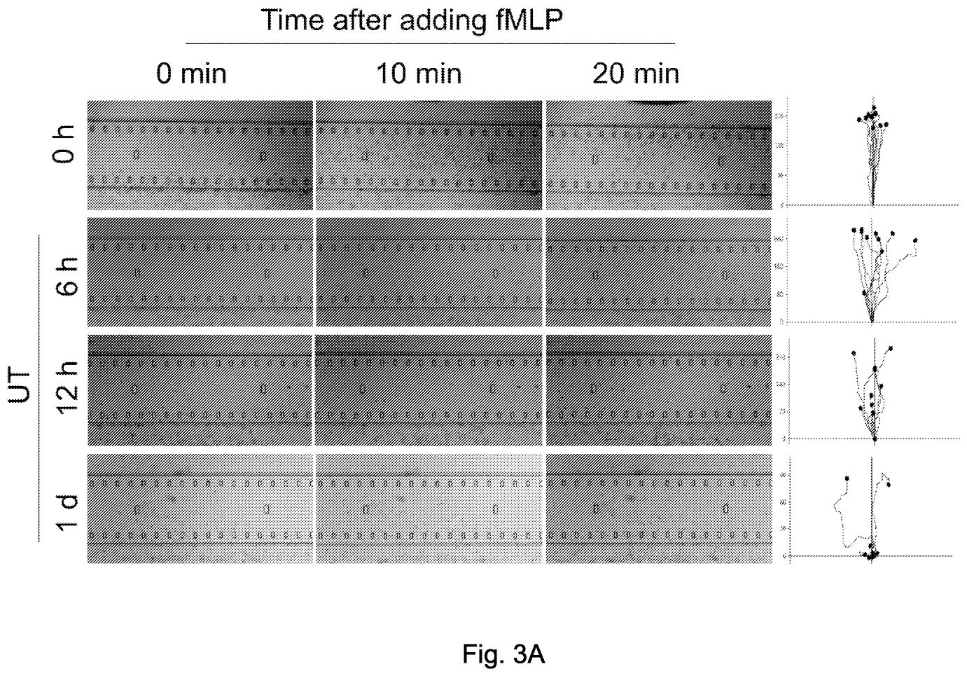

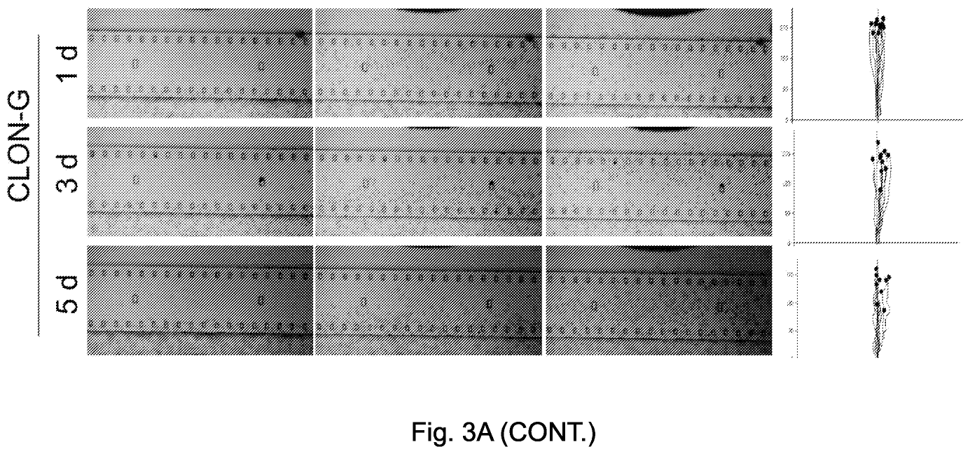

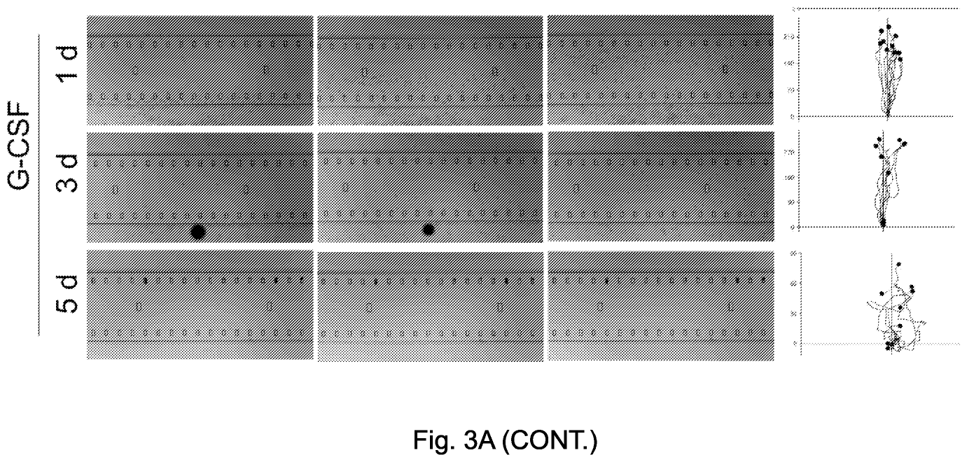

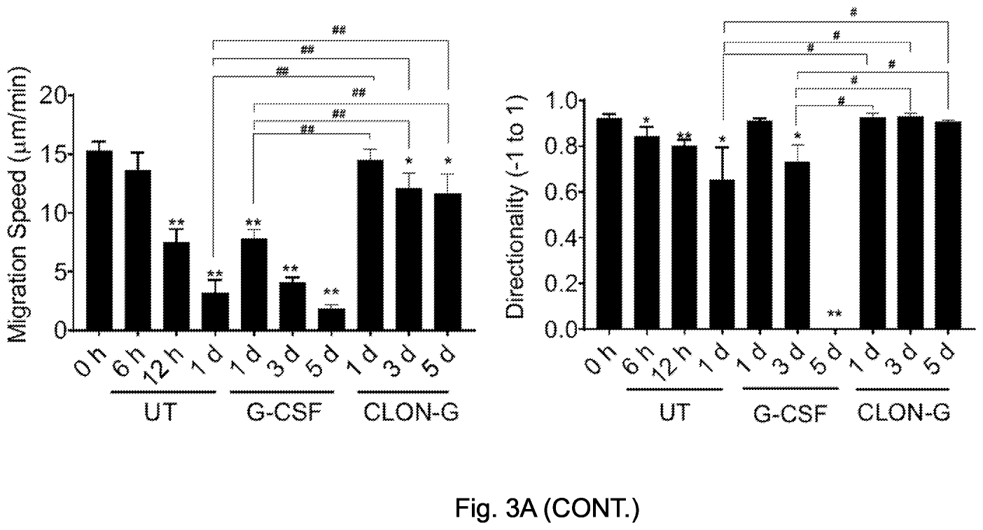

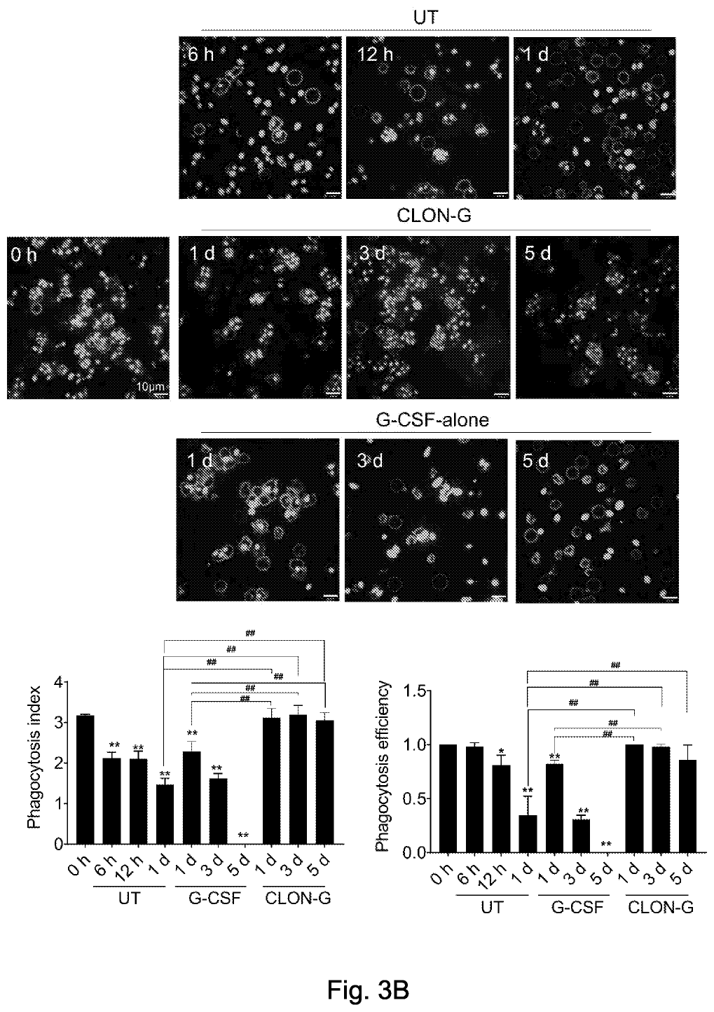

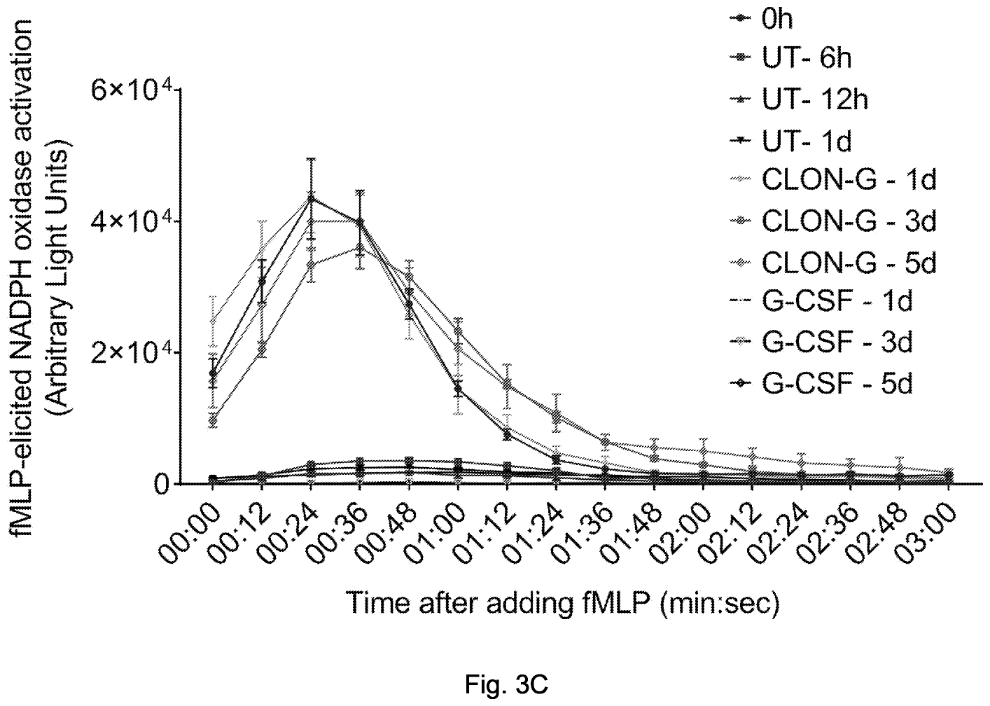

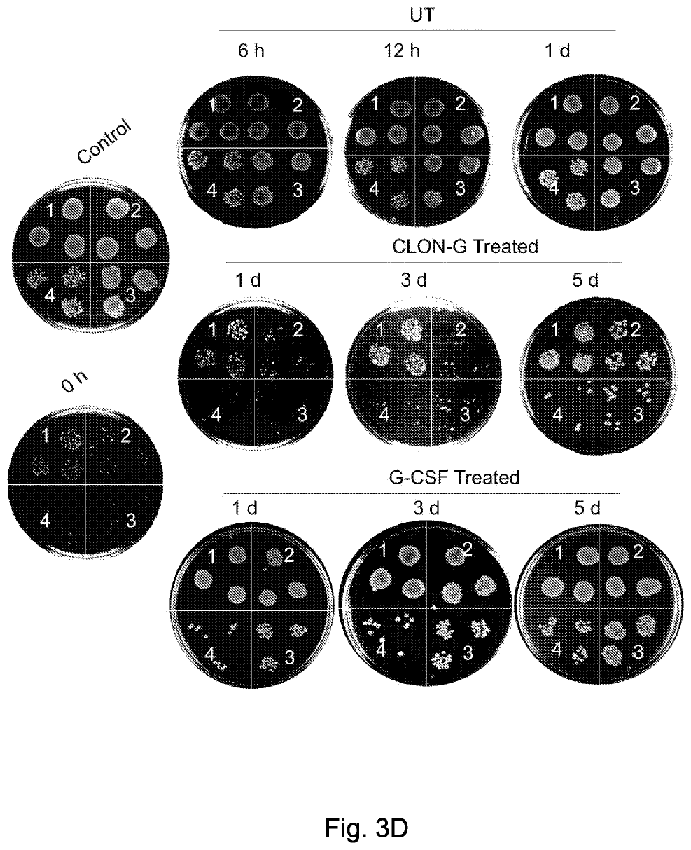

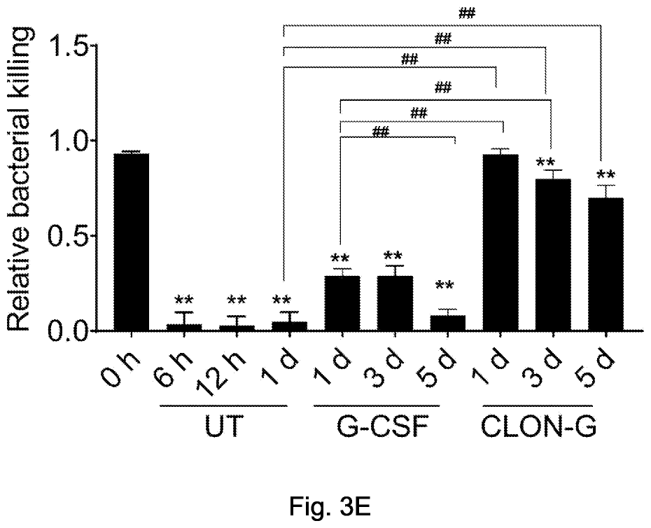

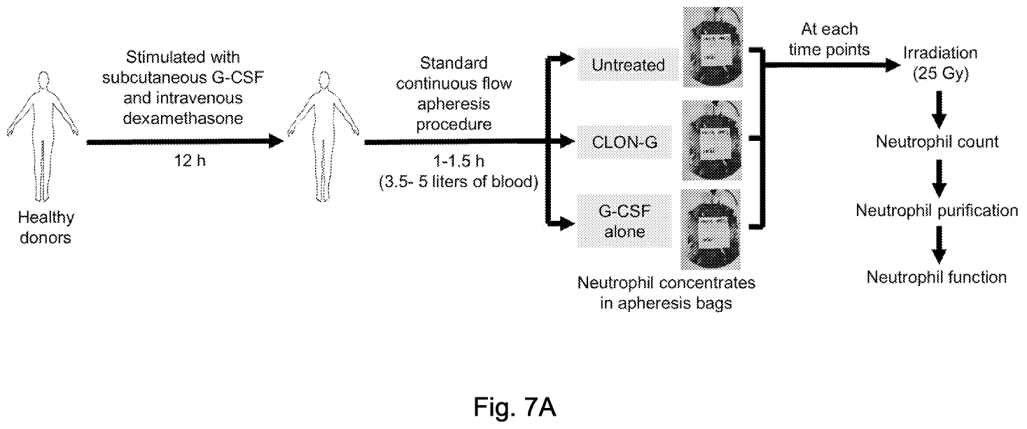

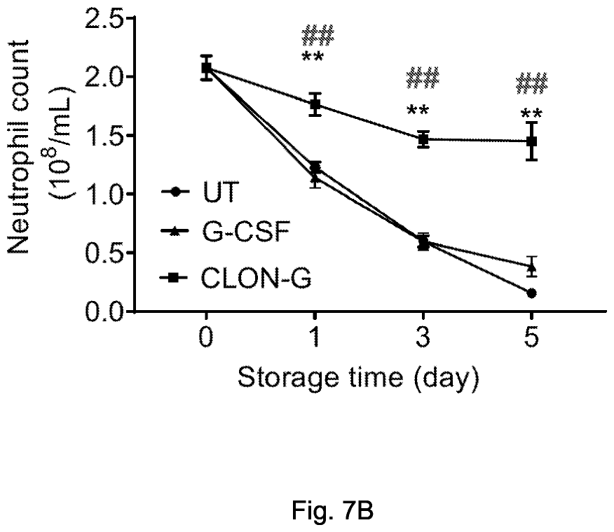

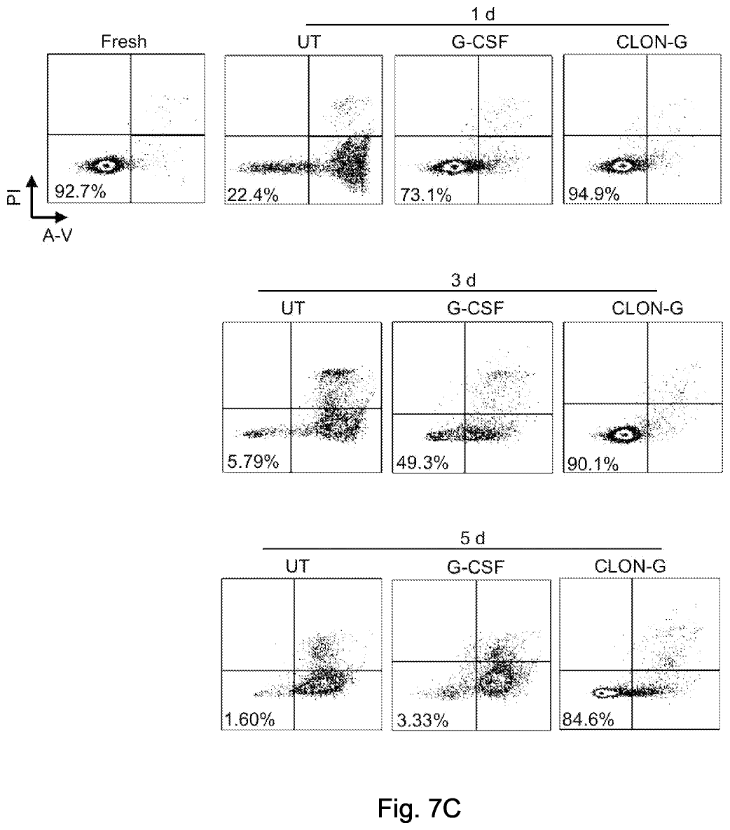

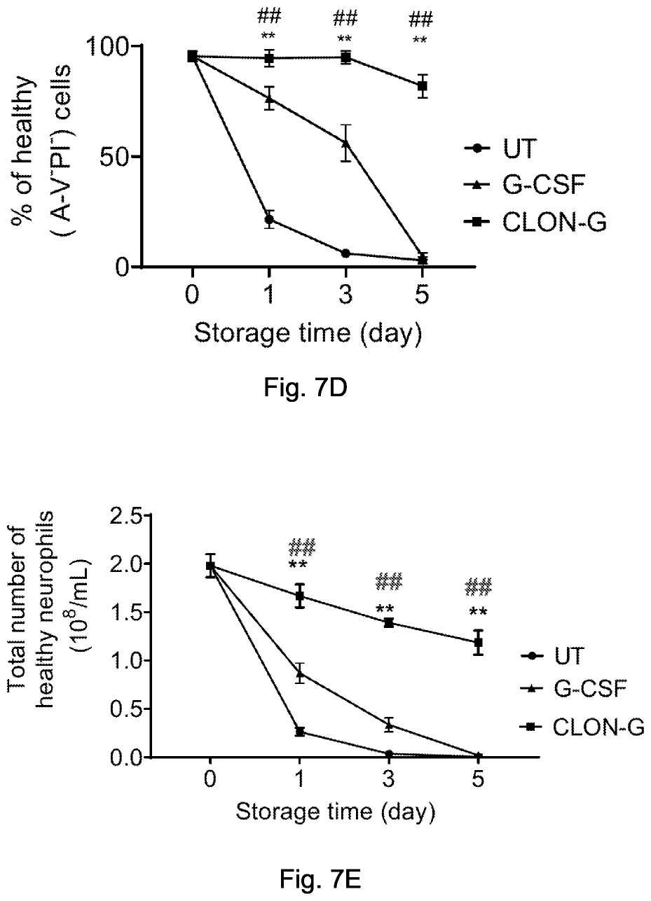

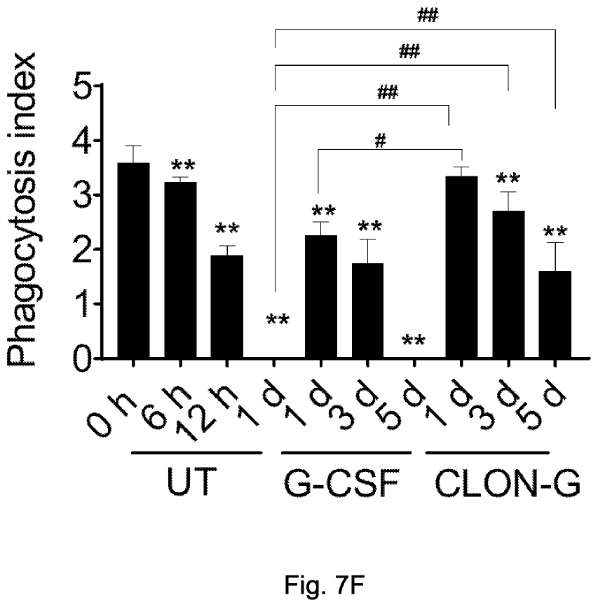

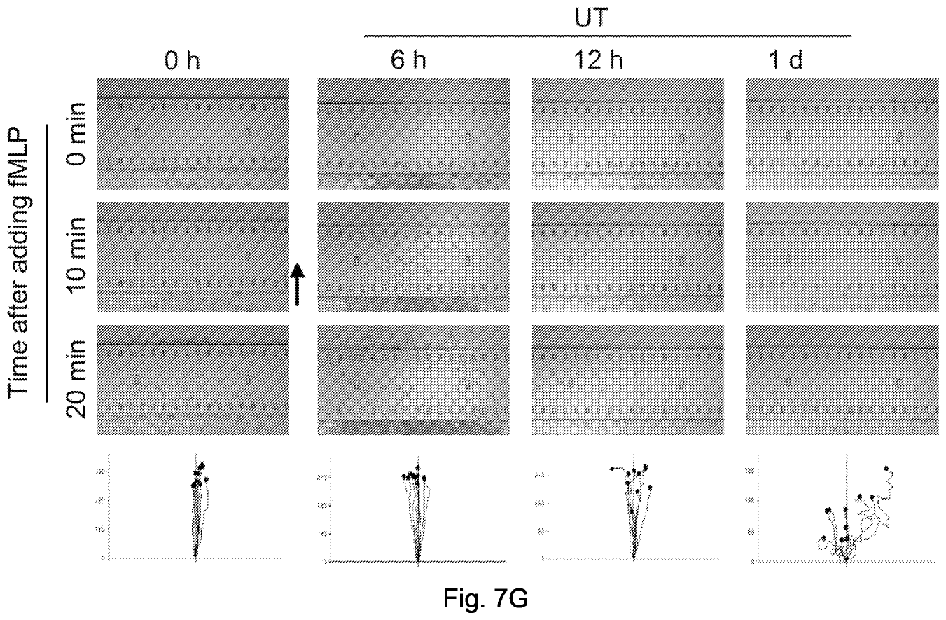

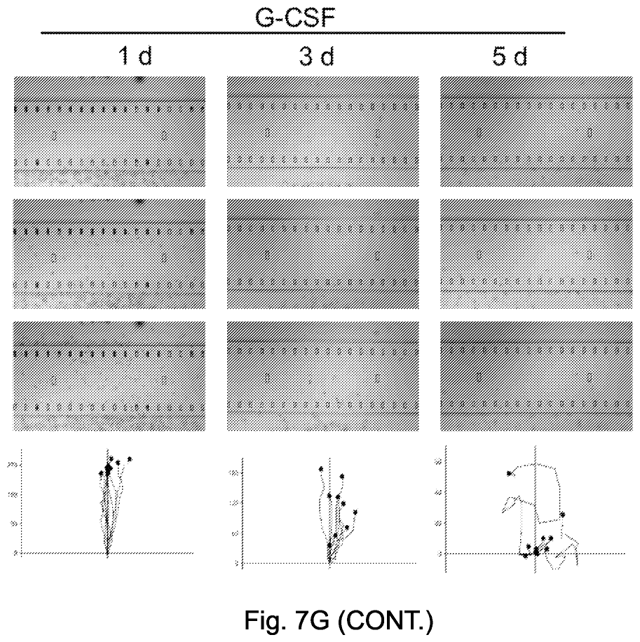

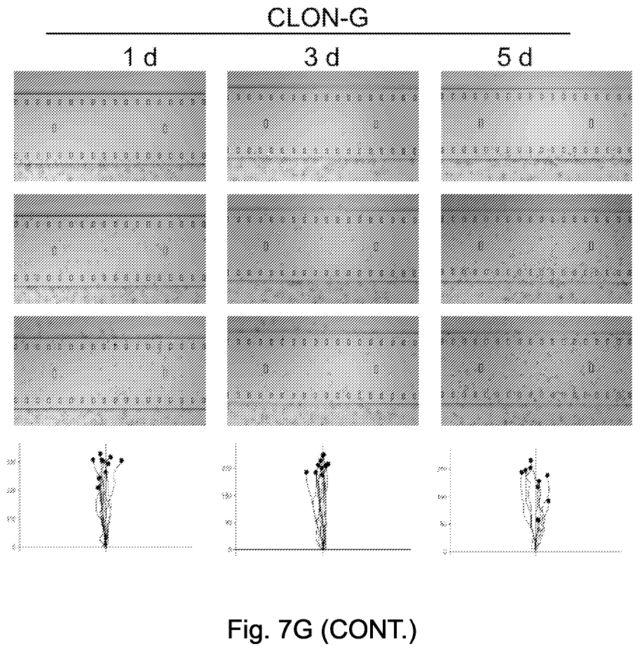

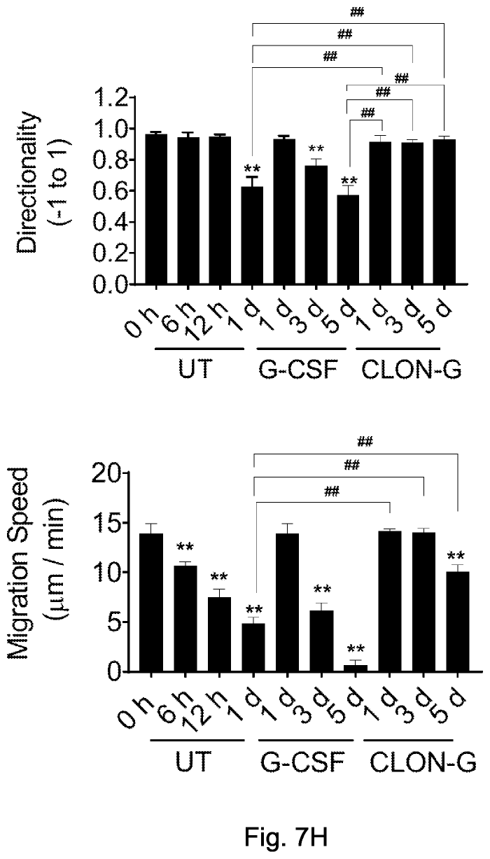

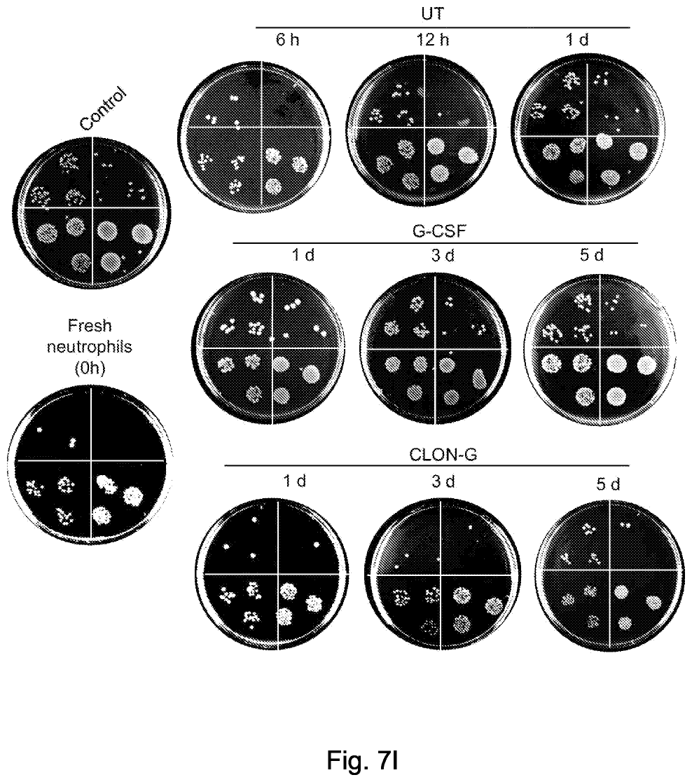

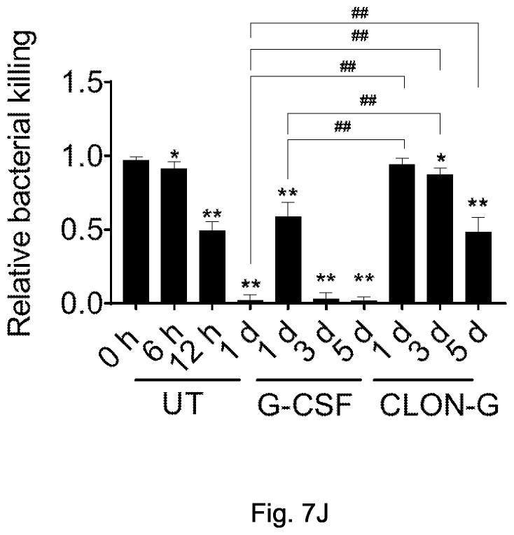

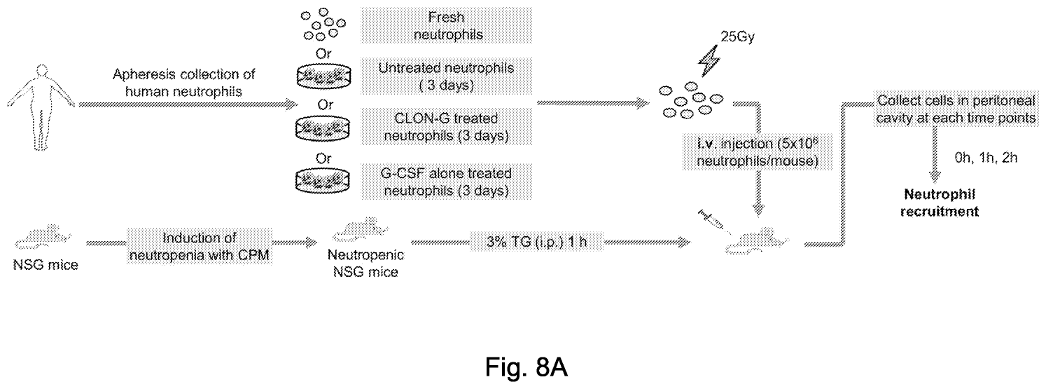

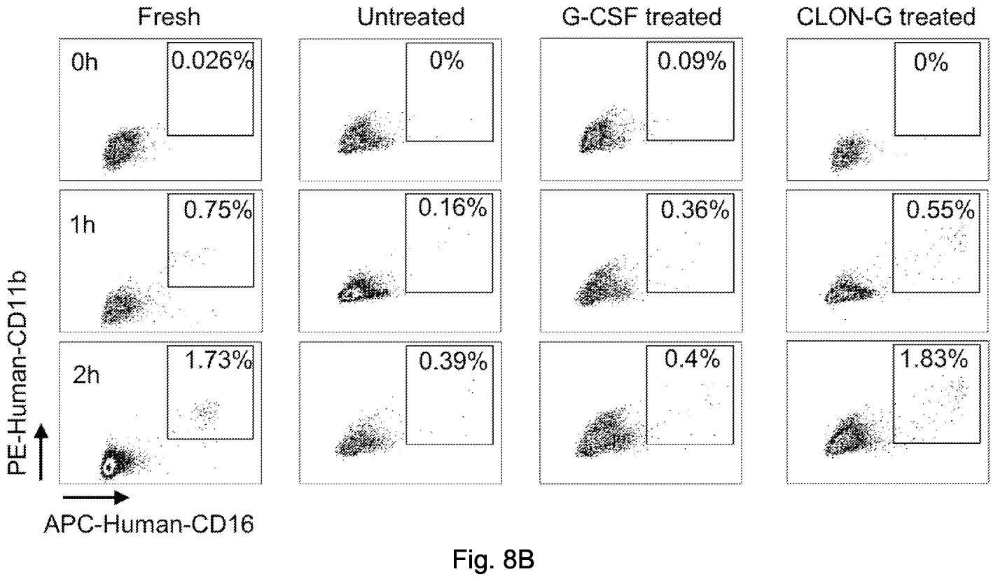

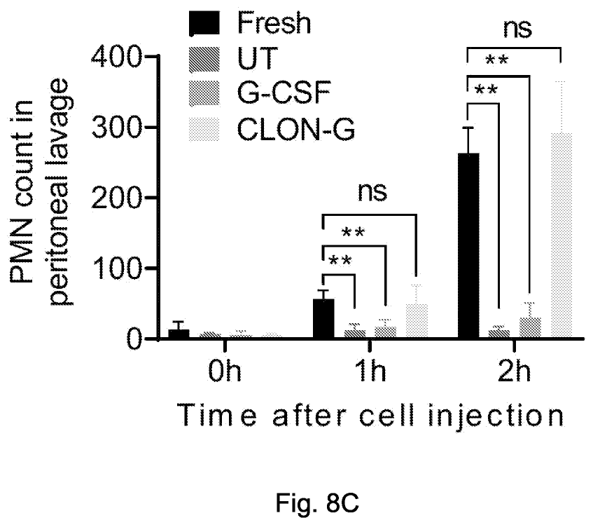

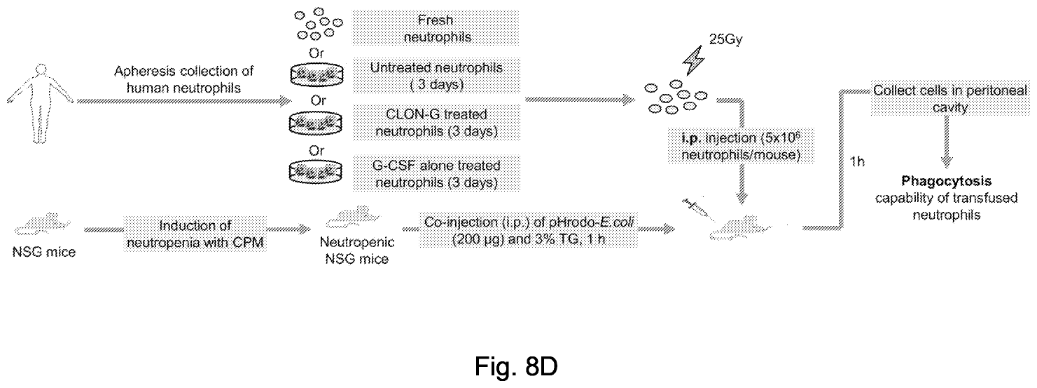

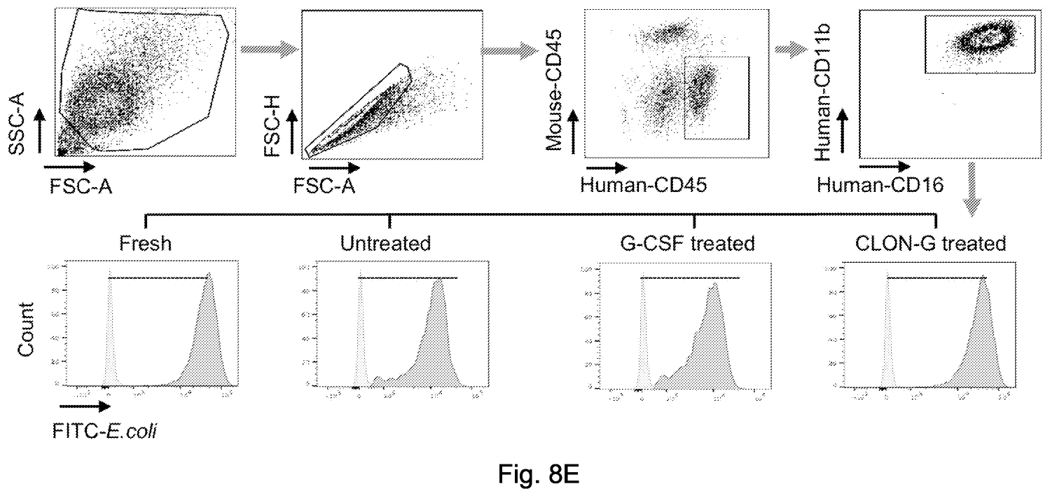

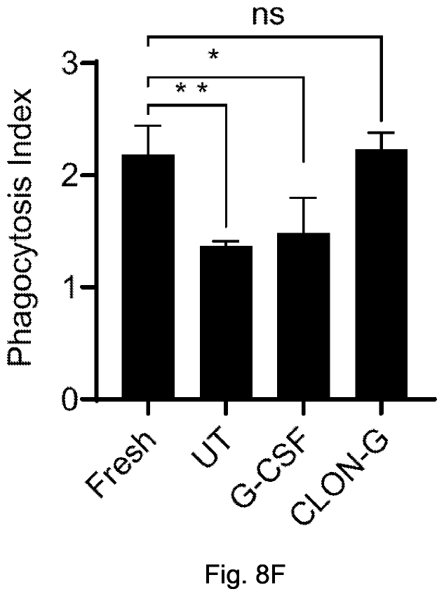

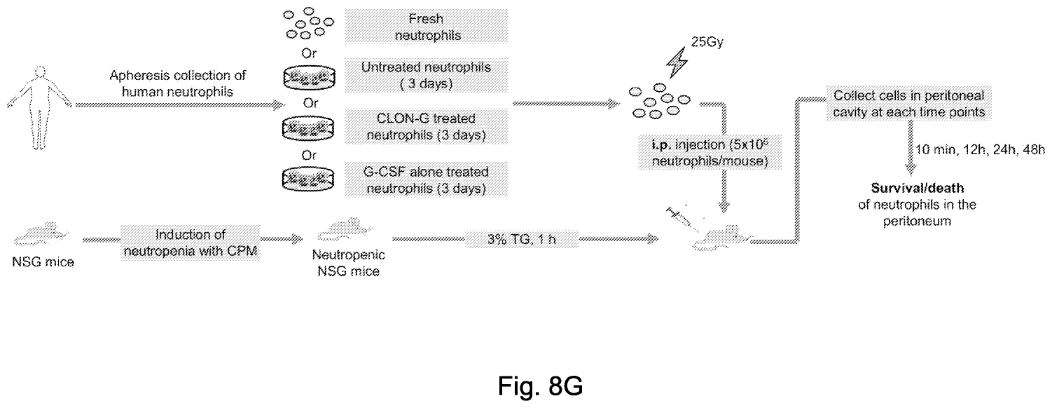

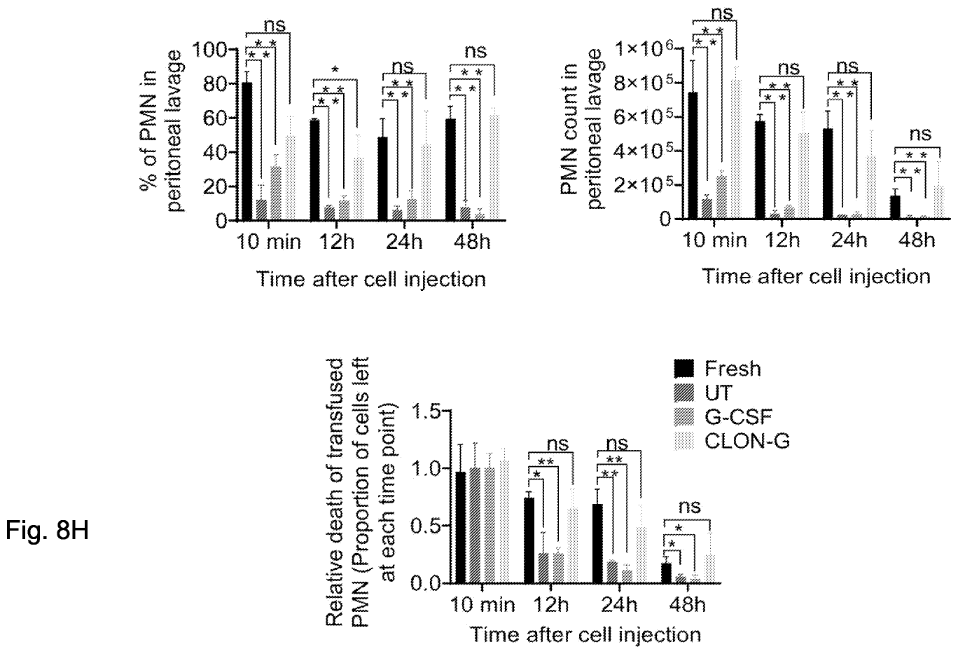

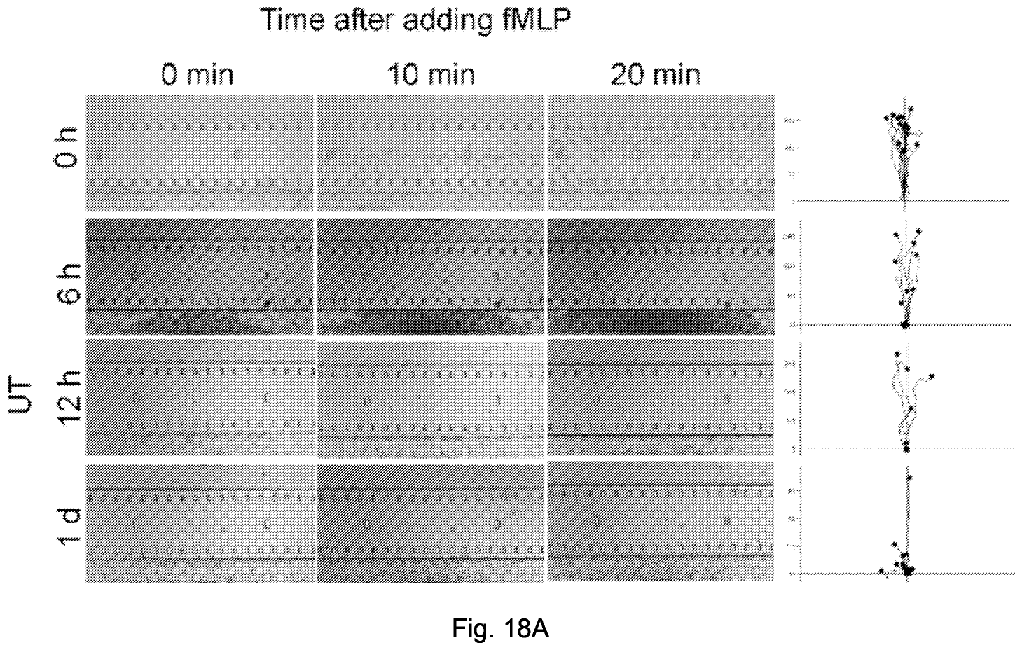

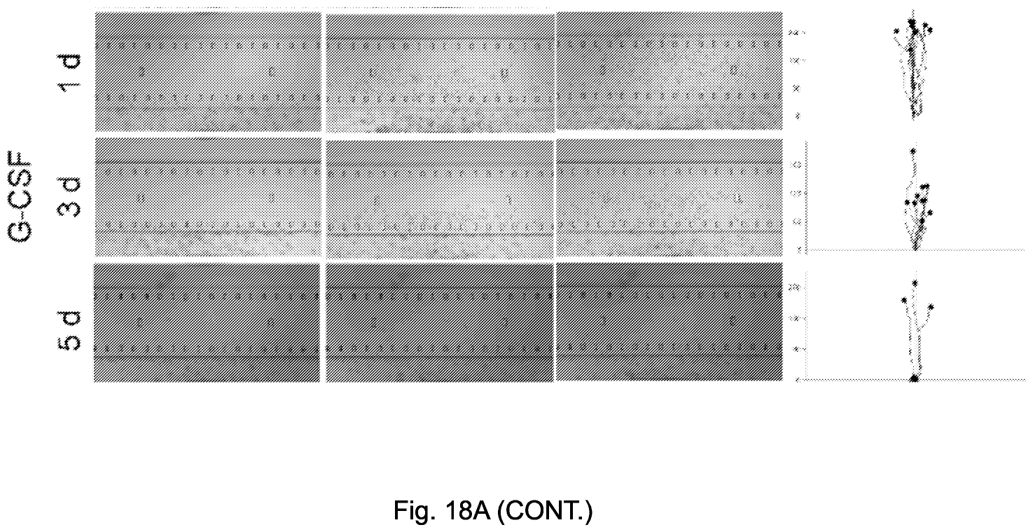

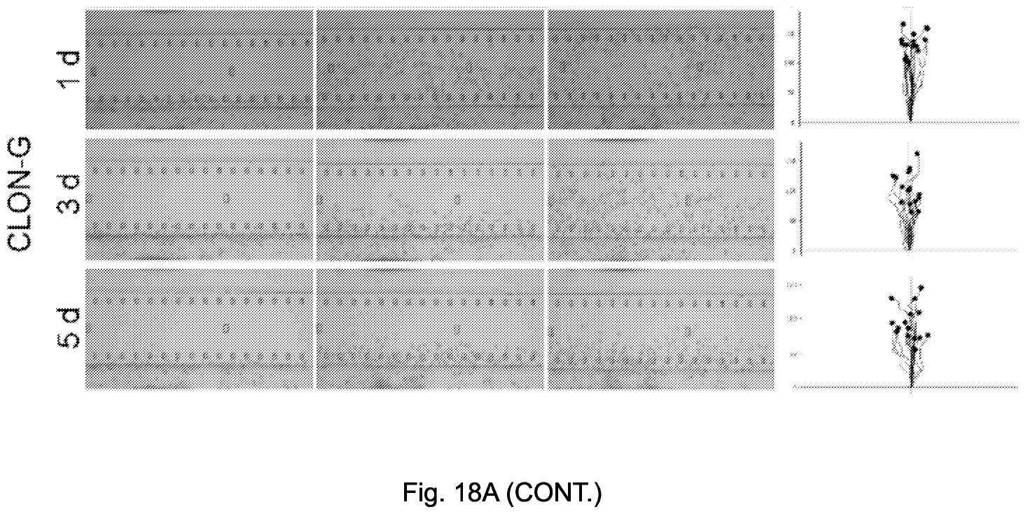

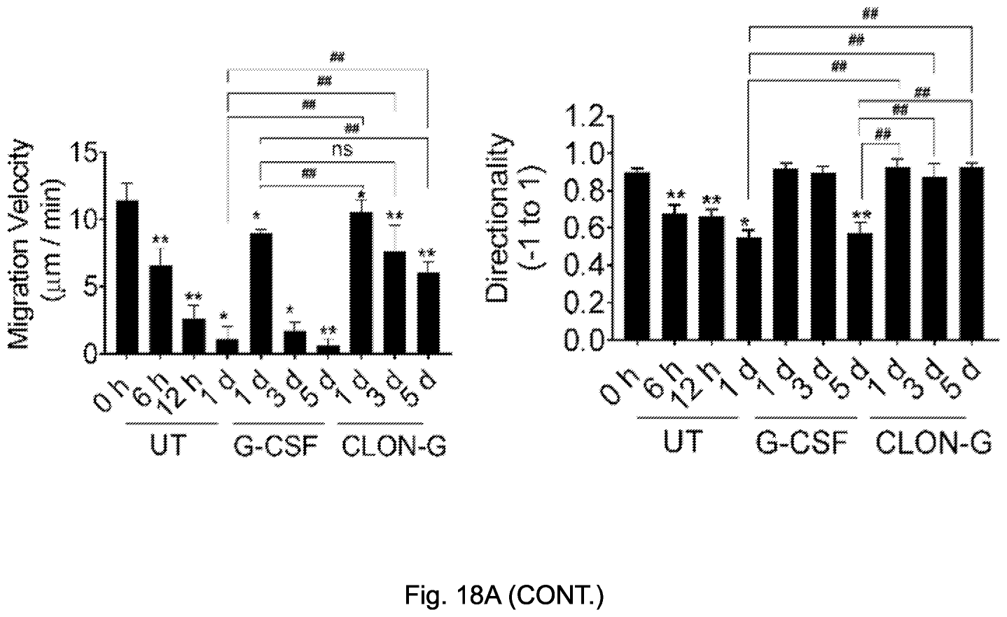

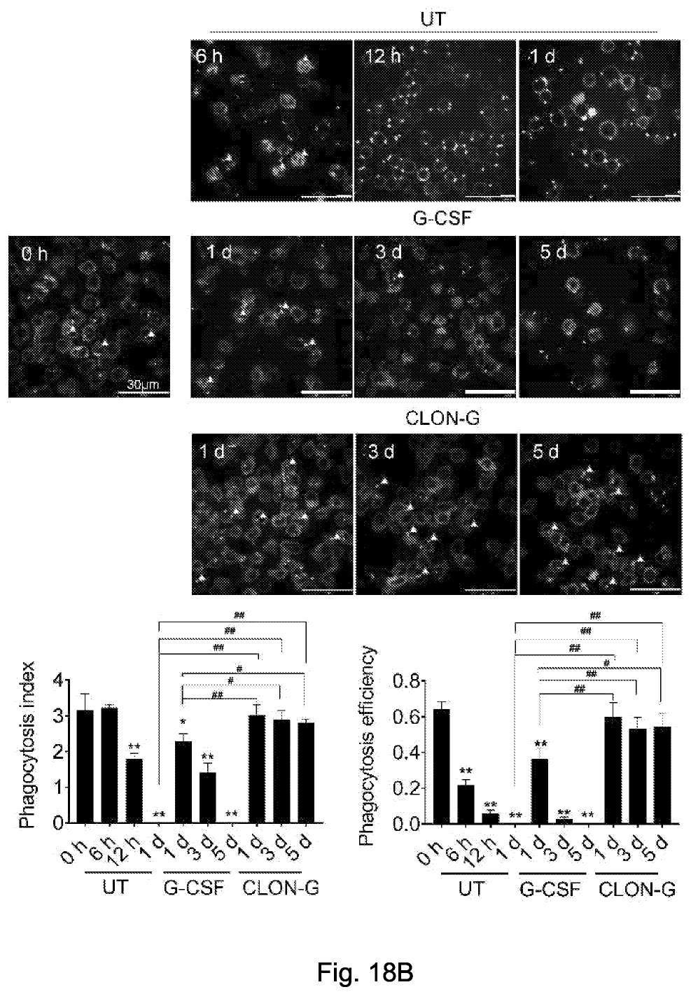

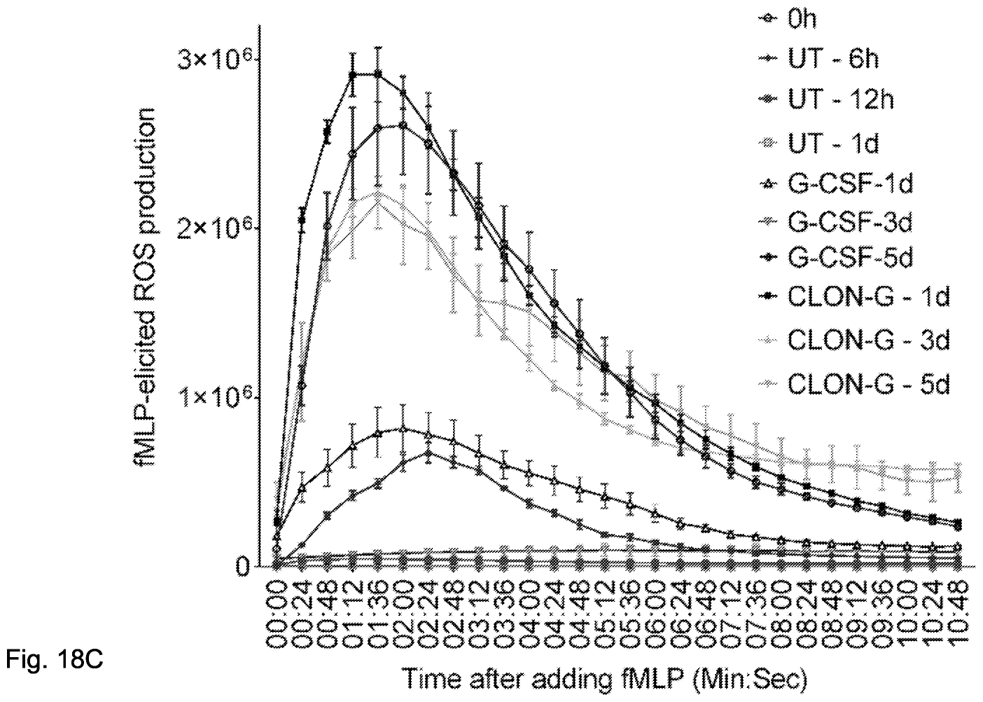

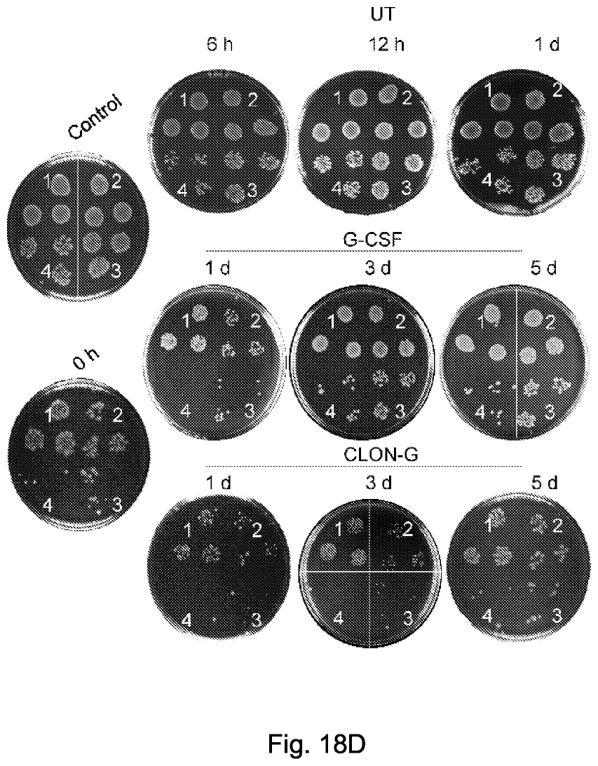

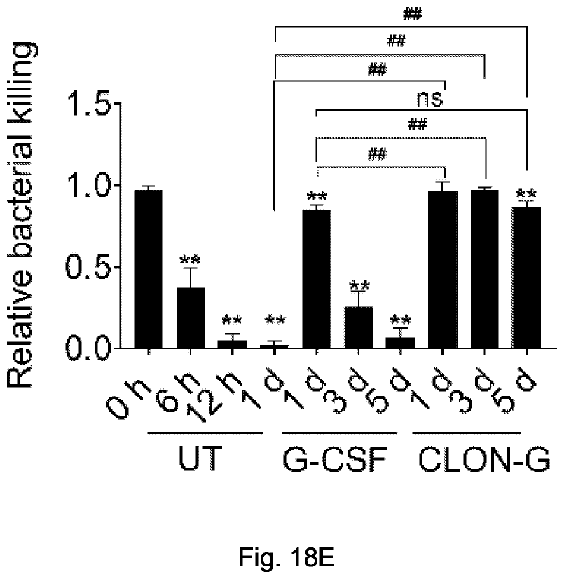

In some embodiments of any of the aspects, the neutrophil, or population thereof, produced using the compositions, cell culture media, and/or methods described herein exhibits enhanced or prolonged functionality compared to untreated neutrophils or compared to neutrophils treated with G-CSF alone. Non-limiting examples of neutrophil functionality include in vitro or in vivo chemotaxis, recruitment, phagocytosis (e.g., as measured by phagocytic index or phagocytic efficacy), nicotinamide adenine dinucleotide phosphate (NADPH) oxidase activation, and/or microbial killing (see e.g., FIG. 3, FIG. 7, FIG. 8, FIG. 18). In some embodiments of any of the aspects, the neutrophil, or population thereof, produced using the compositions, cell culture media, and/or methods described herein exhibits at least one neutrophil functionality that is increased by at least 50%, at least 60%, at least 70%, at least 80%, at least 90%, at least 100%, at least 110%, at least 120%, at least 130%, at least 140%, at least 150%, at least 160%, at least 170%, at least 180%, at least 190%, at least 200%, at least 210%, at least 220%, at least 230%, at least 240%, at least 250%, at least 260%, at least 270%, at least 280%, at least 290%, at least 300%, at least 310%, at least 320%, at least 330%, at least 340%, at least 350%, at least 360%, at least 370%, at least 380%, at least 390%, at least 400%, at least 410%, at least 420%, at least 430%, at least 440%, at least 450%, at least 460%, at least 470%, at least 480%, at least 490%, at least 500%, 100%-200%, 200%-300%, 300%-400%, 400%-500%, or more, compared to the same neutrophil functionality assessed in untreated neutrophils or neutrophils treated with G-CSF alone.

In some embodiments of any of the aspects, the neutrophil, or population thereof, produced using the compositions, cell culture media, and/or methods described herein exhibits at least one neutrophil functionality that is not significantly decreased compared to fresh neutrophils. In some embodiments of any of the aspects, the neutrophil, or population thereof, produced using the compositions, cell culture media, and/or methods described herein exhibits at least one neutrophil functionality that is at most at most 1%, at most 2%, at most 3%, at most 4%, at most 5%, at most 6%, at most 7%, at most 8%, at most 9%, at most 10%, at most 11%, at most 12%, at most 13%, at most 14%, at most 15%, at most 16%, at most 17%, at most 18%, at most 19%, at most 20% decreased compared to the same neutrophil functionality assessed in fresh neutrophils.

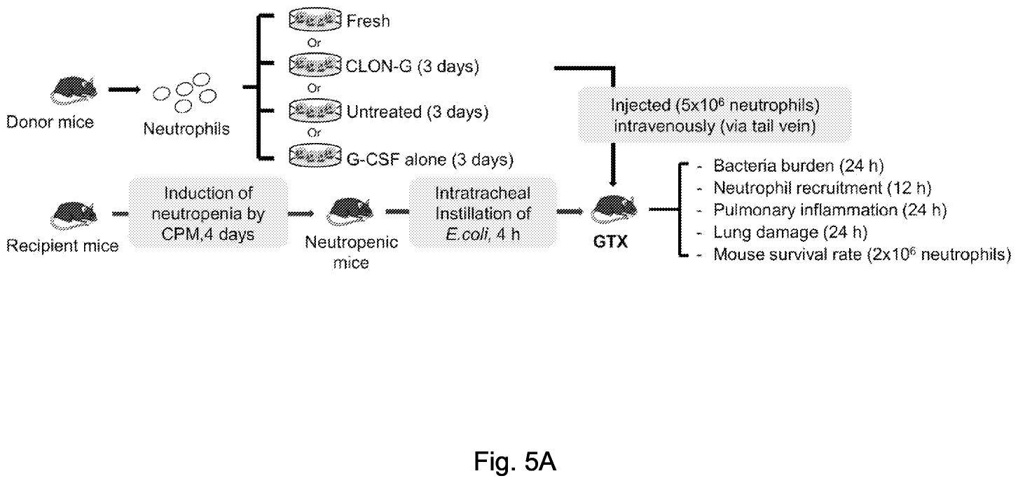

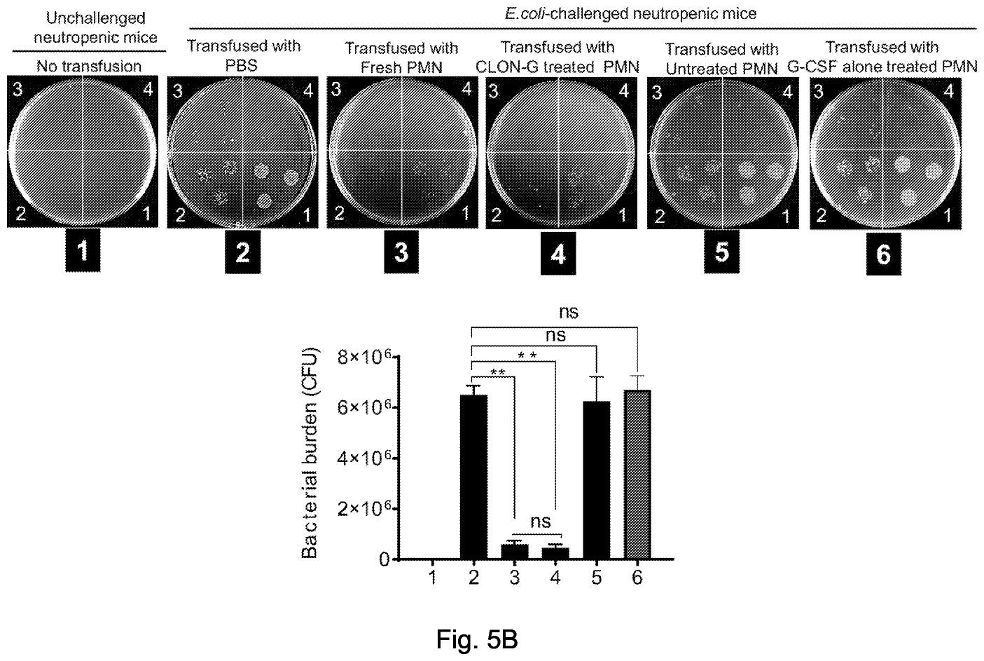

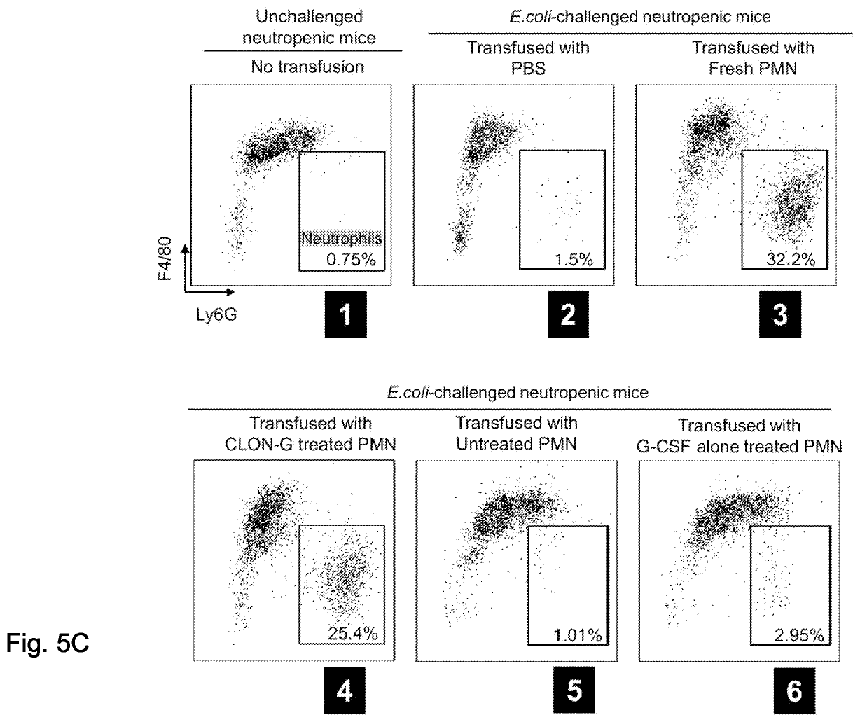

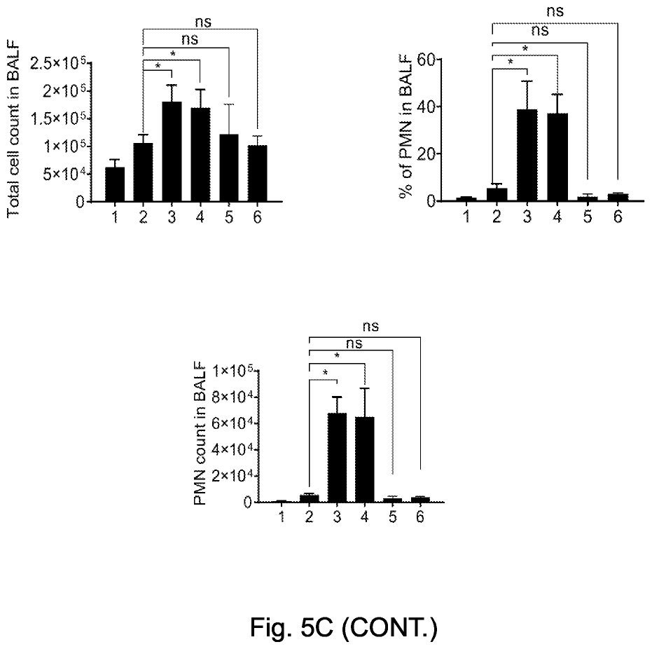

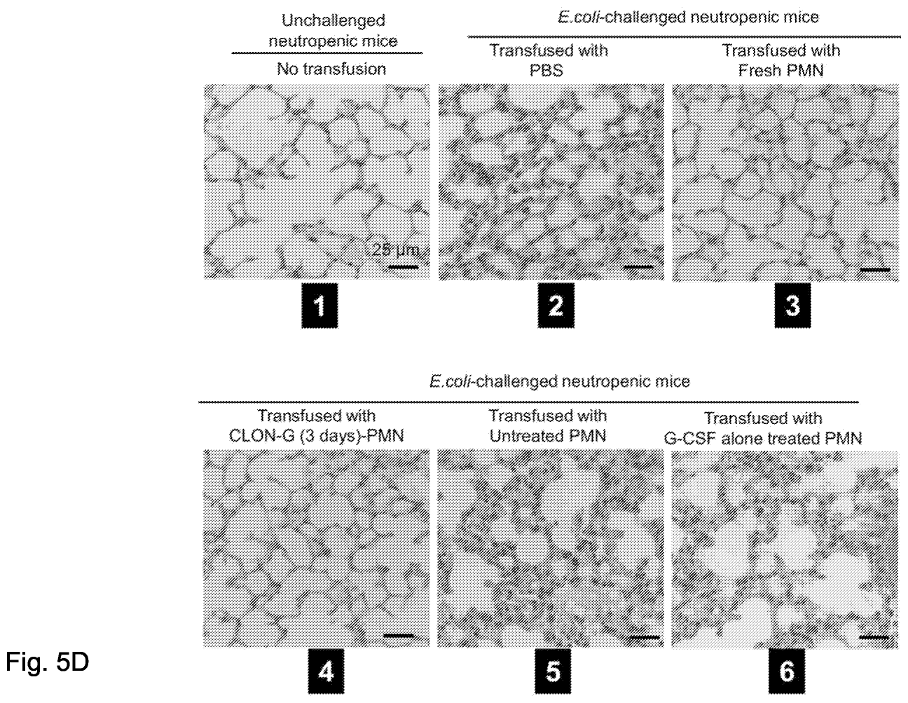

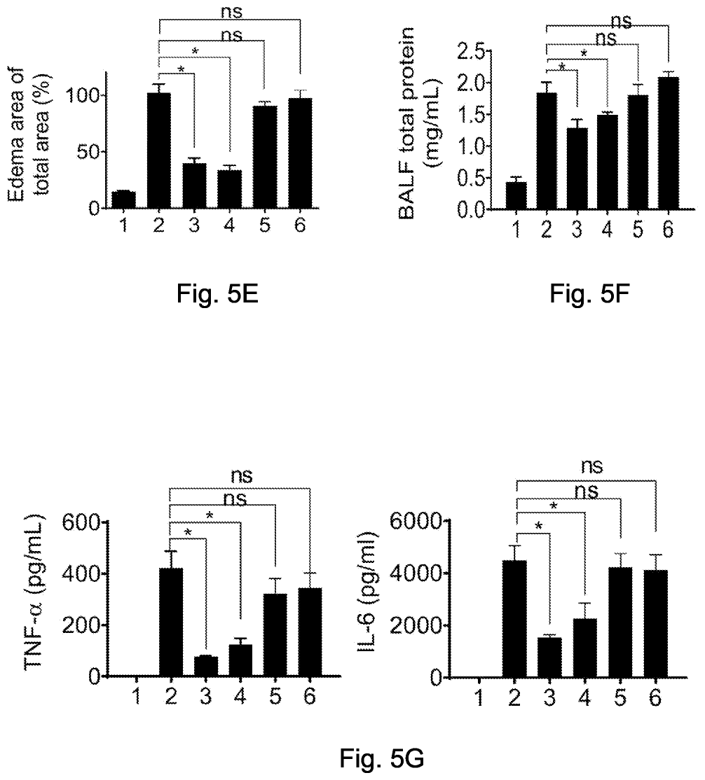

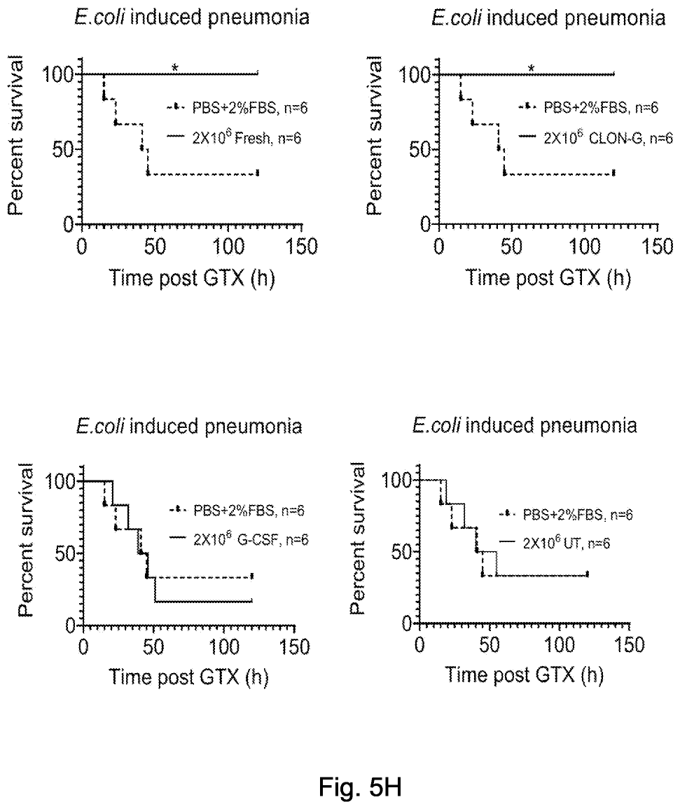

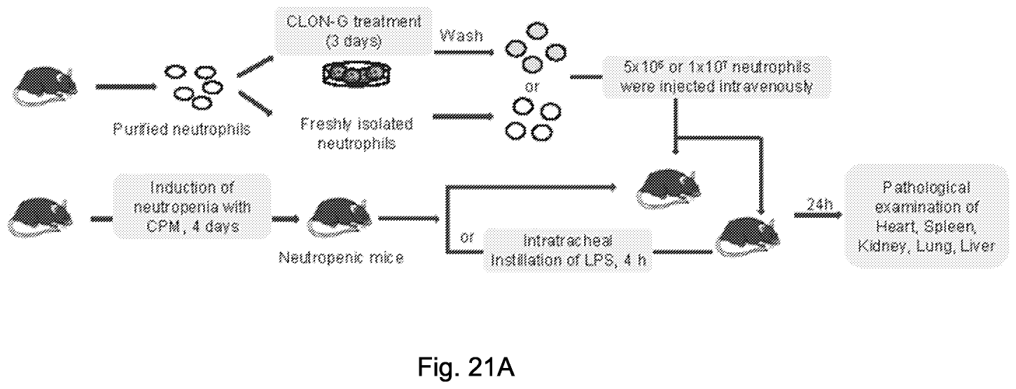

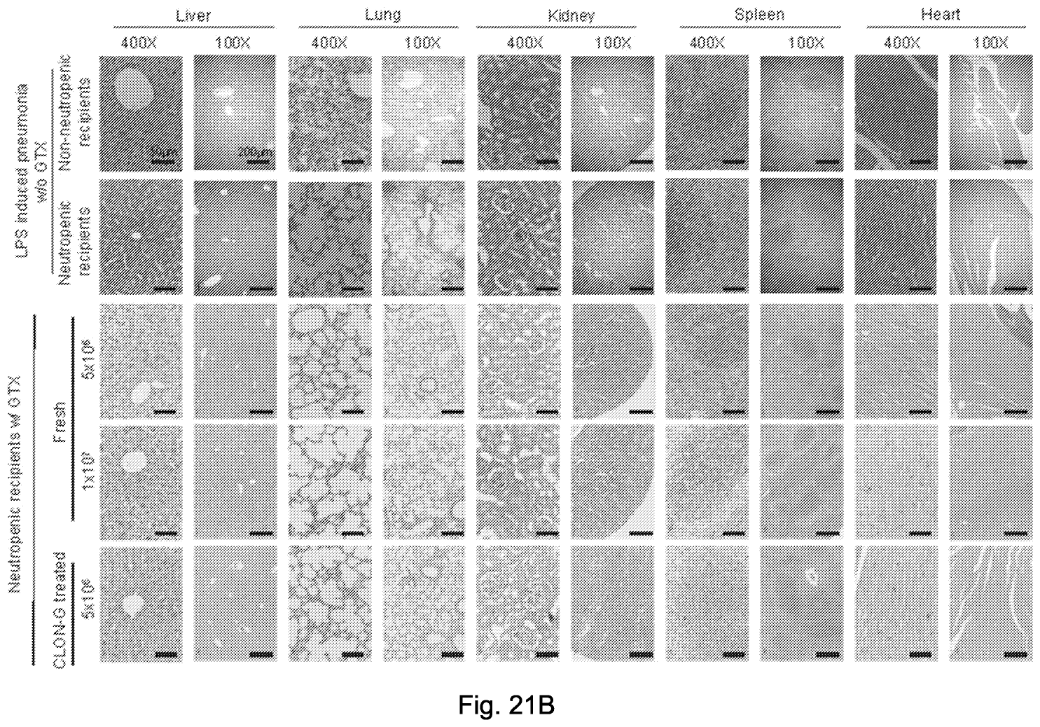

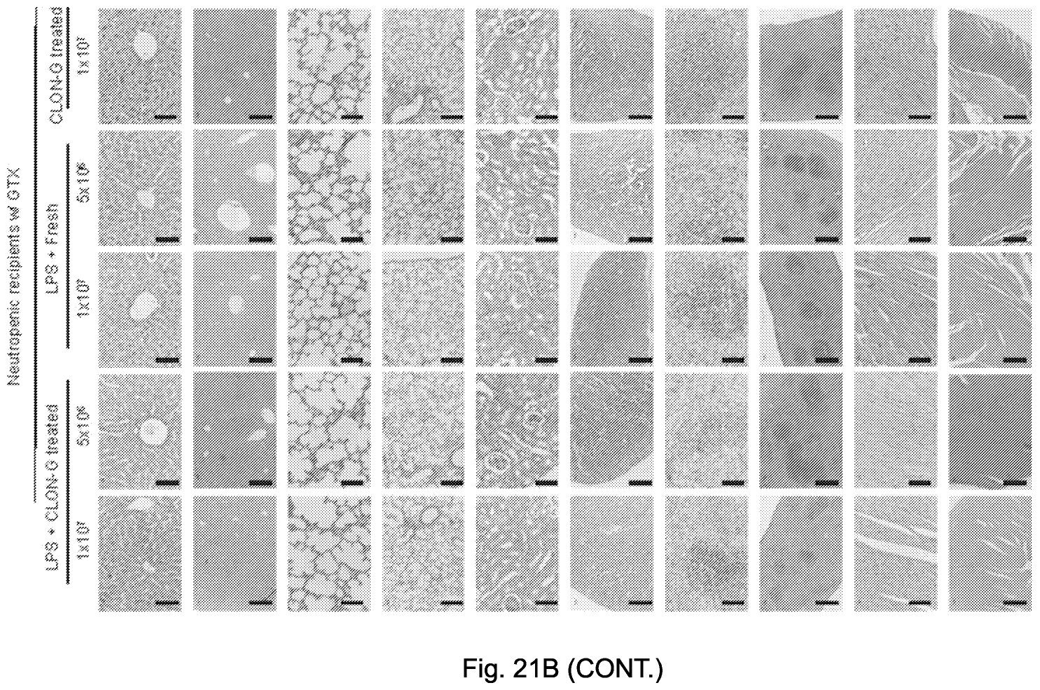

In some embodiments of any of the aspects, the neutrophil, or population thereof, produced using the compositions, cell culture media, and/or methods described herein is not hyper-activated and/or does not cause significant tissue damage, edema, and/or elevated inflammatory cytokines (see e.g., FIG. 5, FIG. 21).

### Compositions and Cell Culture Media

Described herein are compositions and associated cell culture media that increase neutrophil lifespan. In one aspect, described herein is a composition comprising: (a) a caspase inhibitor; (b) a lysosomal membrane permeabilization (LMP) inhibitor; (c) an antioxidant; and (d) a growth factor, or any combination thereof (see e.g., Table 3). In some embodiments, the composition further comprises a necroptosis inhibitor. In some embodiments, the composition is in the form a solid, powder, gel, or liquid. In some embodiments, each of the caspase inhibitor, LMP inhibitor, antioxidant, growth factor, and/or necroptosis inhibitor is in the form a solid, powder, gel, or liquid. In some embodiments, the powder is a freeze-dried (i.e., lyophilized) or spray-dried powder. The caspase inhibitor, LMP inhibitor, antioxidant, growth factor, and/or necroptosis inhibitor can be supplied separately or mixed together in the composition. In some embodiments, the composition is attached to a solid substrate that can be added to a cell culture medium, such as a soluble filter paper.

In some embodiments, the composition further comprises a container. Non-limiting examples of containers that can comprise the composition include: sample collection containers such as blood collection tubes (with or without clot activator(s) or anti-coagulant(s)) or apheresis bags, cell culture plates, cell culture vessels, flasks, multi-well plates, test tubes, centrifuge tubes, cell culture media container, and the like. In some embodiments, the container comprises at least two separate compartments for containing the caspase inhibitor, LMP inhibitor, antioxidant, growth factor, and/or necroptosis inhibitor. The caspase inhibitor, LMP inhibitor, antioxidant, growth factor, and/or necroptosis inhibitor can be present in the same or separate compartments of the container. In some embodiments, the composition is present in a kit, as described further herein.

In some embodiments, the composition or components thereof (e.g., in the form of a solid, powder, or gel) can be dissolved in a liquid (e.g., an aqueous solution, a cell culture medium). In some embodiments, the composition further comprises cell culture medium. In some embodiments, the composition further comprises serum.

In another aspect, described herein is a cell culture medium comprising: (a) a caspase inhibitor; (b) a lysosomal membrane permeabilization (LMP) inhibitor; (c) an antioxidant; (d) a growth factor; and (e) a necroptosis inhibitor, or any combination thereof (see e.g., Table 3). In another aspect, described herein is a cell culture medium or composition comprising a combination from Table 3.

Table 3: Exemplary Component Combinations for a Composition or Cell Culture Medium (“X” indicates inclusion in the composition or cell culture medium)

TABLE 3
Exemplary Component Combinations for a Composition
or Cell Culture Medium (“X” indicates
inclusion in the composition or cell culture medium)

| caspase | LMP | | growth | necroptosis | cell culture | |
|---|---|---|---|---|---|---|
| inhibitor | inhibitor | antioxidant | factor | inhibitor | medium | serum |
| X | | | | | | |
| | X | | | | | |
| X | X | | | | | |
| | | X | | | | |
| X | | X | | | | |
| | X | X | | | | |
| X | X | X | | | | |
| | | | X | | | |
| X | | | X | | | |
| | X | | X | | | |
| X | X | | X | | | |
| | | X | X | | | |
| X | | X | X | | | |
| | X | X | X | | | |
| X | X | X | X | | | |
| | | | | X | | |
| X | | | | X | | |
| | X | | | X | | |
| X | X | | | X | | |
| | | X | | X | | |
| X | | X | | X | | |
| | X | | | X | | |
| X | X | X | | X | | |
| | | | X | X | | |
| X | | | X | X | | |
| | X | | X | X | | |
| X | X | | X | X | | |
| | | X | X | X | | |
| X | | X | X | X | | |
| | X | X | X | X | | |
| X | X | X | X | X | | |
| | | | | | X | |
| X | | | | | X | |
| | X | | | | X | |
| X | X | | | | X | |
| | | X | | | X | |
| X | | X | | | X | |
| | X | X | | | X | |
| X | X | X | | | X | |
| | | | X | | X | |
| X | | | X | | X | |
| | X | | X | | X | |
| X | X | | X | | X | |
| | | X | X | | X | |
| X | | X | X | | X | |
| | X | X | X | | X | |
| X | X | X | X | | X | |
| | | | | X | X | |
| X | | | | X | X | |
| | X | | | X | X | |
| | X | | | X | X | |
| | | X | | X | X | |
| | | X | | X | X | |
| | X | X | | X | X | |
| X | X | X | | X | X | |
| | | | X | X | X | |
| X | | | X | X | X | |
| | X | | X | X | X | |
| X | X | | X | X | X | |
| | | X | X | X | X | |
| | | X | X | X | X | |
| | X | X | X | X | X | |
| X | X | X | X | X | X | |
| | | | | | | X |
| X | | | | | | X |
| | X | | | | | X |
| X | X | | | | | X |
| | | X | | | | X |
| X | | X | | | | X |
| | X | X | | | | X |
| | X | X | | | | X |
| | | | X | | | X |
| | | | X | | | X |
| | X | | X | | | X |
| X | X | | X | | | X |
| | | X | X | | | X |
| X | | X | X | | | X |
| | X | X | X | | | X |
| X | X | X | X | | | X |
| | | | | X | | X |
| | | | | X | | X |
| | X | | | X | | X |
| X | X | | | X | | X |
| | | X | | X | | X |
| X | | X | | X | | X |
| | X | X | | X | | X |
| X | X | X | | X | | X |
| | | | X | X | | X |
| X | | | X | X | | X |
| | X | | X | X | | X |
| X | X | | X | X | | X |
| | | X | X | X | | X |
| X | | X | X | X | | X |
| | X | X | X | X | | X |
| X | X | X | X | X | | X |
| | | | | | X | X |
| X | | | | | X | X |
| | X | | | | X | X |
| X | X | | | | X | X |
| | | X | | | X | X |
| X | | X | | | X | X |
| | X | X | | | X | X |
| X | X | X | | | X | X |
| | | | X | | X | X |
| X | | | X | | X | X |
| | X | | X | | X | X |
| X | X | | X | | X | X |
| | | X | X | | X | X |
| X | | X | X | | X | X |
| | X | X | X | | X | X |
| X | X | X | X | | X | X |
| | | | | X | X | X |
| X | | | | X | X | X |
| | X | | | X | X | X |
| X | X | | | X | X | X |
| | | X | | X | X | X |
| X | | X | | X | X | X |
| | X | X | | X | X | X |
| X | X | X | | X | X | X |
| | | | X | X | X | X |
| X | | | X | X | X | X |
| | X | | X | X | X | X |
| X | X | | X | X | X | X |
| | | X | X | X | X | X |
| X | | X | X | X | X | X |
| | X | X | X | X | X | X |
| X | X | X | X | X | X | X |

In some embodiments of any of the aspects, the composition or cell culture medium described herein comprises at least one caspase inhibitor. In some embodiments, the composition or cell culture medium described herein comprises one caspase inhibitor. In some embodiments, the composition or cell culture medium described herein comprises a plurality of different caspase inhibitors, e.g., at least 2, 3, 4, 5, 6, 7, 8, 9, 10, or more different caspase inhibitors, which can be provided at the same or different concentrations.

Caspases are a family of protease enzymes playing essential roles in programmed cell death (e.g., apoptosis). Caspases exhibit specific cysteine protease activity—a cysteine in the caspase active site nucleophilically attacks and cleaves a target protein only after an aspartic acid residue. In some embodiments of any of the aspects, the caspase inhibitor is an inhibitor of human caspase(s) and/or murine caspase(s). In some embodiments of any of the aspects, the caspase inhibitor is an inhibitor of murine or human Caspase-1/4 (e.g., Z-YVAD-FMK, Belnacasan (VX-765)); murine Caspase-2 (e.g., Z-VDVAD-FMK); murine Caspase-3 (e.g., Z-DQMD-FMK, Boc-D-FMK, Ivachtin); murine or human Caspase-3 (e.g., Z-DEVD-FMK); murine Caspase-4 (e.g., Belnacasan (VX-765)); murine Caspase-5 (e.g., Z-WEHD-FMK); murine Caspase-6 (e.g., Z-VEID-FMK); murine or human Caspase-8 (e.g., Z-IETD-FMK); murine or human Caspase-9 (e.g., Ac-LEHD-CHO); murine Caspase-11 (e.g., Wedelolactone); or any combination thereof.

In some embodiments of any of the aspects, the caspase inhibitor is a pan-caspase inhibitor. As used herein, the term “pan-caspase inhibitor” refers to a compound that can inhibit all or a majority of the known caspases (e.g., human caspases 1-11). In some embodiments of any of the aspects, the pan caspase inhibitor is an inhibitor of all or a majority of human caspase(s) and/or murine caspase(s). In some embodiments of any of the aspects, the pan caspase inhibitor is selected from the group consisting of: Q-VD-Oph; Z-VAD-FMK; Emricasan; and Ac-DEVD-CHO.

In some embodiments of any of the aspects, the pan-caspase inhibitor is Q-VD-Oph, which is also referred to as Quinoline-Val-Asp-Difluorophenoxymethylketone (see e.g., Formula I). Q-VD-Oph can be present in its hydrate form: 5-(2,6-Difluorophenoxy)-3-[[3-methyl-1-oxo-2-[(2-quinolinylcarbonyl)amino]butyl]amino]-4-oxo-pentanoic acid hydrate. Q-VD-Oph is a pan-caspase inhibitor with IC50 ranged from 25 to 400 nM for caspases such as human caspases 1, 3, 8, and 9. Q-VD-OPh can be more effective in preventing apoptosis than the inhibitors ZVAD-fmk and Boc-D-fink. Q-VD-OPh is also equally effective in preventing apoptosis mediated by the three major apoptotic pathways, caspase 9/3, caspase 8/10, and caspase 12. In addition to the increased effectiveness, Q-VD-OPh is not toxic to cells even at extremely high concentrations. See e.g., Caserta et al., “Q-VD-OPh, a broad spectrum caspase inhibitor with potent antiapoptotic properties,” Apoptosis. 2003, 8(4): 345-52, the contents of which are incorporated herein by reference in their entirety. In some embodiments of any of the aspects, the pan-caspase inhibitor is Q-VD-Oph (SELLECKCHEM, S7311) or an analog thereof.

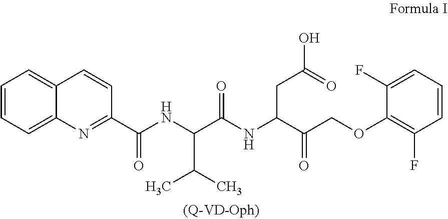 [SMILES: CC(C)C(NC(=O)c1ccc2ccccc2n1)C(=O)NC(CC(=O)O)C(=O)COc1c(F)cccc1F]

In some embodiments of any of the aspects, the pan-caspase inhibitor is Emricasan, which is also referred to as IDN-6556, PF-03491390, or N-[2-(1,1-dimethylethyl)phenyl]-2-oxoglycyl-N-[(1S)-1-(carboxymethyl)-2-oxo-3-(2,3,5,6-tetrafluorophenoxy)propyl]-L-Alaninamide (see e.g., Formula II). Emricasan is a potent, irreversible, pan-caspase inhibitor and has antiapoptotic and anti-inflammatory effects. Emricasan has sub- to nanomolar activity in vitro.

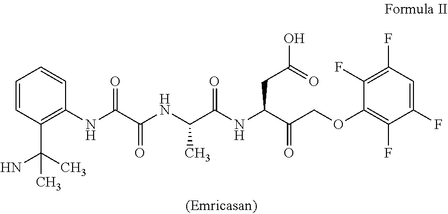 [SMILES: C[C@H](NC(=O)C(=O)Nc1ccccc1C(C)(C)N)C(=O)N[C@@H](CC(=O)O)C(=O)COc1c(F)c(F)cc(F)c1F]

In some embodiments of any of the aspects, the pan-caspase inhibitor is Z-VAD-FMK, which is also referred to as carbobenzoxy-valyl-alanyl-aspartyl-[O-methyl]-fluoromethylketone, or an analog thereof (see e.g., Formula III). Z-VAD-FMK is a cell-permeable pan caspase inhibitor that irreversibly binds to the catalytic site of caspase proteases and can inhibit induction of apoptosis. Z-VAD-FMK potently inhibits human caspase-1 to caspase-10 with the exception of caspase-2; Z-VAD-FMK also inhibits murine caspases, including caspase-1, caspase-3, and caspase-11, the ortholog of human caspase-4 and caspase-5. Z-VAD-FMK inhibits caspases by irreversibly binding to their catalytic site. By inhibiting the activity of multiple caspases, Z-VAD-FMK can block many different biological processes including inflammasome activation and the induction of apoptosis leading to increased cell survival in many different cell types. In some embodiments of any of the aspects, the concentration of Z-VAD-FMK is about 10 μg/ml (20 μM).

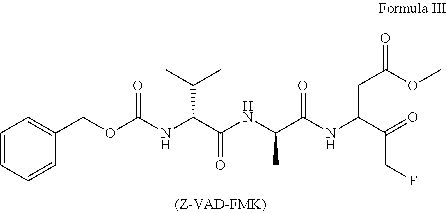 [SMILES: COC(=O)CC(NC(=O)[C@@H](C)NC(=O)[C@H](NC(=O)OCc1ccccc1)C(C)C)C(=O)CF]

In some embodiments of any of the aspects, the pan-caspase inhibitor is Ac-DEVD-CHO, or an analog thereof (see e.g., Formula IV). Ac-DEVD-CHO is also referred to as 169332-60-9; N-acetyl-asp-glu-val-asp-al; 184179-08-6; Ac-Asp-Glu-Val-Asp-Aldehyde; AC-ASP-GLU-VAL-ASP-H; CHEMBL417149; or (4S,7S,10S,13S)-7-(2-carboxyethyl)-4-(carboxymethyl)-13-formyl-10-isopropyl-2,5,8,11-tetraoxo-3,6,9,12-tetraazapentadecan-15-oic acid. Ac-DEVD-CHO is a synthetic peptide aldehyde with the PARP cleavage site DEVD, which is recognized by caspases. Ac-DEVD-CHO blocks PARP cleavage activity and inhibits caspase-3 (Ki=0.23 nM) and caspase-7 (Ki=1.6 nM) in a reversible manner. This inhibition is via the aldehyde group's interaction with the active site cysteine of these caspases. In some embodiments, Ac-DEVD-CHO is used as the trifluoroacetate salt of the molecule. In some embodiments of any of the aspects, the concentration of Z-VAD-FMK is about 0.23 nM to 1.6 nM.

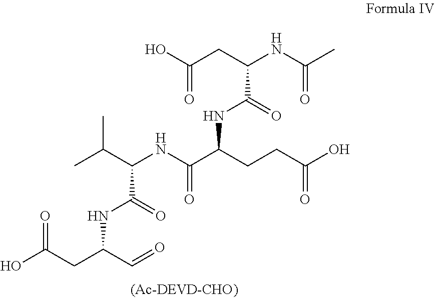 [SMILES: CC(=O)N[C@@H](CC(=O)O)C(=O)N[C@@H](CCC(=O)O)C(=O)N[C@H](C(=O)N[C@H](C=O)CC(=O)O)C(C)C]

In some embodiments of any of the aspects, the caspase inhibitor (e.g., Q-VD-Oph) is at a concentration of at least about 50 μM. In some embodiments of any of the aspects, the caspase inhibitor (e.g., Q-VD-Oph) is at a concentration of at least about 12.5 μM. In some embodiments of any of the aspects, the caspase inhibitor (e.g., Q-VD-Oph) is at a concentration of at least about 25 μM. In some embodiments of any of the aspects, the caspase inhibitor (e.g., Q-VD-Oph) is at a concentration of at least about 100 μM. In some embodiments of any of the aspects, the caspase inhibitor (e.g., Q-VD-Oph) is at a concentration of at least about 200 μM. In some embodiments of any of the aspects, the caspase inhibitor (e.g., Emricasan) is at a concentration of at least about 10 μM. In some embodiments of any of the aspects, the caspase inhibitor (e.g., Emricasan) is at a concentration of at least about 25 μM.

In some embodiments, the caspase inhibitor (e.g., Q-VD-Oph, Emricasan, Z-VAD-FMK, or Ac-DEVD-CHO) is at a concentration of at least 0.1 nM, at least 0.2 nM, at least 0.3 nM, at least 0.4 nM, at least 0.5 nM, at least 0.6 nM, at least 0.7 nM, at least 0.8 nM, at least 0.9 nM, at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 μM, at least 1.25 μM, at least 1.5 μM, at least 1.75 μM, at least 2.0 μM, at least 2.5 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, at least 100 μM, at least 110 μM, at least 120 μM, at least 130 μM, at least 140 μM, at least 150 μM, at least 160 μM, at least 170 μM, at least 180 μM, at least 190 μM, at least 200 μM, at least 250 μM, at least 300 μM, at least 350 μM, at least 400 μM, at least 450 μM, at least 500 μM, or more. In some embodiments, the caspase inhibitor (e.g., Q-VD-Oph, Emricasan, Z-VAD-FMK, or Ac-DEVD-CHO) is at a concentration of 0.1 nM-1 nM, 1 nM-10 nM, 10 nM-50 nM, 50 nM-100 nM, 100 nM-500 nM, 500 nM-1 μM, 1 μM-5 μM, 5 μM-10 μM, 10 μM-25 μM, 12.5 μM-25 μM, 25 μM-50 μM, 50 μM-100 μM, 100 μM-200 μM, or 200 μM-500 μM.

In some embodiments of any of the aspects, the composition or cell culture medium described herein comprises at least one LMP inhibitor (i.e., at least one compound that inhibits lysosomal membrane permeabilization). In some embodiments, the composition or cell culture medium described herein comprises one LMP inhibitor. In some embodiments, the composition or cell culture medium described herein comprises two different LMP inhibitors. In some embodiments, the composition or cell culture medium described herein comprises a plurality of different LMP inhibitors, e.g., at least 2, 3, 4, 5, 6, 7, 8, 9, 10, or more different LMP inhibitors, which can be provided at the same or different concentrations.

The lysosome is a membrane-bound organelle comprising hydrolytic enzymes and a luminal pH of ˜4.5-5.0. Lysosomal membrane permeabilization is one mechanism for the induction of cell death. Complete disruption of lysosomes provokes uncontrolled cell death by necrosis. Partial and selective LMP induces the controlled dismantling of the cell by apoptosis; see e.g., Boya et al., “Lysosomal membrane permeabilization in cell death,” Oncogene 27, 6434-6451 (2008), the contents of which are incorporated herein by reference in their entirety. In some embodiments of any of the aspects, the LMP inhibitor is an inhibitor of human LMP and/or murine LMP. In some embodiments of any of the aspects, the LMP inhibitor is Heat Shock Protein 70 (Hsp70) and/or deferoxamine mesylate (DFO).

In some embodiments of any of the aspects, the LMP inhibitor is Heat Shock Protein 70 (Hsp70). Hsp70 proteins are central components of the cellular network of molecular chaperones and folding catalysts. Hsp70 proteins assist a large variety of protein folding processes in the cell by transient association of their substrate binding domain with short hydrophobic peptide segments within their substrate proteins. Hsp70 is a potent survival protein whose depletion triggers massive caspase-independent cell death. Hsp70 exerts its prosurvival function by inhibiting lysosomal membrane permeabilization. Hsp70 positive lysosomes display increased size and resistance against chemical and physical membrane destabilization; see e.g., Nylandsted et al., “Heat Shock Protein 70 Promotes Cell Survival by Inhibiting Lysosomal Membrane Permeabilization,” J Exp Med. 2004; 200(4): 425-435, the contents of which are incorporated herein by reference in their entirety.

In some embodiments of any of the aspects, the LMP inhibitor is human Hsp70. In some embodiments of any of the aspects, the LMP inhibitor is recombinant human Hsp70 protein (e.g., ABCAM ab78427). In some embodiments of any of the aspects, the human Hsp70 comprises SEQ ID NO: 1 or an amino acid sequence comprising a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 1 or a functional fragment thereof.

MAKAAAIGIDLGTTYSCVGVFQHGKVEIIANDQGNRTTPSYVAFTDTERL
IGDAAKNQVALNPQNTVFDAKRLIGRKFGDPVVQSDMKHWPFQVINDGDK
PKVQVSYKGETKAFYPEEISSMVLTKMKEIAEAYLGYPVTNAVITVPAYF
NDSQRQATKDAGVIAGLNVLRIINEPTAAAIAYGLDRTGKGERNVLIFDL
GGGTFDVSILTIDDGIFEVKATAGDTHLGGEDFDNRLVNHFVEEFKRKHK
KDISQNKRAVRRLRTACERAKRTLSSSTQASLEIDSLFEGIDFYTSITRA
RFEELCSDLFRSTLEPVEKALRDAKLDKAQIHDLVLVGGSTRIPKVQKLL
QDFFNGRDLNKSINPDEAVAYGAAVQAAILMGDKSENVQDLLLLDVAPLS
LGLETAGGVMTALIKRNSTIPTKQTQIFTTYSDNQPGVLIQVYEGERAMT
KDNNLLGRFELSGIPPAPRGVPQIEVTFDIDANGILNVTATDKSTGKANK
ITITNDKGRLSKEEIERMVQEAEKYKAEDEVQRERVSAKNALESYAFNMK
SAVEDEGLKGKISEADKKKVLDKCQEVISWLDANTLAEKDEFEHKRKELE
QVCNPIISGLYQGAGGPGPGGFGAQGPKGGSGSGPTIEEVD

In some embodiments of any of the aspects, the LMP inhibitor is murine Hsp70. In some embodiments of any of the aspects, the LMP inhibitor is recombinant mouse Hsp70 protein (e.g., ABCAM ab113187). In some embodiments of any of the aspects, the murine Hsp70 comprises SEQ ID NO: 2 or an amino acid sequence comprising a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 2 or a functional fragment thereof.

SEQ ID NO: 2, Mus musculus Heat shock 70 kDa protein 1A, 641 aa:

MAKNTAIGIDLGTTYSCVGVFQHGKVEIIANDQGNRTTPSYVAFTDTERL
IGDAAKNQVALNPQNTVFDAKRLIGRKFGDAVVQSDMKHWPFQVVNDGDK
PKVQVNYKGESRSFFPEEISSMVLTKMKEIAEAYLGHPVTNAVITVPAYF
NDSQRQATKDAGVIAGLNVLRIINEPTAAAIAYGLDRTGKGERNVLIFDL
GGGTFDVSILTIDDGIFEVKATAGDTHLGGEDFDNRLVSHFVEEFKRKHK
KDISQNKRAVRRLRTACERAKRTLSSSTQASLEIDSLFEGIDFYTSITRA
RFEELCSDLFRGTLEPVEKALRDAKMDKAQIHDLVLVGGSTRIPKVQKLL
QDFFNGRDLNKSINPDEAVAYGAAVQAAILMGDKSENVQDLLLLDVAPLS
LGLETAGGVMTALIKRNSTIPTKQTQTFTTYSDNQPGVLIQVYEGERAMT
RDNNLLGRFELSGIPPAPRGVPQIEVTFDIDANGILNVTATDKSTGKANK
ITITNDKGRLSKEEIERMVQEAERYKAEDEVQRDRVAAKNALESYAFNMK
SAVEDEGLKGKLSEADKKKVLDKCQEVISWLDSNTLADKEEFVHKREELE
RVCSPIISGLYQGAGAPGAGGFGAQAPKGASGSGPTIEEVD

In some embodiments of any of the aspects, Hsp70 is at a concentration of at least about 10 pM. In some embodiments of any of the aspects, Hsp70 is at a concentration of at least about 100 pM. In some embodiments of any of the aspects, Hsp70 is at a concentration of at least about 1 nM. In some embodiments of any of the aspects, Hsp70 is at a concentration of at least about 10 nM. In some embodiments of any of the aspects, Hsp70 is at a concentration of at least about 100 nM. In some embodiments of any of the aspects, Hsp70 is at a concentration of at least about 200 nM.

In some embodiments of any of the aspects, the LMP inhibitor (e.g., Hsp70) is at a concentration of at least 1 pM, at least 2 pM, at least 3 pM, at least 4 pM, at least 5 pM, at least 6 pM, at least 7 pM, at least 8 pM, at least 9 pM, at least 10 pM, at least 20 pM, at least 30 pM, at least 40 pM, at least 50 pM, at least 60 pM, at least 70 pM, at least 80 pM, at least 90 pM, at least 100 pM, at least 150 pM, at least 200 pM, at least 300 pM, at least 400 pM, at least 500 pM, at least 600 pM, at least 700 pM, at least 800 pM, at least 900 pM, at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 μM, or more. In some embodiments of any of the aspects, the LMP inhibitor (e.g., Hsp70) is at a concentration of 1 pM-10 pM, 10 pM-100 pM, 100 pM-1 nM, 1 nM-10 nM, 10 nM-100 nM, 100 nM-200 nM, or 200 nM-500 nM.

In some embodiments of any of the aspects, the LMP inhibitor is deferoxamine mesylate (DFO) or an analog thereof. Deferoxamine, otherwise known as desferrioxamine or DESFERAL, is a chelating agent used to remove excess iron or aluminum from the body. Specifically, deferoxamine mesylate, is an iron chelator that specifically accumulates inside lysosomes by fluid-phase endocytosis; DFO can inhibit lysosomal-mediated cell death induced by oxidant challenge (see e.g., Doulias et al., 2003, Free Radic Biol Med 35: 719-728; Yu et al., 2003, APMIS 111: 643-652; Kurz et al., 2006, FEBS J 273: 3106-3117; Boya et al. 2008, supra; the contents of each of which are incorporated herein by reference in their entireties). In some embodiments of any of the aspects, the LMP inhibitor is a DFO salt, such as Deferoxamine mesylate salt (e.g., SIGMA-ALDRICH, D9533); see e.g., Formula V below.

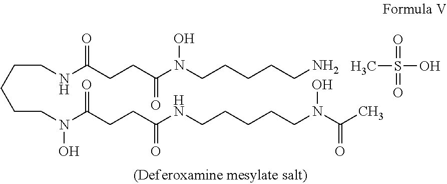

In some embodiments of any of the aspects, DFO is at a concentration of at least about 1 μM. In some embodiments of any of the aspects, DFO is at a concentration of at least about 10 μM. In some embodiments of any of the aspects, DFO is at a concentration of at least about 100 μM. In some embodiments of any of the aspects, DFO is at a concentration of at least about 1 mM. In some embodiments of any of the aspects, DFO is at a concentration of at least about 10 mM.

In some embodiments of any of the aspects, the LMP inhibitor (e.g., DFO) is at a concentration of at least 100 nM, at least 150 nM, at least 200 nM, at least 300 nM, at least 400 nM, at least 500 nM, at least 600 nM, at least 700 nM, at least 800 nM, at least 900 nM, at least 1 μM, at least 2 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, at least 100 μM, at least 150 μM, at least 200 μM, at least 300 μM, at least 400 μM, at least 500 μM, at least 600 μM, at least 700 μM, at least 800 μM, at least 900 μM, at least 1 mM, at least 2 mM, at least 3 mM, at least 4 mM, at least 5 mM, at least 6 mM, at least 7 mM, at least 8 mM, at least 9 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 300 mM, at least 400 mM, at least 500 mM, at least 600 mM, at least 700 mM, at least 800 mM, at least 900 mM, at least 1000 mM, or more. In some embodiments of any of the aspects, the LMP inhibitor (e.g., DFO) is at a concentration of 100 nM-1 μM; 1 μM-10 μM; 10 μM-100 μM; 100 μM-1 mM; 1 mM-10 mM; or 10 mM-100 mM.

In some embodiments of any of the aspects, the composition or cell culture medium described herein comprises at least one antioxidant. In some embodiments, the composition or cell culture medium described herein comprises one antioxidant. In some embodiments, the composition or cell culture medium described herein comprises a plurality of different antioxidants, e.g., at least 2, 3, 4, 5, 6, 7, 8, 9, 10, or more different antioxidants, which can be provided at the same or different concentrations. Antioxidants are substances that can prevent or slow damage to cells caused by free radicals, unstable molecules that the body produces as a reaction to environmental and other pressures. Specifically, antioxidants are compounds that inhibit oxidation, a chemical reaction that can produce free radicals and chain reactions that can damage cells. Antioxidants can also be referred to as “free-radical scavengers.” Non-limiting examples of antioxidants include retinol (vitamin A) and retinol derivatives, ascorbic acid (vitamin C) and ascorbic acid derivatives, butylated hydroxyanisole (BHA), butylated hydroxytoluene (BHT), betacarotene, cysteine, erythorbic acid, hydroquinone, tocopherol (vitamin E) and tocopherol derivatives, N-acetyl cysteine (NAC), glutathione, and the like. In some embodiments of any of the aspects, the antioxidant functions as an antioxidant in humans and/or mice.

In some embodiments of any of the aspects, the antioxidant is N-acetyl cysteine (NAC; see e.g., Formula VI below; e.g., SIGMA-ALDRICH, A9165) or an analog thereof. Chemoprotection by N-acetylcysteine results from inactivation of primary toxicants or reactive electrophiles arising as metabolites or lipid peroxidation products. Other activities that can contribute to chemoprotection by NAC include replenishment of glutathione through cellular delivery of Cys (formed after NAC deacetylation) and the reduction of disulfide bonds in proteins; see e.g., Zhitkovich, “N-Acetylcysteine: Antioxidant, Aldehyde Scavenger, and More,” Chem. Res. Toxicol. 2019, 32, 7, 1318-1319, the contents of which are incorporated herein by reference in their entirety.

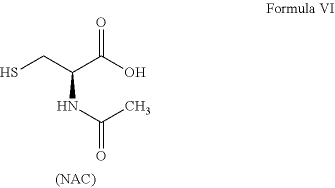

In some embodiments of any of the aspects, the antioxidant (e.g., NAC) is at a concentration of at least about 10 μM. In some embodiments of any of the aspects, the antioxidant (e.g., NAC) is at a concentration of about 10 μM for use with human neutrophils. In some embodiments of any of the aspects, the antioxidant (e.g., NAC) is at a concentration of at least about 1 mM. In some embodiments of any of the aspects, the antioxidant (e.g., NAC) is at a concentration of about 1 mM for use with murine neutrophils. In some embodiments of any of the aspects, the antioxidant (e.g., NAC) is at a concentration of at least about 3.125 mM. In some embodiments of any of the aspects, the antioxidant (e.g., NAC) is at a concentration of at least about 6.25 mM. In some embodiments of any of the aspects, the antioxidant (e.g., NAC) is at a concentration of at least about 12.5 mM. In some embodiments of any of the aspects, the antioxidant (e.g., NAC) is at a concentration of at least about 25 mM. In some embodiments of any of the aspects, the antioxidant (e.g., NAC) is at a concentration of at least about 50 mM.

In some embodiments of any of the aspects, the antioxidant (e.g., NAC) is at a concentration of at least 100 nM, at least 150 nM, at least 200 nM, at least 300 nM, at least 400 nM, at least 500 nM, at least 600 nM, at least 700 nM, at least 800 nM, at least 900 nM, at least 1 μM, at least 2 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, at least 100 μM, at least 150 μM, at least 200 μM, at least 300 μM, at least 400 μM, at least 500 μM, at least 600 μM, at least 700 μM, at least 800 μM, at least 900 μM, at least 1 mM, at least 2 mM, at least 3 mM, at least 4 mM, at least 5 mM, at least 6 mM, at least 7 mM, at least 8 mM, at least 9 mM, at least 10 mM, at least 20 mM, at least 30 mM, at least 40 mM, at least 50 mM, at least 60 mM, at least 70 mM, at least 80 mM, at least 90 mM, at least 100 mM, at least 150 mM, at least 200 mM, at least 300 mM, at least 400 mM, at least 500 mM, at least 600 mM, at least 700 mM, at least 800 mM, at least 900 mM, at least 1000 mM, or more. In some embodiments of any of the aspects, the antioxidant (e.g., NAC) is at a concentration of 100 nM-1 μM; 1 μM-10 μM; 1 μM-20 μM; 10 μM-100 μM; 100 μM-1 mM; 1 mM-10 mM; or 10 mM-100 mM.

In some embodiments of any of the aspects, the composition or cell culture medium described herein comprises at least one growth factor. In some embodiments, the composition or cell culture medium described herein comprises growth factor. In some embodiments, the composition or cell culture medium described herein comprises a plurality of different growth factors, e.g., at least 2, 3, 4, 5, 6, 7, 8, 9, 10, or more different growth factors, which can be provided at the same or different concentrations. In some embodiments of any of the aspects, the growth factor is a human growth factor and/or a murine growth factor.

As used herein, the term “growth factor” refers to a substance that stimulates cell proliferation, wound healing, and/or cellular differentiation. In some embodiments, the growth factor is a myeloid growth factor that stimulates proliferation and/or differentiation of myeloid cells, such as neutrophils. Non-limiting examples of myeloid growth factors include: granulocyte macrophage colony-stimulating factor (GM-CSF), granulocyte colony-stimulating factor (G-CSF), macrophage colony-stimulating factor (M-CSF), and interleukin-3.

In some embodiments, the growth factor is a stimulator of the PI3K/Akt pathway (also referred to herein as a “PI3K/Akt stimulator”). In some embodiments of any of the aspects, the PI3K/Akt stimulator is a stimulator of the human PI3K/Akt pathway and/or murine PI3K/Akt pathway. In some embodiments, the stimulator of the PI3K/Akt pathway is granulocyte colony-stimulating factor (G-CSF). In some embodiments, the growth factor is granulocyte colony-stimulating factor (G-CSF; e.g., AMGEN, NEUPOGEN) or an analog thereof (e.g., FILGRASTIM, LENOGRASTIM, PEGFILGRASTIM).

Granulocyte colony-stimulating factor (G-CSF or GCSF), also known as colony-stimulating factor 3 (CSF 3), is a glycoprotein that stimulates the bone marrow to produce granulocytes and stem cells and release them into the bloodstream. G-CSF also stimulates the survival, proliferation, differentiation, and function of neutrophil precursors and mature neutrophils.

In some embodiments, the growth factor is recombinant G-CSF, such as recombinant human G-CSF (rhG-CSF), also referred to as FILGRASTIM (or NEUPOGEN). FILGRASTIM is a short-acting recombinant, non-PEGylated human granulocyte colony-stimulating factor (G-CSF) analog produced by recombinant DNA technology. FILGRASTIM has an amino acid sequence identical to endogenous G-CSF, but it is non-glycosylated unlike the endogenous G-CSF and has an N-terminal methionine added in the sequence for expression in E. coli. In some embodiments, the G-CSF analog is LENOGRASTIM, which is the is the glycosylated recombinant form of human granulocyte colony stimulating factor. In some embodiments, the G-CSF analog is PEGFILGRASTIM, which is a PEGylated form of the recombinant human granulocyte colony-stimulating factor (G-CSF) analog, FILGRASTIM.

In some embodiments of any of the aspects, the growth factor is human G-CSF. In some embodiments of any of the aspects, the growth factor is recombinant human G-CSF (e.g., AMGEN, NEUPOGEN). In some embodiments of any of the aspects, the human G-CSF comprises SEQ ID NO: 3 or an amino acid sequence comprising a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 3 or a functional fragment thereof.

SEQ ID NO: 3, granulocyte-colony stimulating factor, Homo sapiens, 175 aa:

(M)TPLGPASSLPQSFLLKCLEQVRKIQGDGAALQEKLCATYKLCHPEEL
VLLGHSLGIPWAPLSSCPSQALQLAGCLSQLHSGLFLYQGLLQALEGISP
ELGPTLDTLQLDVADFATTIWQQMEELGMAPALQPTQGAMPAFASAFQRR
AGGVLVASHLQSFLEVSYRVLRHLAQP

In some embodiments of any of the aspects, the growth factor is murine G-CSF. In some embodiments of any of the aspects, the growth factor is recombinant murine G-CSF. In some embodiments of any of the aspects, the murine G-CSF comprises SEQ ID NO: 4 or an amino acid sequence comprising a sequence that is at least 70%, at least 75%, at least 80%, at least 85%, at least 90%, at least 91%, at least 92%, at least 93%, at least 94%, at least 95%, at least 96%, at least 97%, at least 98%, at least 99%, at least 99.5%, at least 99.9%, or more identical to SEQ ID NO: 4 or a functional fragment thereof.

SEQ ID NO: 4, granulocyte-colony stimulating factor, Mus musculus, 208 aa (the signal sequence of SEQ ID NO: 4 (e.g., residues 1-30 of SEQ ID NO: 4) can be removed and optionally replaced with an N-terminal methionine):

MAQLSAQRRMKLMALQLLLWQSALWSGREAVPLVTVSALPPSLPLPRSFL
LKSLEQVRKIQASGSVLLEQLCATYKLCHPEELVLLGHSLGIPKASLSGC
SSQALQQTQCLSQLHSGLCLYQGLLQALSGISPALAPTLDLLQLDVANFA
TTIWQQMENLGVAPTVQPTQSAMPAFTSAFQRRAGGVLAISYLQGFLETA
RLALHHLA

In some embodiments of any of the aspects, the growth factor (e.g., G-CSF) is at a concentration of at least about 10 ng/mL. In some embodiments of any of the aspects, the growth factor (e.g., G-CSF) is at a concentration of at least about 1 ng/ml. In some embodiments of any of the aspects, the growth factor (e.g., G-CSF) is at a concentration of at least about 100 ng/mL. In some embodiments of any of the aspects, the growth factor (e.g., G-CSF) is at a concentration of at least about 200 ng/mL. In some embodiments of any of the aspects, the growth factor (e.g., G-CSF) is at a concentration of at least about 1 μg/mL. In some embodiments of any of the aspects, the growth factor (e.g., G-CSF) is at a concentration of at least about 10 μg/mL.

In some embodiments of any of the aspects, the growth factor (e.g., G-CSF) is at a concentration of at least 1 ng/mL, at least 2 ng/mL, at least 3 ng/mL, at least 4 ng/mL, at least 5 ng/mL, at least 6 ng/mL, at least 7 ng/mL, at least 8 ng/mL, at least 9 ng/mL, at least 10 ng/mL, at least 20 ng/mL, at least 30 ng/mL, at least 40 ng/mL, at least 50 ng/mL, at least 60 ng/mL, at least 70 ng/mL, at least 80 ng/mL, at least 90 ng/mL, at least 100 ng/mL, at least 150 ng/mL, at least 200 ng/mL, at least 300 ng/mL, at least 400 ng/mL, at least 500 ng/mL, at least 600 ng/mL, at least 700 ng/mL, at least 800 ng/mL, at least 900 ng/mL, at least 1 μg/mL, at least 2 μg/mL, at least 3 μg/mL, at least 4 μg/mL, at least 5 μg/mL, at least 6 μg/mL, at least 7 μg/mL, at least 8 μg/mL, at least 9 μg/mL, at least 10 μg/mL, at least 20 μg/mL, at least 30 μg/mL, at least 40 μg/mL, at least 50 μg/mL, at least 60 μg/mL, at least 70 μg/mL, at least 80 μg/mL, at least 90 μg/mL, at least 100 μg/mL, at least 150 μg/mL, at least 200 μg/mL, at least 300 μg/mL, at least 400 μg/mL, at least 500 μg/mL, at least 600 μg/mL, at least 700 μg/mL, at least 800 μg/mL, at least 900 μg/mL, at least 1000 μg/mL or more. In some embodiments of any of the aspects, the growth factor (e.g., G-CSF) is at a concentration of 1 ng/mL-10 ng/mL; 10 ng/mL-100 ng/mL; 100 ng/mL-200 ng/mL; 200 ng/mL-1 μg/mL; 1 μg/mL-10 μg/mL; or 10 μg/mL-100 μg/mL.

In some embodiments of any of the aspects, the composition or cell culture medium described herein comprises at least one necroptosis inhibitor. In some embodiments, the composition or cell culture medium described herein comprises one necroptosis inhibitor. In some embodiments, the composition or cell culture medium described herein comprises a plurality of different necroptosis inhibitors, e.g., at least 2, 3, 4, 5, 6, 7, 8, 9, 10, or more different necroptosis inhibitors, which can be provided at the same or different concentrations. In some embodiments of any of the aspects, the necroptosis inhibitor is an inhibitor of human necroptosis and/or murine necroptosis.

Necroptosis is a kind of necrosis that triggers innate immune responses by rupturing dead cells and releasing intracellular components; it can be caused by Toll-like receptor (TLR)-3 and TLR-4 agonists, tumor necrosis factor (TNF), certain microbial infections, and T cell receptors. Necroptosis is a programmed form of necrosis, or inflammatory cell death. Conventionally, necrosis is associated with un-programmed cell death resulting from cellular damage or infiltration by pathogens, in contrast to orderly, programmed cell death via apoptosis. Necroptosis signaling is modulated by receptor-interacting protein kinase (RIPK) 1 when the activity of caspase-8 becomes compromised. Activated death receptors (DRs) cause the activation of RIPK1 and the RIPK1 kinase activity-dependent formation of an RIPK1-RIPK3-mixed lineage kinase domain-like protein (MLKL), which is complex II. RIPK3 phosphorylates MLKL, ultimately leading to necrosis through plasma membrane disruption and cell lysis; see e.g., Yu et al., “Necroptosis: A Novel Pathway in Neuroinflammation,” Front. Pharmacol., 2021, the contents of which are incorporated herein by reference in their entirety.

In some embodiments, the necroptosis inhibitor inhibits human RIPK1, murine RIPK1, human RIPK3, or murine RIPK3. Non-limiting examples of necroptosis inhibitors include: Nec-1s, NECROX-2, NECROX-5, Nec-1, Necrosulfonamide, GSK′872, and RIPA 56. In some embodiments, the necroptosis inhibitor is Nec-1s, NECROX-2, or NECROX-5.

In some embodiments, the necroptosis inhibitor is Necrostatin-1s (also referred to as Nec-1s or 7-Cl-O-Nec-1; e.g., EMD MILLIPORE, 852391-15-2; see e.g., Formula VII) or an analog thereof. Nec-1s is a potent and selective inhibitor of necroptosis. Nec-1s is an analog of Necrostatin-1 (Nec-1), and Nec-1s has been shown to be a specific receptor-interacting protein kinase (RIPK) 1 inhibitor that is metabolically more stable than the other Nec-1 variants. Nec-1s is more selective than Nec-1 and Nec-1i (inactive variant) due to the fact that both molecules have an inhibitory effect on indoleamine 2,3-dioxygenase (IDO), a potent immunosuppressive molecule. Nec-1s is about two-times more effective at RIPK1 inhibition than Nec-1 (IC50=210 nM vs. 494 nM)

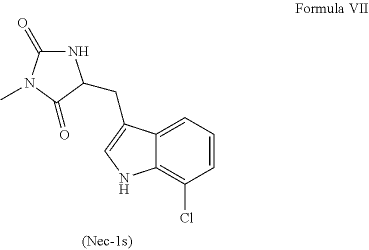 [SMILES: CN1C(=O)NC(Cc2c[nH]c3c(Cl)cccc23)C1=O]

In some embodiments of any of the aspects, the necroptosis inhibitor (e.g., Nec-1s) is at a concentration of at least 10 μM. In some embodiments of any of the aspects, the necroptosis inhibitor (e.g., Nec-1s) is at a concentration of at least 10 nM. In some embodiments of any of the aspects, the necroptosis inhibitor (e.g., Nec-1s) is at a concentration of at least 100 nM. In some embodiments of any of the aspects, the necroptosis inhibitor (e.g., Nec-1s) is at a concentration of at least 1 μM. In some embodiments of any of the aspects, the necroptosis inhibitor (e.g., Nec-1s) is at a concentration of at least 10-μM. In some embodiments of any of the aspects, the necroptosis inhibitor (e.g., Nec-1s) is at a concentration of at least 200 μM.

In some embodiments, the necroptosis inhibitor (e.g., Nec-1s) is at a concentration of at least 1 nM, at least 2 nM, at least 3 nM, at least 4 nM, at least 5 nM, at least 6 nM, at least 7 nM, at least 8 nM, at least 9 nM, at least 10 nM, at least 20 nM, at least 30 nM, at least 40 nM, at least 50 nM, at least 60 nM, at least 70 nM, at least 80 nM, at least 90 nM, at least 100 nM, at least 150 nM, at least 200 nM, at least 300 nM, at least 400 nM, at least 500 nM, at least 600 nM, at least 700 nM, at least 800 nM, at least 900 nM, at least 1.0 μM, at least 1.25 μM, at least 1.5 μM, at least 1.75 μM, at least 2.0 μM, at least 2.5 μM, at least 3 μM, at least 4 μM, at least 5 μM, at least 6 μM, at least 7 μM, at least 8 μM, at least 9 μM, at least 10 μM, at least 20 μM, at least 30 μM, at least 40 μM, at least 50 μM, at least 60 μM, at least 70 μM, at least 80 μM, at least 90 μM, at least 100 μM, at least 110 μM, at least 120 μM, at least 130 μM, at least 140 μM, at least 150 μM, at least 160 μM, at least 170 μM, at least 180 μM, at least 190 μM, at least 200 μM, at least 250 μM, at least 300 μM, at least 350 μM, at least 400 μM, at least 450 μM, at least 500 μM, at least 600 μM, at least 700 μM, at least 800 μM, at least 900 μM, at least 1000 μM, or more. In some embodiments, the necroptosis inhibitor (e.g., Nec-1s) is at a concentration of 1 nM-10 nM, 10 nM-50 nM, 50 nM-100 nM, 100 nM-500 nM, 500 nM-1 μM, 1 μM-5 μM, 5 μM-10 μM, 12.5 μM-25 μM, 25 μM-50 μM, 50 μM-100 μM, 100 μM-200 μM, or 200 μM-500 μM.

In some embodiments of any of the aspects, the composition or cell culture medium described herein comprises at least one cell culture medium. In some embodiments, the composition or cell culture medium described herein comprises one cell culture medium. In some embodiments, the composition or cell culture medium described herein comprises a plurality of different cell culture mediums, e.g., at least 2, 3, 4, 5, 6, 7, 8, 9, 10, or more different cell culture mediums, which can be provided at the same or different concentrations.

Cell culture medium is a substrate comprising an appropriate source of energy and compounds which regulate the cell cycle. In some embodiments, the culture medium comprises amino acids, vitamins, inorganic salts, glucose, and serum as a source of growth factors, hormones, and/or attachment factors. Components of a cell culture medium can include carbohydrates, amino acids, vitamins, minerals, and a pH buffer system. In some embodiments, the culture medium is a liquid or a solid. In some embodiments, the culture medium comprises components to support the growth of mammalian (e.g., human, murine) cells, including immune cells. Non-limiting examples of cell culture media include Roswell Park Memorial Institute (RPMI) media, Minimum Essential Media (MEM; also referred to as Eagle's MEM), Dulbecco's Modified Eagle Medium (DMEM), and Iscove's Modified Dulbecco's Medium (IMDM).

In some embodiments, the cell culture medium comprises RPMI, also known as RPMI-1640 medium. RPMI 1640 Medium comprises the reducing agent glutathione and high concentrations of vitamins. RPMI 1640 Medium contains biotin, vitamin B12, and para-aminobenzoic acid (PABA), which are not found in MEM or DMEM. In addition, the vitamins inositol and choline are present in very high concentrations. RPMI 1640 Medium contains no proteins, lipids, or growth factors. In some embodiments, RPMI 1640 Medium comprises supplements, such as Fetal Bovine Serum (FBS). RPMI 1640 Medium uses a sodium bicarbonate buffer system (2.0 g/L), and thus can use a 5-10% CO₂ environment to maintain physiological pH.

In some embodiments, the cell culture medium (e.g., RPMI) comprises at least one of the following amino acid components: Glycine, L-Arginine, L-Asparagine, L-Aspartic acid, L-Cystine 2HCl, L-Glutamic Acid, L-Glutamine, L-Histidine, L-Hydroxyproline, L-Isoleucine, L-Leucine, L-Lysine hydrochloride, L-Methionine, L-Phenylalanine, L-Proline, L-Serine, L-Threonine, L-Tryptophan, L-Tyrosine disodium salt dihydrate, and/or L-Valine. In some embodiments, the cell culture medium (e.g., RPMI) comprises at least one of the following vitamin components: Vitamin B, Choline chloride, Vitamin D-Calcium pantothenate, Folic Acid, Niacinamide, and Para-Aminobenzoic Acid. In some embodiments, the cell culture medium further comprises antibiotics (e.g., penicillin-streptomycin), L-glutamine, Phenol Red, and/or HEPES (4-(2-hydroxyethyl)-1-piperazineethanesulfonic acid).

In some embodiments of any of the aspects, the composition or cell culture medium described herein comprises at least one type of serum. In some embodiments, the composition or cell culture medium described herein comprises one type of serum. In some embodiments, the composition or cell culture medium described herein comprises a plurality of different serums, e.g., at least 2, 3, 4, 5, 6, 7, 8, 9, 10, or more different serums, which can be provided at the same or different concentrations.

Serum, which is typically derived from the blood of mammal, is a complex mix of albumins, growth factors and growth inhibitors. Non-limiting examples of serums include fetal bovine serum (FBS), newborn calf serum, and horse serum. In some embodiments, the serum is fetal bovine serum (FBS). In some embodiments, the serum (e.g., FBS) is at a concentration of at least 20%. In some embodiments, the serum (e.g., FBS) is at a concentration of at least 1%, at least 2%, at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, at least 40%, at least 41%, at least 42%, at least 43%, at least 44%, at least 45%, at least 46%, at least 47%, at least 48%, at least 49%, at least 50%, or more. In some embodiments, the serum (e.g., FBS) is at a concentration of 1%-5%, 5%-10%, 10%-15%, 15%-20%, 15%-25%, 20%-25%, 25%-30%, 30%-40%, or 40%-50%.

In some embodiments of any of the aspects, the composition or cell culture medium described herein further comprises at least one component selected from the group consisting of: a serine protease inhibitor (e.g., Diisopropylfluorophosphate (DFP)); an NADPH oxidase inhibitor (e.g., Diphenyleneiodonium chloride (DPI)); and a Rho GTPase inhibitor (e.g., NSC23766 (Rac inhibitor)). In some embodiments of any of the aspects, the composition or cell culture medium described herein does not comprise at least one component selected from the group consisting of: a serine protease inhibitor (e.g., Diisopropylfluorophosphate (DFP)); an NADPH oxidase inhibitor (e.g., Diphenyleneiodonium chloride (DPI)); and a Rho GTPase inhibitor (e.g., NSC23766 (Rac inhibitor)).

In one aspect, described herein is a cell culture medium (or composition) for increasing the lifespan of human neutrophils, the cell culture medium (or composition) comprising: (a) at least about 50 μM of a pan-caspase inhibitor; (b) at least about 1 μM of a first LMP inhibitor; (c) at least about 10 μm of a second LMP inhibitor; (d) at least about 10 μM of an antioxidant; (e) at least about 10 ng/mL a growth factor; and (f) at least about 10 μM of a necroptosis inhibitor.

In one aspect, described herein is a cell culture medium (or composition) for increasing the lifespan of human neutrophils, the cell culture medium (or composition) comprising: (a) at least about 50 μM of Q-VD-Oph; (b) at least about 1 μM of DFO; (b) at least about 10 pm of Hsp70; (c) at least about 10 μM of NAC; (d) at least about 10 ng/mL of G-CSF; and (e) at least about 10 μM of Nec-1s.

In one aspect, described herein is a cell culture medium (or composition) for increasing the lifespan of human neutrophils, the cell culture medium (or composition) comprising: (a) about 50 μM of Q-VD-Oph; (b) about 1 μM of DFO; (b) about 10 pm of Hsp70; (c) about 10 μM of NAC; (d) about 10 ng/mL of G-CSF; and (e) about 10 μM of Nec-1s.

In one aspect, described herein is a cell culture medium (or composition) for increasing the lifespan of murine neutrophils, the cell culture medium (or composition) comprising: (a) at least about 50 μM of a pan-caspase inhibitor; (b) at least about 1 μM of a first LMP inhibitor; (c) at least about 10 pm of a second LMP inhibitor; (d) at least about 1 mM of an antioxidant; (e) at least about 10 ng/mL of a growth factor; and (f) at least about 10 μM of a necroptosis inhibitor.

In one aspect, described herein is a cell culture medium (or composition) for increasing the lifespan of neutrophils, the cell culture medium (or composition) comprising: (a) at least about 10 μM of a pan-caspase inhibitor; (b) at least about 1 μM of a first LMP inhibitor; (c) at least about 10 μm of a second LMP inhibitor; (d) at least about 1 mM of an antioxidant; (e) at least about 10 ng/mL of a growth factor; and (f) at least about 10 μM of a necroptosis inhibitor.

In one aspect, described herein is a cell culture medium (or composition) for increasing the lifespan of neutrophils, the cell culture medium (or composition) comprising: (a) at least about 10 μM of a pan-caspase inhibitor; (b) at least about 1 μM of a first LMP inhibitor; (c) at least about 10 pm of a second LMP inhibitor; (d) at least about 1 mM of an antioxidant; and (e) at least about 10 ng/mL of a growth factor.

In one aspect, described herein is a cell culture medium (or composition) for increasing the lifespan of neutrophils, the cell culture medium (or composition) comprising: (a) at least about 25 μM of a pan-caspase inhibitor; (b) at least about 1 μM of a first LMP inhibitor; (c) at least about 10 pm of a second LMP inhibitor; (d) at least about 1 mM of an antioxidant; (e) at least about 10 ng/mL of a growth factor; and (f) at least about 10 μM of a necroptosis inhibitor.

In one aspect, described herein is a cell culture medium (or composition) for increasing the lifespan of murine neutrophils, the cell culture medium (or composition) comprising: (a) at least about 50 μM of Q-VD-Oph; (b) at least about 1 μM of DFO; (c) at least about 10 pm of Hsp70; (d) at least about 1 mM of NAC; (e) at least about 10 ng/mL of G-CSF; and (f) at least about 10 μM of Nec-1s.

In one aspect, described herein is a cell culture medium (or composition) for increasing the lifespan of murine neutrophils, the cell culture medium (or composition) comprising: (a) about 50 μM of Q-VD-Oph; (b) about 1 μM of DFO; (c) about 10 pm of Hsp70; (d) about 1 mM of NAC; (e) about 10 ng/mL of G-CSF; and (f) about 10 μM of Nec-1s.

In one aspect, described herein is a cell culture medium (or composition) for increasing the lifespan of neutrophils, the cell culture medium (or composition) comprising: (a) about 10 μM of Emricasan; (b) about 1 μM of DFO; (c) about 10 pm of Hsp70; (d) about 1 mM of NAC; (e) about 10 ng/mL of G-CSF; and (f) about 10 μM of Nec-1s.

In one aspect, described herein is a cell culture medium (or composition) for increasing the lifespan of neutrophils, the cell culture medium (or composition) comprising: (a) about 10 μM of Emricasan; (b) about 1 μM of DFO; (c) about 10 pm of Hsp70; (d) about 1 mM of NAC; and (e) about 10 ng/mL of G-CSF.

In one aspect, described herein is a cell culture medium (or composition) for increasing the lifespan of neutrophils, the cell culture medium (or composition) comprising: (a) about 25 μM of Emricasan; (b) about 1 μM of DFO; (c) about 10 pm of Hsp70; (d) about 1 mM of NAC; and (e) about 10 ng/mL of G-CSF.

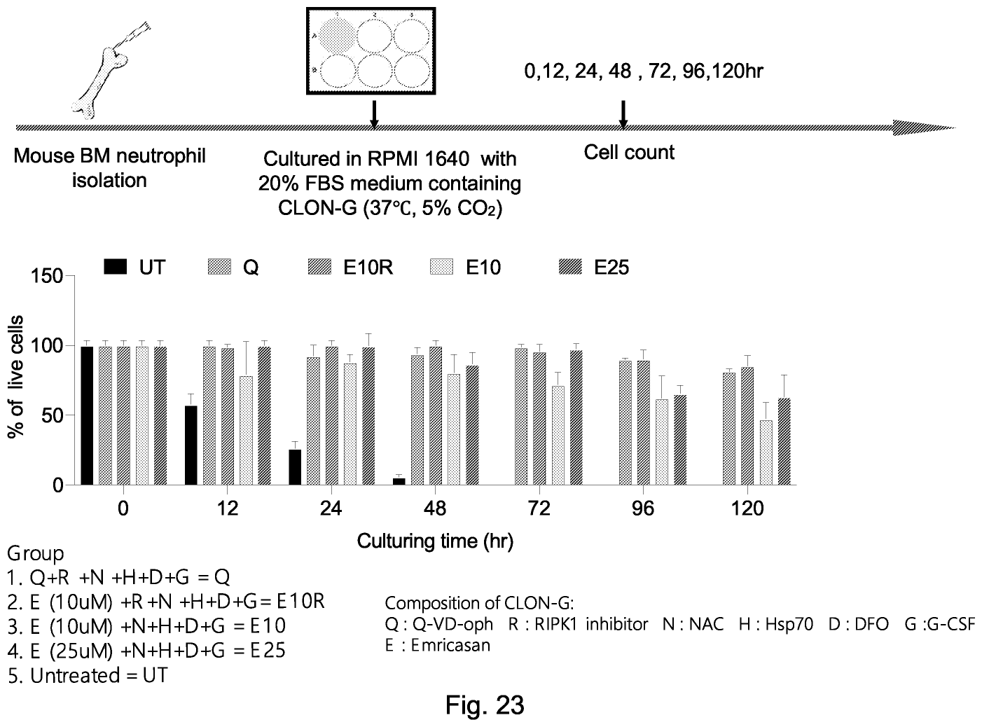

In any embodiment of the aspects described below, the cell culture medium (or composition) can comprise an effective amount (e.g., effective concentration) of each of the indicated components, e.g., an amount sufficient to increase the half-life of cultured human or murine neutrophils. In one aspect, described herein is a cell culture medium (or composition) comprising: Q-VD-Oph and NAC. In one aspect, described herein is a cell culture medium (or composition) comprising: Q-VD-Oph and DFO. In one aspect, described herein is a cell culture medium (or composition) comprising: Q-VD-Oph and Hsp70. In one aspect, described herein is a cell culture medium (or composition) comprising: Q-VD-Oph and Nec-1s. In one aspect, described herein is a cell culture medium (or composition) comprising: Q-VD-Oph and G-CSF. In one aspect, described herein is a cell culture medium (or composition) comprising: Z-VAD-FMK, DFO, DPI, and G-CSF. In one aspect, described herein is a cell culture medium (or composition) comprising: Emricasan, DFO, DPI, and G-CSF. In one aspect, described herein is a cell culture medium (or composition) comprising: Ac-DEVD-CHO, DFO, DPI, and G-CSF. In one aspect, described herein is a cell culture medium (or composition) comprising: Z-DEVD-FMK, Hsp70, NAC, and G-CSF. In one aspect, described herein is a cell culture medium (or composition) comprising: Z-VAD-FMK, Hsp70, NAC, and G-CSF. In one aspect, described herein is a cell culture medium (or composition) comprising: Ac-DEVD-CHO, Hsp70, NAC, and G-CSF. In one aspect, described herein is a cell culture medium (or composition) comprising: Q-VD-Oph, G-CSF, and NAC. In one aspect, described herein is a cell culture medium (or composition) comprising: Q-VD-Oph, G-CSF, and DFO. In one aspect, described herein is a cell culture medium (or composition) comprising: Q-VD-Oph, G-CSF, and Hsp70. In one aspect, described herein is a cell culture medium (or composition) comprising: Q-VD-Oph, G-CSF, and Nec-1s. In one aspect, described herein is a cell culture medium (or composition) comprising: Q-VD-Oph, G-CSF, and NECROX-2. In one aspect, described herein is a cell culture medium (or composition) comprising: Q-VD-Oph, G-CSF, and NECROX-5. In one aspect, described herein is a cell culture medium (or composition) comprising: Q-VD-Oph, DFO, DPI, and G-CSF. In one aspect, described herein is a cell culture medium (or composition) comprising: Q-VD-Oph, human Hsp70, DPI, and G-CSF. In one aspect, described herein is a cell culture medium (or composition) comprising: Q-VD-Oph, DFO, NAC, and G-CSF. In one aspect, described herein is a cell culture medium (or composition) comprising: Q-VD-Oph, mouse Hsp70, DPI, and G-CSF. In one aspect, described herein is a cell culture medium (or composition) comprising: Q-VD-Oph, Hsp70, NAC, and G-CSF. In one aspect, described herein is a cell culture medium (or composition) comprising: Q-VD-Oph, DFO, NAC, DFP, and G-CSF. In one aspect, described herein is a cell culture medium (or composition) comprising: Q-VD-Oph, Hsp70, DPI, DFP, and G-CSF. In one aspect, described herein is a cell culture medium (or composition) comprising: Q-VD-Oph, Hsp70, NAC, DFP, and G-CSF. In one aspect, described herein is a cell culture medium (or composition) comprising: Q-VD-Oph, Hsp70, NAC, DFO, G-CSF, and Nec-1s. In one aspect, described herein is a cell culture medium (or composition) comprising: Q-VD-Oph, Hsp70, NAC, DFO, G-CSF, Nec-1s, and NSC23766 (see e.g., Tables 1-2). In one aspect, described herein is a cell culture medium (or composition) comprising: Emricasan; DFO; Hsp70; NAC; G-CSF; and Nec-1s. In one aspect, described herein is a cell culture medium (or composition) comprising: Emricasan; DFO; Hsp70; NAC; and G-CSF (see e.g., FIG. 23). In one aspect, described herein is a cell culture medium (or composition) comprising: Z-VAD-FMK; DFO; Hsp70; NAC; and G-CSF. In one aspect, described herein is a cell culture medium (or composition) comprising: Ac-DEVD-CHO; DFO; Hsp70; NAC; and G-CSF. In one aspect, described herein is a cell culture medium (or composition) comprising: Z-VAD-FMK; Ac-DEVD-CHO; DFO; Hsp70; NAC; and G-CSF.

### Kits

Another aspect of the technology described herein relates to kits for prolonging the life span of neutrophils. Described herein are kit components that can be included in one or more of the kits described herein. In one aspect, described herein a kit comprising a composition as described herein. In one aspect, described herein a kit comprising a cell culture medium as described herein. In some embodiments, the kit comprises any combination of components as described in Table 3.

In some embodiments, the kit comprises an effective amount of the caspase inhibitor, LMP inhibitor, antioxidant, growth factor, necroptosis inhibitor, cell culture medium, and/or serum. As will be appreciated by one of skill in the art, components, including but not limited to the caspase inhibitor, LMP inhibitor, antioxidant, growth factor, necroptosis inhibitor, can be supplied in a lyophilized form or a concentrated form that can diluted or suspended in liquid prior to use with cultured cells. Preferred formulations include those that are non-toxic to the cells and/or does not affect growth rate or viability etc. The caspase inhibitor, LMP inhibitor, antioxidant, growth factor, necroptosis inhibitor, cell culture medium, and/or serum can be supplied in aliquots or in unit doses.

In some embodiments, the components described herein can be provided singularly or in any combination as a kit. In some embodiments, the kit furthermore comprises reagents and materials for isolating neutrophils. In some embodiments, the kit furthermore comprises materials for culturing neutrophils, such as culture vessels. Such kits can optionally include one or more agents that permit the detection of neutrophils, including mature or activated neutrophils. In addition, the kit optionally comprises informational material.

In some embodiments, the compositions in the kit can be provided in a watertight or gas tight container which in some embodiments is substantially free of other components of the kit. For example, the composition or cell culture medium described herein can be supplied in more than one container, e.g., it can be supplied in a container having sufficient reagent for a predetermined number of cultures, e.g., 1, 2, 3 or greater. One or more components as described herein can be provided in any form, e.g., liquid, dried or lyophilized form. It is preferred that the components described herein are substantially pure and/or sterile. When the components described herein are provided in a liquid solution, the liquid solution preferably is an aqueous solution, with a sterile aqueous solution being preferred.

The informational material can be descriptive, instructional, marketing or other material that relates to the methods described herein. The informational material of the kits is not limited in its form. In one embodiment, the informational material can include information about production of the compositions or cell culture media described herein, concentration, date of expiration, batch or production site information, and so forth. In one embodiment, the informational material relates to methods for using the components of the kit.

The kit can include a component for the detection of a marker for neutrophils. In addition, the kit can include one or more antibodies that bind a cell marker, or primers for an RT-PCR or PCR reaction, e.g., a semi-quantitative or quantitative RT-PCR or PCR reaction. Such components can be used to assess the activation of neutrophil markers. If the detection reagent is an antibody, it can be supplied in dry preparation, e.g., lyophilized, or in a solution. The antibody or other detection reagent can be linked to a label, e.g., a radiological, fluorescent (e.g., GFP) or colorimetric label for use in detection. If the detection reagent is a primer, it can be supplied in dry preparation, e.g., lyophilized, or in a solution.

The kit will typically be provided with its various elements included in one package, e.g., a fiber-based, e.g., a cardboard, or polymeric, e.g., a Styrofoam box. The enclosure can be configured so as to maintain a temperature differential between the interior and the exterior, e.g., it can provide insulating properties to keep the reagents at a preselected temperature for a preselected time.

### Culture Methods

In multiple aspects, described herein are methods of increasing the lifespan of a neutrophil. In one aspect, described herein is a method comprising: contacting the neutrophil, or population thereof, with a composition as described herein. In one aspect, described herein is a method comprising: contacting the neutrophil, or population thereof, with a cell culture medium as described herein. In one aspect, described herein is a method comprising: contacting the neutrophil, or population thereof, with a composition comprising: (a) a caspase inhibitor; (b) a lysosomal membrane permeabilization (LMP) inhibitor; (c) an antioxidant; and (d) a growth factor, or any combination thereof (see e.g., Table 3). In one aspect, described herein is a method comprising: contacting the neutrophil, or population thereof, with a cell culture medium comprising: (a) a caspase inhibitor; (b) a lysosomal membrane permeabilization (LMP) inhibitor; (c) an antioxidant; (d) a growth factor; and (e) a necroptosis inhibitor, or any combination thereof (see e.g., Table 3).

In some embodiments of any of the aspects, the neutrophil, or population thereof, is a peripheral blood neutrophil (PMN). In some embodiments of any of the aspects, the neutrophil, or population thereof, is isolated from the peripheral blood of a subject. In some embodiments of any of the aspects, the neutrophil, or population thereof, is isolated from blood of a subject using apheresis. In some embodiments of any of the aspects, the neutrophil, or population thereof, is isolated from the bone marrow of a subject.

In some embodiments of any of the aspects, the neutrophil, or population thereof, is contacted with the composition or the cell culture medium for a sufficient amount of time to increase neutrophil lifespan. In some embodiments, the sufficient amount of time to increase neutrophil lifespan is at least 3 days. In some embodiments, the sufficient amount of time to increase neutrophil lifespan is at least 1 hour. In some embodiments, the sufficient amount of time to increase neutrophil lifespan is at least 1 hour, at least 2 hours, at least 3 hours, at least 4 hours, at least 5 hours, at least 6 hours, at least 7 hours, at least 8 hours, at least 9 hours, at least 10 hours, at least 11 hours, at least 12 hours, at least 13 hours, at least 14 hours, at least 15 hours, at least 16 hours, at least 17 hours, at least 18 hours, at least 19 hours, at least 20 hours, at least 21 hours, at least 22 hours, at least 23 hours, at least 24 hours, at least 25 hours, at least 26 hours, at least 27 hours, at least 28 hours, at least 29 hours, at least 30 hours, at least 31 hours, at least 32 hours, at least 33 hours, at least 34 hours, at least 35 hours, at least 36 hours, at least 37 hours, at least 38 hours, at least 39 hours, at least 40 hours, at least 41 hours, at least 42 hours, at least 43 hours, at least 44 hours, at least 45 hours, at least 46 hours, at least 47 hours, at least 48 hours, at least 49 hours, at least 50 hours, at least 51 hours, at least 52 hours, at least 53 hours, at least 54 hours, at least 55 hours, at least 56 hours, at least 57 hours, at least 58 hours, at least 59 hours, at least 60 hours, at least 61 hours, at least 62 hours, at least 63 hours, at least 64 hours, at least 65 hours, at least 66 hours, at least 67 hours, at least 68 hours, at least 69 hours, at least 70 hours, at least 71 hours, at least 72 hours, at least 73 hours, at least 74 hours, at least 75 hours, at least 76 hours, at least 77 hours, at least 78 hours, at least 79 hours, at least 80 hours, at least 81 hours, at least 82 hours, at least 83 hours, at least 84 hours, at least 85 hours, at least 86 hours, at least 87 hours, at least 88 hours, at least 89 hours, at least 90 hours, at least 91 hours, at least 92 hours, at least 93 hours, at least 94 hours, at least 95 hours, at least 96 hours, at least 97 hours, at least 98 hours, at least 99 hours, at least 100 hours, at least 101 hours, at least 102 hours, at least 103 hours, at least 104 hours, at least 105 hours, at least 106 hours, at least 107 hours, at least 108 hours, at least 109 hours, at least 110 hours, at least 111 hours, at least 112 hours, at least 113 hours, at least 114 hours, at least 115 hours, at least 116 hours, at least 117 hours, at least 118 hours, at least 119 hours, at least 120 hours, or more. In some embodiments, the sufficient amount of time to increase neutrophil lifespan is 1 hour-12 hours, 12 hours-24 hours, 1 day-2 days, 2 days-3 days, 3 days-4 days, or 4 days-5 days.

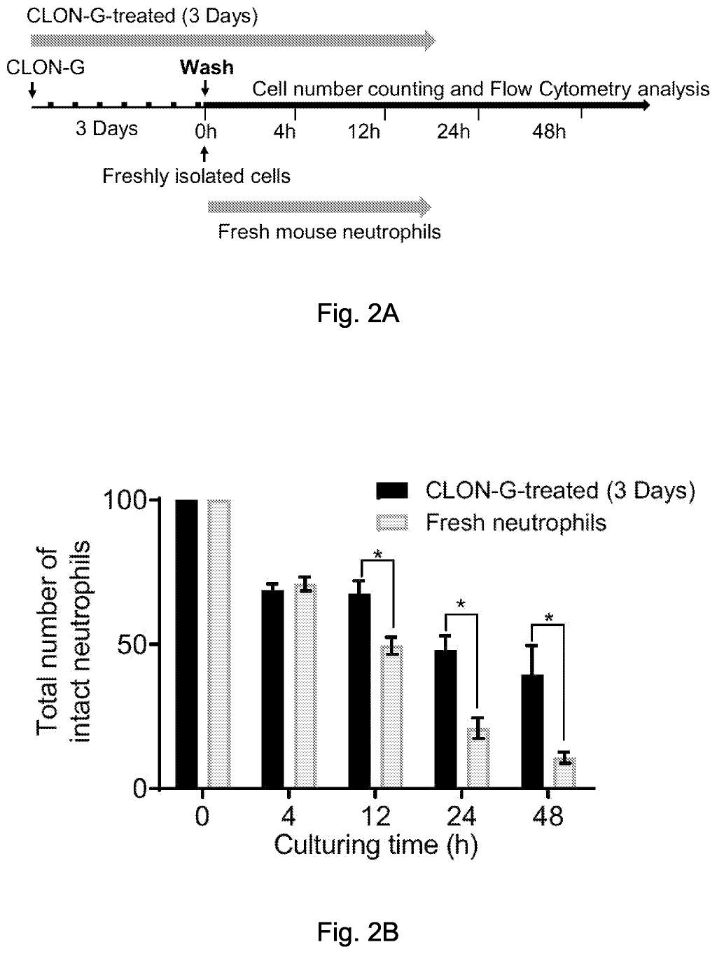

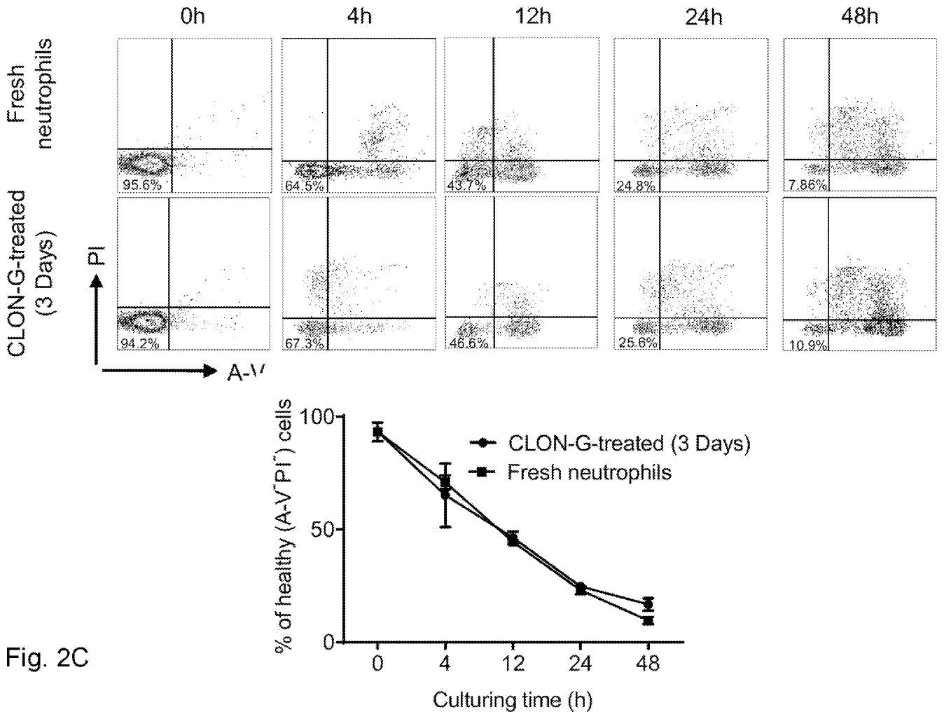

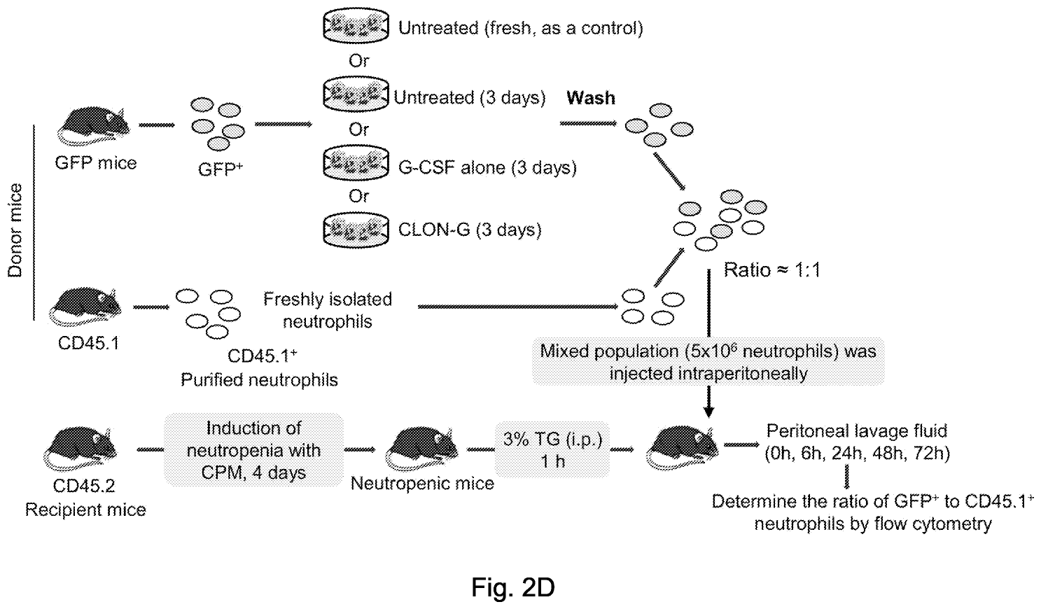

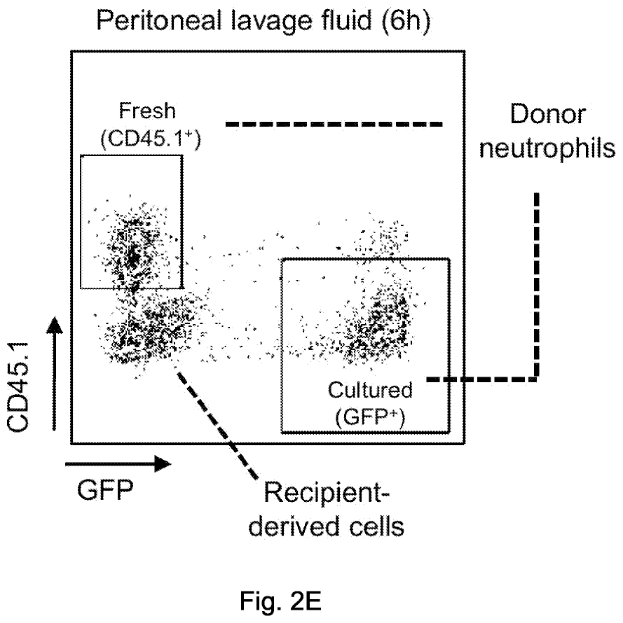

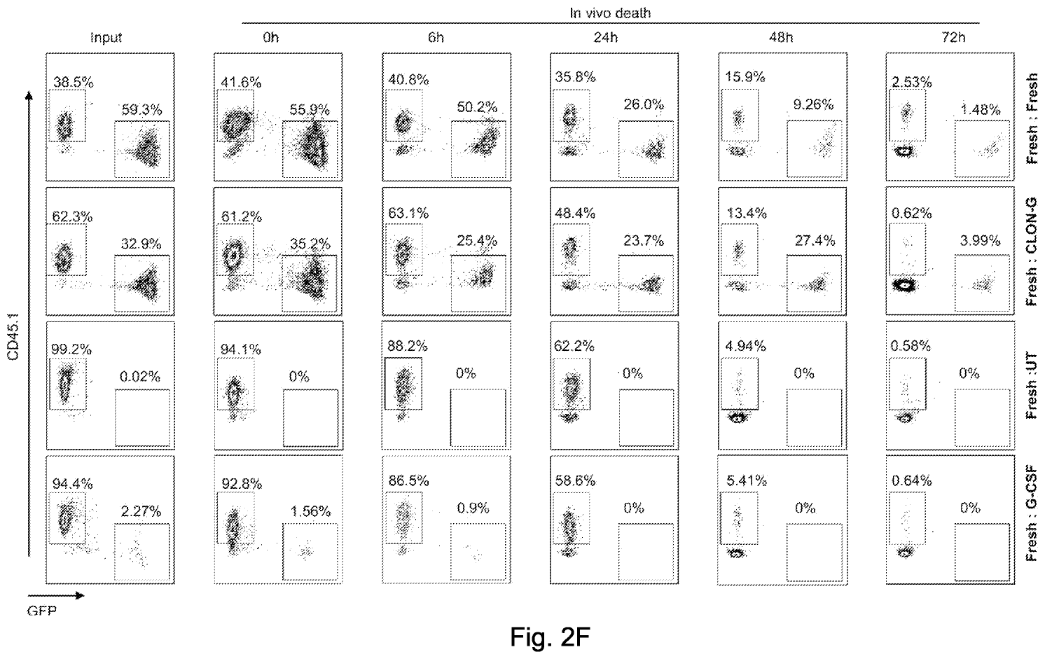

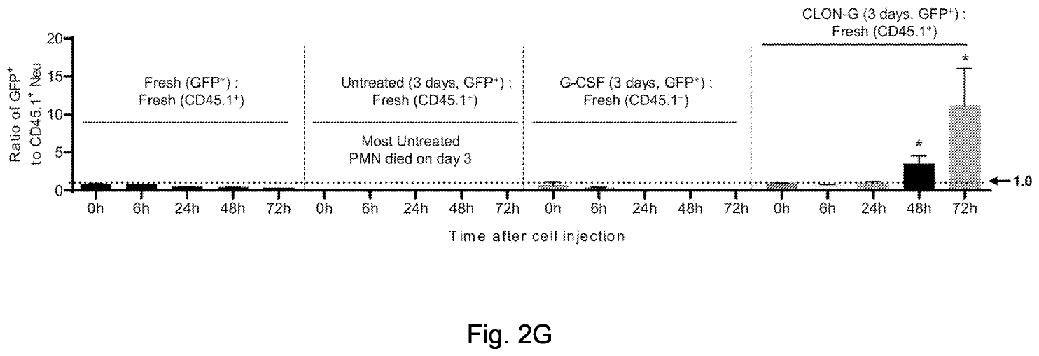

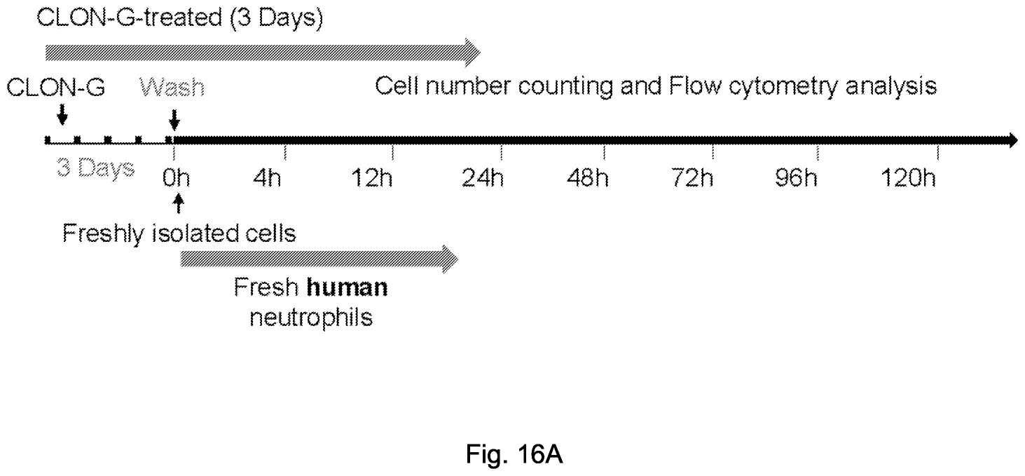

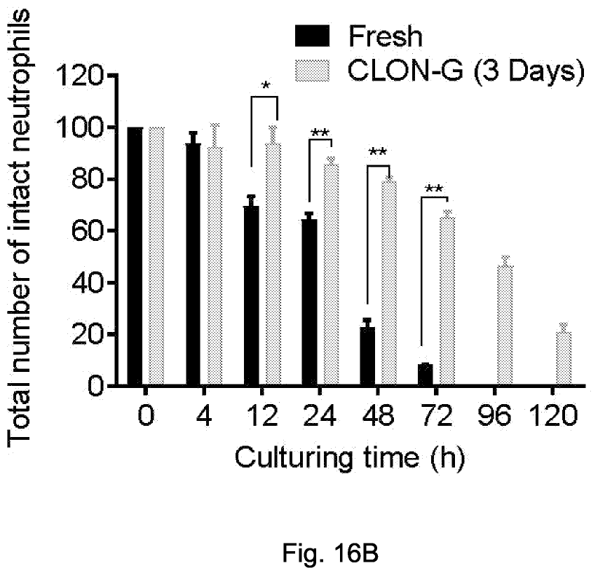

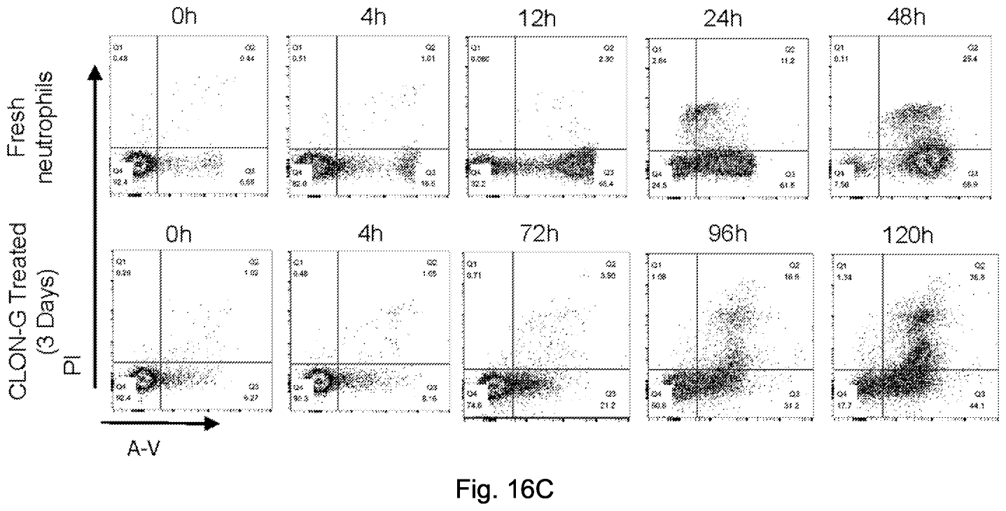

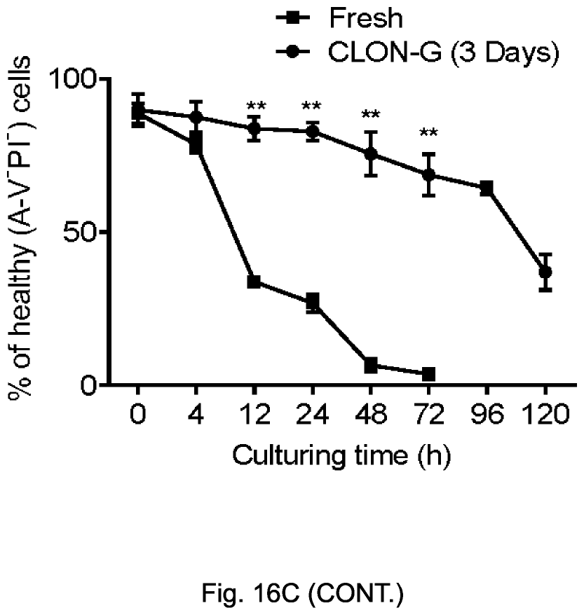

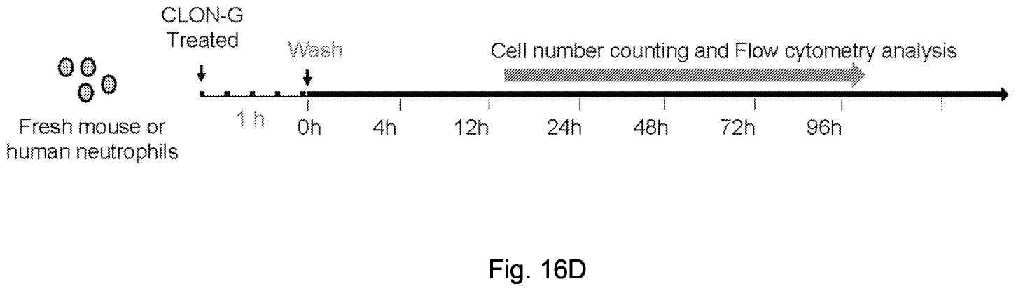

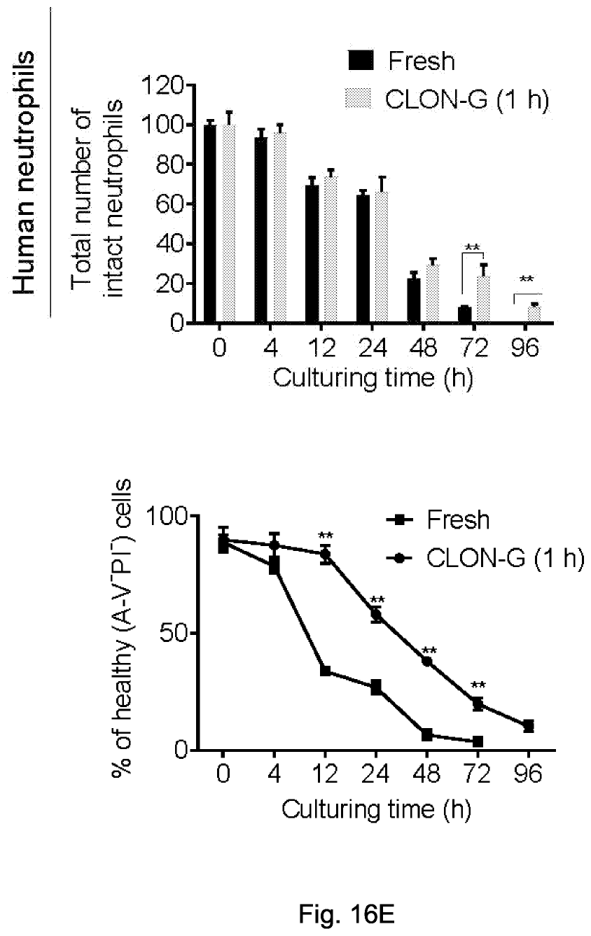

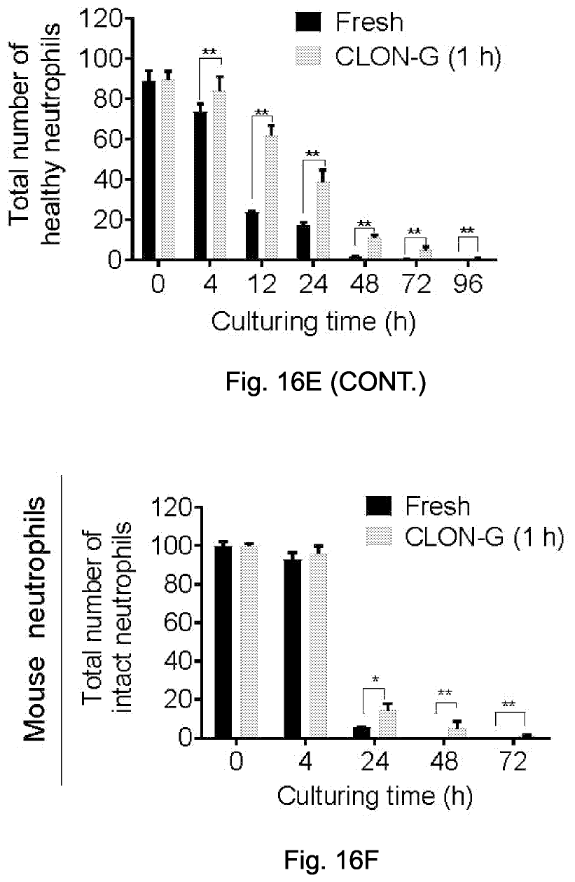

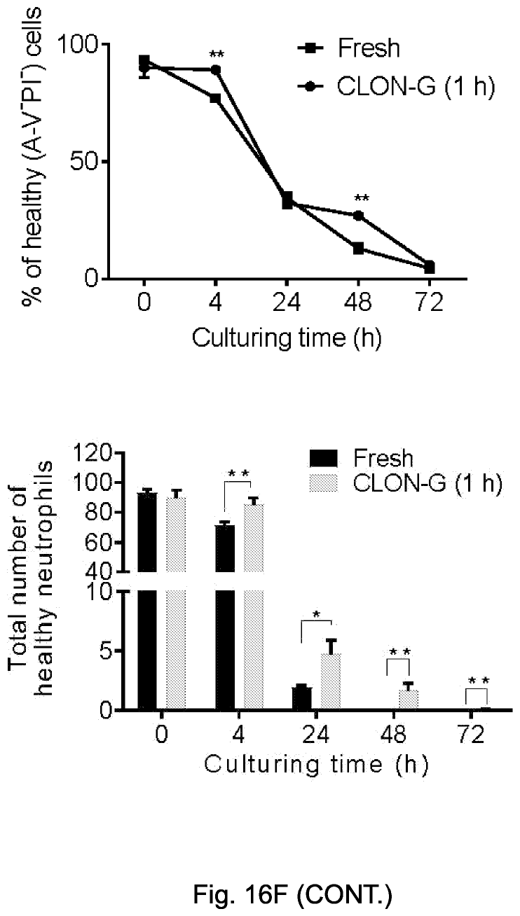

In some embodiments of any of the aspects, the method further comprises removing the composition or the cell culture medium as described herein from the neutrophil, or population thereof, after the sufficient amount of time to increase neutrophil lifespan. In some embodiments, the composition or cell culture medium comprising the caspase inhibitor, LMP inhibitor, antioxidant, growth factor, and/or necroptosis inhibitor is removed from the neutrophil, or population thereof after the sufficient amount of time to increase neutrophil lifespan. In some embodiments, the composition or the cell culture medium is removed by washing the neutrophil, or population thereof, such washing can be performed with a liquid that does not comprise the composition or cell culture medium as described, e.g., does not comprise the caspase inhibitor, LMP inhibitor, antioxidant, growth factor, and/or necroptosis inhibitor (see e.g., FIG. 2, FIG. 16). In some embodiments, after the removing (e.g., washing) step, the neutrophil, or population thereof, is contacted with a composition or cell culture medium that does not comprise the caspase inhibitor, LMP inhibitor, antioxidant, growth factor, and/or necroptosis inhibitor.

In some embodiments, the half-life of the neutrophil, or population thereof, is increased to at least 5 days after using the culture method described herein. In some embodiments, the half-life of the neutrophil, or population thereof, is increased to 1 day to 2 days, 2 days to 3 days, 3 days to 4 days, 4 days to 5 days, 5 days to 6 days, 6 days to 7 days, 3 days to 7 days, or 5 days to 7 days after using the culture method described herein.

In some embodiments, the neutrophil, or population thereof, is contacted with the composition as described herein ex vivo. In some embodiments, the neutrophil, or population thereof, is contacted with the cell culture medium as described herein ex vivo. The term “ex vivo” refers to a medical procedure in which an organ, cells, or tissue are taken from a living subject for a treatment or procedure, and then returned to the living subject.

In some embodiments, the contacting step comprises culturing. As used herein, the term “culture” or “culturing” refers to maintaining (tissue cells, bacteria, etc.; e.g., neutrophils) in conditions suitable for growth. In some embodiments, the neutrophils are cultured at a concentration of about 1×10⁶ cells/mL. In some embodiments, the neutrophils are cultured at a concentration of at least about 1×10³ cells/mL, at least about 1×10⁴ cells/mL, at least about 1×10⁵ cells/mL, at least about 1×10⁶ cells/mL, at least about 1×10⁷ cells/mL, at least about 1×10⁸ cells/mL, at least about 1×10⁹ cells/mL, at least about 1×10¹⁰ cells/mL, at least about 1×10¹¹ cells/mL, at least about 1×10¹² cells/mL, or more. In some embodiments, the neutrophils are cultured at a temperature of 37° C., e.g., about 35° C., about 36° C., about 37° C., about 38° C., about 39° C., about 40° C., 35° C.-37° C., 36° C.-38° C., or 37° C.-40° C. In some embodiments, the neutrophils are cultured at 5% CO₂, e.g., about 2.5% CO₂, about 3.0% CO₂, about 3.5% CO₂, about 4.0% CO₂, about 4.5% CO₂, about 5.0% CO₂, about 5.5% CO₂, about 6.0% CO₂, about 6.5% CO₂, about 7.0% CO₂. In some embodiments, the neutrophils are cultured in an incubator. In some embodiments, the neutrophils are cultured in a bioreactor.

### Treatment Methods

In multiple aspects, described herein are methods of treating neutropenia, or a neutropenia-associated disease or disorder, or a microbial infection in a subject in need thereof. In one aspect, the method comprises administering to a recipient subject in need thereof an effective amount of a neutrophil produced using the compositions, cell culture media, and/or methods described herein, or population thereof. In one aspect, the method comprises administering to a recipient subject in need thereof an effective amount of a composition comprising a neutrophil produced using the compositions, cell culture media, and/or methods described herein, or population thereof. In one aspect, the method comprises administering to a recipient subject in need thereof an effective amount of a pharmaceutical composition comprising a neutrophil produced using the compositions, cell culture media, and/or methods described herein, or population thereof.

In one aspect, the method comprises: (a) isolating a neutrophil or population thereof from a donor subject; (b) contacting the neutrophil, or population thereof, with a composition or cell culture medium as described herein; and (c) administering an effective amount of the cultured neutrophil, or population thereof, to a recipient subject in need thereof. In some embodiments, the donor subject is the recipient subject.

In some embodiments, the neutrophil, or population thereof, is obtained from a human. In some embodiments, the neutrophil, or population thereof, is obtained from a mouse. In some embodiments, the neutrophil, or population thereof, is obtained from the subject. In some embodiments, the treatment method is referred to as a granulocyte transfusion (GTX).

The neutrophils can be autologous/autogenic (“self”) or non-autologous (“non-self,” e.g., allogeneic, syngeneic or xenogeneic) in relation to the recipient of the cells. “Autologous,” as used herein, refers to cells from the same subject. “Allogeneic,” as used herein, refers to cells of the same species that differ genetically to the cell in comparison. “Syngeneic,” as used herein, refers to cells of a different subject that are genetically identical to the cell in comparison. “Xenogeneic,” as used herein, refers to cells of a different species to the cell in comparison. In some embodiments, the neutrophils are allogeneic. In various embodiments, the neutrophils to be implanted into a subject in need thereof is autologous or allogeneic to the subject. In various embodiments, the neutrophils described herein can be derived from one or more donors, or can be obtained from an autologous source. In some embodiments, the neutrophils are expanded in culture prior to administration to a subject in need thereof.

In some embodiments, the microbial infection is a bacterial infection (e.g., E. coli). In some embodiments, the microbial infection is a fungal infection (e.g., Candida albicans or other Candida species). In some embodiments, the microbial infection is a viral infection. In some embodiments, the microbial infection is a parasitic infection. In some embodiments, the microbial infection is a viral infection. In some embodiments, the microbial infection is associated with neutropenia. As a non-limiting example, viral infections are associated with neutropenia; exemplary viruses include Epstein-Barr virus, cytomegalovirus, hepatitis A and B viruses, parvovirus, Influenza virus species, and measles. Non-limiting examples of bacterial infections associated with neutropenia include Staphylococcus aureus (including Methicillin-resistant S. aureus), Enterococcus species (including Vancomycin-resistant Enterococci), Viridans streptococci, Streptococcus pneumoniae, Corynebacteria, Bacillus species, Clostridium septicum, Clostridium tertium, Clostridium difficile, Pseudomonas, Escherichia coli, Klebsiella, Acinetobacter, Enterobacter, Stenotrophomonas maltophilia, or Bacteroides species.

As described herein, levels of neutrophils can be decreased in subjects with neutropenia and/or a microbial infection. Accordingly, in one aspect of any of the embodiments, described herein is a method of treating neutropenia and/or a microbial infection in a subject in need thereof, the method comprising administering a composition comprising cultured neutrophils produced using the compositions, cell culture media, and/or methods described herein to a subject determined to have a level of neutrophils that is decreased relative to a reference. In one aspect of any of the embodiments, described herein is a method of treating neutropenia and/or a microbial infection in a subject in need thereof, the method comprising: a) determining the level of neutrophils in a sample obtained from a subject; and b) administering a composition comprising cultured neutrophils produced using the compositions, cell culture media, and/or methods described herein to the subject if the level of neutrophils is decreased relative to a reference.

In some embodiments of any of the aspects, the method comprises administering a composition comprising cultured neutrophils produced using the compositions, cell culture media, and/or methods described herein to a subject previously determined to have a level of neutrophils that is decreased relative to a reference. In some embodiments of any of the aspects, described herein is a method of treating neutropenia and/or a microbial infection in a subject in need thereof, the method comprising: a) first determining the level of neutrophils in a sample obtained from a subject; and b) then administering a composition comprising cultured neutrophils produced using the compositions, cell culture media, and/or methods described herein to the subject if the level of neutrophils is decreased relative to a reference.

In one aspect of any of the embodiments, described herein is a method of treating neutropenia and/or a microbial infection in a subject in need thereof, the method comprising: a) determining if the subject has a decreased level of neutrophils; and b) administering a composition comprising cultured neutrophils produced using the compositions, cell culture media, and/or methods described herein to the subject if the level of neutrophils is decreased relative to a reference. In some embodiments of any of the aspects, the step of determining if the subject has a decreased level of neutrophils can comprise i) obtaining or having obtained a sample from the subject and ii) performing or having performed an assay on the sample obtained from the subject to determine/measure the level of neutrophils in the subject. In some embodiments of any of the aspects, the step of determining if the subject has a decreased level of neutrophils can comprise performing or having performed an assay on a sample obtained from the subject to determine/measure the level of neutrophils in the subject. In some embodiments of any of the aspects, the step of determining if the subject has a decreased level of neutrophils can comprise ordering or requesting an assay on a sample obtained from the subject to determine/measure the level of neutrophils in the subject. In some embodiments of any of the aspects, the step of determining if the subject has a decreased level of neutrophils can comprise receiving the results of an assay on a sample obtained from the subject to determine/measure the level of neutrophils in the subject. In some embodiments of any of the aspects, the step of determining if the subject has a decreased level of neutrophils can comprise receiving a report, results, or other means of identifying the subject as a subject with a decreased level of neutrophils.

In one aspect of any of the embodiments, described herein is a method of treating neutropenia and/or a microbial infection in a subject in need thereof, the method comprising: a) determining if the subject has a decreased level of neutrophils; and b) instructing or directing that the subject be administered a composition comprising cultured neutrophils produced using the compositions, cell culture media, and/or methods described herein if the level of neutrophils is decreased relative to a reference. In some embodiments of any of the aspects, the step of determining if the subject has a decreased level of neutrophils can comprise i) obtaining or having obtained a sample from the subject and ii) performing or having performed an assay on the sample obtained from the subject to determine/measure the level of neutrophils in the subject. In some embodiments of any of the aspects, the step of determining if the subject has a decreased level of neutrophils can comprise performing or having performed an assay on a sample obtained from the subject to determine/measure the level of neutrophils in the subject. In some embodiments of any of the aspects, the step of determining if the subject has a decreased level of neutrophils can comprise ordering or requesting an assay on a sample obtained from the subject to determine/measure the level of neutrophils in the subject. In some embodiments of any of the aspects, the step of instructing or directing that the subject be administered a particular treatment can comprise providing a report of the assay results. In some embodiments of any of the aspects, the step of instructing or directing that the subject be administered a particular treatment can comprise providing a report of the assay results and/or treatment recommendations in view of the assay results.

The term “sample” or “test sample” as used herein denotes a sample taken or isolated from a biological organism, e.g., a blood or plasma sample from a subject. In some embodiments of any of the aspects, the technology described herein encompasses several examples of a biological sample. In some embodiments of any of the aspects, the biological sample is cells, or tissue, or peripheral blood, or bodily fluid. Exemplary biological samples include, but are not limited to, a biopsy, a tumor sample, biofluid sample; blood; serum; plasma; urine; semen; mucus; tissue biopsy; organ biopsy; synovial fluid; bile fluid; cerebrospinal fluid; mucosal secretion; effusion; sweat; saliva; and/or tissue sample etc. The term also includes a mixture of the above-mentioned samples. The term “test sample” also includes untreated or pretreated (or pre-processed) biological samples. In some embodiments of any of the aspects, a test sample can comprise cells from a subject.

In some embodiments of any of the aspects, the reference can be a level of neutrophils in a population of subjects who do not have or are not diagnosed as having, and/or do not exhibit signs or symptoms of neutropenia and/or a microbial infection. In some embodiments of any of the aspects, the reference can also be a level of neutrophils in a control sample, a pooled sample of control individuals or a numeric value or range of values based on the same. In some embodiments of any of the aspects, the reference can be the level neutrophils in a sample obtained from the same subject at an earlier point in time.

### Pharmaceutical Compositions and Administration

In multiple aspects, described herein are compositions comprising a neutrophil, or population thereof, produced using the compositions, cell culture media, and/or methods described herein. In one aspect, described herein is a pharmaceutical composition comprising a neutrophil as described herein, or population thereof, and a pharmaceutically acceptable carrier. In some embodiments, the pharmaceutical composition is for use in treating neutropenia or a microbial infection in a subject.

In some embodiments, the methods described herein relate to treating a subject having or diagnosed as having neutropenia with a composition comprising neutrophils produced using the compositions, cell culture media, and/or methods described herein. Neutropenia is defined by the presence of abnormally few neutrophils in the blood, leading to increased susceptibility to infection. Neutropenia can be caused by cancer or cancer treatment such as chemotherapy or radiation. Neutropenia can also be caused by bone marrow suppression or to peripheral destruction of neutrophils. Subjects having neutropenia can be identified by a physician using current methods of diagnosing neutropenia. Normal neutrophil counts depend on different factors such as age, but generally, a low neutrophil level is less than 45% of total white blood cells or 1,5000 neutrophils per microliter. A normal neutrophil level is between 1,500 and 8,000 neutrophils per microliter. Symptoms and/or complications of neutropenia which characterize these conditions and aid in diagnosis are well known in the art and include but are not limited to, a fever, which is a temperature of 100.5° F. (38° C.) or higher; chills or sweating; sore throat, sores in the mouth, or a toothache; abdominal pain; pain near the anus; pain or burning when urinating, or urinating often; diarrhea or sores around the anus; a cough or shortness of breath; any redness, swelling, or pain (especially around a cut, wound, or catheter); and/or unusual vaginal discharge or itching. Tests that may aid in a diagnosis of, e.g. neutropenia include, but are not limited to, a blood smear or flow cytometry. A family history of neutropenia, or exposure to risk factors for neutropenia (e.g. elevated age, pre-exiting immunodeficiency from HIV or an organ transplant) can also aid in determining if a subject is likely to have neutropenia or in making a diagnosis of neutropenia.

In some embodiments, the methods described herein relate to treating a subject having or diagnosed as having a microbial infection with a composition comprising neutrophils produced using the compositions, cell culture media, and/or methods described herein. In some embodiments, the microbial infection is caused by a bacterium, fungus, virus, or parasite. Subjects having a microbial infection can be identified by a physician using current methods of diagnosing microbial infections. Symptoms and/or complications of a microbial infection depend on the specific microorganism(s) but can include, but are not limited to, fever, chills, sweats, swollen lymph nodes, new or sudden worsening of pain, unexplained exhaustion, headache, skin flushing, swelling, soreness, and/or gastrointestinal symptoms (such as nausea, vomiting, diarrhea, abdominal or rectal pain). Tests that may aid in a diagnosis of, e.g. a microbial infection include, but are not limited to, detection of pathogen associated molecular patterns (PAMPs) such as microbe-specific proteins, nucleic acids, or lipids, or detection of microbe-specific antibodies, T-cells, or B-cells. A family history of microbial infections, or immunodeficiency, or exposure to risk factors for microbial infections (e.g., advanced age, young age, malnutrition, chronic diseases, cognitive deficits that may complicate compliance with basic sanitary practices, diminished ability to complain of or self-identify symptoms, functional impairments such as incontinence or immobility, blunted febrile response to infection, or use of invasive devices like catheters, ventilators, or feeding tubes) can also aid in determining if a subject is likely to have a microbial infection or in making a diagnosis of a microbial infection.

The compositions described herein can be administered to a subject having or diagnosed as having neutropenia and/or a microbial infection. In some embodiments, the methods described herein comprise administering an effective amount of compositions described herein, e.g., a neutrophil, or population thereof, produced using the compositions, cell culture media, and/or methods described herein, to a subject in order to alleviate a symptom of neutropenia and/or a microbial infection. As used herein, “alleviating a symptom of neutropenia and/or a microbial infection” is ameliorating any condition or symptom associated with the neutropenia and/or a microbial infection. As compared with an equivalent untreated control, such reduction is by at least 5%, 10%, 20%, 40%, 50%, 60%, 80%, 90%, 95%, 99% or more as measured by any standard technique. A variety of means for administering the compositions described herein to subjects are known to those of skill in the art. Such methods can include, but are not limited to intravenous, intramuscular, intraperitoneal, or intratumoral administration. Administration can be local or systemic.

The term “effective amount” as used herein refers to the amount of composition comprising the neutrophils needed to alleviate at least one or more symptom of the disease or disorder, and relates to a sufficient amount of pharmacological composition to provide the desired effect. The term “therapeutically effective amount” therefore refers to an amount of the composition comprising neutrophils that is sufficient to provide a particular anti-neutropenia and/or antimicrobial effect when administered to a typical subject. An effective amount as used herein, in various contexts, would also include an amount sufficient to delay the development of a symptom of the disease, alter the course of a symptom disease (for example but not limited to, slowing the progression of a symptom of the disease), or reverse a symptom of the disease. Thus, it is not generally practicable to specify an exact “effective amount”. However, for any given case, an appropriate “effective amount” can be determined by one of ordinary skill in the art using only routine experimentation.

Effective amounts, toxicity, and therapeutic efficacy can be determined by standard pharmaceutical procedures in cell cultures or experimental animals, e.g., for determining the ED50 (the dose therapeutically effective in 50% of the population). The dosage can vary depending upon the dosage form employed and the route of administration utilized. A therapeutically effective dose can be estimated initially from cell culture assays. Also, a dose can be formulated in animal models to achieve a circulating blood concentration range that includes the IC50 (i.e., the concentration of neutrophils which achieves a half-maximal inhibition of symptoms) as determined in cell culture, or in an appropriate animal model. Levels of neutrophils in blood can be measured, for example, by blood smears or flow cytometry. The effects of any particular dosage can be monitored by a suitable bioassay, e.g., assay for neutrophil functionality (e.g., in vitro or in vivo chemotaxis, recruitment, NADPH oxidase activation, and/or microbial killing) among others. The dosage can be determined by a physician and adjusted, as necessary, to suit observed effects of the treatment.

In some embodiments, the technology described herein relates to a pharmaceutical composition comprising neutrophils produced using the compositions, cell culture media, and/or methods described as described herein, and optionally a pharmaceutically acceptable carrier. In some embodiments, the active ingredients of the pharmaceutical composition comprise the neutrophils as described herein. In some embodiments, the active ingredients of the pharmaceutical composition consist essentially of the neutrophils as described herein. In some embodiments, the active ingredients of the pharmaceutical composition consist of the neutrophils as described herein. Pharmaceutically acceptable carriers and diluents include saline, aqueous buffer solutions, solvents and/or dispersion media. The use of such carriers and diluents is well known in the art. Some non-limiting examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids; (23) serum component, such as serum albumin, HDL and LDL; (24) C₂-C₁₂ alcohols; and (25) other non-toxic compatible substances employed in pharmaceutical formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein. In some embodiments, the carrier inhibits the death or lysis of the neutrophils as described herein.

In some embodiments of any of the aspects, the composition comprising neutrophils described herein is administered as a monotherapy, e.g., another treatment for the neutropenia and/or microbial infection is not administered to the subject.

In some embodiments of any of the aspects, the methods described herein can further comprise administering a second agent and/or treatment to the subject, e.g. as part of a combinatorial therapy. Non-limiting examples of a second agent and/or treatment can include a cancer therapy selected from the group consisting of: radiation therapy, surgery, gemcitabine, cisplatin, paclitaxel, carboplatin, bortezomib, AMG479, vorinostat, rituximab, temozolomide, rapamycin, ABT-737, PI-103; alkylating agents such as thiotepa and CYTOXAN® cyclophosphamide; alkyl sulfonates such as busulfan, improsulfan and piposulfan; aziridines such as benzodopa, carboquone, meturedopa, and uredopa; ethylenimines and methylmelamines including altretamine, triethylenemelamine, trietylenephosphoramide, triethylenethiophosphoramide and trimethylol melamine; acetogenins (especially bullatacin and bullatacinone); a camptothecin (including the synthetic analogue topotecan); bryostatin; callystatin; CC-1065 (including its adozelesin, carzelesin and bizelesin synthetic analogues); cryptophycins (particularly cryptophycin 1 and cryptophycin 8); dolastatin; duocarmycin (including the synthetic analogues, KW-2189 and CB1-TM1); eleutherobin; pancratistatin; a sarcodictyin; spongistatin; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimustine; antibiotics such as the enediyne antibiotics (e.g., calicheamicin, especially calicheamicin gamma1I and calicheamicin omegaI1 (see, e.g., Agnew, Chem. Intl. Ed. Engl., 33: 183-186 (1994)); dynemicin, including dynemicin A; bisphosphonates, such as clodronate; an esperamicin; as well as neocarzinostatin chromophore and related chromoprotein enediyne antibiotic chromophores), aclacinomycins, actinomycin, authramycin, azaserine, bleomycins, cactinomycin, carabicin, caminomycin, carzinophilin, chromomycins, dactinomycin, daunorubicin, detorubicin, 6-diazo-5-oxo-L-norleucine, ADRIAMYCIN® doxorubicin (including morpholino-doxorubicin, cyanomorpholino-doxorubicin, 2-pyrrolino-doxorubicin and deoxydoxorubicin), epirubicin, esorubicin, idarubicin, marcellomycin, mitomycins such as mitomycin C, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elformithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, Oreg.); razoxane; rhizoxin; sizofuran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; taxoids, e.g., TAXOL® paclitaxel (Bristol-Myers Squibb Oncology, Princeton, N.J.), ABRAXANE® Cremophor-free, albumin-engineered nanoparticle formulation of paclitaxel (American Pharmaceutical Partners, Schaumberg, Ill.), and TAXOTERE® doxetaxel (Rhone-Poulenc Rorer, Antony, France); chloranbucil; GEMZAR® gemcitabine; 6-thioguanine; mercaptopurine; methotrexate; platinum analogs such as cisplatin, oxaliplatin and carboplatin; vinblastine; platinum; etoposide (VP-16); ifosfamide; mitoxantrone; vincristine; NAVELBINE® vinorelbine; novantrone; teniposide; edatrexate; daunomycin; aminopterin; xeloda; ibandronate; irinotecan (Camptosar, CPT-11) (including the treatment regimen of irinotecan with 5-FU and leucovorin); topoisomerase inhibitor RFS 2000; difluoromethylornithine (DMFO); retinoids such as retinoic acid; capecitabine; combretastatin; leucovorin (LV); oxaliplatin, including the oxaliplatin treatment regimen (FOLFOX); lapatinib (Tykerb®); inhibitors of PKC-alpha, Raf, H-Ras, EGFR (e.g., erlotinib (Tarceva®)) and VEGF-A that reduce cell proliferation and pharmaceutically acceptable salts, acids or derivatives of any of the above.

One of skill in the art can readily identify a chemotherapeutic agent of use (e.g. see Physicians' Cancer Chemotherapy Drug Manual 2014, Edward Chu, Vincent T. DeVita Jr., Jones & Bartlett Learning; Principles of Cancer Therapy, Chapter 85 in Harrison's Principles of Internal Medicine, 18th edition; Therapeutic Targeting of Cancer Cells: Era of Molecularly Targeted Agents and Cancer Pharmacology, Chs. 28-29 in Abeloff's Clinical Oncology, 2013 Elsevier; and Fischer D S (ed): The Cancer Chemotherapy Handbook, 4th ed. St. Louis, Mosby-Year Book, 2003). In addition, the methods of treatment can further include the use of radiation or radiation therapy. Further, the methods of treatment can further include the use of surgical treatments.

The methods described herein can further comprise administering a second agent and/or treatment to the subject, e.g. as part of a combinatorial therapy. By way of non-limiting example, if a subject is to be treated for pain or inflammation according to the methods described herein, the subject can also be administered a second agent and/or treatment known to be beneficial for subjects suffering from pain or inflammation. Examples of such agents and/or treatments include, but are not limited to, non-steroidal anti-inflammatory drugs (NSAIDs—such as aspirin, ibuprofen, or naproxen); corticosteroids, including glucocorticoids (e.g. cortisol, prednisone, prednisolone, methylprednisolone, dexamethasone, betamethasone, triamcinolone, and beclometasone); methotrexate; sulfasalazine; leflunomide; anti-TNF medications; cyclophosphamide; pro-resolving drugs; mycophenolate; or opiates (e.g. endorphins, enkephalins, and dynorphin), steroids, analgesics, barbiturates, oxycodone, morphine, lidocaine, and the like.

In certain embodiments, an effective dose of a composition comprising neutrophils as described herein can be administered to a patient once. In certain embodiments, an effective dose of a composition comprising neutrophils can be administered to a patient repeatedly. For systemic administration, subjects can be administered a therapeutic amount of a composition comprising neutrophils, such as, at least about 1×10³ cells/mL, at least about 1×10⁴ cells/mL, at least about 1×10⁵ cells/mL, at least about 1×10⁶ cells/mL, at least about 1×10⁷ cells/mL, at least about 1×10⁸ cells/mL, at least about 1×10⁹ cells/mL, at least about 1×10¹⁰ cells/mL, at least about 1×10¹¹ cells/mL, at least about 1×10¹² cells/mL, or more.

In some embodiments, after an initial treatment regimen, the treatments can be administered on a less frequent basis. For example, after treatment biweekly for three months, treatment can be repeated once per month, for six months or a year or longer. Treatment according to the methods described herein can reduce levels of a marker or symptom of a condition, e.g., apoptotic or lytic neutrophils, microbial concentrations, etc., by at least 10%, at least 15%, at least 20%, at least 25%, at least 30%, at least 40%, at least 50%, at least 60%, at least 70%, at least 80% or at least 90% or more.

The dosage of a composition as described herein can be determined by a physician and adjusted, as necessary, to suit observed effects of the treatment. With respect to duration and frequency of treatment, it is typical for skilled clinicians to monitor subjects in order to determine when the treatment is providing therapeutic benefit, and to determine whether to increase or decrease dosage, increase or decrease administration frequency, discontinue treatment, resume treatment, or make other alterations to the treatment regimen. The dosing schedule can vary from once a week to daily depending on a number of clinical factors, such as the subject's sensitivity to the administered neutrophils. The desired dose or amount can be administered at one time or divided into subdoses, e.g., 2-4 subdoses and administered over a period of time, e.g., at appropriate intervals through the day or other appropriate schedule. In some embodiments, administration can be chronic, e.g., one or more doses and/or treatments daily over a period of weeks or months. Examples of dosing and/or treatment schedules are administration daily, twice daily, three times daily or four or more times daily over a period of 1 week, 2 weeks, 3 weeks, 4 weeks, 1 month, 2 months, 3 months, 4 months, 5 months, or 6 months, or more. A composition comprising neutrophils can be administered over a period of time, such as over a 5 minute, 10 minute, 15 minute, 20 minute, or 25 minute period.

The dosage ranges for the administration of neutrophils, according to the methods described herein depend upon, for example, the extent to which symptoms, markers, or indicators of a condition described herein are desired to be reduced. The dosage should not be so large as to cause adverse side effects, such as hyperactive neutrophils, tissue damage, edema, and/or elevated inflammatory cytokines. Generally, the dosage will vary with the age, condition, and sex of the patient and can be determined by one of skill in the art. The dosage can also be adjusted by the individual physician in the event of any complication.

The efficacy of compositions comprising neutrophils in, e.g., the treatment of a condition described herein can be determined by the skilled clinician. However, a treatment is considered “effective treatment,” as the term is used herein, if one or more of the signs or symptoms of a condition described herein are altered in a beneficial manner, other clinically accepted symptoms are improved, or even ameliorated, or a desired response is induced e.g., by at least 10% following treatment according to the methods described herein. Efficacy can be assessed, for example, by measuring a marker, indicator, symptom, and/or the incidence of a condition treated according to the methods described herein or any other measurable parameter appropriate, e.g., the level of viable circulating neutrophils. Efficacy can also be measured by a failure of an individual to worsen as assessed by hospitalization, or need for medical interventions (i.e., progression of the disease is halted). Methods of measuring these indicators are known to those of skill in the art and/or are described herein. Treatment includes any treatment of a disease in an individual or an animal (some non-limiting examples include a human or an animal) and includes: (1) inhibiting the disease, e.g., preventing a worsening of symptoms (e.g., pain or inflammation); or (2) relieving the severity of the disease, e.g., causing regression of symptoms. An effective amount for the treatment of a disease means that amount which, when administered to a subject in need thereof, is sufficient to result in effective treatment as that term is defined herein, for that disease. Efficacy of an agent can be determined by assessing physical indicators of a condition or desired response. It is well within the ability of one skilled in the art to monitor efficacy of administration and/or treatment by measuring any one of such parameters, or any combination of parameters. Efficacy can be assessed in animal models of a condition described herein, for example treatment of neutropenia and/or a microbial infection. When using an experimental animal model, efficacy of treatment is evidenced when a statistically significant change in a marker is observed, e.g. neutrophils levels.

In vitro and animal model assays are provided herein which allow the assessment of a given dose of neutrophils. By way of non-limiting example, the effects of a dose of neutrophils can be assessed by neutrophil functionality tests, such as in vitro or in vivo chemotaxis, recruitment, NADPH oxidase activation, and/or microbial killing. The efficacy of a given dosage can also be assessed in an animal model, e.g., wild-type C57BL/6 or NOD.Cg-Prkdc^{scid} Il2rgtm1^{Wjl}/SzJ (NSG) mice. For example, neutropenia can be induced in mice, such as NSG mice, with thioglycolate (TG) injection.

### Definitions

For convenience, the meaning of some terms and phrases used in the specification, examples, and appended claims, are provided below. Unless stated otherwise, or implicit from context, the following terms and phrases include the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs. If there is an apparent discrepancy between the usage of a term in the art and its definition provided herein, the definition provided within the specification shall prevail.

The terms “decrease”, “reduced”, “reduction”, or “inhibit” are all used herein to mean a decrease by a statistically significant amount. In some embodiments, “reduce,” “reduction” or “decrease” or “inhibit” typically means a decrease by at least 10% as compared to a reference level (e.g. the absence of a given treatment or agent) and can include, for example, a decrease by at least about 10%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, at least about 98%, at least about 99%, or more. As used herein, “reduction” or “inhibition” does not encompass a complete inhibition or reduction as compared to a reference level. “Complete inhibition” is a 100% inhibition as compared to a reference level. A decrease can be preferably down to a level accepted as within the range of normal, e.g., for an individual without a given disorder.

The terms “increased”, “increase”, “enhance”, or “activate” are all used herein to mean an increase by a statically significant amount. In some embodiments, the terms “increased”, “increase”, “enhance”, or “activate” can mean an increase of at least 10% as compared to a reference level, for example an increase of at least about 20%, or at least about 30%, or at least about 40%, or at least about 50%, or at least about 60%, or at least about 70%, or at least about 80%, or at least about 90% or up to and including a 100% increase or any increase between 10-100% as compared to a reference level, or at least about a 2-fold, or at least about a 3-fold, or at least about a 4-fold, or at least about a 5-fold or at least about a 10-fold increase, or any increase between 2-fold and 10-fold or greater as compared to a reference level. In the context of a marker or symptom, an “increase” is a statistically significant increase in such level.

As used herein, a “subject” means a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In some embodiments, the subject is a mammal, e.g., a primate, e.g., a human. The terms, “individual,” “patient” and “subject” are used interchangeably herein.

Preferably, the subject is a mammal. The mammal can be a human, non-human primate, mouse, rat, dog, cat, horse, or cow, but is not limited to these examples. Mammals other than humans can be advantageously used as subjects that represent animal models of neutropenia and/or a microbial infection. A subject can be male or female.

A subject can be one who has been previously diagnosed with or identified as suffering from or having a condition in need of treatment (e.g., neutropenia and/or a microbial infection) or one or more complications related to such a condition, and optionally, have already undergone treatment for neutropenia and/or a microbial infection or the one or more complications related to neutropenia and/or a microbial infection. Alternatively, a subject can also be one who has not been previously diagnosed as having neutropenia and/or a microbial infection or one or more complications related to neutropenia and/or a microbial infection. For example, a subject can be one who exhibits one or more risk factors for neutropenia and/or a microbial infection or one or more complications related to neutropenia and/or a microbial infection or a subject who does not exhibit risk factors.

A “subject in need” of treatment for a particular condition can be a subject having that condition, diagnosed as having that condition, or at risk of developing that condition.

As used herein, the terms “protein” and “polypeptide” are used interchangeably to designate a series of amino acid residues, connected to each other by peptide bonds between the alpha-amino and carboxy groups of adjacent residues. The terms “protein”, and “polypeptide” refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to a gene product and fragments thereof. Thus, exemplary polypeptides or proteins include gene products, naturally occurring proteins, homologs, orthologs, paralogs, fragments and other equivalents, variants, fragments, and analogs of the foregoing.

In the various embodiments described herein, it is further contemplated that variants (naturally occurring or otherwise), alleles, homologs, conservatively modified variants, and/or conservative substitution variants of any of the particular polypeptides described are encompassed. As to amino acid sequences, one of skill will recognize that individual substitutions, deletions or additions to a nucleic acid, peptide, polypeptide, or protein sequence which alters a single amino acid or a small percentage of amino acids in the encoded sequence is a “conservatively modified variant” where the alteration results in the substitution of an amino acid with a chemically similar amino acid and retains the desired activity of the polypeptide. Such conservatively modified variants are in addition to and do not exclude polymorphic variants, interspecies homologs, and alleles consistent with the disclosure.

A given amino acid can be replaced by a residue having similar physiochemical characteristics, e.g., substituting one aliphatic residue for another (such as Ile, Val, Leu, or Ala for one another), or substitution of one polar residue for another (such as between Lys and Arg; Glu and Asp; or Gln and Asn). Other such conservative substitutions, e.g., substitutions of entire regions having similar hydrophobicity characteristics, are well known. Polypeptides comprising conservative amino acid substitutions can be tested in any one of the methods or assays described herein to confirm that a desired activity, e.g. prolonging neutrophil lifespan and/or specificity of a native or reference polypeptide is retained.

Amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); (4) basic: Lys (K), Arg (R), His (H). Alternatively, naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; (6) aromatic: Trp, Tyr, Phe. Non-conservative substitutions will entail exchanging a member of one of these classes for another class. Particular conservative substitutions include, for example; Ala into Gly or into Ser; Arg into Lys; Asn into Gln or into His; Asp into Glu; Cys into Ser; Gln into Asn; Glu into Asp; Gly into Ala or into Pro; His into Asn or into Gln; Ile into Leu or into Val; Leu into Ile or into Val; Lys into Arg, into Gln or into Glu; Met into Leu, into Tyr or into Ile; Phe into Met, into Leu or into Tyr; Ser into Thr; Thr into Ser; Trp into Tyr; Tyr into Trp; and/or Phe into Val, into Ile or into Leu.

In some embodiments, the polypeptide described herein (or a nucleic acid encoding such a polypeptide) can be a functional fragment of one of the amino acid sequences described herein. As used herein, a “functional fragment” is a fragment or segment of a polypeptide which retains at least 50% of the wild-type reference polypeptide's activity according to the methods and/or assays described below herein. A functional fragment can comprise conservative substitutions of the sequences disclosed herein.

In some embodiments, the polypeptide described herein can be a variant of a polypeptide sequence described herein. In some embodiments, the variant is a conservatively modified variant. Conservative substitution variants can be obtained by mutations of native nucleotide sequences, for example. A “variant,” as referred to herein, is a polypeptide substantially homologous to a native or reference polypeptide, but which has an amino acid sequence different from that of the native or reference polypeptide because of one or a plurality of deletions, insertions or substitutions. Variant polypeptide-encoding DNA sequences encompass sequences that comprise one or more additions, deletions, or substitutions of nucleotides when compared to a native or reference DNA sequence, but that encode a protein or fragment thereof that retains activity of the native or reference polypeptide. A wide variety of, for example, PCR-based, site-specific mutagenesis approaches are known in the art and can be applied by the ordinarily skilled artisan to generate and test artificial variants.

A variant amino acid or DNA sequence can be at least 80%, at least 85%, at least 90%, at least 91%, at least 92%, at least 93%, at least 94%, at least 95%, at least 96%, at least 97%, at least 98%, at least 99%, or more, identical to a native or reference sequence. The degree of homology (percent identity) between a native and a mutant sequence can be determined, for example, by comparing the two sequences using freely available computer programs commonly employed for this purpose on the world wide web (e.g. BLASTp or BLASTn with default settings).

A variant amino acid sequence can be at least 50%, at least 60%, at least 70%, at least 80%, at least 90%, at least 91%, at least 92%, at least 93%, at least 94%, at least 95%, at least 96%, at least 97%, at least 98%, at least 99%, or more, similar to a native or reference sequence. As used herein, “similarity” refers to an identical amino acid or a conservatively substituted amino acid, as described herein. Accordingly, the percentage of “sequence similarity” is the percentage of amino acids which is either identical or conservatively changed; e.g., “sequence similarity”=(% sequence identity)+(% conservative changes). It should be understood that a sequence that has a specified percent similarity to a reference sequence necessarily encompasses a sequence with the same specified percent identity to that reference sequence. The skilled person will be aware of various computer programs, using different mathematical algorithms, that are available to determine the identity or similarity between two sequences. For instance, use can be made of a computer program employing the Needleman and Wunsch algorithm (Needleman et al. (1970)); the GAP program in the Accelrys GCG software package (Accelerys Inc., San Diego U.S.A.); the algorithm of E. Meyers and W. Miller (Meyers et al. (1989)) which has been incorporated into the ALIGN program (version 2.0); or more preferably the BLAST (Basic Local Alignment Tool using default parameters); see e.g., U.S. Pat. No. 10,023,890, the content of which is incorporated by reference herein in its entirety.

Alterations of the native amino acid sequence can be accomplished by any of a number of techniques known to one of skill in the art. Mutations can be introduced, for example, at particular loci by synthesizing oligonucleotides containing a mutant sequence, flanked by restriction sites enabling ligation to fragments of the native sequence. Following ligation, the resulting reconstructed sequence encodes an analog having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion required. A wide variety of, site-specific mutagenesis approaches, e.g., Kunkel's method, cassette mutagenesis, PCR site-directed mutagenesis (e.g., traditional PCR, primer extension, or inverse PCR), whole plasmid mutagenesis, in vivo site-directed mutagenesis, CRISPR/Cas-guided mutagenesis, are known in the art and can be applied by the ordinarily skilled artisan to introduce mutations into specific nucleic acid loci. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); Braman, Jeff, ed. (2002) In Vitro Mutagenesis Protocols, Methods in Molecular Biology, Vol. 182 (2nd ed.); Khudyakov and Fields (2002), Artificial DNA: Methods and Applications, CRC Press; Hsu et al. (2014), Cell 157 (6): 1262-78; Cerchione et al. (2020) PLOS ONE 15 (4): e0231716; and U.S. Pat. Nos. 4,518,584 and 4,737,462, which are herein incorporated by reference in their entireties. Any cysteine residue not involved in maintaining the proper conformation of the polypeptide also can be substituted, generally with serine, to improve the oxidative stability of the molecule and prevent aberrant crosslinking. Conversely, cysteine bond(s) can be added to the polypeptide to improve its stability or facilitate oligomerization.

As used herein, the term “nucleic acid” or “nucleic acid sequence” refers to any molecule, preferably a polymeric molecule, incorporating units of ribonucleic acid, deoxyribonucleic acid or an analog thereof. The nucleic acid can be either single-stranded or double-stranded. A single-stranded nucleic acid can be one nucleic acid strand of a denatured double-stranded DNA. Alternatively, it can be a single-stranded nucleic acid not derived from any double-stranded DNA. In one aspect, the nucleic acid can be DNA. In another aspect, the nucleic acid can be RNA. Suitable DNA can include, e.g., genomic DNA or cDNA. Suitable RNA can include, e.g., mRNA.

As used herein, the terms “treat,” “treatment,” “treating,” or “amelioration” refer to therapeutic treatments, wherein the object is to reverse, alleviate, ameliorate, inhibit, slow down or stop the progression or severity of a condition associated with a disease or disorder, e.g. neutropenia and/or a microbial infection. The term “treating” includes reducing or alleviating at least one adverse effect or symptom of a condition, disease or disorder associated with neutropenia and/or a microbial infection. Treatment is generally “effective” if one or more symptoms or clinical markers are reduced. Alternatively, treatment is “effective” if the progression of a disease is reduced or halted. That is, “treatment” includes not just the improvement of symptoms or markers, but also a cessation of, or at least slowing of, progress or worsening of symptoms compared to what would be expected in the absence of treatment. Beneficial or desired clinical results include, but are not limited to, alleviation of one or more symptom(s), diminishment of extent of disease, stabilized (i.e., not worsening) state of disease, delay or slowing of disease progression, amelioration or palliation of the disease state, remission (whether partial or total), and/or decreased mortality, whether detectable or undetectable. The term “treatment” of a disease also includes providing relief from the symptoms or side-effects of the disease (including palliative treatment).

As used herein, the term “pharmaceutical composition” refers to the active agent in combination with a pharmaceutically acceptable carrier e.g. a carrier commonly used in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. In some embodiments of any of the aspects, a pharmaceutically acceptable carrier can be a carrier other than water. In some embodiments of any of the aspects, a pharmaceutically acceptable carrier can be a cream, emulsion, gel, liposome, nanoparticle, and/or ointment. In some embodiments of any of the aspects, a pharmaceutically acceptable carrier can be an artificial or engineered carrier, e.g., a carrier that the active ingredient would not be found to occur in or within nature.

As used herein, the term “administering,” refers to the placement of a compound as disclosed herein into a subject by a method or route which results in at least partial delivery of the agent at a desired site. Pharmaceutical compositions comprising the compounds disclosed herein can be administered by any appropriate route which results in an effective treatment in the subject. In some embodiments, administration comprises physical human activity, e.g., an injection, act of ingestion, an act of application, and/or manipulation of a delivery device or machine. Such activity can be performed, e.g., by a medical professional and/or the subject being treated.

As used herein, “contacting” refers to any suitable means for delivering, or exposing, an agent to at least one cell. Exemplary delivery methods include, but are not limited to, direct delivery to cell culture medium, transfection, transduction, perfusion, injection, or other delivery method known to one skilled in the art. In some embodiments, contacting comprises physical human activity, e.g., an injection; an act of dispensing, mixing, and/or decanting; and/or manipulation of a delivery device or machine.

In some embodiments of any of the aspects, the cells can be maintained in culture. As used herein, “maintaining” refers to continuing the viability of a cell or population of cells. A maintained population of cells will have at least a subpopulation of metabolically active cells.

As used herein, the term “analog” refers to a substance that shares one or more particular structural features, elements, components, or moieties with a reference substance. Typically, an “analog” shows significant structural similarity with the reference substance, for example sharing a core or consensus structure, but also differs in certain discrete ways. In some embodiments, an analog is a substance that can be generated from the reference substance, e.g., by chemical manipulation of the reference substance.

The term “statistically significant” or “significantly” refers to statistical significance and generally means a two standard deviation (2SD) or greater difference.

Other than in the operating examples, or where otherwise indicated, all numbers expressing quantities of ingredients or reaction conditions used herein should be understood as modified in all instances by the term “about.” The term “about” when used in connection with percentages or concentrations can mean±1%.

As used herein, the term “comprising” means that other elements can also be present in addition to the defined elements presented. The use of “comprising” indicates inclusion rather than limitation.

The term “consisting of” refers to compositions, methods, and respective components thereof as described herein, which are exclusive of any element not recited in that description of the embodiment.

As used herein the term “consisting essentially of” refers to those elements required for a given embodiment. The term permits the presence of additional elements that do not materially affect the basic and novel or functional characteristic(s) of that embodiment of the invention.

The singular terms “a,” “an,” and “the” include plural referents unless context clearly indicates otherwise. Similarly, the word “or” is intended to include “and” unless the context clearly indicates otherwise. Although methods and materials similar or equivalent to those described herein can be used in the practice or testing of this disclosure, suitable methods and materials are described below. The abbreviation, “e.g.” is derived from the Latin exempli gratia, and is used herein to indicate a non-limiting example. Thus, the abbreviation “e.g.” is synonymous with the term “for example.”

Groupings of alternative elements or embodiments of the invention disclosed herein are not to be construed as limitations. Each group member can be referred to and claimed individually or in any combination with other members of the group or other elements found herein. One or more members of a group can be included in, or deleted from, a group for reasons of convenience and/or patentability. When any such inclusion or deletion occurs, the specification is herein deemed to contain the group as modified thus fulfilling the written description of all Markush groups used in the appended claims.

Unless otherwise defined herein, scientific and technical terms used in connection with the present application shall have the meanings that are commonly understood by those of ordinary skill in the art to which this disclosure belongs. It should be understood that this invention is not limited to the particular methodology, protocols, and reagents, etc., described herein and as such can vary. The terminology used herein is for the purpose of describing particular embodiments only, and is not intended to limit the scope of the present invention, which is defined solely by the claims. Definitions of common terms in cell biology, immunology, and molecular biology can be found in The Merck Manual of Diagnosis and Therapy, 20th Edition, published by Merck Sharp & Dohme Corp., 2018 (ISBN 0911910190, 978-0911910421); Robert S. Porter et al. (eds.), The Encyclopedia of Molecular Cell Biology and Molecular Medicine, published by Blackwell Science Ltd., 1999-2012 (ISBN 9783527600908); and Robert A. Meyers (ed.), Molecular Biology and Biotechnology: a Comprehensive Desk Reference, published by VCH Publishers, Inc., 1995 (ISBN 1-56081-569-8); Immunology by Werner Luttmann, published by Elsevier, 2006; Janeway's Immunobiology, Kenneth Murphy, Allan Mowat, Casey Weaver (eds.), W. W. Norton & Company, 2016 (ISBN 0815345054, 978-0815345053); Lewin's Genes XI, published by Jones & Bartlett Publishers, 2014 (ISBN-1449659055); Michael Richard Green and Joseph Sambrook, Molecular Cloning: A Laboratory Manual, 4th ed., Cold Spring Harbor Laboratory Press, Cold Spring Harbor, N.Y., USA (2012) (ISBN 1936113414); Davis et al., Basic Methods in Molecular Biology, Elsevier Science Publishing, Inc., New York, USA (2012) (ISBN 044460149X); Laboratory Methods in Enzymology: DNA, Jon Lorsch (ed.) Elsevier, 2013 (ISBN 0124199542); Current Protocols in Molecular Biology (CPMB), Frederick M. Ausubel (ed.), John Wiley and Sons, 2014 (ISBN 047150338X, 9780471503385), Current Protocols in Protein Science (CPPS), John E. Coligan (ed.), John Wiley and Sons, Inc., 2005; and Current Protocols in Immunology (CPI) (John E. Coligan, ADA M Kruisbeek, David H Margulies, Ethan M Shevach, Warren Strobe, (eds.) John Wiley and Sons, Inc., 2003 (ISBN 0471142735, 9780471142737), the contents of which are all incorporated by reference herein in their entireties.

Other terms are defined herein within the description of the various aspects of the invention.

All patents and other publications; including literature references, issued patents, published patent applications, and co-pending patent applications; cited throughout this application are expressly incorporated herein by reference for the purpose of describing and disclosing, for example, the methodologies described in such publications that might be used in connection with the technology described herein. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. For example, while method steps or functions are presented in a given order, alternative embodiments may perform functions in a different order, or functions may be performed substantially concurrently. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. Moreover, due to biological functional equivalency considerations, some changes can be made in protein structure without affecting the biological or chemical action in kind or amount. These and other changes can be made to the disclosure in light of the detailed description. All such modifications are intended to be included within the scope of the appended claims.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure.

The technology described herein is further illustrated by the following examples which in no way should be construed as being further limiting.

Some embodiments of the technology described herein can be defined according to any of the following numbered paragraphs:
1. A composition comprising:
   a) a caspase inhibitor;
   b) a lysosomal membrane permeabilization (LMP) inhibitor;
   c) an antioxidant; and
   d) a growth factor.
2. The composition of paragraph 1, wherein the caspase inhibitor is a pan-caspase inhibitor.
3. The composition of paragraph 2, wherein the pan-caspase inhibitor is selected from the group consisting of: Q-VD-Oph; Z-VAD-FMK; Emricasan; and Ac-DEVD-CHO.
4. The composition of paragraph 2 or 3, wherein the pan-caspase inhibitor is Q-VD-Oph.
5. The composition of paragraph 4, wherein Q-VD-Oph is at a concentration of at least 50 μM.
6. The composition of paragraph 2, wherein the pan-caspase inhibitor is Emricasan.
7. The composition of paragraph 6, wherein Emricasan is at a concentration of at least 10 μM.
8. The composition of paragraph 6, wherein Emricasan is at a concentration of at least 25 μM.
9. The composition of paragraph 1, wherein the LMP inhibitor is Heat Shock Protein 70 (Hsp70) and/or deferoxamine mesylate (DFO).
10. The composition of paragraph 9, wherein Hsp70 is at a concentration of at least 10 μM.
11. The composition of paragraph 9, wherein DFO is at a concentration of at least 1 μM.
12. The composition of paragraph 1, wherein the antioxidant is N-acetyl cysteine (NAC).
13. The composition of paragraph 12, wherein NAC is at a concentration of at least 10 μM.
14. The composition of paragraph 1, wherein the growth factor is a stimulator of the phosphoinositide 3-kinase (PI3K) and Akt pathway.
15. The composition of paragraph 14, wherein the stimulator of the PI3K/Akt pathway is granulocyte colony-stimulating factor (G-CSF).
16. The composition of paragraph 15, wherein G-CSF is at a concentration of at least 10 ng/mL.
17. The composition of any one of paragraphs 1-16, further comprising a necroptosis inhibitor.
18. The composition of paragraph 17, wherein the necroptosis inhibitor is Nec-1s.
19. The composition of paragraph 18, wherein Nec-1s is at a concentration of at least 10 μM.
20. The composition of any one of paragraphs 1-19, further comprising cell culture medium and/or serum.
21. The composition of paragraph 20, wherein the cell culture medium comprises RPMI.
22. The composition of paragraph 20, wherein the serum is fetal bovine serum (FBS).
23. A cell culture medium comprising:
   a) a caspase inhibitor;
   b) a lysosomal membrane permeabilization (LMP) inhibitor;
   c) an antioxidant;
   d) a growth factor; and
   e) a necroptosis inhibitor.
24. The cell culture medium of paragraph 23, wherein the cell culture medium comprises RPMI.
25. The cell culture medium of paragraph 23, wherein the serum is fetal bovine serum (FBS).
26. The cell culture medium of paragraph 25, wherein the FBS is at a concentration of at least 20%.
27. A cell culture medium for increasing the lifespan of human neutrophils, the cell culture medium comprising:
   a) at least about 50 μM of a pan-caspase inhibitor;
   b) at least about 1 μM of a first LMP inhibitor;
   c) at least about 10 pm of a second LMP inhibitor;
   d) at least about 10 μM of an antioxidant;
   e) at least about 10 ng/mL a growth factor; and
   f) at least about 10 μM of a necroptosis inhibitor.
28. A cell culture medium for increasing the lifespan of neutrophils, the cell culture medium comprising effective concentrations of: Q-VD-Oph; DFO; Hsp70; NAC; G-CSF; and Nec-1s.
29. A cell culture medium for increasing the lifespan of neutrophils, the cell culture medium comprising effective concentrations of: Q-VD-Oph; DFO; Hsp70; NAC; and G-CSF.
30. A cell culture medium for increasing the lifespan of neutrophils, the cell culture medium comprising effective concentrations of: Emricasan; DFO; Hsp70; NAC; G-CSF; and Nec-1s.
31. A cell culture medium for increasing the lifespan of neutrophils, the cell culture medium comprising effective concentrations of: Emricasan; DFO; Hsp70; NAC; and G-CSF.
32. A cell culture medium for increasing the lifespan of human neutrophils, the cell culture medium comprising:
   a) at least about 50 μM of Q-VD-Oph;
   b) at least about 1 μM of DFO;
   c) at least about 10 pm of Hsp70;
   d) at least about 10 μM of NAC;
   e) at least about 10 ng/mL of G-CSF; and
   f) at least about 10 μM of Nec-1s.
33. A cell culture medium for increasing the lifespan of human neutrophils, the cell culture medium comprising:
   a) about 50 μM of Q-VD-Oph;
   b) about 1 μM of DFO;
   c) about 10 pm of Hsp70;
   d) about 10 μM of NAC;
   e) about 10 ng/mL of G-CSF; and
   f) about 10 μM of Nec-1s.
34. The composition of any one of paragraphs 1-22 or the cell culture medium of any one of paragraphs 23-33, in combination with a neutrophil.
35. The combination of paragraph 34, wherein the neutrophil is a human neutrophil.
36. A kit comprising the composition of any one of paragraphs 1-22 or the cell culture medium of any one of paragraphs 23-33.
37. A method of increasing the lifespan of a neutrophil, the method comprising: contacting the neutrophil, or population thereof, with the composition of any one of paragraphs 1-22 or the cell culture medium of any one of paragraphs 23-33.
38. The method of paragraph 37, wherein the neutrophil, or population thereof, is a peripheral blood neutrophil (PMN).
39. The method of paragraph 37, wherein the neutrophil, or population thereof, is isolated from the peripheral blood of a subject.
40. The method of paragraph 37, wherein the neutrophil, or population thereof, is isolated from blood of a subject using apheresis.
41. The method of paragraph 37, wherein the neutrophil, or population thereof, is isolated from the bone marrow of a subject.
42. The method of any one of paragraphs 37-41, wherein the neutrophil, or population thereof, is contacted with the composition or the cell culture medium for a sufficient amount of time to increase neutrophil lifespan.
43. The method of paragraph 42, wherein the sufficient amount of time is at least 3 days.
44. The method of paragraph 42, wherein the sufficient amount of time is at least 1 hour.
45. The method of any one of paragraphs 37-44, wherein the method further comprises removing the composition or the cell culture medium from the neutrophil, or population thereof, after the sufficient amount of time to increase neutrophil lifespan.
46. The method of any one of paragraphs 37-45, wherein the half-life of the neutrophil, or population thereof, is increased to at least 5 days.
47. The method of any one of paragraphs 37-46, wherein the neutrophil, or population thereof, is contacted with the composition or culture medium ex vivo.
48. The method of any one of paragraphs 37-47, wherein contacting comprises culturing.
49. A neutrophil produced by any one of the methods of paragraphs 37-48.
50. A pharmaceutical composition comprising the neutrophil of paragraph 49, or population thereof, and a pharmaceutically acceptable carrier.
51. The pharmaceutical composition of paragraph 50 for use in treating neutropenia in a subject.
52. The pharmaceutical composition of paragraph 50 for use in treating a microbial infection in a subject.
53. A method of treating neutropenia or a neutropenia-associated disease or disorder, the method: comprising administering an effective amount of a neutrophil of paragraph 49, or population thereof, or a pharmaceutical composition of paragraph 50 to a recipient subject in need thereof.
54. The method of paragraph 53, wherein the neutrophil is obtained from a human.
55. The method of paragraph 53, wherein the neutrophil is obtained from the subject.
56. A method of treating a microbial infection, the method comprising: administering an effective amount of a neutrophil of paragraph 49, or population thereof, or a pharmaceutical composition of paragraph 50 to a recipient subject in need thereof.
57. The method of paragraph 56, wherein the neutrophil is obtained from a human.
58. The method of paragraph 56, wherein the neutrophil is obtained from the subject.
59. A method of treating neutropenia or a neutropenia-associated disease or disorder, the method comprising:
   a) isolating a neutrophil or population thereof from a donor subject;
   b) contacting the neutrophil, or population thereof, with the composition of any one of paragraphs 1-22 or the cell culture medium of any one of paragraphs 23-33; and
   c) administering an effective amount of the cultured neutrophil, or population thereof, to a recipient subject in need thereof.
60. The method of paragraph 59, wherein the donor subject is the recipient subject.

Some embodiments of the technology described herein can be defined according to any of the following numbered paragraphs:
101. A composition comprising:
   a) a caspase inhibitor;
   b) a lysosomal membrane permeabilization (LMP) inhibitor;
   c) an antioxidant; and
   d) a stimulator of the phosphoinositide 3-kinase (PI3K) and Akt pathway.
102. The composition of paragraph 101, wherein the caspase inhibitor is a pan-caspase inhibitor.
103. The composition of paragraph 102, wherein the pan-caspase inhibitor is Q-VD-Oph.
104. The composition of paragraph 103, wherein Q-VD-Oph is at a concentration of at least 50 μM.
105. The composition of paragraph 101, wherein the LMP inhibitor is Heat Shock Protein 70 (Hsp70) and/or deferoxamine mesylate (DFO).
106. The composition of paragraph 105, wherein Hsp70 is at a concentration of at least 10 pM.
107. The composition of paragraph 105, wherein DFO is at a concentration of at least 1 μM.
108. The composition of paragraph 101, wherein the antioxidant is N-acetyl cysteine (NAC).
109. The composition of paragraph 108, wherein NAC is at a concentration of at least 10 μM.
110. The composition of paragraph 108, wherein NAC is at a concentration of at least 1 mM.
111. The composition of paragraph 101, wherein the stimulator of the PI3K/Akt pathway is granulocyte colony-stimulating factor (G-CSF).
112. The composition of paragraph 111, wherein G-CSF is at a concentration of at least 10 ng/mL.
113. The composition of any one of paragraphs 101-112, further comprising a necroptosis inhibitor.
114. The composition of paragraph 113, wherein the necroptosis inhibitor is Nec-1s.
115. The composition of paragraph 114, wherein Nec-1s is at a concentration of at least 10 μM.
116. The composition of any one of paragraphs 101-115, further comprising cell culture medium and/or serum.
117. A cell culture medium comprising:
   a) a caspase inhibitor;
   b) a lysosomal membrane permeabilization (LMP) inhibitor;
   c) an antioxidant;
   d) a stimulator of the phosphoinositide 3-kinase (PI3K) and Akt pathway; and
   e) a necroptosis inhibitor.
118. The composition of paragraph 116 or culture medium of paragraph 117, wherein the cell culture medium is RPMI.
119. The composition of paragraph 116 or culture medium of paragraph 117, wherein the serum is fetal bovine serum (FBS).
120. The cell culture medium of paragraph 119, wherein the FBS is at a concentration of at least 20%.
121. The composition of any one of paragraphs 101-116 or the cell culture medium of any one of paragraphs 17-20, in combination with a neutrophil.
122. The composition or the cell culture medium of paragraph 121, wherein the neutrophil is a human neutrophil or a mouse neutrophil.
123. A kit comprising the composition or the cell culture medium of any one of paragraphs 101-122.
124. A method of increasing the lifespan of a neutrophil, comprising contacting the neutrophil with the composition or the cell culture medium of any one of paragraphs 101-122.
125. The method of paragraph 124, wherein the lifespan of the neutrophil is increased to at least 5 days.
126. The method of paragraph 124 or 125, wherein the neutrophil is contacted with the composition ex vivo.
127. The method of any one of paragraphs 124-126, wherein contacting is culturing.
128. The method of any one of paragraphs 124-127, wherein the neutrophil is contacted with the composition for at least 3 days.
129. A neutrophil produced by any of the methods of paragraphs 24-28.
130. A composition comprising the neutrophil of paragraph 129, or population thereof.
131. The composition of paragraph 130, further comprising a pharmaceutically acceptable carrier.
132. A pharmaceutical composition comprising the neutrophil of paragraph 129, or population thereof, and a pharmaceutically acceptable carrier.
133. The pharmaceutical composition of paragraph 132 for use in treating neutropenia in a subject.
134. A method of treating neutropenia or a neutropenia-associated disease or disorder in a subject in need thereof the method comprising administering an effective amount of a neutrophil of paragraph 129, or population thereof, or a composition of paragraphs 130-131, or a pharmaceutical composition of paragraphs 132-133 to a recipient subject in need thereof.
135. The method of any one of paragraphs 124-128 or 134, wherein the neutrophil is obtained from a human or mouse.
136. The method of paragraph 134 or 135, wherein the neutrophil is obtained from the subject.

### EXAMPLES

### Example 1: Targeting Multiple Cell Death Pathways Extended the Shelf-Life and Preserved the Function of Human and Mouse Neutrophils for Transfusion

Described herein are compositions and methods wherein the survival of neutrophils is prolonged and their function is preserved by simultaneously targeting multiple cell death pathways.

Clinical outcomes from granulocyte transfusion (GTX) are disadvantaged by the short shelf-life and compromised function of donor neutrophils. Spontaneous neutrophil death is heterogeneous and mediated by multiple pathways. Leveraging mechanistic knowledge and pharmacological screening, a combined treatment was identified comprising: caspases-lysosomal membrane permeabilization-oxidant-necroptosis inhibition plus granulocyte colony-stimulating factor (CLON-G); CLON-G which altered neutrophil fate by simultaneously targeting multiple cell death pathways. CLON-G prolonged human and mouse neutrophil half-life in vitro from less than 1 day to greater than 5 days. CLON-G-treated aged neutrophils had equivalent morphology and function to fresh neutrophils, with no impairment to critical effector functions including phagocytosis, bacterial killing, chemotaxis, and reactive oxygen species production. Transfusion with stored CLON-G-treated 3-day-old neutrophils enhanced host defenses, alleviated infection-induced tissue damage, and prolonged survival as effectively as transfusion with fresh neutrophils in a clinically relevant murine GTX model of neutropenia-related bacterial pneumonia and systemic candidiasis. Last, CLON-G-treatment prolonged the shelf-life and preserved the function of apheresis-collected human granulocyte transfusion products both ex vivo and in vivo in immunodeficient mice. Thus, CLON-G treatment is an effective and applicable clinical procedure for the storage and application of neutrophils in transfusion medicine, providing a therapeutic strategy for improving granulocyte transfusion (GTX) efficacy.

Pharmacological Screening Identified a Combined Treatment that Delayed Neutrophil Death by Simultaneously Targeting Multiple Death Pathways.

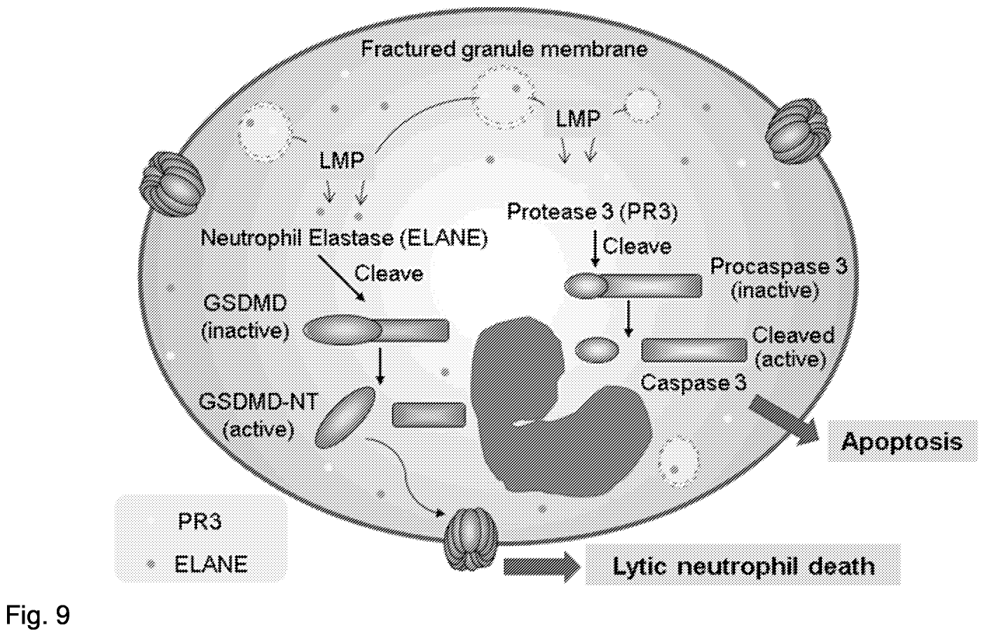

Programed neutrophil death is a heterogeneous process mediated by multiple pathways (see e.g., FIG. 9). Human or murine neutrophils were treated with various inhibitors of neutrophil death-related pathways including caspases, LMP, serine proteases, ROS/NADPH (reduced form of nicotinamide adenine dinucleotide phosphate) oxidase, and G-CSF (see e.g., Tables 1-2). Alone, the pan-caspase inhibitor Q-VD-Oph had the strongest effect on neutrophil death; most other drugs were ineffective on their own. For maximal inhibition, multiple death pathways were simultaneously blocked. Co-treatment with the antioxidant N-acetyl cysteine (NAC), LMP inhibitors Heat shock protein 70 (HSP70) and deferoxamine mesylate (DFO, an iron chelator), phosphoinositide 3-kinase (PI3K)/Akt pathway stimulator G-CSF, and pan-caspase inhibitor Q-VD-Oph prolonged both human (see e.g., Table 1) and mouse (see e.g., Table 2) neutrophil survival. This treatment was named CLO-G (caspases-LMP-oxidant inhibition plus G-CSF).

The full role of necroptosis in neutrophil spontaneous death is unclear. However, necroptosis is often induced when caspases are inhibited, so although treatment with the pan-caspase inhibitor Q-VD-OPh prolonged neutrophil survival, it may also induce necroptosis to ultimately contribute to the death of CLO-G-treated neutrophils. Neutrophil death was further inhibited by combined treatment with CLO-G and a pharmacological inhibitor of necroptosis, Nec-1s, which is a highly specific small molecule antagonist of receptor-interacting protein kinase 1 (RIPK1). This treatment (Q-VD-Oph, G-CSF, NAC, HSP70, DFO, and Nec-1s), named CLON-G (caspases-LMP-oxidant-necroptosis inhibition plus G-CSF), showed the best anti-death activity, increasing human and mouse neutrophil survival to greater than 90% after 3 days of culture (see e.g., Tables 1-2). The pan caspase inhibitor Emricasan, in combination with NAC, Hsp70, DFO, G-CSF, and optionally a RIPK1 inhibitor, also prolonged the lifespan of cultured neutrophils (see e.g., FIG. 23). Additional pan caspase inhibitors, Z-VAD-FMK or Ac-DEVD-CHO, combined with Hsp70, NAC and G-CSF increased neutrophil lifespan such that over 700 of neutrophils survived after culturing for 3 days (data not shown).

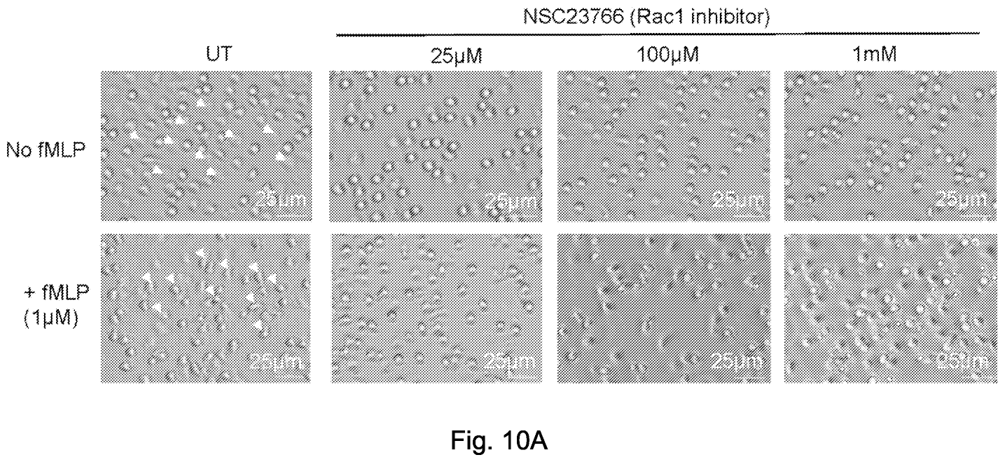

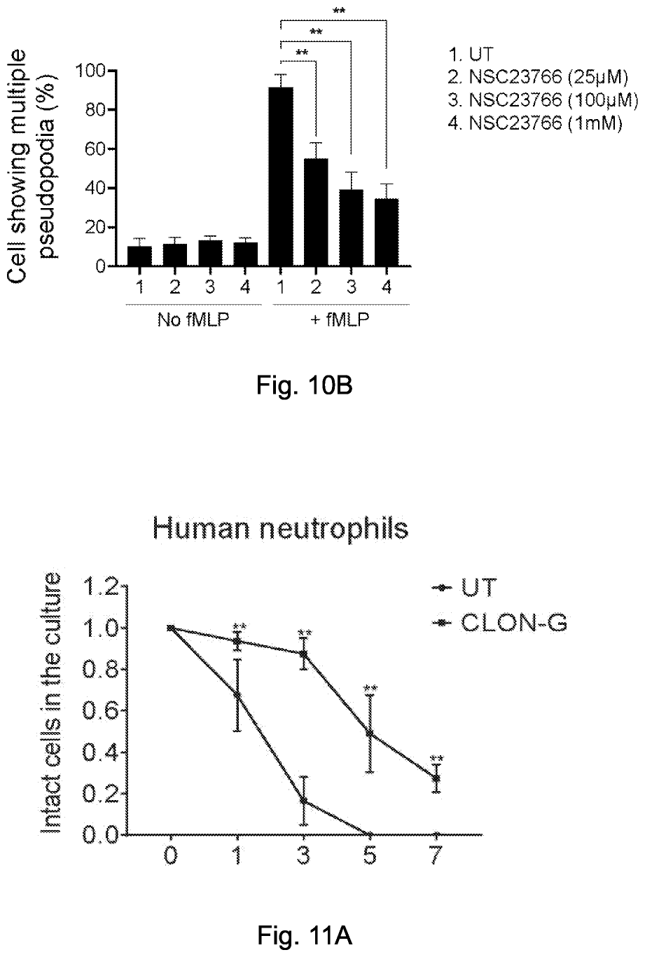

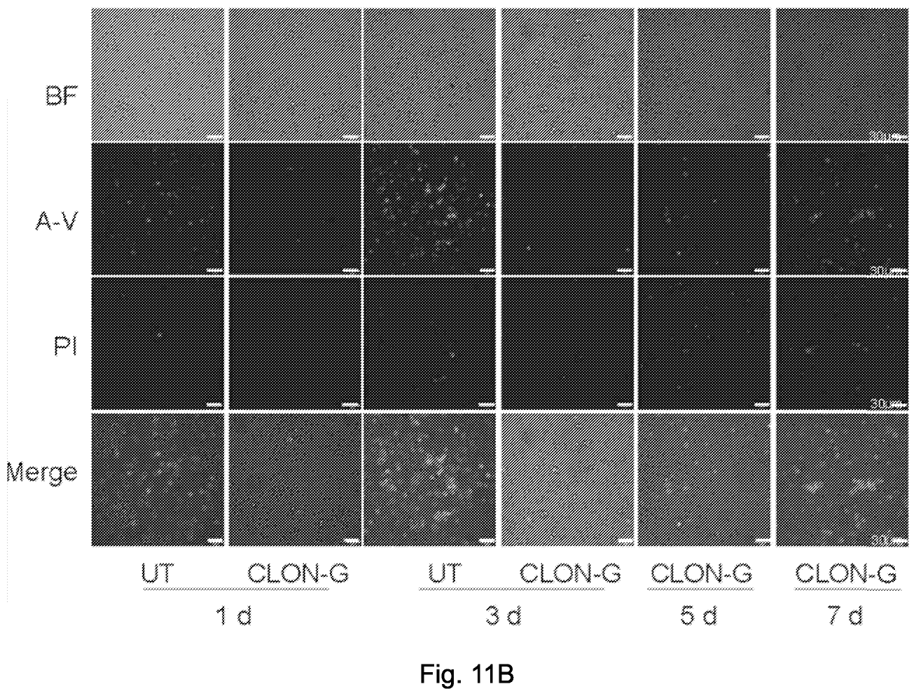

Cell adhesion can trigger integrin-mediated ROS production and subsequently accelerated neutrophil death. However, although a Rac guanosine triphosphatase (GTPase) inhibitor, NSC23766, effectively inhibited N-formyl-Met-Leu-Phe (fMLP)-induced neutrophil polarization (see e.g., FIG. 10), it did not alter neutrophil half-life on its own or in combination with other inhibitors, indicating that cell adhesion may not be a substantial factor in this system in which high serum concentrations kept most neutrophils non-adherent (see e.g., Tables 1-2); see e.g., Gao et al., PNAS 101, 7618-7623 (2004), the content of which is incorporated herein by reference in its entirety.

TABLE 1
TABLE 1: Screening for treatments that delayed human neutrophil death. Neutrophil
survival after 3 days assessed as described in FIG. 1 was normalized to untreated
fresh neutrophils (counted as 100). LMP, lysosomal membrane permeabilization.
All data are represented as mean ± SD of at least three experiments.

| | | | | | Neutrophil survival |
|---|---|---|---|---|---|
| | | | | | after 3 days - |
| | | | Concentration | | normalized to fresh |
| Targets | Inhibitor/drug | Source | (Final) | | neutrophils (100) |
| Untreated | | | | | <25 |
| (Control) | | | | | |
| Caspase-1/4 | Z-YVAD-FMK | ADOOQ | 12.5 | μM | <25 |
| | | BIOSCIENCE | 25 | μM | <25 |
| | | | 50 | μM | <25 |
| | | | 100 | μM | <25 |
| | | | 200 | μM | <25 |
| | Belnacasan (VX-765) | SELLECK CHEM | 12.5 | μM | <25 |
| | | | 25 | μM | <25 |
| | | | 50 | μM | <25 |
| | | | 100 | μM | <25 |
| | | | 200 | μM | <25 |
| Caspase-2 | Z-VDVAD-FMK | ADOOQ | 25 | μM | <25 |
| | | BIOSCIENCE | | | |
| Caspase-3 | Z-DQMD-FMK | ADOOQ | 25 | μM | <25 |
| | | BIOSCIENCE | | | |
| | Boc-D-FMK | ADOOQ | 25 | μM | <25 |
| | | BIOSCIENCE | | | |
| | Ivachtin | ADOOQ | 25 | μM | <25 |
| | | BIOSCIENCE | | | |
| | Z-DEVD-FMK | SELLECK CHEM | 12.5 | μM | <25 |
| | | | 25 | μM | <25 |
| | | | 50 | μM | <25 |
| | | | 100 | μM | <25 |
| | | | 200 | μM | <25 |
| Caspase-4 | Belnacasan (VX-765) | SELLECK CHEM | 12.5 | μM | <25 |
| | | | 25 | μM | <25 |
| | | | 50 | μM | <25 |
| | | | 100 | μM | <25 |
| | | | 200 | μM | <25 |
| Caspase-5 | Z-WEHD-FMK | ADOOQ | 25 | μM | <25 |
| | | BIOSCIENCE | | | |
| Caspase-6 | Z-VEID-FMK | ADOOQ | 25 | μM | <25 |
| | | BIOSCIENCE | | | |
| Caspase-8 | Z-IETD-FMK | SELLECK CHEM | 12.5 | μM | <25 |
| | | | 25 | μM | <25 |
| | | | 50 | μM | <25 |
| | | | 100 | μM | <25 |
| | | | 200 | μM | <25 |
| Caspase-9 | Ac-LEHD-CHO | SIGMA-ALDRICH | 12.5 | μM | <25 |
| | | | 25 | μM | <25 |
| | | | 50 | μM | <25 |
| | | | 100 | μM | <25 |
| | | | 200 | μM | <25 |
| Caspase-11 | Wedelolactone | ADOOQ | 25 | μM | <25 |
| | | BIOSCIENCE | | | |
| Pan- | Q-VD-Oph | SELLECK CHEM | 12.5 | μM | 65 ± 9.6 |
| caspase | | | 25 | μM | 60 ± 0.8 |
| | | | 50 | μM | 65 ± 3.9 |
| | | | 100 | μM | 62 ± 4.9 |
| | | | 200 | μM | 47 ± 8.3 |
| | Z-VAD-FMK | SELLECK CHEM | 12.5 | μM | <25 |
| | | | 25 | μM | <25 |
| | | | 50 | μM | <25 |
| | | | 100 | μM | <25 |
| | | | 200 | μM | <25 |
| | Emricasan | SELLECK CHEM | 100 | nM | <25 |
| | | | 1 | μM | <25 |
| | | | 10 | μM | <25 |
| | | | 100 | μM | <25 |
| | | | 200 | μM | <25 |
| | Ac-DEVD-CHO | SELLECK CHEM | 100 | nM | <25 |
| | | | 1 | μM | <25 |
| | | | 10 | μM | <25 |
| | | | 25 | μM | <25 |
| | | | 100 | μM | <25 |
| | | | 200 | μM | <25 |
| LMP | DFO | SIGMA-ALDRICH | 1 | μM | <25 |
| | | | 10 | μM | <25 |
| | | | 100 | μM | <25 |
| | | | 1 | mM | <25 |
| | | | 10 | mM | <25 |
| | Hsp70 | ENZO LIFE | 10 | pM | <25 |
| | | SCIENCES | 100 | pM | <25 |
| | | | 1 | nM | <25 |
| | | | 10 | nM | <25 |
| | | | 100 | nM | <25 |
| | | | 200 | nM | <25 |
| Serine | DFP | SIGMA-ALDRICH | 1 | nM | <25 |
| protease | | | 10 | nM | <25 |
| | | | 100 | nM | <25 |
| | | | 1 | μM | <25 |
| | | | 10 | μM | <25 |
| NADPH | DPI | SIGMA-ALDRICH | 1 | nM | <25 |
| oxidase | | | 10 | nM | <25 |
| | | | 100 | nM | <25 |
| | | | 1 | μM | <25 |
| | | | 10 | μM | <25 |
| | | | 50 | μM | <25 |
| Antioxidant | N-acetylcysteine | SIGMA-ALDRICH | 3.125 | mM | <25 |
| | (NAC) | | 6.25 | mM | <25 |
| | | | 12.5 | mM | <25 |
| | | | 25 | mM | <25 |
| | | | 50 | mM | <25 |
| Necroptosis | Nec-1s | EMD MILLIPORE | 100 | nM | <25 |
| | | (852391-15-2) | 1 | μM | <25 |
| | | | 10 | μM | <25 |
| | | | 100 | μM | <25 |
| | | | 200 | μM | <25 |
| | NECROX-2 | ENZO LIFE | 10 | nM | <25 |
| | | SCIENCES | 100 | nM | <25 |
| | | (ALX-430-166) | 1 | μM | <25 |
| | | | 10 | μM | <25 |
| | | | 100 | μM | <25 |
| | NECROX-5 | ENZO LIFE | 10 | nM | <25 |
| | | SCIENCES | 100 | nM | <25 |
| | | (ALX-430-167) | 1 | μM | <25 |
| | | | 10 | μM | <25 |
| | | | 100 | μM | <25 |
| Growth | G-CSF | AMGEN | 1 | ng/ml | 31 ± 2.1 |
| factor | | | 10 | ng/ml | 35 ± 4.5 |
| | | | 100 | ng/ml | 30 ± 5.5 |
| | | | 200 | ng/ml | 31 ± 2.1 |
| | | | 1 | μg/ml | 34 ± 3.2 |
| | | | 10 | μg/ml | 32 ± 5.4 |
| Rho- | NSC23766 | in house | 100 | nM | <25 |
| GTPase | (Rac inhibitor) | | 1 | μM | <25 |
| | | | 10 | μM | <25 |
| | | | 100 | μM | <25 |

| | | |
|---|---|---|
| Combined | Z-YVAD-FMK + Z-DEVD-FMK | <25 |
| treatment | Belnacasan (VX-765) + Z-DEVD-FMK | <25 |
| | Q-VD-Oph + NAC | 74 ± 5.2 |
| | Q-VD-Oph + DFO | 74 ± 5.0 |
| | Q-VD-Oph + Hsp70 | 76 ± 2.2 |
| | Q-VD-Oph + Nec-1s | 75 ± 4.9 |
| | Q-VD-Oph + G-CSF | 70 ± 2.4 |
| | Z-VAD-FMK + DFO + DPI + G-CSF | 35 ± 3.8 |
| | Emricasan + DFO + DPI + G-CSF | 36 ± 9.1 |
| | Ac-DEVD-CHO + DFO + DPI + G-CSF | 28 ± 10.3 |
| | Z-DEVD-FMK + Hsp70 + NAC + G-CSF | 26 ± 9.6 |
| | Z-VAD-FMK + Hsp70 + NAC + G-CSF | 74 ± 13.1 |
| | Ac-DEVD-CHO + Hsp70 + NAC + G-CSF | 79 ± 6.8 |
| | Q-VD-Oph + DFO + DPI + G-CSF | 39 ± 7.7 |
| | Q-VD-Oph + human Hsp70 + DPI + G-CSF | 43 ± 9.4 |
| | Q-VD-Oph + DFO + NAC + G-CSF | 56 ± 7.5 |
| | Q-VD-Oph + mouse Hsp70 + DPI + G-CSF | 51 ± 5.4 |
| | Q-VD-Oph + Hsp70 + NAC + G-CSF | 85 ± 10.5 |
| | Q-VD-Oph + DFO + NAC + DFP + G-CSF | 51 ± 12.5 |
| | Q-VD-Oph + Hsp70 + DPI + DFP + G-CSF | 55 ± 7.0 |
| | Q-VD-Oph + Hsp70 + NAC + DFP + G-CSF | 63 ± 16.2 |
| | Q-VD-Oph + Hsp70 + NAC + DFO + G-CSF + Nec-1s | 91 ± 6.6 |
| | Q-VD-Oph + Hsp70 + NAC + DFO + G-CSF + Nec-1s + NSC23766 | 83 ± 8.5 |

TABLE 2
TABLE 2: Screening for treatments that delayed murine neutrophil death. Neutrophil survival
after 3 days assessed as described in FIG. 1 was normalized to untreated fresh neutrophils
(counted as 100). All data are represented as mean ± SD of at least three experiments.

| | | | | | Neutrophil survival |
|---|---|---|---|---|---|
| | | | | | after 3 days - |
| | | | Concentration | | normalized to fresh |
| Targets | Inhibitor/drug | Source | (Final) | | neutrophils (100) |
| Untreated | | | | | <10 |
| (Control) | | | | | |
| Caspase-1/4 | Z-YVAD-FMK | ADOOQ | 12.5 | μM | <10 |
| | | BIOSCIENCE | 25 | μM | <10 |
| | | | 50 | μM | <10 |
| | | | 100 | μM | <10 |
| | | | 200 | μM | <10 |
| | Belnacasan (VX-765) | SELLECK CHEM | 12.5 | μM | <10 |
| | | | 25 | μM | <10 |
| | | | 50 | μM | <10 |
| | | | 100 | μM | <10 |
| | | | 200 | μM | <10 |
| Caspase-3 | Z-DEVD-FMK | SELLECK CHEM | 12.5 | μM | <10 |
| | | | 25 | μM | <10 |
| | | | 50 | μM | <10 |
| | | | 100 | μM | <10 |
| | | | 200 | μM | <10 |
| Caspase-8 | Z-IETD-FMK | SELLECK CHEM | 12.5 | μM | <10 |
| | | | 25 | μM | <10 |
| | | | 50 | μM | <10 |
| | | | 100 | μM | <10 |
| | | | 200 | μM | <10 |
| Caspase-9 | Ac-LEHD-CHO | SIGMA-ALDRICH | 12.5 | μM | <10 |
| | | | 25 | μM | <10 |
| | | | 50 | μM | <10 |
| | | | 100 | μM | <10 |
| | | | 200 | μM | <10 |
| Pan- | Q-VD-Oph | SELLECK CHEM | 12.5 | μM | 49 ± 4.4 |
| caspase | | | 25 | μM | 51 ± 2.5 |
| | | | 50 | μM | 52 ± 0.2 |
| | | | 100 | μM | 46 ± 2.4 |
| | | | 200 | μM | 50 ± 3.6 |
| | Z-VAD-FMK | SELLECK CHEM | 12.5 | μM | <10 |
| | | | 25 | μM | <10 |
| | | | 50 | μM | <10 |
| | | | 100 | μM | <10 |
| | | | 200 | μM | 14 ± 0.3 |
| | Emricasan | SELLECK CHEM | 100 | nM | <10 |
| | | | 1 | μM | <10 |
| | | | 10 | μM | <10 |
| | | | 100 | μM | <10 |
| | | | 200 | μM | <10 |
| | Ac-DEVD-CHO | SELLECK CHEM | 100 | nM | <10 |
| | | | 1 | μM | <10 |
| | | | 10 | μM | <10 |
| | | | 100 | μM | <10 |
| | | | 200 | μM | <10 |
| LMP | DFO | SIGMA-ALDRICH | 1 | μM | <10 |
| | | | 10 | μM | <10 |
| | | | 100 | μM | <10 |
| | | | 1 | mM | <10 |
| | | | 10 | mM | <10 |
| | Hsp70 | ABCAM | 10 | pM | <10 |
| | | | 100 | pM | <10 |
| | | | 1 | nM | <10 |
| | | | 10 | nM | <10 |
| | | | 100 | nM | <10 |
| | | | 200 | nM | <10 |
| Serine | DFP | SIGMA-ALDRICH | 1 | nM | <10 |
| protease | | | 10 | nM | <10 |
| | | | 100 | nM | <10 |
| | | | 1 | μM | <10 |
| | | | 10 | μM | <10 |
| NADPH | DPI | SIGMA-ALDRICH | 1 | nM | <10 |
| oxidase | | | 10 | nM | <10 |
| | | | 100 | nM | <10 |
| | | | 1 | μM | <10 |
| | | | 10 | μM | <10 |
| | | | 50 | μM | <10 |
| Antioxidant | N-acetylcysteine | SIGMA-ALDRICH | 3.125 | mM | <10 |
| | (NAC) | | 6.25 | mM | <10 |
| | | | 12.5 | mM | <10 |
| | | | 25 | mM | <10 |
| | | | 50 | mM | <10 |
| Necroptosis | Nec-1s | EMD MILLIPORE | 100 | nM | <10 |
| | | (852391-15-2) | 1 | μM | <10 |
| | | | 10 | μM | <10 |
| | | | 100 | μM | <10 |
| | | | 200 | μM | <10 |
| | NECROX-2 | ENZO LIFE | 10 | nM | <10 |
| | | SCIENCES | 100 | nM | <10 |
| | | (ALX-430-166) | 1 | μM | <10 |
| | | | 10 | μM | <10 |
| | | | 100 | μM | <10 |
| | NECROX-5 | ENZO LIFE | 10 | nM | <10 |
| | | SCIENCES | 100 | nM | <10 |
| | | (ALX-430-167) | 1 | μM | <10 |
| | | | 10 | μM | <10 |
| | | | 100 | μM | <10 |
| Growth | G-CSF | AMGEN | 1 | ng/ml | 19 ± 2.3 |
| factor | | | 10 | ng/ml | 21 ± 2.3 |
| | | | 100 | ng/ml | 21 ± 1.5 |
| | | | 1 | μg/ml | 21 ± 1.8 |
| | | | 10 | μg/ml | 21 ± 1.2 |
| Rho- | NSC23766 | in house | 100 | nM | <10 |
| GTPase | (Rac inhibitor) | | 1 | μM | <10 |
| | | | 10 | μM | <10 |
| | | | 100 | μM | <10 |

| | | |
|---|---|---|
| Combined | Belnacasan (VX-765) + Z-DEVD-FMK | <10 |
| treatment | Z-DEVD-FMK + Z-IETD-FMK | <10 |
| | Z-DEVD-FMK + Ac-LEHD-CHO | <10 |
| | VX-765 + Z-DEVD-FMK + Z-IETD-FMK + Ac-LEHD-CHO | <10 |
| | Q-VD-Oph + NAC | 61 ± 7.0 |
| | Q-VD-Oph + DFO | 64 ± 9.0 |
| | Q-VD-Oph + Hsp70 | 64 ± 11.3 |
| | Q-VD-Oph + Nec-1s | 67 ± 11.7 |
| | Q-VD-Oph + G-CSF | 66 ± 0.9 |
| | Q-VD-Oph + G-CSF + NAC | 63 ± 4.3 |
| | Q-VD-Oph + G-CSF + DFO | 77 ± 6.0 |
| | Q-VD-Oph + G-CSF + Hsp70 | 73 ± 4.8 |
| | Q-VD-Oph + G-CSF + Nec-1s | 87 ± 4.1 |
| | Q-VD-Oph + G-CSF + NECROX-2 | 70 ± 8.6 |
| | Q-VD-Oph + G-CSF + NECROX-5 | 72 ± 3.6 |
| | Q-VD-Oph + Hsp70 + NAC + DFO + G-CSF + Nec-1s | 90 ± 0.4 |
| | Q-VD-Oph + Hsp70 + NAC + DFO + G-CSF + Nec-1s + NSC23766 | 78 ± 6.2 |

CLON-G Treatment Prolonged the Ex Vivo Half-Life of Human and Mouse Neutrophils from Less than 24 Hours to Greater than 5 Days.

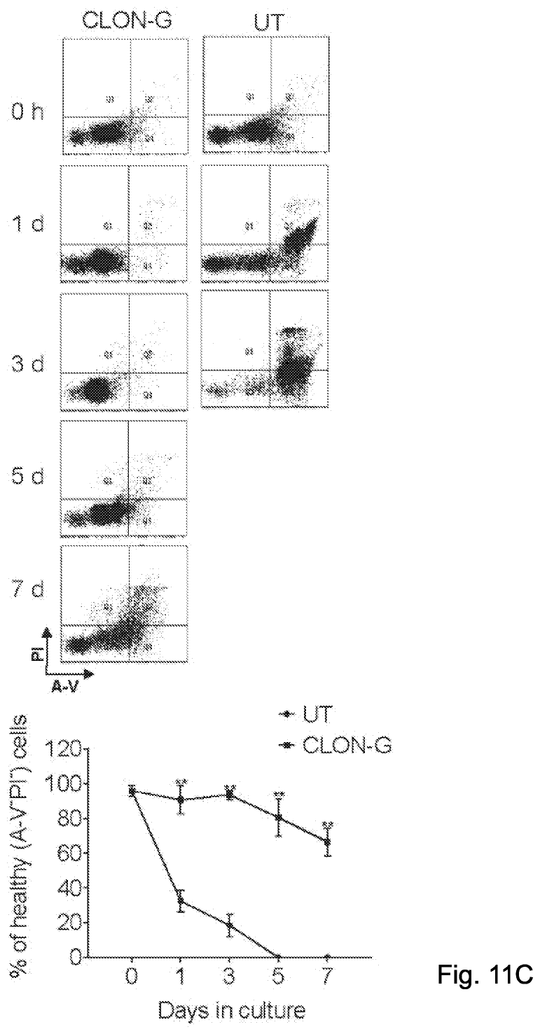

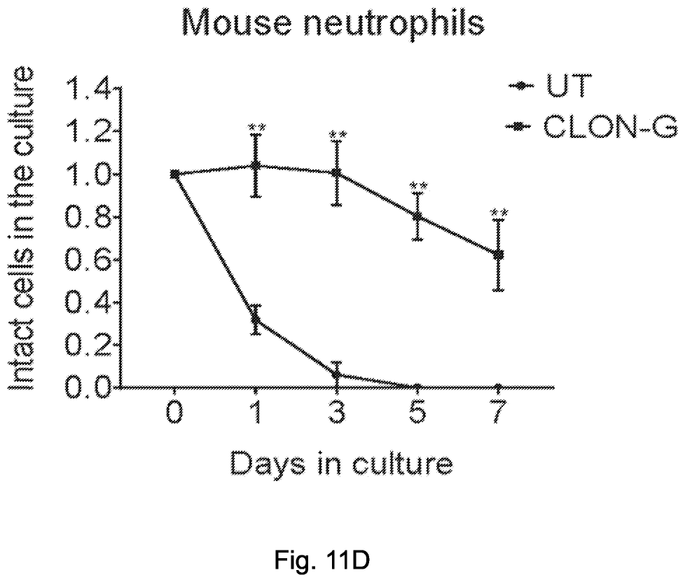

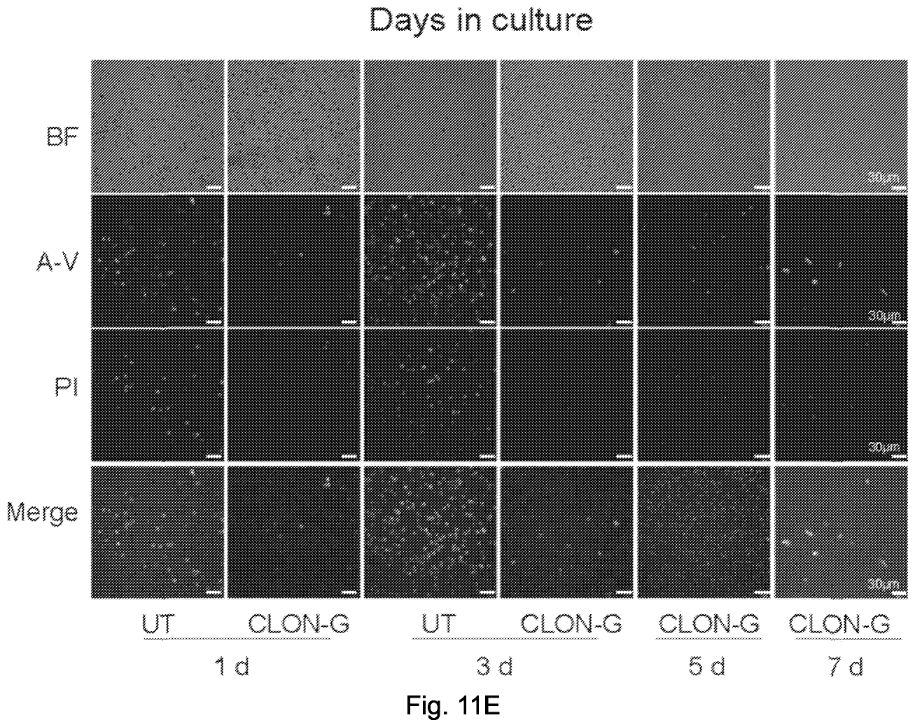

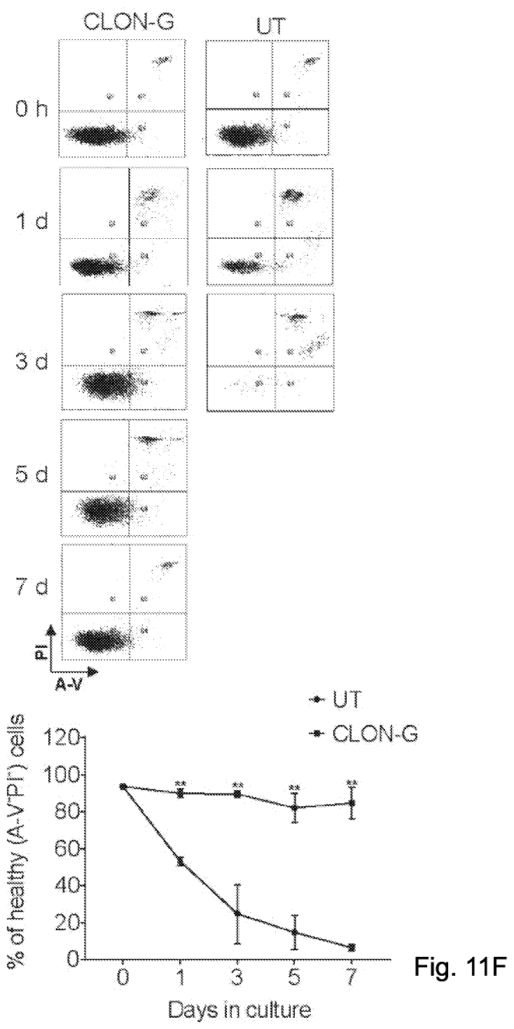

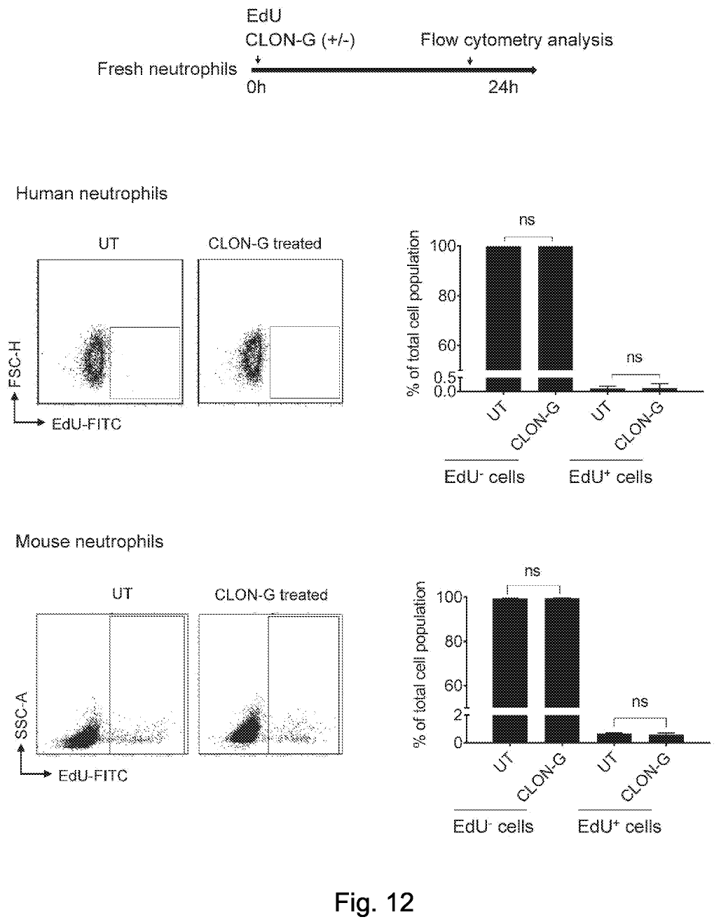

Having determined that CLON-G effectively delayed neutrophil spontaneous death, its effect on cultured neutrophils was next examined in detail. Since many neutrophils died by lytic cell death, the total number of detectable neutrophils, including healthy and dying cells, decreased gradually (see e.g., FIG. 1A and FIG. 11A). For human neutrophils, about 94% and 90% cells remained after 1 and 3 days of CLON-G treatment, respectively, whereas only 68% and 17% cells remained after 1 day and 3 days in the untreated group (see e.g., FIG. 11A). Fluorescein isothiocyanate (FITC)-Annexin V (AV) was used to detect phosphatidylsine (PS) exteriorization and propidium iodide (PI), a membrane impermeable dye, to monitor cell membrane integrity and to quantify the percentage of dying cells in the remaining intact neutrophils (see e.g., FIG. 1B and FIG. 11B). CLON-G reduced the percentage of dying mouse or human neutrophils (PI⁺AV⁺ and PI⁻AV⁺) at each time point (see e.g., FIG. 1C and FIG. 1C). Untreated, only <22% healthy human and mouse neutrophils remained after 24 hours, indicating a half-life of less than 24 hours. CLON-G treatment increased neutrophil half-life to more than 5 days, with about 90% survival of neutrophils after 3 days of culture compared to >95% cell disappearance or death in untreated controls (see e.g., FIG. 1D). Similar results were observed in mouse neutrophils (see e.g., FIG. 1E-1H and FIG. 11D-11E). The CLON-G effect was not due to augmented cell division, as minimal cell proliferation was detected in neutrophil cultures, and CLON-G treatment did not influence the proliferating capacity of contaminating neutrophil progenitors (see e.g., FIG. 12). For mouse neutrophils, even after 7 days of culture, >60% of the initial neutrophil count remained in the CLON-G-treated group (see e.g., FIG. 1H), equivalent to a greater than 7-day half-life.

A previous study showed that G-CSF treatment alone can prolong neutrophil life; see e.g., Luo et al., Am J Hematol 83, 288-295 (2008), the contents of which are incorporated herein by reference in their entirety. However, compared to CLON-G treatment, G-CSF only modestly improved neutrophil survival with about 55% and 15% human (see e.g., FIG. 13A-13D) and 40% and 15% mouse (see e.g., FIG. 13E-13H) healthy neutrophils remaining after 1 and 3 days of treatment, respectively. This confirms that the pro-survival effect elicited by CLON-G treatment was not solely due to G-CSF (see e.g., Tables 1-2, FIG. 13).

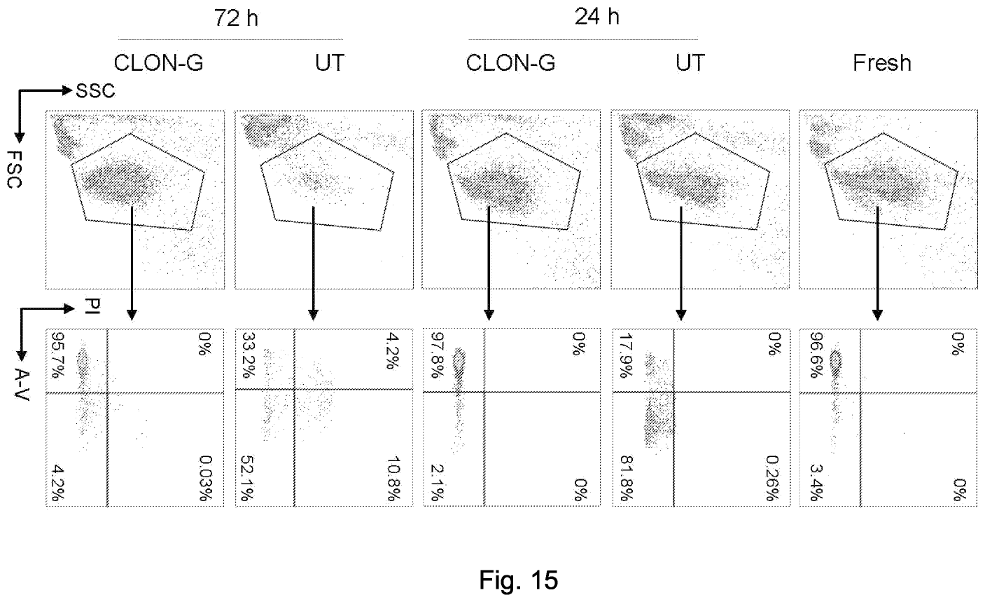

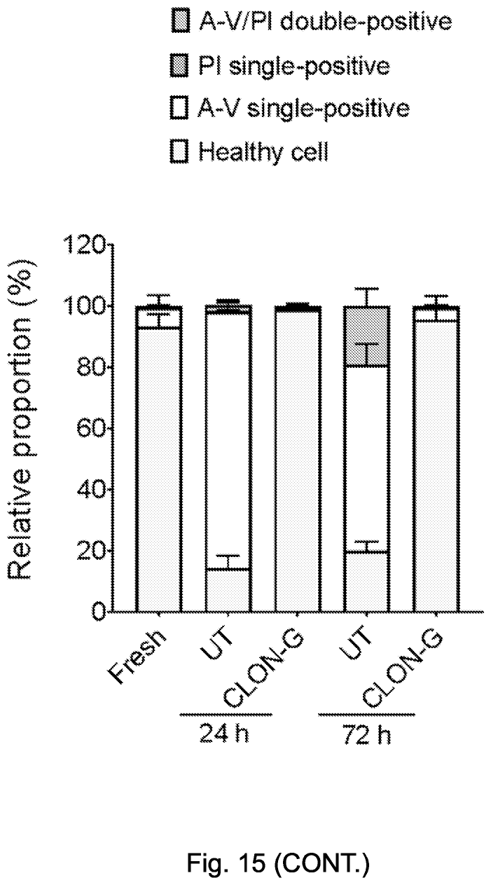

Physical stimuli, such as pipetting, easily fragment neutrophils undergoing lytic cell death, so a live imaging technology was utilized to simultaneously analyze apoptotic and lytic neutrophil death; see e.g., Teng et al. 2017, supra. Mouse neutrophils with enlarged and swollen morphology (“puffed” cells) were a predominant (˜15% after 24 hours) population of dead cells (see e.g., FIG. 14A), with two populations observable by microscopy: PI single-positive cells and AV/PI double-positive cells, with PI positivity in all cells suggesting that membrane integrity was always disrupted. Puffed cells were larger (15-20 m) than average neutrophils (˜8-10 m). Based on these morphological features, puffed cells were distinct from apoptotic neutrophils and hypothesized to be cells undergoing lytic cell death and lost during sample preparation so undetectable by flow cytometry. Flow cytometry analysis mainly detected non-lytic apoptotic neutrophils including PI single-positive cells, AV single-positive cells, and AV/PI double-positive cells (see e.g., FIG. 14B). There was a progressive increase in both puffed and apoptotic cells during neutrophil aging, and both populations decreased with CLON-G treatment (see e.g., FIG. 14A-14B). A similar effect was also apparent in human neutrophils (see e.g., FIG. 15). Collectively, CLON-G treatment suppressed both apoptotic and lytic neutrophil death, consistent with CLON-G-induced inhibition of multiple death pathways. Overall, CLON-G treatment prolonged neutrophil survival in vitro, increasing their half-life from less than 24 hours to greater than 5 days.

### Drug Removal Did not Accelerate the Death of CLON-G-Treated Neutrophils.

For clinical application, neutrophil lifespan-prolonging drugs often need to be removed prior to GTX. To establish whether temporary CLON-G treatment sufficiently maintains neutrophil survival, the lifespan of pharmacologically treated neutrophils was examined in the absence of drugs (see e.g., FIG. 2A and FIG. 16A). After 3 days of CLON-G treatment, washed mouse neutrophils displayed a similar rate of decrease in the total number of healthy cells (see e.g., FIG. 2B) and a similar percentage of AV⁻PI⁻ cells (see e.g., FIG. 2C) as untreated fresh neutrophils in the first 4 hours. However, at and after 12 hours, the number of healthy cells in the 3-day-old CLON-G-treated neutrophil population was higher than in the untreated fresh neutrophil population. This prolonged pro-survival effect was apparent in both mouse (see e.g., FIG. 2B) and human (see e.g., FIG. 16B-16C) neutrophils, hypothesized to be due to prolonged intracellular drug retention. To confirm this, fresh neutrophils were treated with CLON-G for 1 hour and then examined the survival of the wash neutrophils overtime (see e.g., FIG. 16D). Similarly, the pro-survival effect of CLON-G lasted for more than 3 days after the treated human (see e.g., FIG. 16E) or mouse (see e.g., FIG. 16F) neutrophils were washed with drug-free medium. These results indicate that CLON-G treatment can not only increase the ex vivo shelf life of the collected granulocytes, but can also delay in vivo death of transfused granulocytes due to the prolonged intracellular retention of the drugs (or drug effects).

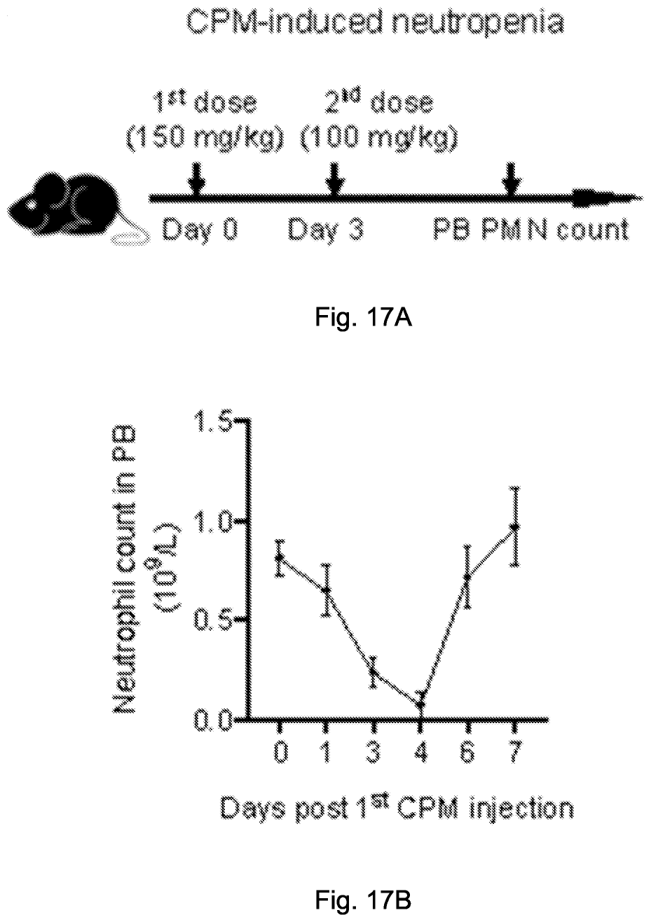

Next, it was investigated whether washed, CLON-G-treated 3-day-old and untreated fresh neutrophils had a similar lifespan at inflammatory sites in vivo. GTX is mainly used clinically for life-threatening bacterial and fungal infections in severely neutropenic patients. To mimic this clinical scenario, a mouse model was established in which neutropenia was induced by the widely used chemotherapy drug cyclophosphamide (CPM) (see e.g., FIG. 17A); see e.g., Li et al., Blood 113, 4930-4941 (2009), the content of which is incorporated herein by reference in its entirety. On day 4, CPM-treated wild-type mice contained approximately 95% fewer circulating neutrophils than untreated (day 0) control (see e.g., FIG. 17B). The lower limit of normal neutrophil numbers in humans is 1500 cells/mm³; in patients receiving chemotherapy or radiotherapy, neutrophil counts can be as low as 100/mm³ (93.3% reduction). Thus, the 95% reduction achieved in CPM-induced neutropenia is comparable to neutropenia in patients receiving chemotherapy. In this experiment, the CLON-G-treated 3-day-old neutrophils were isolated from a green fluorescent protein (GFP)-expressing mouse and were thus positive for GFP expression. Untreated fresh neutrophils were prepared from a CD45.1 donor mouse. A mixed population (5 million) was intraperitoneally (i.p.) injected into a CD45.2 recipient mouse (see e.g., FIG. 2D). Drug treated and untreated fresh neutrophils were identified by expression of CD45.1 or GFP using flow cytometry analysis (see e.g., FIG. 2E). At early time points, neutrophil death does not play a major role in inflammation resolution, so the ratio of the two neutrophil populations remained unaltered at 6 hours. The relative half-life of untreated and CLON-G-treated neutrophils in the inflamed peritoneal cavity was also assessed at later time points, when considerable neutrophil death had occurred. The ratio of CLON-G-treated 3-day-old neutrophils to fresh neutrophils started to increase at 24 hours and reached a ratio of greater than 10:1 three days after the adoptive transfer, indicating that the CLON-G-treated 3-day-old neutrophils had a lower death rate than untreated fresh neutrophils at the inflammatory site (see e.g., FIG. 2F-2G). This result confirms that CLON-G treatment can promote the survival of transfused granulocytes in vivo due to the prolonged intracellular retention of the drugs. In contrast, consistent with the in vitro results (see e.g., FIG. 13), treatment with G-CSF alone failed to effectively protect neutrophils from aging, with G-CSF-treated 3-day-old neutrophils showing similar survival at the inflammatory site compared to fresh neutrophils (see e.g., FIG. 2F-2G). In this in vivo assay, the survival of untreated neutrophils in the recipient mice could not be assessed after 3 days of ex vivo culture, when most neutrophils had already died (see e.g., FIG. 1 and FIG. 13).

Stored CLON-G-Treated Neutrophils Functioned Normally and were Recruited to the Site of Infection as Efficiently as Transfused Fresh Neutrophils.

It was next explored whether CLON-G treatment affected mouse and human primary neutrophil functions critical for innate immunity and host defenses. First, it was investigated whether CLON-G treatment impaired neutrophil chemotaxis using the EZ-TAXISCAN apparatus, in which a stable chemoattractant gradient is formed in a 260 μm-wide channel, permitting direct visualization of neutrophil chemotaxis; see e.g., Li et al. 2009, supra; Prasad et al., Nat Immunol 12, 752-760 (2011), the content of which is incorporated herein by reference in its entirety. CLON-G-treated neutrophils were exposed to an fMLP (N-formyl-L-methionyl-L-leucyl-phenylalanine) gradient, and cell movement was recorded by time-lapse microscopy and analyzed using the Tracking Tool software. Both mouse and human neutrophils displayed the same directionality as freshly isolated neutrophils even after 5 days of CLON-G treatment (see e.g., FIG. 3A and FIG. 18A), and neutrophils treated for 1 day moved at the same speed as untreated fresh neutrophils. Neutrophils treated with CLON-G for 5 days migrated much faster than untreated or G-CSF alone-treated neutrophils cultured for only 1 day (see e.g., FIG. 3A and FIG. 18A).

Next, phagocytic capability was assessed by measuring neutrophil engulfment of opsonized fluorescein-conjugated Escherichia coli or zymosan (S. cerevisiae) bioparticles. CLON-G-treated murine neutrophils had a comparable phagocytosis efficiency (the percentage of cells that engulfed at least one bioparticle) and phagocytosis index (the number of internalized particles per 100 cells) to fresh neutrophils, even after 5 days of culture (see e.g., FIG. 3B); see e.g., Kambara et al. 2018, supra; Li et al. 2009, supra; Prasad et al., supra; Sakai et al., Immunity 37, 1037-1049 (2012), the content of which is incorporated herein by reference in its entirety. In contrast, the phagocytosis efficiency of untreated or G-CSF alone-treated neutrophils was lower even after one day of culture. Thus, CLON-G treatment augmented phagocytosis capability of aging neutrophils. Similar results were observed in human neutrophils (see e.g., FIG. 18B). Phagocytes utilize NADPH oxidase-dependent ROS release to clear pathogenic organisms during host defense responses. Studies have also demonstrated unconventional roles for ROS and NADPH oxidase activation in signal transduction and cell function. Similarly, fMLP-induced NADPH oxidase activation in stored CLON-G-treated cells, but not untreated or G-CSF alone-treated cells, was similar to that in freshly purified neutrophils, even after 5 days of culture (see e.g., FIG. 3C and FIG. 18C).

The effect of CLON-G treatment on the in vitro bacterial killing capability of neutrophils was investigated by measuring the reduction in colony-forming units (cfu) in the presence of mouse or human neutrophils. Stored CLON-G-treated, but not untreated or G-CSF alone-treated neutrophils, were as efficient as untreated fresh neutrophils in clearing live E. coli (see e.g., FIG. 3D-3E and FIG. 18D-18E).

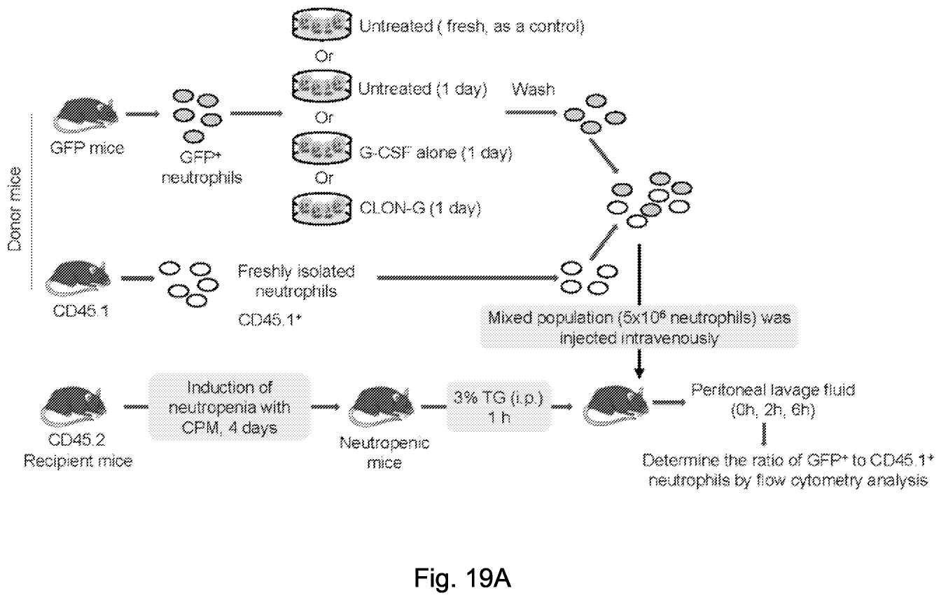

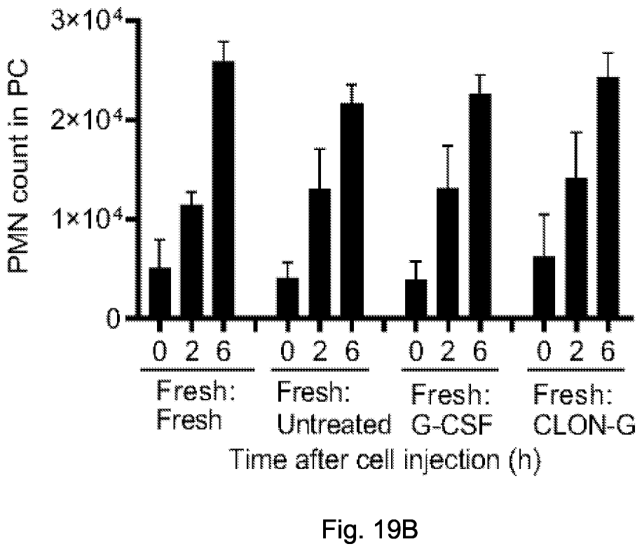

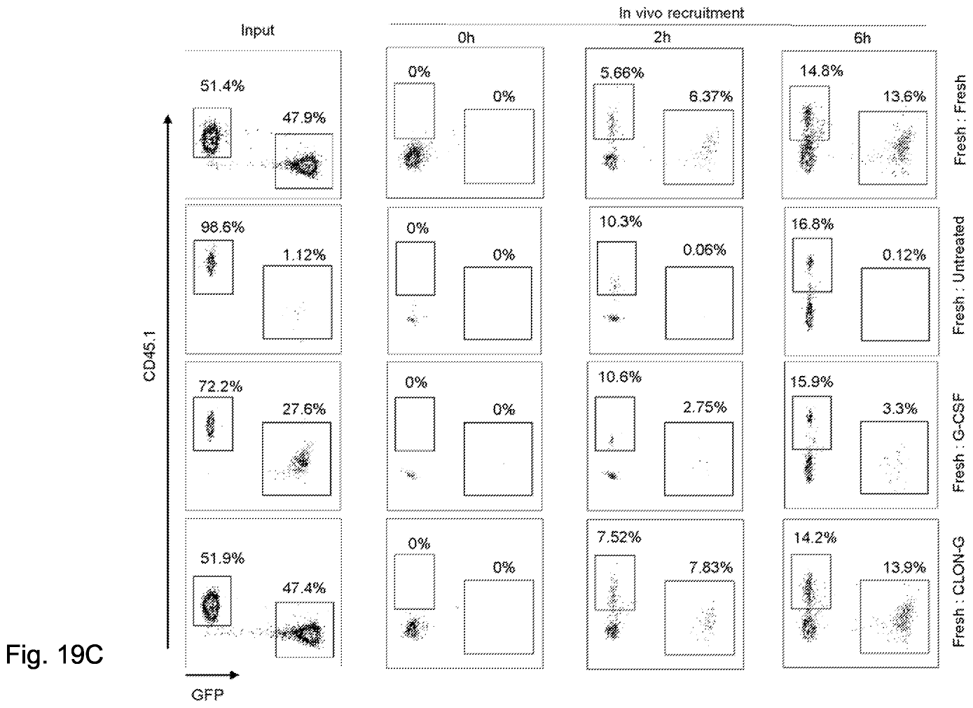

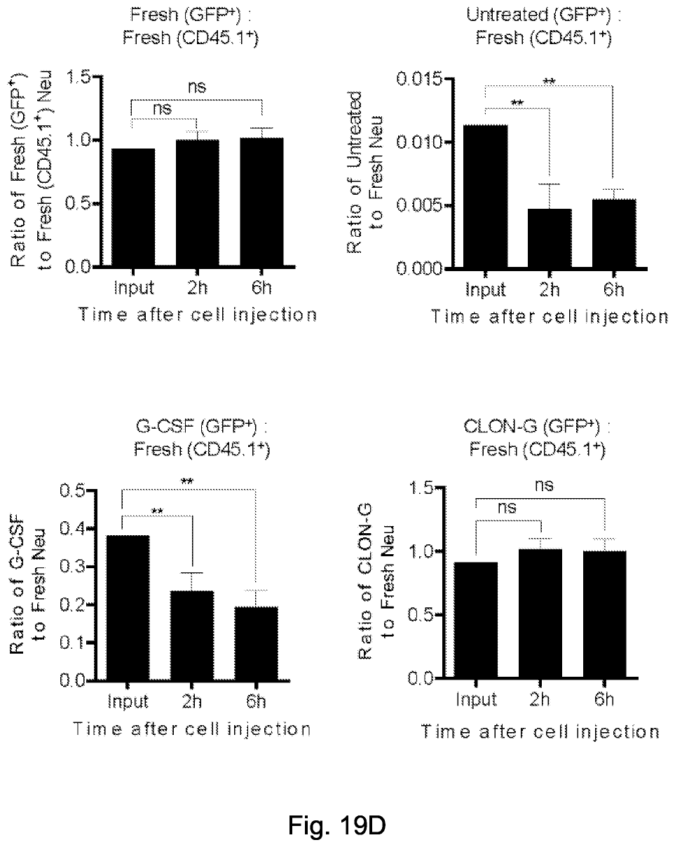

Finally, in vivo recruitment of transfused neutrophils to the inflammatory site was investigated using the peritonitis model. The trafficking of transfused CLON-G-treated 1-day-old GFP⁺ and untreated fresh CD45.1⁺ neutrophils was assessed in the same CD45.2 recipient mice (see e.g., FIG. 19A). At early time points (2 and 6 hours post transfusion) when neutrophil death did not play a major role, the number of transfused neutrophils in the peritoneal cavity solely depended on the recruitment efficiency. It increased gradually, with ˜3×10⁴transfused neutrophils detected in the peritoneal exudate 6 hours after neutrophil intravenous injection (see e.g., FIG. 19B). The ratio of transfused CLON-G-treated to untreated fresh neutrophils in the peritoneal cavity remained unaltered, indicating that the two neutrophil populations were recruited to the inflamed site with a similar efficiency (see e.g., FIG. 19C-19D). Consistent with the ex vivo study, untreated and G-CSF-treated 1-day-old neutrophils exhibited reduced recruitment efficiency compared to fresh neutrophils (see e.g., FIG. 19C-19D).

Transfused CLON-G-Treated 3-Day-Old Neutrophils were Recruited to Inflamed Lungs as Efficiently as Transfused Fresh Neutrophils in Neutropenia-Related Pneumonia.

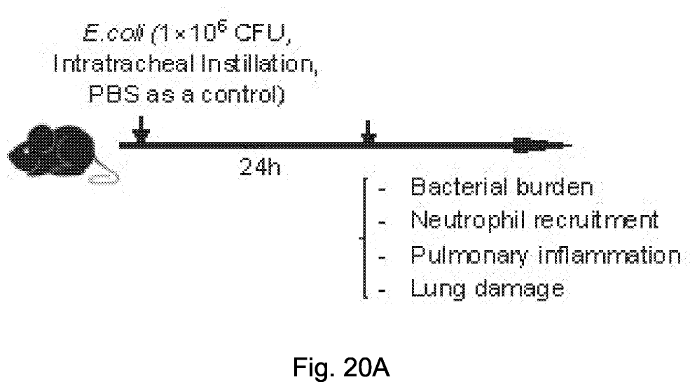

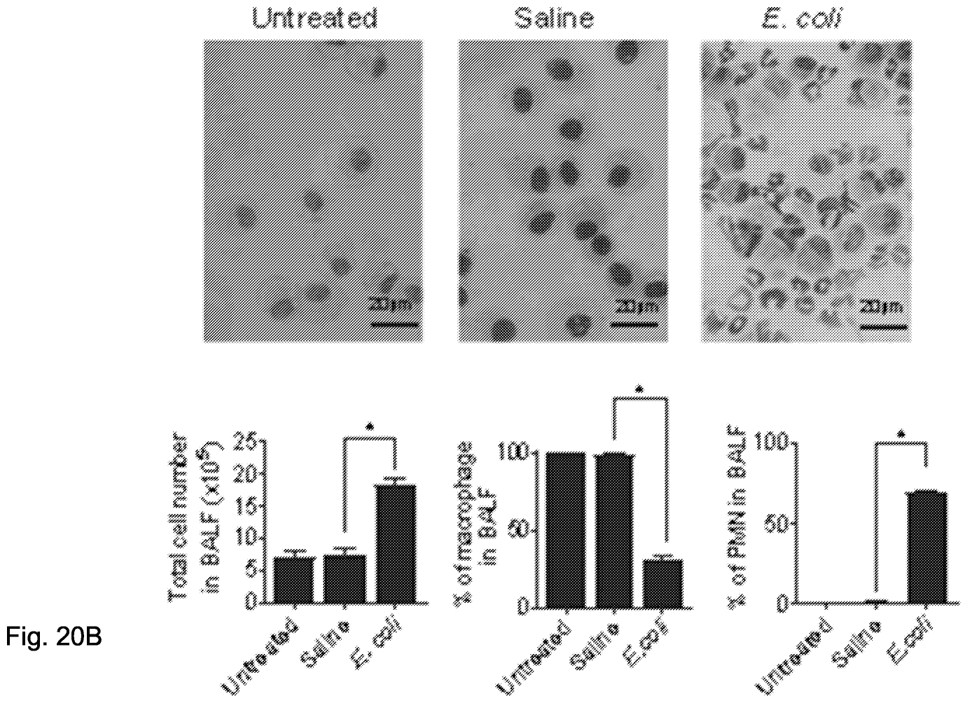

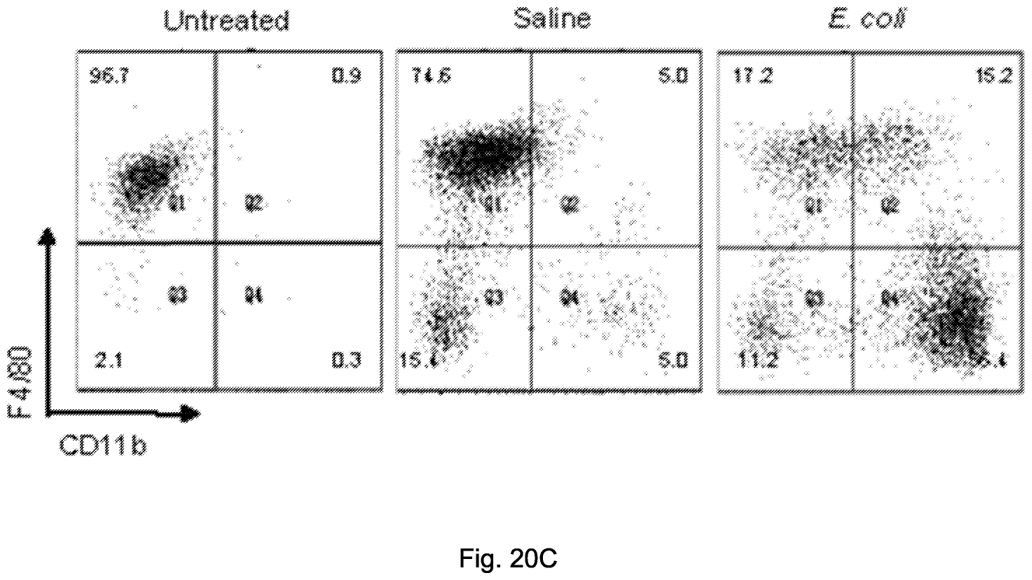

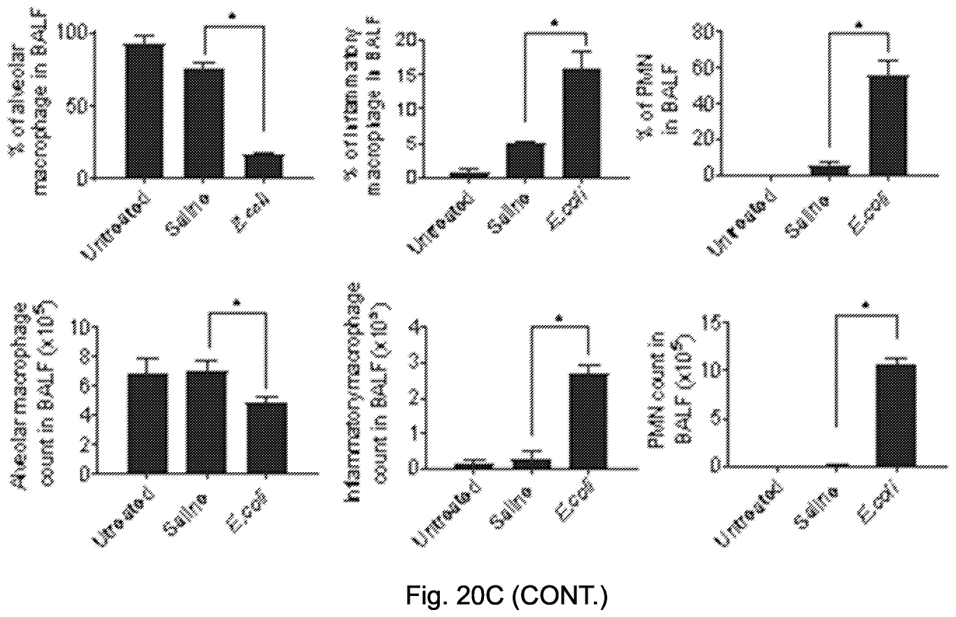

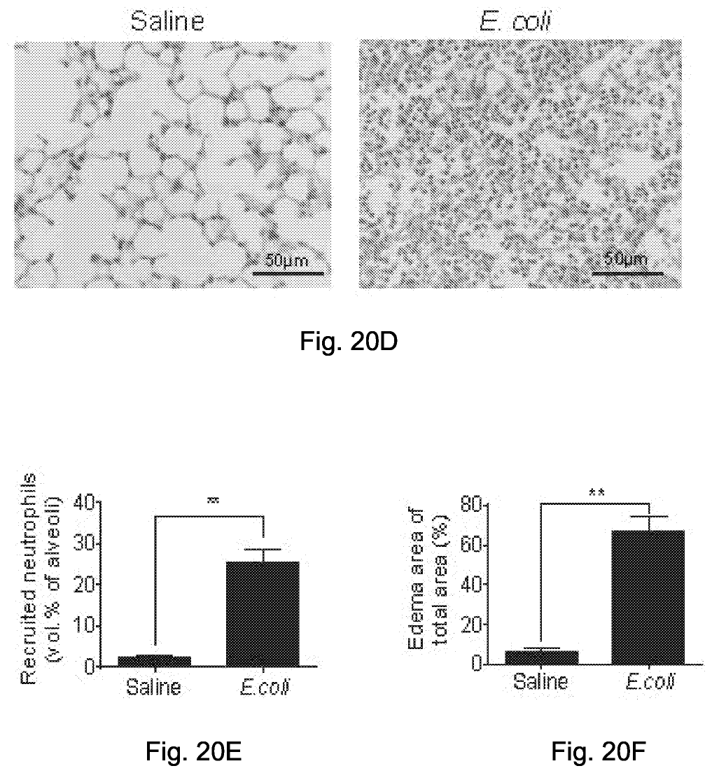

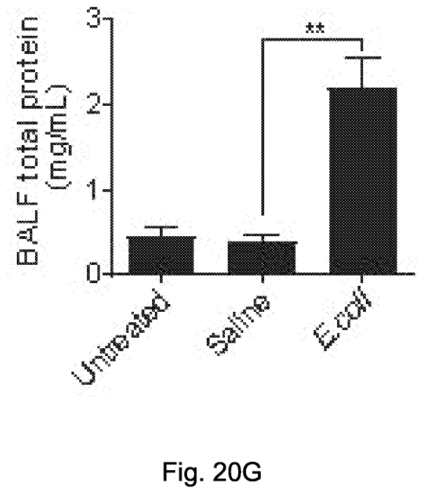

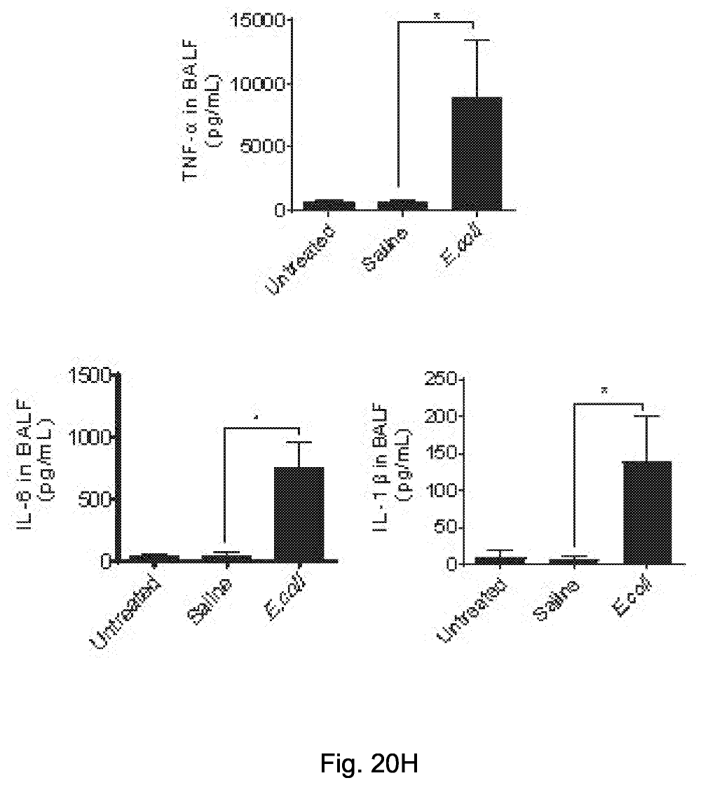

Next, the survival and function of transfused neutrophils in vivo was investigated in a clinically relevant mouse E. coli pneumonia model (see e.g., FIG. 20A); see e.g., Li et al. 2009, supra; Li et al., Blood 117, 6702-6713 (2011), the content of which is incorporated herein by reference in its entirety. Very few neutrophils were detected in the lungs of unchallenged mice. ˜1×10⁶ neutrophils were detected in the bronchoalveolar lavage fluid (BALF) 24 hours after bacteria instillation (see e.g., FIG. 20B-20C). Neutrophil recruitment was also apparent when the number of emigrated neutrophils in alveolar air spaces was quantified by histomorphometric analysis of lung sections (see e.g., FIG. 20D-20E). E. coli-induced pulmonary inflammation was associated with edema formation (see e.g., FIG. 20F-20G) and inflammatory cytokine production (see e.g., FIG. 20H).

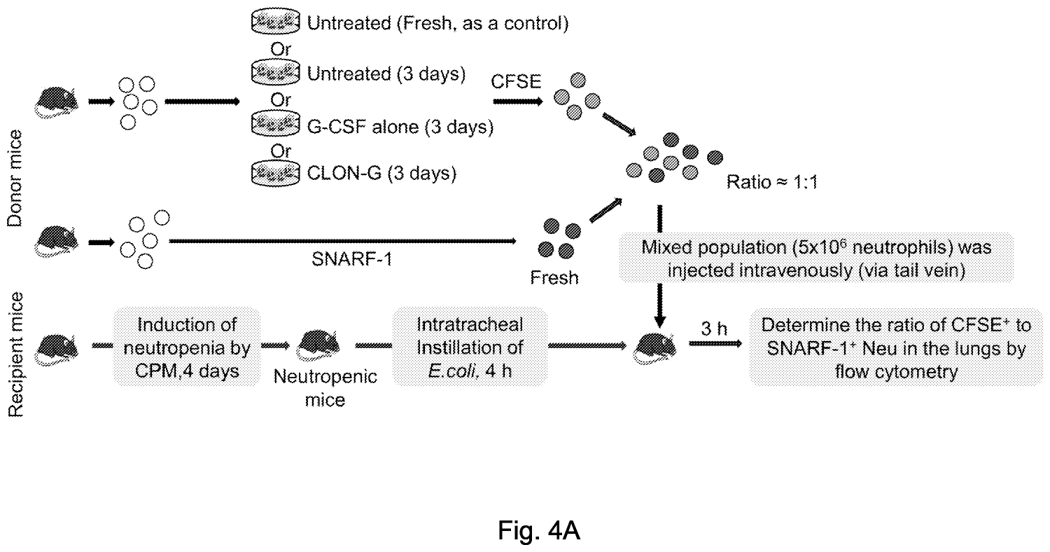

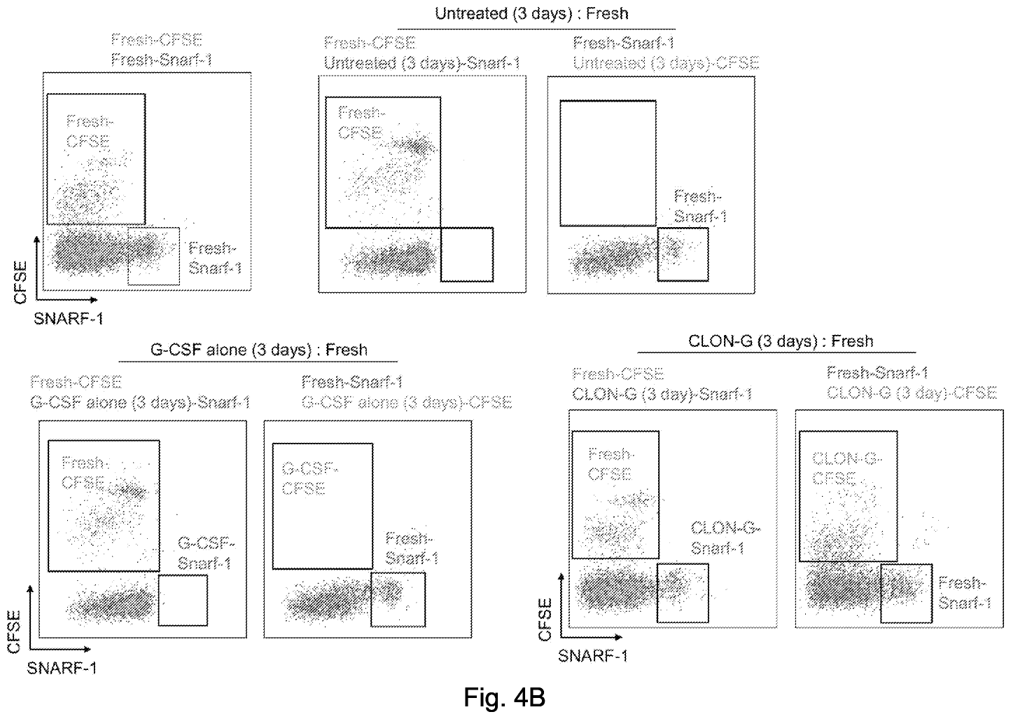

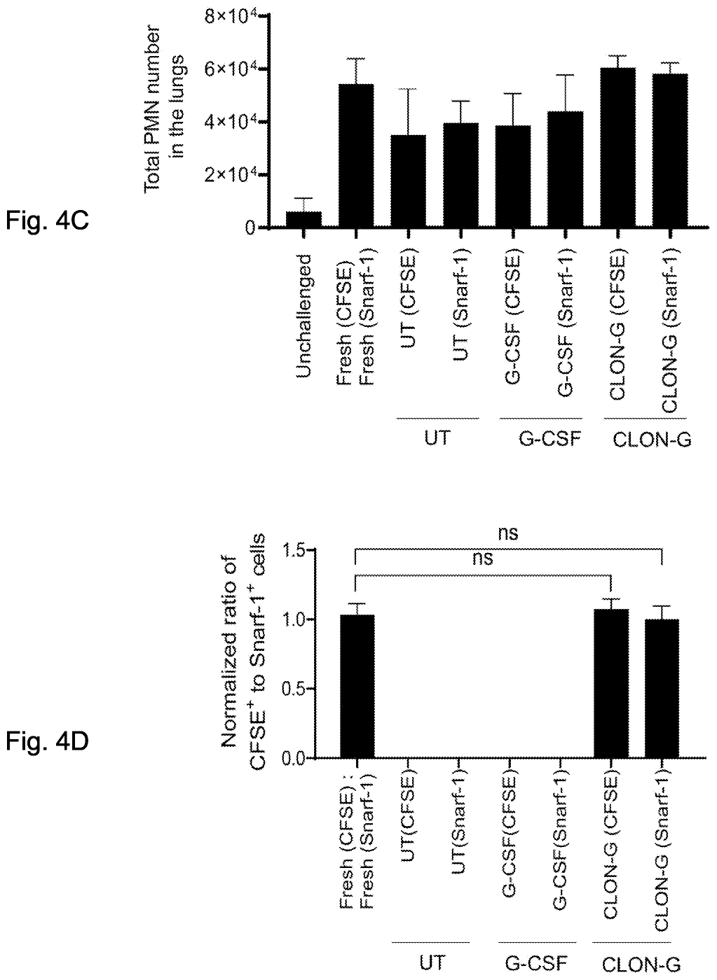

The unsatisfying clinical outcome of GTX is largely due to inefficient accumulation of transfused neutrophils at sites of infection. It was therefore examined whether storing neutrophils in CLON-G-containing medium altered the recruitment of transfused neutrophils to inflamed lungs. To distinguish transfused from endogenous neutrophils (about 5-10% of normal abundance), untreated fresh neutrophils were labeled with the intracellular fluorescent dye 5-(and -6)-carboxyfluorescein diacetate succinimidyl esters (CFSE; which fluoresced green) and CLON-G-treated 3-day-old neutrophils with 5-(and -6)-chloromethyl seminaphtorhodafluor-1-acetoxymethylester (SNARF-1) acetate (which fluoresced red), or vice versa. Cell labeling does not alter neutrophil life span or function; see e.g., Li et al. 2009, supra; Jia et al., Immunity 27, 453-467 (2007), the content of which is incorporated herein by reference in its entirety. Clinically, GTX is often administered after an infection has been detected. Accordingly, the mixed (1:1) population was transfused into the same neutropenic recipient mouse 4 hours after E. coli instillation (see e.g., FIG. 4A), allowing the comparison of the trafficking of the two neutrophil types in exactly the same environment. Untreated fresh and CLON-G-treated 3-day-old neutrophils were handled identically before being mixed and studied in parallel, thereby controlling for the effects of ex vivo manipulation. E. coli challenge triggered neutrophil accumulation in the lungs (see e.g., FIG. 4B). Transfused untreated fresh and CLON-G-treated 3-day-old neutrophils accumulating in the lungs were identified by their distinct fluorescent labels by flow cytometry analysis (see e.g., FIG. 4C). At an early time point (3 hours), when neutrophil death does not play a major role in inflammation resolution, there remained a 1:1 ratio of transfused 3-day-old CLON-G-treated to transfused untreated fresh neutrophils in the lungs (see e.g., FIG. 4D), indicating that CLON-G-treatment did not compromise recruitment of transfused neutrophils to the lungs in vivo in neutropenic mice. Instead, CLON-G-treatment prevented aging-related reduction of neutrophil recruitment capability. As a further comparison, untreated and G-CSF-treated neutrophils exhibited reduced recruitment efficiency compared to fresh neutrophils (see e.g., FIG. 4B-4D).

Exaggerated accumulation or hyperactivation of neutrophils can lead to unwanted tissue damage. Induction of neutrophil apoptosis has been proposed to be an effective strategy for terminating or resolving deleterious inflammatory responses; see e.g., Duffin et al., Immunol Rev 236, 28-40 (2010), the content of which is incorporated herein by reference in its entirety. However, GTX is mainly used clinically for life-threatening bacterial and fungal infections in severely neutropenic patients. Neutrophil-induced tissue damage is a less of a concern in neutropenic patients with drastically reduced neutrophil numbers in whom the release of harmful compounds such as oxidants, proteases, and DNA is minimal. Consistently, no tissue damage elicited by transfusion of untreated or CLON-G-treated neutrophils was observed in neutropenic recipient mice (see e.g., FIG. 21). Additionally, it was investigated whether transfusion of CLON-G treated neutrophils exacerbated lung injury in a lipopolysaccharide (LPS)-induced acute lung inflammation model. Again, no detrimental effect of CLON-G treated neutrophils were detected after their transfusion to neutropenic hosts (see e.g., FIG. 21).

Transfusion with Stored CLON-G-Treated Neutrophils Enhanced Host Defenses and Alleviated Infection-Induced Lung Damage in Neutropenia-Related Pneumonia.

The efficacy of transfused neutrophils in GTX is ultimately reflected by the recipient's capability to clear invading pathogens. Thus, it was examined whether transfusion with stored CLON-G-treated aged neutrophils had a similar bactericidal capacity to untreated fresh neutrophils (see e.g., FIG. 5A) in mice challenged with 5×10³ cfu live E. coli. As expected, due to the lack of neutrophils in CPM-induced neutropenic mice, bacterial numbers gradually increased after initial instillation, reaching 6×10⁶ after 24 hours (see e.g., FIG. 5B). GTX enhanced bacterial killing in neutropenic recipient mice. When a substantial number of transfused neutrophils accumulated in the lungs, bacterial burden (the number of live bacteria in the lungs) decreased drastically, reflecting the bacteria-killing capability of transfused neutrophils (see e.g., FIG. 5B-5C). Transfusion with CLON-G-treated 3-day-old neutrophils enhanced bacteria-killing capacity in neutropenic recipient mice as effectively as transfusion with fresh neutrophils, whereas transfusion with untreated or G-CSF alone-treated neutrophils was ineffective (see e.g., FIG. 5B-5C).

To assess the severity of neutropenia-related pneumonia, lung tissue sections were examined by microscopy (see e.g., FIG. 5D). Edema, a sign of lung inflammation, accumulated in the lungs as assessed by microscopy and quantified by morphometry. Transfusion with CLON-G-treated 3-day-old or untreated fresh neutrophils inhibited edema formation, with no difference detected between the two populations. However, transfusion with untreated or G-CSF alone-treated neutrophils failed to produce the same protective effect (see e.g., FIG. 5D-5E). Severe pneumonia is often accompanied by vascular leakage, with increased BALF protein concentrations used as an indicator of vascular leakage and a key parameter of inflammatory lung injury. Similarly, neutrophil transfusion reduced BALF protein concentrations, with no detectable difference in mice transfused with CLON-G-treated 3-day-old neutrophils and mice transfused with fresh neutrophils. BALF protein concentrations remained high when untreated or G-CSF alone-treated neutrophils were transfused (see e.g., FIG. 5F). Finally, the overall inflammatory response was evaluated by assessing concentrations of tumor necrosis factor-α (TNF-α) and interleukin (IL)-6 in inflamed lungs by enzyme-linked immunosorbent assay (ELISA). Transfusion with CLON-G-treated 3-day-old neutrophils and untreated fresh neutrophils both reduced inflammatory cytokine concentrations in E. coli-challenged neutropenic recipients. In contrast, transfusion with untreated or G-CSF alone-treated neutrophils was unable to reduce inflammatory cytokine concentrations in E. coli-challenged neutropenic recipients (see e.g., FIG. 5G).

Patients with neutropenia-related pneumonia can die from severe lung infections. Accordingly, it was investigated whether transfusion with stored CLON-G-treated neutrophils increased the survival rate of challenged mice. When challenged with 5×10³ live E. coli, >60% of neutropenic mice mock treated with phosphate-buffered saline (PBS) died within two days. GTX increased the survival rate of challenged mice. Similar to fresh neutrophils, transfusion with stored CLON-G-treated 3-day-old neutrophils, but not untreated or G-CSF alone-treated neutrophils, completely rescued E. coli-challenged neutropenic recipients (see e.g., FIG. 5H). Taken together, consistent with the in vitro data, CLON-G treatment did not impair the bacterial killing capability of transfused neutrophils in vivo. Transfusion with stored CLON-G-treated neutrophils enhanced host defenses and alleviated infection-induced tissue damage as effectively as transfusion with fresh neutrophils.

Transfusion with Stored CLON-G-Treated 3-Day-Old Neutrophils Enhanced Host Defenses as Effectively as Transfusion with Fresh Neutrophils in Neutropenia-Related Fungal Infection.

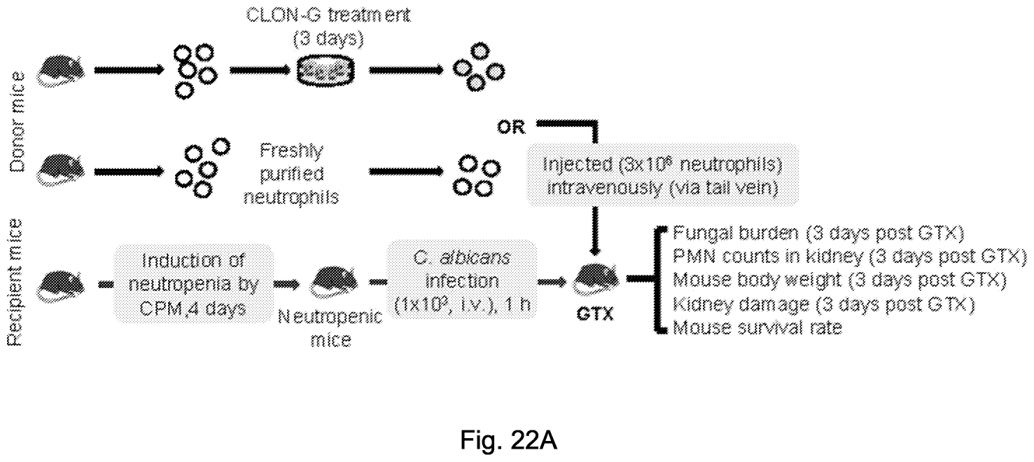

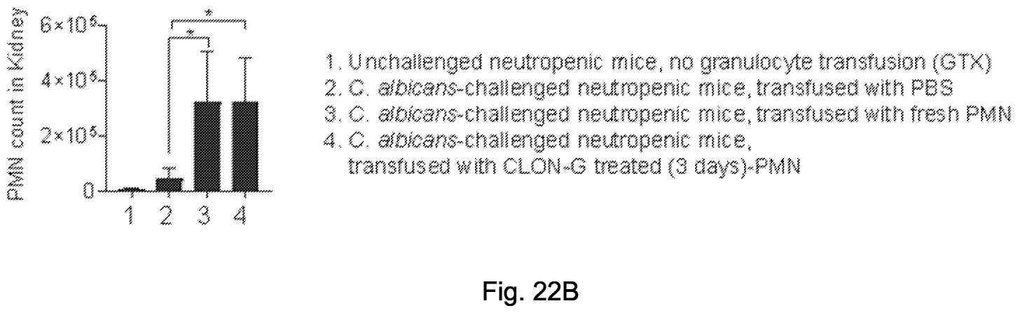

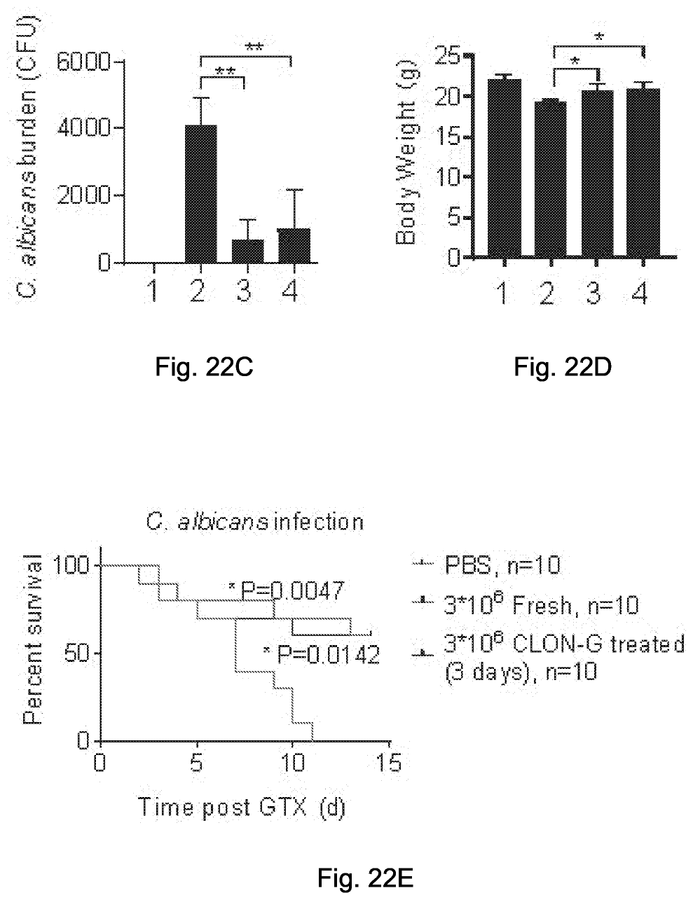

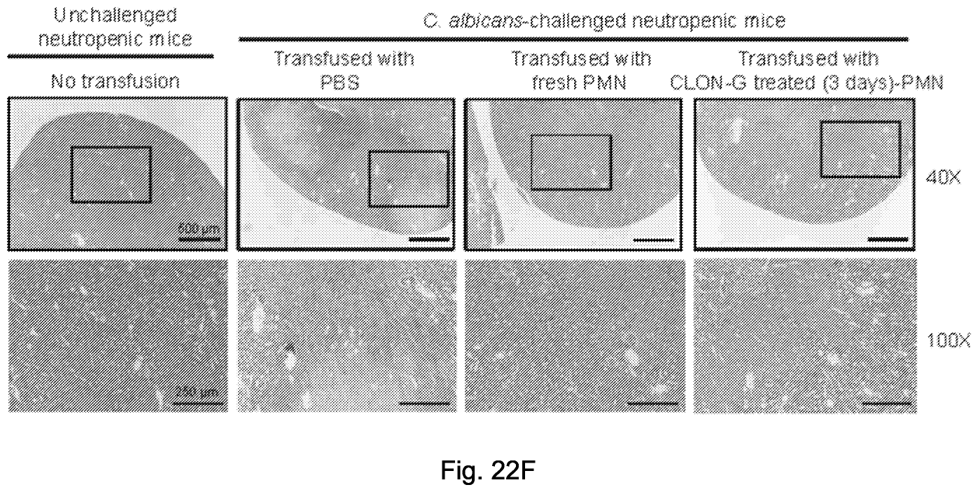

Neutrophils are the principal effector immune cells against Candida albicans infection, which is more frequent in patients with neutropenia; see e.g., Nesher et al., Infection 42, 5-13 (2014), the contents of which are incorporated herein by reference in their entirety. Utilizing a mouse model of systemic candidiasis, it was assessed whether transfusion with stored CLON-G-treated neutrophils enhanced host defenses in neutropenia-related fungal infection as effectively as transfusion with untreated fresh neutrophils (see e.g., FIG. 22A). GTX increased neutrophil accumulation in the kidney, a major organ contributing to host defenses against C. albicans infection (see e.g., FIG. 22B). Consistently, neutrophil-transfused mice had a decreased fungal burden (see e.g., FIG. 22C), reversal of infection-induced body weight loss (see e.g., FIG. 22D), and improved survival (see e.g., FIG. 22E) compared to mice without GTX. Mice infected with C. albicans developed kidney necrosis and hemorrhage. Mice receiving GTX showed reduced kidney necrosis (see e.g., FIG. 22F), further indicating that GTX conferred a degree of protection against C. albicans-induced sepsis and disease. Transfusion with stored CLON-G-treated 3-day-old neutrophils was as effective as transfusion with fresh neutrophils (see e.g., FIG. 22B-22F). Therefore, CLON-G treatment represents a general strategy to alter neutrophil fate and improve GTX in neutropenia-related infections.

### CLON-G Treatment Prolonged the Shelf-Life of Granulocyte Apheresis Concentrates.

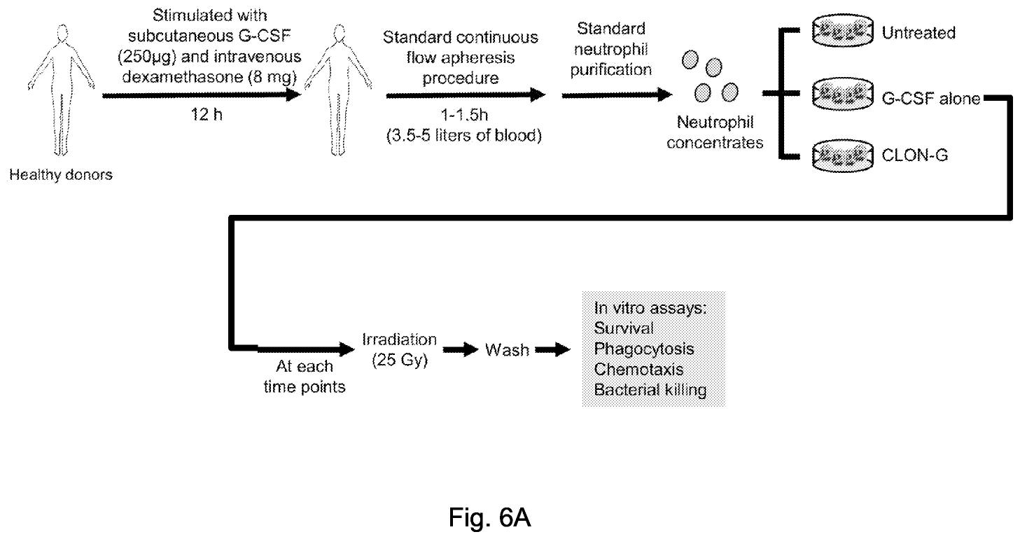

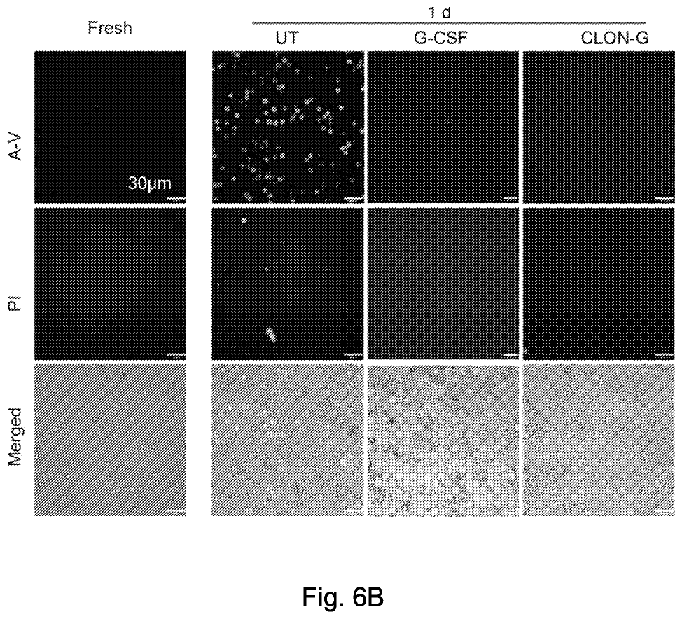

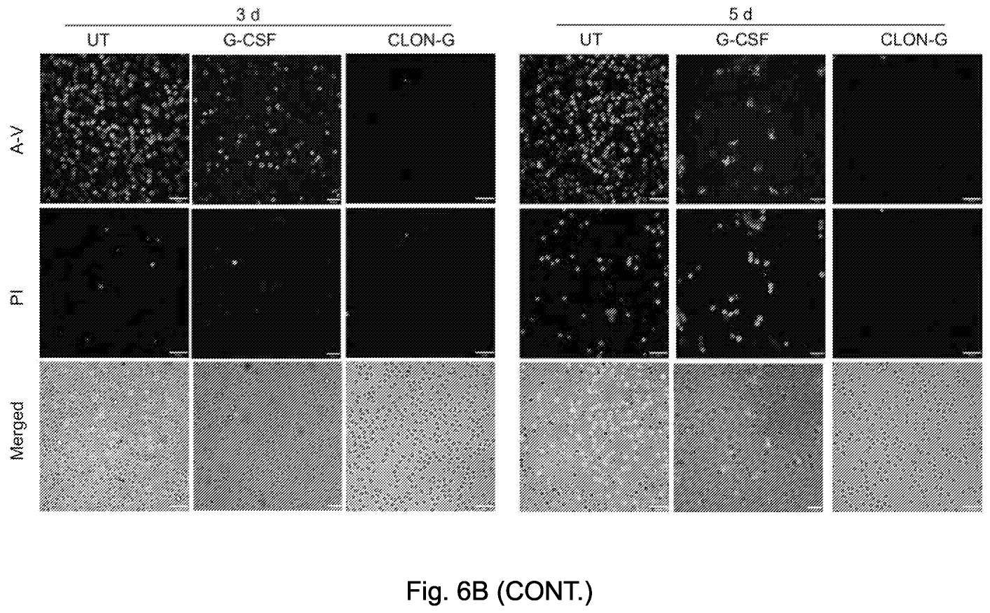

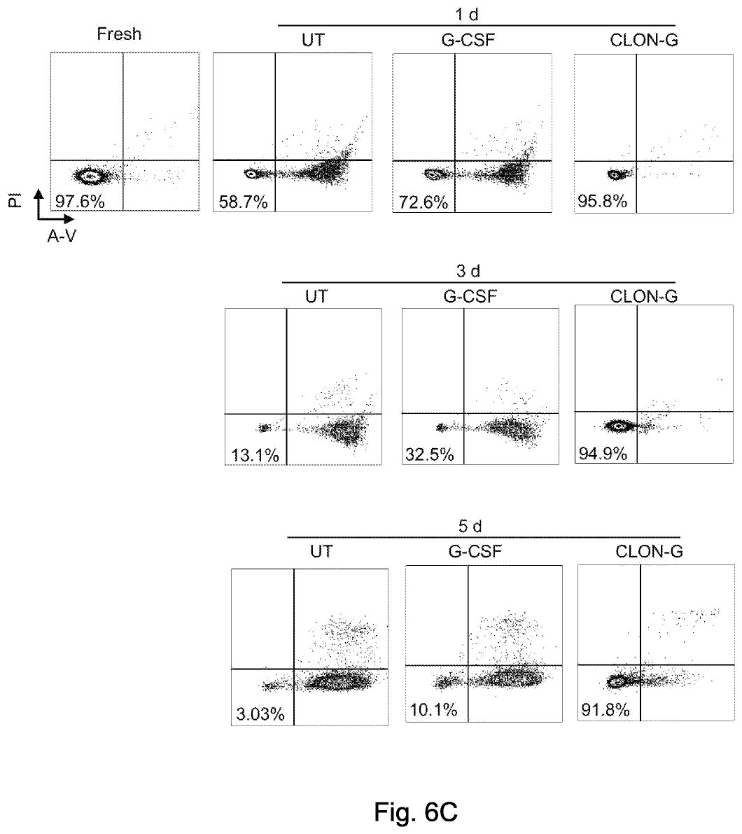

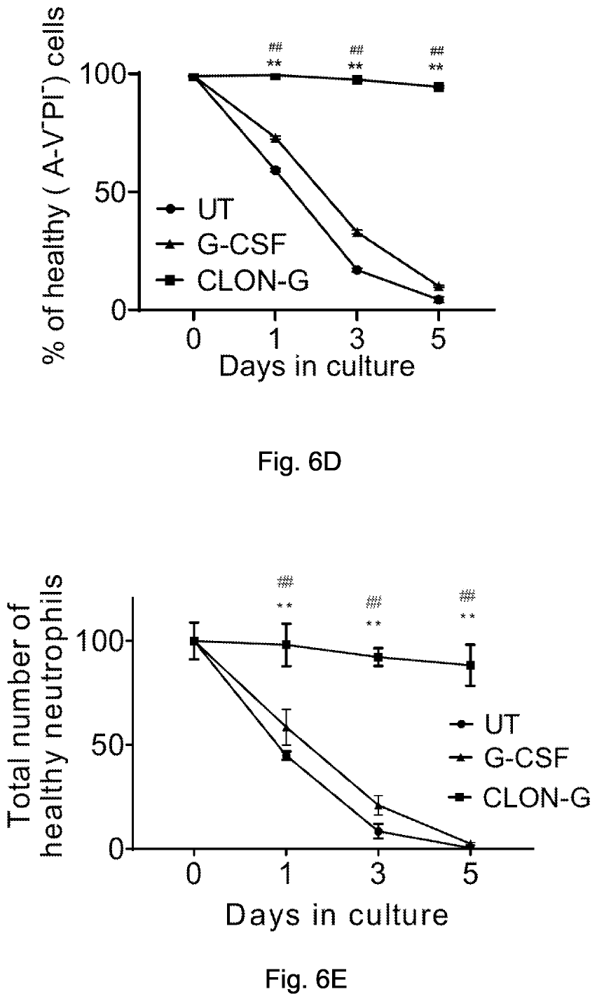

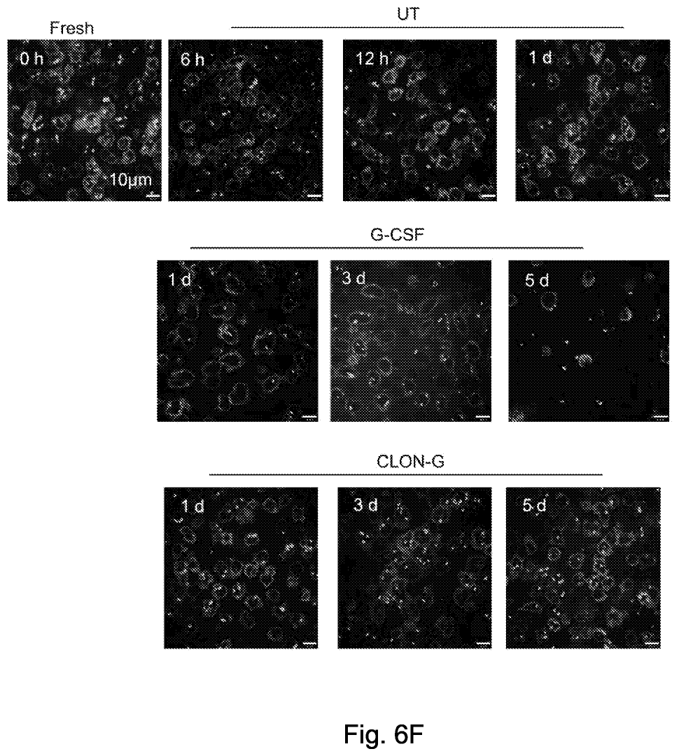

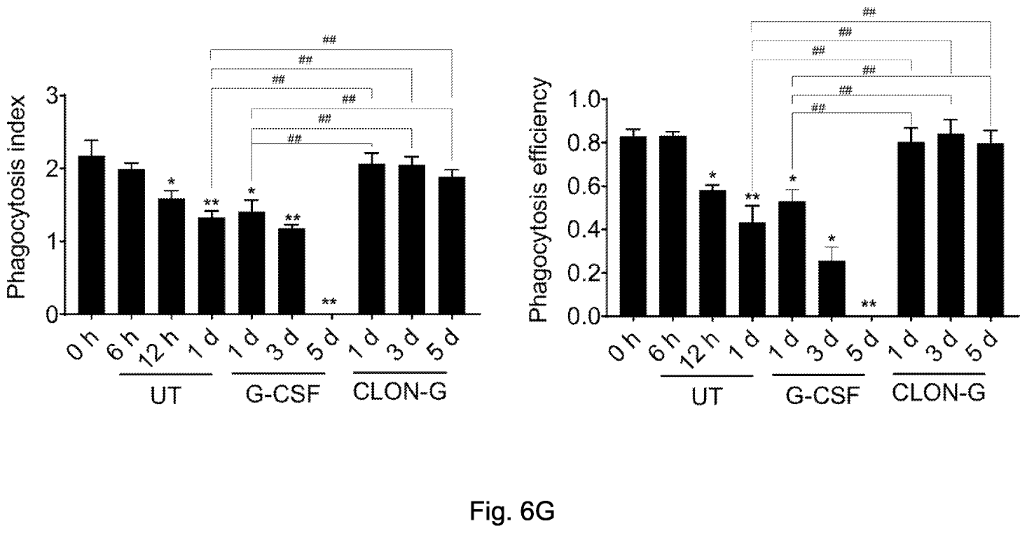

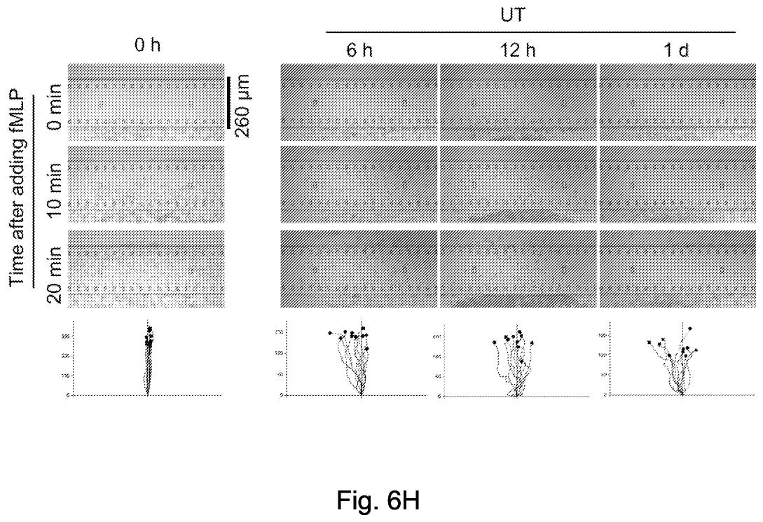

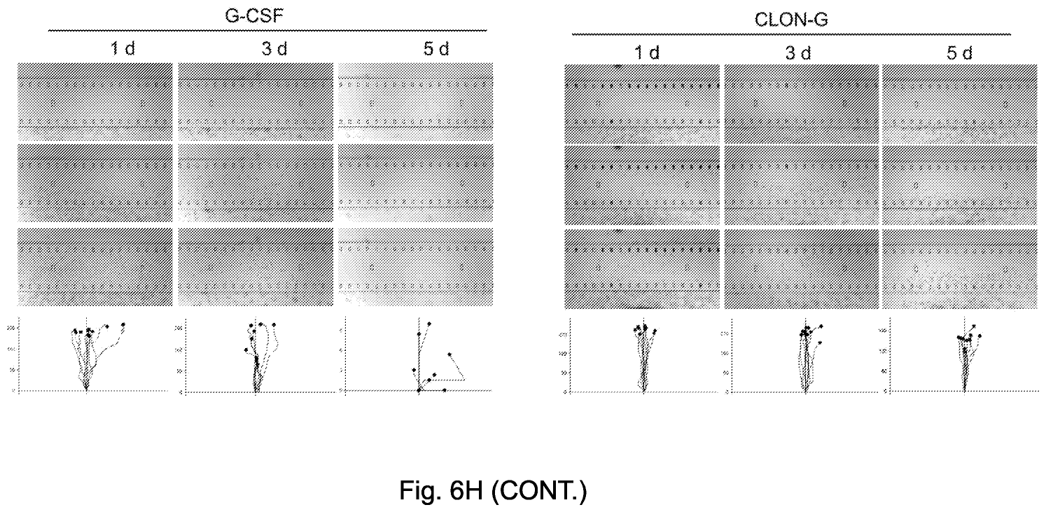

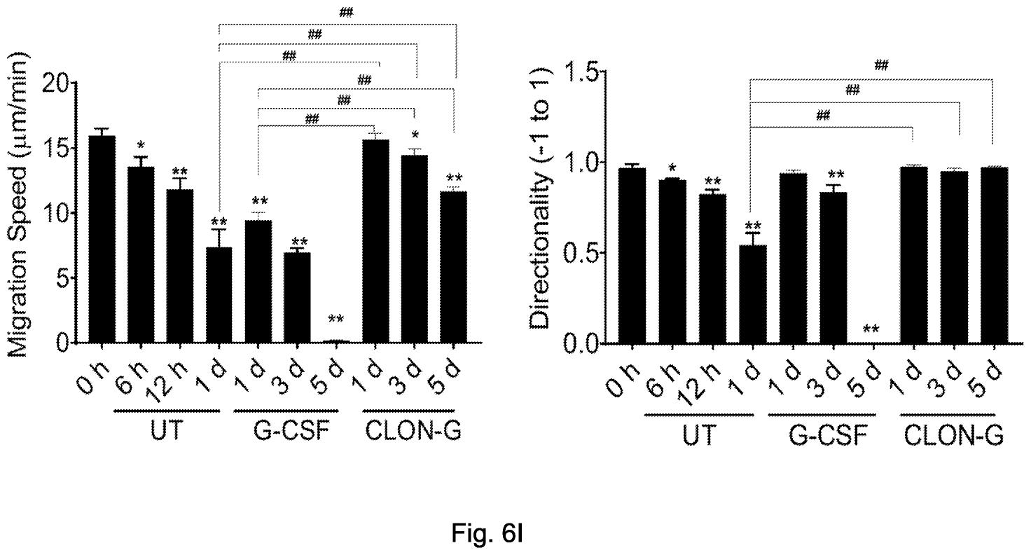

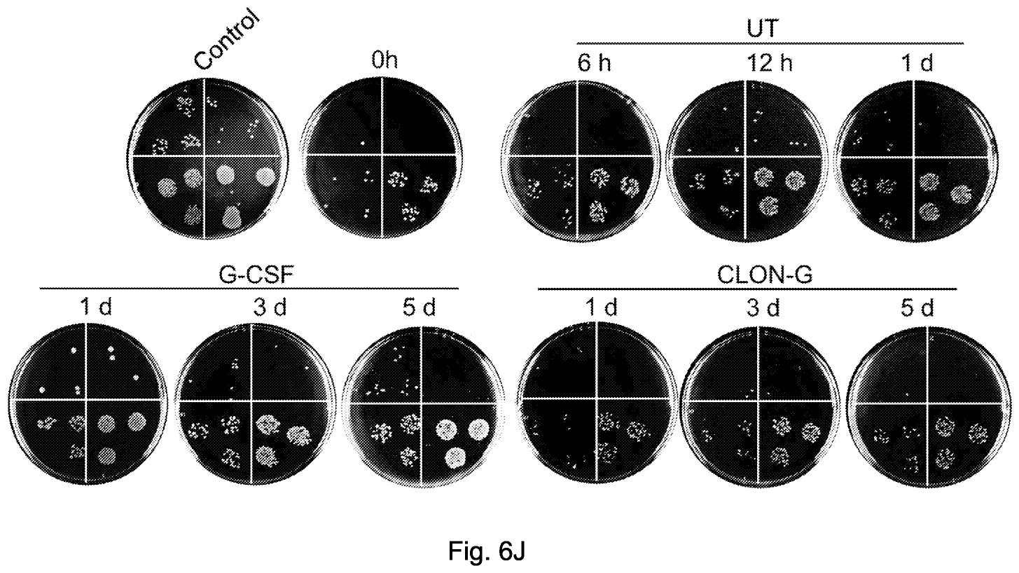

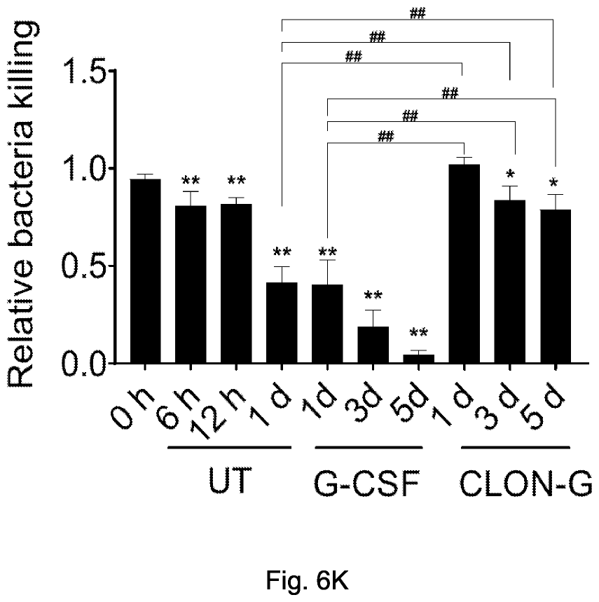

Neutrophils used for GTX are collected from the blood of healthy donors by automatic apheresis. The donors are often stimulated with G-CSF and dexamethasone to increase the abundance of circulating peripheral blood neutrophils by promoting demargination of neutrophils from the endothelial surface of blood vessels and mobilization of neutrophils from the bone marrow. In addition, neutrophils collected after the administration of G-CSF appear to be of better quality. The effect of CLON-G on the survival and function of neutrophils was investigated in the granulocyte apheresis concentrates collected and processed following a standard GTX protocol (see e.g., FIG. 6A). Clinically, apheresis concentrates are also routinely irradiated (25 grays (Gy)) prior to transfusion in order to render lymphocytes in the product incapable of dividing and eliminate the risk of transfusion-associated graft-versus-host disease (ta-GVHD). The irradiation procedure does not interfere with neutrophil function. To mimic this clinical situation, neutrophils were irradiated prior to each experiment (see e.g., FIG. 6A). CLON-G treatment prolonged the survival of neutrophils in the granulocyte apheresis concentrates (see e.g., FIG. 6B-6E). Stored CLON-G-treated 3-day-old, but not untreated or G-CSF alone-treated 1-day-old neutrophils, were as efficient as fresh neutrophils in undergoing phagocytosis (see e.g., FIG. 6F-6G), chemotaxis (see e.g., FIG. 6H-6I) and clearing live E. coli (see e.g., FIG. 6J and K). Neutrophils retained the capacity to kill bacteria even after 5 days of CLON-G treatment, only showing a moderate 15% reduction in relative bacterial killing capability, compared to a more than 50% reduction in untreated or G-CSF alone-treated neutrophils cultured for only 1 day (see e.g., FIG. 6K).

Moreover, to examine whether CLON-G-treatment prolonged the shelf-life of a clinical granulocyte transfusion product, the compounds were added directly to apheresis collection bags. Neutrophils in the granulocyte apheresis concentrates were isolated at each time point and their survival and function were assessed using the same approaches described above (see e.g., FIG. 7A). Similarly, CLON-G-treated, but not untreated or G-CSF alone-treated, neutrophils showed improved survival (see e.g., FIG. 7B-7E). Neutrophil functions, including phagocytosis (see e.g., FIG. 7F), chemotaxis (see e.g., FIG. 7G-7H), and bacteria clearance (see e.g., FIG. 7I-7J) were also better preserved in the presence of CLON-G. Thus, CLON-G treatment prolonged the lifespan of granulocytes in unmanipulated apheresis concentrates. This approach is applicable to the goal of prolonging the shelf-life of the clinical standard granulocyte transfusion products.

### CLON-G Pre-Treatment Prolonged Half-Life of Transfused Human Neutrophils In Vivo in NOD-Scid IL2Rgammanull (NSG) Mice.

To investigate the in vivo survival and function of transfused human neutrophils, a model was utilized in which the survival, migration and phagocytosis of transfused human neutrophils were assessed during peritonitis in neutrophil-depleted NOD-scid IL2Rgamma^{null} (NSG) mice; see e.g., Trump et al., Stem cells translational medicine 8, 557-567 (2019), the content of which is incorporated herein by reference in its entirety. Human neutrophils were isolated from apheresis-collected granulocyte concentrates (see e.g., FIG. 8A). The transfused human neutrophils in the NSG recipient mice were identified by their unique surface markers including human-CD16 and human-CD11b (see e.g., FIG. 8B). Migration of transfused human neutrophils to inflammatory site was assessed at early time points (1 and 2 hours post transfusion) when neutrophil death had not yet started. CLON-G-treated 3-day-old human neutrophils were recruited to the inflamed peritoneal cavity as efficiently as fresh human neutrophils, indicating that CLON-G-treatment did not affect recruitment of human neutrophils. In contrast, due to substantial death of cultured untreated or G-CSF alone-treated human neutrophils, their numbers were lower in the inflamed peritoneal cavity in the NSG recipients (see e.g., FIG. 8B-8C). The in vivo phagocytosis capability of the transfused human neutrophils was assessed in peritonitis induced by pHrodo-E. coli, a nonviable fluorescently labeled bacterium (see e.g., FIG. 8D). Transfused CLON-G-treated 3-day-old human neutrophils displayed a similar phagocytosis index compared to transfused fresh human neutrophils. In contrast, the phagocytosis efficiency of transfused untreated or G-CSF alone-treated neutrophils was about 50% lower than that of fresh human neutrophils (see e.g., FIG. 8E-8F). Finally, to measure human neutrophil death at the inflammatory site in recipients, fresh or cultured apheresis-collected granulocyte concentrates were injected into the inflamed peritoneal cavity directly (see e.g., FIG. 8G). At each time point examined, the numbers of CLON-G-treated 3-day-old human neutrophils left in the peritoneal cavity were comparable to those of fresh neutrophils, whereas the untreated and G-CSF alone-treated human neutrophils showed faster rates of cell death (see e.g., FIG. 8H). Collectively, these results demonstrate that CLON-G treatment prolonged half-life of human neutrophils. The transfused CLON-G-treated human neutrophils functioned normally and were recruited to the site of infection as efficiently as transfused fresh neutrophils in vivo in NSG mice.

Neutrophils are generally regarded as short-lived cells that die quickly without stimulation. Increasing neutrophil viability represents a major challenge for clinical application. In one reported method, neutrophil half-life was increased in vitro from about 1 day to 2 days when conditioned for 20 hours at 37° C. in anoxic culture medium supplemented with 3 mM glucose and 32 mg/mL dimethyloxalylglycine (DMOG); see e.g., Monceaux et al., Blood 128, 993-1002 (2016), the contents of which are incorporated herein by reference in their entirety. In contrast, by targeting multiple death mechanisms, described herein is a treatment, CLON-G treatment, that altered neutrophil fate and prolonged the survival of both human and mouse neutrophils, increasing their half-life from less than 24 hours to greater than 5 days. The morphological, physiological, and bactericidal functions of the neutrophils were unaltered by CLON-G treatment. Transfusion with stored CLON-G-treated neutrophils enhanced host defenses and alleviated infection-induced tissue damage as effectively as transfusion with untreated fresh neutrophils in a clinically relevant murine GTX model in neutropenia-related bacterial pneumonia and systemic candidiasis. CLON-G-treatment prolonged the shelf-life and preserved the function of apheresis-collected human clinical granulocyte transfusion products both ex vivo and in vivo in NSG mice.

In GTX therapy, there is a great need to use stored granulocyte products. Even though granulocytes for transfusion are collected on an “as needed” basis, it is nevertheless inevitable that granulocyte concentrates are stored for a period of time prior to infusion. First, the time from mobilization and collection of granulocytes to transfusion is rarely less than 12 hours, as it takes time to collect, process, and transfuse. It also takes a tremendous effort to get pre-transfusion tests, including ABO and RhD typing and testing for Transfusion Transmitted Disease (TTD) markers, done in time for GTX. Thus, when preparation is taken into account, using neutrophils stored for 24 hours for GTX is not uncommon. In addition, in many cases, neutrophils used for GTX are collected off-site, delaying transfusion. In house granulocyte collection can only be performed in a small number of medical centers. Most granulocyte collections are not carried out in hospitals where they are transfused. It is necessary to ship patient samples from one location to another. Finally, developing a product that can be stored for longer time may help more patients get the GTX therapy as leukapheresis donors are difficult to recruit. The possibility of using stored granulocytes is particularly relevant in small pediatric patients, since it would allow concentrates to be split for other patients.

The inability to store the granulocyte products with effective maintenance of neutrophil functional integrity has been well-documented and has limited the use of GTX therapy. Current GTX practice stipulates using granulocytes collected the same day. Longer storage periods are avoided because of poor neutrophil survival and function. Storage of granulocyte concentrate using a commonly used procedure (e.g., in plastic bags, with autologous plasma, citrate anticoagulant, unagitated, at room temperature) is associated with impairment in neutrophil function within 24 hours from the time of collection. Studies examining neutrophils collected by centrifugation leukapheresis demonstrated substantial decreases in neutrophil functions such as ROS production, phagocytosis, bactericidal activity, chemotaxis, and in vivo migration, with storage up to 24 hours. These observations led to the current clinical practice in which granulocytes are transfused as soon as possible after collection, but definitely within 24 hours of collection. Human neutrophils stored for 24 hours have reduced bactericidal capability, which can contribute to the suboptimal and inconsistent outcomes of GTX. See e.g., Marfin et al., J Intensive Care Med 30, 79-88 (2015); Lane, Transfus Med Rev 4, 23-34 (1990); Price et al., Blood 54, 977-986 (1979); Glasser et al., Journal of clinical apheresis 1, 179-184 (1983); the contents of each of which are incorporated herein by reference in their entireties.

A major goal of the blood bank is to collect, store, and provide an adequate number of functional granulocytes. Optimal methods of granulocyte generation are important. Donor stimulation with a combination of G-CSF and dexamethasone has become the standard procedure to increase the number of circulating neutrophils in leukapheresis donors and, thus, to collect a sufficient number of cells for the preparation of granulocyte concentrates. The granulocytes from the leukapheresis product, obtained after in vivo G-CSF/dexamethasone mobilization, are functionally indistinguishable from those isolated from untreated donors. Multiple studies showed that pre-collection treatment with G-CSF plus dexamethasone is an effective way to maintain the functional properties of the collected neutrophils for GTX therapy. Pretreatment with G-CSF increases granulocyte yield and prolongs granulocyte survival time. It was reported that without agitation granulocyte concentrates can be stored at room temperature for a maximum of 24 hours. Neutrophil morphology after 24 hours of storage showed no differences from that of fresh control cells on cytospins. When assessed by means of FLUOROBLOK inserts or a fluorescence-based assay with 96-well chemotaxis chamber plates, neutrophil in vitro chemotaxis capability appeared to be intact after 24 hours of storage. Previous studies showed that some functional and biochemical characteristics (such as phorbol 12-myristate 13-acetate (PMA)-induced ROS production) of G-CSF-mobilized neutrophils can be retained for at least 24 hours of storage by adding additional G-CSF in apheresis bags. Nevertheless, these G-CSF-treated 1-day-old neutrophils still displayed a lower platelet-activating factor (PAF) and fMLP response compared fresh neutrophils. In addition, using a modified Boyden chamber technique, it was revealed that there was a reduction in nondirected migration after 24 hours of storage, although there was not an apparent difference in chemotaxis (directed migration) when compared to that at time zero. As described herein, using a more sensitive EZ-TAXISCAN technique, it was shown that neutrophil migration speed decreased, although the directionality was unaffected, when neutrophils were cultured in the presence of G-CSF alone for 24 hours. Moreover, consistent with previous studies, it was found that G-CSF treatment alone could indeed prolong neutrophil life. However, compared to CLON-G treatment, G-CSF only modestly improved neutrophil survival with most neutrophils still dying within 2 days of treatment. See e.g., Drewniak et al., Haematologica 93, 1058-1067 (2008); Dale et al., Transfusion 38, 713-721 (1998); Caspar et al., Blood 81, 2866-2871 (1993); Bensinger et al., Blood 81, 1883-1888 (1993); Colotta et al., Blood 80, 2012-2020 (1992); Leavey et al., Transfusion 40, 414-419 (2000); Hubel et al., Transfusion 45, 1876-1889 (2005); van Raam et al., Blood 112, 2046-2054 (2008); Maianski et al., Blood 99, 672-679 (2002); the contents of each of which are incorporated herein by reference in their entireties.

CLON-G-treatment can prolong the shelf-life and preserve the function of apheresis-collected human granulocyte transfusion products. In vivo experiments described herein were conducted in immunodeficient mice. Whether transfusion with stored CLON-G-treated human neutrophils can effectively enhance host defense in neutropenic patients can be further investigated in clinical trials. Additional studies can also assess the potential side effects of CLON-G in clinical application. The cellular processes inhibited by CLON-G, including cell death, LMP, and oxidative stress, are common therapeutic targets in many diseases. The related molecules such as caspase inhibitors, necroptosis inhibitors, antioxidants, G-CSF, HSP70, and DFO are either approved drugs or already in clinical trials. Although these compounds can be individually applied to patients, their combined use (e.g., in neutrophil culture) can be evaluated for the risk of unwanted side effects. To eliminate or minimize these potential side effects on the recipients, for clinical GTX application, these drugs used to prolong neutrophil lifespan can be removed through rinsing prior to transfusion. After drug withdrawal, washed CLON-G-treated 3-day-old mouse neutrophils displayed similar or even longer lifespans than untreated fresh neutrophils, which persisted at inflammatory sites in vivo in live animals. This feature was much more prominent in human neutrophils than in mouse neutrophils. The pro-survival effect of CLON-G treatment lasted as long as 5 days after the treated human neutrophils were washed with drug-free medium, which is hypothesized to be due to intracellular drug retention or persistent drug effects. This indicates that CLON-G treatment can not only increase the ex vivo shelf-life of human granulocytes but also delay in vivo death of transfused granulocytes and therefore improve GTX efficacy in neutropenic patients.

In summary, both the yield and efficacy of stored granulocytes need to be improved for successful GTX. CLON-G treatment not only facilitates GTX by prolonging the ex vivo shelf life of collected neutrophils but also maintains their function for clinical benefit. Thus, CLON-G represents a therapeutic strategy for improving GTX efficacy and provides an approach for treating neutropenia-related pneumonia and systemic candidiasis, a necessary adjunct to current antibiotics and G-CSF therapies. The present studies focused on neutropenia-related pneumonia and candidiasis, and the same GTX strategy can readily be applied to other neutropenia-related infectious diseases.

### Materials and Methods

Study design: The objective of this study was to explore therapeutic strategies for improving the ex vivo and in vivo survival of neutrophils in GTX. First screening was conducted to identify a treatment (CLON-G) that prolonged ex vivo survival of both human and mouse neutrophils, increasing their half-life from about 24 hours to more than 5 days. Next, the function of CLON-G-treated neutrophils was evaluated, showing that CLON-G-treated neutrophils displayed normal ex vivo functions, including chemotaxis, ROS production, phagocytosis, and bacteria killing. Additionally, the function of transfused neutrophils was assessed in vivo in a clinically relevant murine GTX model of neutropenia-related bacterial pneumonia and systemic candidiasis, which demonstrated that transfusion with stored CLON-G-treated 3-day-old neutrophils enhanced host defenses, alleviated infection-induced tissue damage, and prolonged survival as effectively as transfusion with fresh neutrophils. Finally, experiments were conducted to show that CLON-G-treatment can prolong the shelf-life and preserve the function of apheresis-collected human granulocyte transfusion products both ex vivo and in vivo in NSG mice. All experiments were performed at least three times independently and successfully reproduced. Reproducibility of the experiments and statistical significance of the results are shown in detail in the drawing descriptions or the materials and methods section. Sample size was determined based on prior experience of good sample sizes to ensure adequate data for reliable assessments. The investigators were blinded to group allocation during data collection and analysis. No data was excluded from the analysis. All animal experiments were conducted in accordance with Animal Welfare Guidelines. An Animal Care and Use Committee approved and monitored all procedures. No randomization was used in this study, because all experimental mice were kept under the same environment. An Ethics Committee approved the protocol for human related studies. All participating blood donors provided written informed consent for sample collection and data analysis.

Mice: Wild-type C57BL/6 and NOD.Cg-Prkdc^{scid} Il2rgtm1^{Wjl}/SzJ (NSG) mice were purchased from the JACKSON LABORATORY. Eight to 12-week-old mice were used for all experiments. All necessary measures were taken to avoid or minimize any discomfort, pain, or distress to the animals. During pathogen instillation surgery, mice were anesthetized by intraperitoneal injection of ketamine (100 mg/kg body weight) and xylazine (10 mg/kg body weight) in 100 μl saline. Sufficient anesthesia was verified by frequent monitoring of hind limb withdrawal and determination of muscular tone or arterial blood pressure when feasible. After surgery, buprenorphine (0.05-0.1 mg/kg) was injected subcutaneously every 8 to 12 hours to alleviate pain and distress. In the pneumonia model, mice were euthanized with an overdose of sodium pentobarbital at the conclusion of each experiment. All other mice were euthanized by asphyxiation by inhalation of CO₂. In experiments where the survival rates needed to be determined, the death of the experimental mice was determined based on cessation of vital signs (including heartbeat and respiration) or hypothermia (a ventral surface temperature below 27° C.). All mice were housed and cared for in approved veterinary facilities, which provide sterile isolator cages with fresh food, water, and bedding weekly. All animal experiments were conducted in accordance with Animal Welfare Guidelines. An Animal Care and Use Committee approved and monitored all procedures.

Human neutrophil isolation: Human primary neutrophils were isolated from venous blood from discarded white blood cell filters (PALL CORPORATION) provided by a Blood Bank Lab. 1-3×10⁸ neutrophils were routinely obtained from one filter (450 ml blood from a healthy donor). Collecting neutrophils through a filter (compared to obtaining them by venipuncture and storing in anticoagulant testing tubes) does not impair neutrophil function (such as chemotaxis, phagocytosis, H₂O₂ production, and the time course of cell death; see e.g., Loison et al. 2010, supra). Blood bank technicians were coordinated to ensure that all filters were prepared using the same standard procedure and delivered in time. All blood was drawn from healthy blood donors. Donors from whom the blood was obtained were unidentifiable, and this research did not involve an intervention or interaction with living individuals or identifiable personal information. Thus this research is not classified as human subjects research. Institutional Review Boards (IRB) approved the protocol.

Preparation of human granulocyte apheresis concentrates: Circulating neutrophil counts in G-CSF (250 ug)- and dexamethasone (8 mg)-stimulated donors are maximal at 12 hours after treatment. Accordingly, the donors were stimulated in the evening and granulocyte collection was carried out the next morning. Neutrophil harvesting was accomplished through the process of apheresis in which red blood cells (RBCs) and plasma were returned to the donor. To collect enough cells for GTX, standard continuous flow apheresis was performed for 1 to 1.5 hours to process 3.5 to 5 liters of blood. Citrate was used as the anticoagulant during collection and calcium gluconate was added to the return line to counteract the citrate-induced hypocalcemic symptoms in the donor. Hydroxyethyl starch (HES), a sedimenting agent, was added to the donor's blood as it entered the centrifuge to facilitate separation of granulocytes from RBCs and improve collection. To perform functional assays, neutrophils were purified at indicated time points after the CLON-G treatment; see e.g., see e.g., Loison et al. 2010, supra. The purity was >97% as determined by both Wright-Giemsa staining and flow cytometry analysis with CD15 and CD16 antibodies. The Wright-Giemsa staining was performed using a Wright-Giemsa Stain Solution (SOLARBIO, #G1020) following a protocol provided by the manufacturer. For flow cytometry analysis, the isolated neutrophils were suspended in 100 μL ice-cold PBS, and stained with FITC-CD15 (BIOLEGEND, MC-480, 1 μg/mL) and allophycocyanin (APC)-CD16 (BIOLEGEND, 51.1, 1 μg/mL) at 4° C. for 15 minutes. Samples were examined with a FACSCANTO II flow cytometer (BECTON DICKINSON). Neutrophils were gated by their forward-scatter and side-scatter characteristics and their CD15 and CD16 expression pattern. No contamination of hematopoietic progenitor cells (HPCs) was detected. Granulocyte-macrophage colony-forming units (CFU-GM) were undetectable in the isolated neutrophil population. An Ethics Committee approved the study protocol. All participating blood donors provided written informed consent for sample collection and data analysis.

Mouse neutrophil isolation: Mouse bone marrow neutrophils were prepared; see e.g., Kambara et al. 2018, supra. Briefly, bone marrow from the femurs and tibias was flushed out with 5 ml Hank's Balanced Salt Solution (HBSS)/EDTA/Bovine serum albumin (BSA) (without Ca²⁺/Mg²⁺ salts, 0.5% low endotoxin BSA, 15 mM EDTA). Cells were spun down (400×g, 10 min, room temperature (RT)), resuspended in 1 ml of HBSS/EDTA/BSA, layered over discontinuous PERCOLL/HBSS gradients (52%, 62%, 76%), and centrifuged (1060×g, 30 min, RT). The interface between the 62% and 76% layers containing neutrophils was harvested and washed with 5 ml of HBSS/EDTA/BSA. Red blood cells were removed by resuspension in HISTOPAQUE-1119 (SIGMA-ALDRICH) and centrifuged (1600×g, 30 min, RT). The interface between the cell suspension and HISTOPAQUE-1119 layers containing neutrophils was harvested and washed with 5 ml of HBSS/EDTA/BSA, and cells were spun down (400×g, 5 min, RT) and resuspended in the buffer required for each assay. Using this method, 10-15 million neutrophils were routinely obtained per mouse, >95% of which were morphologically mature (confirmed by flow cytometry analysis using Mac-1 and Ly6G antibodies). For flow cytometry analysis, the isolated neutrophils were suspended with 100 μL ice-cold PBS and stained with APC-Mac-1 (BIOLEGEND, M1/70, 1 μg/mL) and phycoerythrin (PE)-Ly6G (BIOLEGEND, 1A8, 1 μg/mL) at 4° C. for 15 minutes. Samples were examined with a FACSCANTO II flow cytometer (BECTON DICKINSON). Cell viability was usually >98%. No contamination with HPCs was detected. CFU-GM were undetectable in the isolated neutrophil population.

Neutrophil culture and treatment: Murine or human neutrophils were cultured in RPMI-1640 supplemented with 20% heat-inactivated fetal bovine serum (FBS; GIBCO) and 1% penicillin-streptomycin antibiotics at a density of 1×10⁶ cells/mL at 37° C. in a 5% CO₂ incubator. Neutrophils were treated with different concentrations of Z-FA-FMK (SELLECKCHEM, S7391), Z-YVAD-FMK (ADOOQ BIOSCIENCE, A16317), belnacasan (VX-765) (SELLECKCHEM, S2228), Z-DEVD-FMK (SELLECKCHEM, S7312), Z-IETD-FMK (SELLECKCHEM, S7314), Ac-LEHD-CHO (SIGMA ALDRICH, SCP0095), Q-VD-Oph (SELLECKCHEM, S7311), Z-VAD-FMK (SELLECKCHEM, S7023), Emricasan (SELLECKCHEM, S7775), Ac-DEVD-CHO (SELLECKCHEM, S7901), DFO (SIGMA-ALDRICH, D9533), Mouse Hsp70 (ABCAM, ab113187), human Hsp70 (ABCAM, ab78427), Diisopropylfluorophosphate (DFP) (SIGMA-ALDRICH, D0879), Diphenyleneiodonium chloride (DPI) (SIGMA-ALDRICH, D2926), NAC (SIGMA-ALDRICH, A9165), G-CSF (AMGEN, NEUPOGEN), Nec-1s (EMD MILLIPORE, 852391-15-2), NECROX-2 (ENZO LIFE SCIENCES, ALX-430-166), and NECROX-5 (ENZO LIFE SCIENCES ALX-430-167). CLON-G treatment was defined as combined treatment with Q-VD-Oph (50 μM, caspase inhibitor), DFO (1 μM, LMP inhibitor), Hsp70 (10 pM, LMP inhibitor), NAC (10 μM for human neutrophils, 1 mM for mouse neutrophils), Nec-1s (10 μM, necroptosis inhibitor), and G-CSF (10 ng/mL).

Analysis of cell proliferation by EdU incorporation: Cell proliferation in vitro was determined using the CLICK-IT PLUS EdU Flow Cytometry Assay Kit (INVITROGEN). EdU was added to the cell culture medium (1 μg per well in 1 mL medium). At the indicated time points, cells were collected and stained with allophycocyanin (APC)-CD16 (BIOLEGEND, 51.1, 1 μg/mL) (for human cells) or APC-Ly6G (BIOLEGEND, 1A8, 1 μg/mL) (for mouse cells) at 4° C. for 15 minutes and then fixed, permeabilized, and stained with azide dye (INVITROGEN) following a protocol provided by the manufacturer. Finally, the samples were washed and analyzed using a BD FACSCANTO II flow cytometer (BD BIOSCIENCES).

In vitro neutrophil death: Isolated neutrophils were cultured at a density of 1×10⁶ cells/mL. The total initial cell number was counted using a hemocytometer before culture or treatment. At each indicated time point, the total cell number was counted again. Since the neutrophils undergoing lytic cell death (puffed cells) were destroyed by pipetting, the total number of cells in the culture gradually decreased. The morphology of cultured neutrophils was recorded by light microscopy (600×) at the indicated time points. Apoptotic cells were detected by Annexin V-fluorescein isothiocyanate (FITC) and propidium iodide (PI) staining using an Annexin V Detection Kit (BD BIOSCIENCES) following the manufacturer's protocol. Flow cytometry was performed using a FACSCANTO II flow cytometer and analyzed using FACSDIVA software (BD BIOSCIENCES). Annexin V and PI double-negative cells were defined as healthy cells.

Chemotaxis assay: The EZ-TAXISCAN device (EFFECTOR CELL INSTITUTE) was used to visualize the details of neutrophil chemotaxis. The EZ-TAXISCAN chamber was assembled with a 260 μm-wide×4 μm-thick silicon chip on a 2 mm untreated glass base as described by the manufacturer and filled with filtered RPMI-1640 medium. 1 μL freshly isolated or treated neutrophils (1×10⁷/mL) were added to the lower reservoir and allowed to line up by pulling medium from the upper reservoir. 1 μl fMLP (1 μM for mouse neutrophils and 100 nM for human neutrophils) was then added to the upper reservoir. Neutrophil migration (at 37° C.) in each channel was captured sequentially every 30 seconds for 20 minutes using a 10× lens. Migrating cells were tracked using TRACKING TOOL software. Chemotaxis velocity and directionality were analyzed; see e.g., Jia et al. 2007, supra.

In vitro phagocytosis assay: Fluorescein-conjugated pHrodo Red E. coli BioParticles or Zymosan A S. cerevisiae BioParticles (INVITROGEN) were reconstituted in phosphate-buffered saline (PBS) and opsonized with 10% serum at 37° C. for 30 minutes. Neutrophils were incubated with serum-opsonized bioparticles at a ratio of 1:10 (neutrophils:bioparticles) at 37° C. for 0.5 hours. APC-Ly6G (BIOLEGEND, 1A8, 0.2 μg/mL) or APC-CD16 (BIOLEGEND, 51.1, 0.2 μg/mL) antibody was added to the sample to label mouse or human neutrophil membranes. The labeling was achieved by incubating the samples at room temperature for 3 minutes. The number of internalized particles was counted under a spinning disk confocal microscope (ULTRAVIEW VOX). Phagocytosis efficiency was expressed as the percentage of neutrophils that engulfed at least one bioparticle. Phagocytosis index was expressed as the average number of internalized particles per cell. At least 200 cells were counted from random fields per coverslip for each group.

Chemoattractant-elicited NADPH oxidase activation: ROS, particularly superoxide anions, produced during NADPH oxidase activation were detected using luminol chemiluminescence; see e.g., Subramanian et al., Blood 109, 4028-4037 (2007); Su et al., J Immunol 180, 6947-6953 (2008); the contents of each of which are incorporated herein by reference in their entireties. To determine fMLP-induced NADPH oxidase activation, 0.5×10⁶ mouse or human neutrophils were resuspended in 100 μL saline containing 1% BSA and then loaded into a 96-well MAXISORP plate (NUNC). Chemiluminescence was measured using a TRISTAR LB941 microplate luminometer (BERTHOLD TECHNOLOGIES USA). Saline (100 μL) containing 4 U/ml HRP, 50 μM luminol, and 500 nM fMLP was injected into the mixture via the injection port of the luminometer. Luminescence (arbitrary light units) was recorded every 12 seconds. Data are represented as mean±SD of n=3 wells assayed simultaneously.

In vitro bacterial killing assay: Freshly isolated, G-CSF-treated, or CLON-G-treated aged mouse or human neutrophils were washed with saline and resuspended in RPMI-1640 medium without antibiotics at 1×10⁶ cells/ml. Cells were incubated with opsonized E. coli (strain 19138, AMERICAN TYPE CULTURE COLLECTION (ATCC); Multiplicity of infection (MOI)=5) at 37° C. for one hour. After incubation, neutrophils were lysed by adding distilled H₂O. The samples were then serially diluted and spread onto Luria-Bertani (LB) agar plates. The colony-forming units (cfus) were counted after incubating the plates overnight at 37° C. Bacterial suspension without incubating with neutrophils was used as input control. In vitro bacterial killing capabilities were reflected by the decrease in cfu.

Establishment and confirmation of CPM-induced neutropenia: Cyclophosphamide powder (Cytoxan/CPM; BRISTOL-MYERS SQUIBB) was dissolved in saline for injection at a final concentration of 20 mg/mL. Cyclophosphamide was injected intraperitoneally (i.p.) at a total dose of 250 mg/kg (150 mg/kg on day 0 and 100 mg/kg on day 3). Blood samples (20 μL) were taken from the retroorbital sinuses of anesthetized mice using heparinized capillary tubes (MODULOHM). Total and differential white blood cell counts (neutrophils, lymphocytes, and monocytes) were performed using a HEMAVET 850 hematology system (DREW SCIENTIFIC), which is a multiparameter, automated hematology analyzer designed for in vitro diagnostic use.

Relative death of transfused neutrophils in inflamed peritoneal cavity: Freshly isolated CD45.1⁺ or CLON-G-treated (3 days) green fluorescent protein (GFP)+bone marrow neutrophils were mixed (1:1, a total of 5×10⁶/mouse) and injected i.p. into neutropenic recipient mice challenged with thioglycollate (TG) (3% in 1 mL PBS, i.p. injected) for 1 hour. Peritoneal lavage fluid was harvested at the indicated timepoints after cell injection. Cells in peritoneal lavage fluid were collected and resuspended in 100 μL ice-cold PBS and stained with phycoerythrin (PE)-CD45.1 (BIOLEGEND, A20, 2 μg/mL) at 4° C. for 15 minutes. The relative amount of transfused freshly isolated CD45.1⁺ and CLON-G-treated (3 days) GFP⁺ neutrophils were analyzed by flow cytometry using a BD FACSCANTO II flow cytometer.

In vivo recruitment of transfused neutrophils in a mouse peritonitis model: Freshly isolated CD45.1⁺ or CLON-G-treated (1 day) GFP⁺ bone marrow neutrophils were mixed (1:1, a total of 5×10⁶/mouse) and injected intravenously into neutropenic recipient mice challenged with TG for 1 hour. At the indicated time points after cell injection, peritoneal cells were harvested by peritoneal cavity lavage with 5 mL of ice-cold PBS/15 mM EDTA, flushed back and forth three times. The relative amount of transfused freshly isolated CD45.1⁺ and CLON-G-treated (1 day) GFP⁺ neutrophils were analyzed by flow cytometry using a BD FACSCANTO II flow cytometer as described above.

Neutropenia-related E. coli-induced pneumonia model: Mice were anesthetized by ketamine hydrochloride (100 mg/kg intraperitoneally) and xylazine (10 mg/kg intraperitoneally) injection, mice trachea were surgically exposed, and a dose of 5×10³ cfu (for neutropenic mice) or 1×10⁶ (for normal mice) E. coli (strain 19138; ATCC) per mouse was instilled intratracheally to the left bronchus (total volume 50 μL). The mice were placed on warm heating pads during post-surgery recovery. As soon as they were ambulatory (approximately 5 to 15 minutes), they were returned to the home cage with immediate access to food and water. At the end of the experiments, mice were euthanized with CO₂.

Bacterial burden: Freshly isolated or CLON-G-treated bone marrow neutrophils (5×10⁶/mouse) were injected into neutropenic mice challenged with 5×10³ cfu E. coli for 4 hours. For the bacterial burden assay (the number of live bacteria in the lungs), mice were euthanized at 24 hours after bacterial challenge, bronchoalveolar lavage fluids (BALF) were collected using 1 ml of cold PBS/15 mM EDTA flushed back and forth for three times, cells in BALF were lysed by adding distilled H₂O, then BALF was diluted and spread onto Luria broth (LB) agar plates. The cfu were counted manually by an independent blinded examiner after incubating the plates overnight at 37° C.

BALF cytokine and total protein concentrations: BALF samples were obtained from mice 24 hours after E. coli challenge using 1 ml of cold PBS/15 mM EDTA flushed back and forth three times. TNF-α and IL-6 concentrations in BALF were measured with ELISA kits following a protocol provided by the manufacturer (R&D SYSTEMS). Protein concentration was measured using the BIO-RAD protein assay reagent (BIO-RAD LABORATORIES). The standard curve was constructed using BSA.

Histopathology: In pneumonia model, lungs were fixed by intratracheal instillation of Bouin's solution at 23 cmH₂O pressure. Tissues were embedded in paraffin, and 6 m sections were stained with hematoxylin and eosin (H&E) and then examined by light microscopy. Non-quantitative histological analysis was performed by a pathologist blinded to the groups. IMAGEJ software (National Institutes of Health) was used to manually trace edema and neutrophil-containing regions of the tissue section. The pixel area of each edema and neutrophil-containing region was calculated using IMAGEJ software. Edema formation was calculated as the percentage of pixel area of all the edema-containing regions relative to the pixel area of the whole image.

Examination of GTX-induced organ damage: After anesthesia with ketamine hydrochloride (100 mg/kg i.p.) and xylazine (10 mg/kg i.p.), mouse tracheas were surgically exposed and a total volume of 50 μl of saline or LPS (5 mg/kg body weight) was instilled intratracheally via an angiocatheter inserted through the trachea and into the left bronchus. After surgery and wound closure, mice were suspended by their front legs to help deliver the instillate deep into the left lobe before being placed back into the cage with soft and warm bedding for recovery. GTX was performed via intravenous injection 4 hours after the LPS instillation. Mice were euthanized by CO₂ 24 hours after neutrophil transfusion. Kidneys, livers, spleens, hearts, and lungs were dissected, fixed in 10% neutral buffered formalin (SIGMA-ALDRICH), and then embedded in paraffin. Paraffin-embedded sections (˜6 μm thick) were stained with H&E and examined by light microscopy.

In vivo recruitment of transfused neutrophils in bacterial pneumonia: Freshly isolated or CLON-G-treated bone marrow neutrophils were labeled with carboxyfluorescein diacetate succinimidyl ester (CFSE, 5 μM) or seminaphthorhodafluor-1 acetate (SNARF-1, 5 μM) at 37° C. for 10 minutes; labeled cells were mixed (1:1, a total of 5×10⁶/mouse) and transfused via the tail vein into the same neutropenic recipient mice challenged with 5×10³ cfu E. coli for 4 hours. Whole lungs were homogenized 3 hours after neutrophil transfusion. The number of adoptively transferred neutrophils recruited to the lungs was analyzed using a FACSCANTO II flow cytometer and FACSDIVA software. Relative recruitments of CLON-G-treated and freshly isolated neutrophils were calculated as the ratio of the indicated populations.

Neutrophil accumulation in inflamed lungs: Neutropenic mice were anesthetized and instilled with bacteria as described above. GTX was performed 4 hours after E. coli instillation. At the indicated time points, mice were euthanized by CO₂. The chest cavity was opened and a catheter was tied to the trachea. Bronchoalveolar lavage (BAL) was performed (1 mL of PBS/15 mM EDTA) in each group. The BALF was centrifuged at 450×g for 10 minutes, and the total and differential cell counts were determined from the pelleted cell fraction by flow cytometry analysis. For flow cytometry, the cells were suspended in 100 μL ice-cold PBS and stained with FITC-F4/80 (BIOLEGEND, QA17A29, 1 μg/mL) and APC-Ly6G (BIOLEGEND, 1A8, 1 μg/mL) at 4° C. for 15 minutes. Neutrophil numbers were determined with the FACSCANTO II flow cytometer and FACSDIVA software (BD BIOSCIENCES).

Candida albicans infection: Neutropenic mice were injected intravenously with C. albicans blastospores in a 200 μL volume of sterile pyrogen-free PBS (1×10³, strain SC5314; ATCC). Survival was assessed daily (for about 14 days). For histologic analysis, mice were euthanized by CO₂, and kidneys of subgroups were fixed in 10% neutral buffered formalin (SIGMA-ALDRICH). Paraffin-embedded sections were stained with H&E as described above.

Fungal burden: Freshly isolated or CLON-G-treated bone marrow neutrophils (3×10⁶/mouse) were injected into neutropenic mice infected with C. albicans as described above for one hour. Mice were euthanized 3 days later. Whole kidneys were ground, filtered, and resuspended in 5 mL ice-cold PBS/15 mM EDTA, cells were lysed by adding distilled H₂O, then the fluids were diluted and spread on Yeast Extract-Peptone-Dextros (YPD) agar plates. cfu were counted manually by an independent blinded examiner after incubating the plates overnight at 37° C.

Examination of the survival and function of transfused human neutrophils in vivo in NSG mice: The experiment was conducted essentially as described by Trump et al. 2019, supra. Briefly, to deplete mice neutrophils, cyclophosphamide (CPM) was injected i.p. at a total dose of 250 mg/kg (150 mg/kg on day 0 and 100 mg/kg on day 3). Twenty-four hours after the last dose of CPM, untreated fresh or drug-treated human neutrophils isolated from apheresis-collected granulocyte concentrates were injected intravenously or intraperitoneally into neutropenic NSG mice in a final volume of 200 μl of PBS. To measure phagocytosis, pHrodo-E. coli, a nonviable fluorescently labeled bacteria (200 μg in 200 μl PBS, INVITROGEN MOLECULAR PROBES), and the stimulant TG (3% in 800 μl distilled water) were co-injected intraperitoneally. At the conclusion of each experiment, the animals were euthanized by CO₂ asphyxiation and peritoneal exudates were harvested in two successive washes with 10 ml cold PBS containing 15 mM EDTA and 0.2% BSA. Neutrophil recruitment, phagocytosis, and survival were assessed as described above. Transfused human neutrophils were identified by their unique surface markers by flow cytometry analysis. Cells in peritoneal lavage fluid were suspended in 100 μL ice-cold PBS and stained with PE-Cy7-anti-mouse-CD45 (BIOLEGEND, 30-F11, 1 μg/mL), APC-Cy7-anti-human-CD45 (BIOLEGEND, 2D1, 1 g/mL), PE-anti-human-CD11b (BIOLEGEND, LM2, 1 μg/mL), and APC-anti-human-CD16 (BIOLEGEND, 51.1, 1 μg/mL) at 4° C. for 15 minutes. Samples were analyzed with a FACSCANTO II flow cytometer (BECTON DICKINSON). Transfused human neutrophils were gated by their forward-scatter and side-scatter characteristics and their CD45/CD11b/CD16 expression patterns (see e.g., FIG. 8B and FIG. 8E).

Statistical analyses: Normality of data distribution was tested and confirmed using the Shapiro-Wilk test. For most experiments, unless stated otherwise, comparisons were made using a two-tailed, unpaired, Student's t-test. Values shown in each figure represent mean±standard deviation (SD). A p-value<0.05 was considered statistically significant. In vitro experiments were repeated at least three times. For in vivo experiments, to perform reliable statistical analysis, at least five mice from each treatment group were utilized for each data point. This number was chosen based on a power analysis which was conducted using Simple Interactive Statistical Analysis (SISA) (see e.g., home.clara.net/sisa/sampshlp.htm or quantitativeskills.com/sisa, both of which are available on the world wide web). Based on the preliminary data and experience, the SD was set to 10% of the average. To detect a 20% difference (average 1=100, average 2=80, SD1=10, SD2=8, allocation ratio=1) with a power level of 90% (90% chance to discover a real difference in the sample) and an alpha of 0.05, n=5 mice needed to be used from each genotype or treatment group for sufficient double-sided power. For the survival analysis, Kaplan-Meier survival curves were generated and comparisons were performed between groups by log-rank analysis using PRISM (GRAPHPAD SOFTWARE).

Additional information can be found in Fan et al., “Targeting multiple cell death pathways extends the shelf-life and preserves the function of human and mouse neutrophils for transfusion,” Sci Transl Med. 2021 Jul. 28; 13(604): eabb1069, the contents of which are incorporated herein by reference in their entirety.

1. A composition comprising:
a) a caspase inhibitor;
b) a lysosomal membrane permeabilization (LMP) inhibitor;
c) an antioxidant; and
d) a growth factor.

2. The composition of claim 1, wherein:
a) the caspase inhibitor is a pan-caspase inhibitor selected from the group consisting of: Q-VD-Oph; Z-VAD-FMK; Emricasan; and Ac-DEVD-CHO;
b) the LMP inhibitor is Heat Shock Protein 70 (Hsp70) and/or deferoxamine mesylate (DFO);
c) the antioxidant is N-acetyl cysteine (NAC); and/or
d) the growth factor is a stimulator of the phosphoinositide 3-kinase (PI3K) and Akt pathway.

3-4. (canceled)

5. The composition of claim 2, wherein Q-VD-Oph is at a concentration of at least 50 μM; Emricasan is at a concentration of at least 10 μM; or Emricasan is at a concentration of at least 25 μM.

6-9. (canceled)

10. The composition of claim 2, wherein Hsp70 is at a concentration of at least 10 pM; or DFO is at a concentration of at least 1 μM.

11-12. (canceled)

13. The composition of claim 2, wherein NAC is at a concentration of at least 10 μM.

14. (canceled)

15. The composition of claim 2, wherein the stimulator of the PI3K/Akt pathway is granulocyte colony-stimulating factor (G-CSF).

16. The composition of claim 15, wherein G-CSF is at a concentration of at least 10 ng/mL.

17. The composition of claim 1, further comprising a necroptosis inhibitor.

18. The composition of claim 17, wherein the necroptosis inhibitor is Nec-1s.

19. The composition of claim 18, wherein Nec-1s is at a concentration of at least 10 μM.

20. The composition of claim 1, further comprising cell culture medium and/or serum.

21-22. (canceled)

23. A cell culture medium comprising:
a) a caspase inhibitor;
b) a lysosomal membrane permeabilization (LMP) inhibitor;
c) an antioxidant;
d) a growth factor; and
e) a necroptosis inhibitor.

24-27. (canceled)

28. A cell culture medium for increasing the lifespan of neutrophils, the cell culture medium comprising effective concentrations of:
a) Q-VD-Oph; DFO; Hsp70; NAC; G-CSF; and Nec-1s;
b) Q-VD-Oph; DFO; Hsp70; NAC; and G-CSF;
c) Emricasan; DFO; Hsp70; NAC; G-CSF; and Nec-1s; or
d) Emricasan; DFO; Hsp70; NAC; and G-CSF.

29-33. (canceled)

34. The composition of claim 1, in combination with a neutrophil.

35. (canceled)

36. A kit comprising the composition of claim 1.

37. A method of increasing the lifespan of a neutrophil, the method comprising: contacting the neutrophil, or population thereof, with the composition of claim.

38-48. (canceled)

49. A neutrophil produced by the method of claim 37.

50. A pharmaceutical composition comprising the neutrophil of claim 49, or population thereof, and a pharmaceutically acceptable carrier.

51-52. (canceled)

53. A method of treating neutropenia or a neutropenia-associated disease or disorder, the method: comprising administering an effective amount of a neutrophil of claim 49, or population thereof, to a recipient subject in need thereof.

54-58. (canceled)

59. A method of treating neutropenia or a neutropenia-associated disease or disorder, the method comprising:
a) isolating a neutrophil or population thereof from a donor subject;
b) contacting the neutrophil, or population thereof, with the composition of claim 1; and
c) administering an effective amount of the cultured neutrophil, or population thereof, to a recipient subject in need thereof.

60. (canceled)
